SEQUENCE LISTING (240 sequences)

SEQ ID NO: 1 - protein, 89 aa, Homo sapiens
GHETGRLSGAADTQALLRNDQVYQPLRDRDDAQYSHLGGNWARNKGGSRSKRSRLLHSDYMNMTPRRPGPTRKHYQPYAPPRDFAAYRS

SEQ ID NO: 2 - protein, 99 aa, Homo sapiens
KNRKAKAKPVTRGAGAGGRQRGQNKERPPPVPNPDYEPIRKGQRDLYSGLNQRRIGGSRSKRSRLLHSDYMNMTPRRPGPTRKHYQPYAPPRDFAAYRS

SEQ ID NO: 3 - protein, 89 aa, Homo sapiens
GQDGVRQSRASDKQTLLPNDQLYQPLKDREDDQYSHLQGNQLRRNGGSRSKRSRLLHSDYMNMTPRRPGPTRKHYQPYAPPRDFAAYRS

SEQ ID NO: 4 - protein, 10 aa, Homo sapiens
MAMVKRKDSR

SEQ ID NO: 5 - protein, 45 aa, Homo sapiens
TTTPAPRPPTPAPTIASQPLSLRPEACRPAAGGAVHTRGLDFACD

SEQ ID NO: 6 - protein, 20 aa, Homo sapiens
GIIVTDVIATLLLAGVFCFA

SEQ ID NO: 7 - protein, 26 aa, Homo sapiens
VMSVATIVIVDICITGGLLLLVYYWS

SEQ ID NO: 8 - protein, 21 aa, Homo sapiens
GFLFAEIVSIFVLAVGVYFIA

SEQ ID NO: 9 - protein, 23 aa, Homo sapiens
VIGFRILLLKVAGFNLLMTLRLW

SEQ ID NO: 10 - protein, 23 aa, Homo sapiens
TILYEILLGKATLYAVLVSALVL

SEQ ID NO: 11 - protein, 41 aa, Homo sapiens
RSKRSRLLHSDYMNMTPRRPGPTRKHYQPYAPPRDFAAYRS

SEQ ID NO: 12 - protein, 42 aa, Homo sapiens
KRGRKKLLYIFKQPFMRPVQTTQEEDGCSCRFPEEEEGGCEL

SEQ ID NO: 13 - protein, 40 aa, Homo sapiens
CVRCRHRRRQAERMSQIKRLLSEKKTCQCPHRFQKTCSPI

SEQ ID NO: 14 - protein, 32 aa, Homo sapiens
LYCNHRNRRRVCKCPRPVVKSGDKPSLSARYV

SEQ ID NO: 15 - protein, 537 aa, Homo sapiens
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 16 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 17 - protein, 619 aa, Homo sapiens
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 18 - protein, 5 aa, Artificial Sequence
  linker
GGGGS

SEQ ID NO: 19 - protein, 10 aa, Artificial Sequence
  linker
GGGGSGGGGS

SEQ ID NO: 20 - protein, 15 aa, Artificial Sequence
  linker
GGGGSGGGGSGGGGS

SEQ ID NO: 21 - protein, 20 aa, Artificial Sequence
  linker
GGGGSGGGGSGGGGSGGGGS

SEQ ID NO: 22 - DNA, 135 bp, Homo sapiens
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 23 - protein, 41 aa, Homo sapiens
CTIEVMYPPPYLDNEKSNGTIIHVKGKHLCPSPLFPGPSKP

SEQ ID NO: 24 - DNA, 123 bp, Homo sapiens
tgtaccattgaagttatgtatcctcctccttacctagacaatgagaagagcaatggaaccattatccatgtgaaagggaaacacctttgtccaagtcccctatttcccggaccttctaagccc

SEQ ID NO: 25 - protein, 233 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 26 - protein, 533 aa, Homo sapiens
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 27 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 29 - protein, 140 aa, Macaca fascicularis
IQNPDPAVYQLKGSKSNDTSVCLFTDFDSVMNVSQSKDSDVHITDKTVLDMRSMDFKSNGAVAWSNKSDFACTSAFKDSVIPADTFFPGTESVCDANLVEKSFETDMNLNFQNLSVIGFRILLLKVAGFNLLMTLRLWSS

SEQ ID NO: 30 - protein, 135 aa, Cavia porcellus
IENPEPAVYQLRDPKSRNTSVCLFTDFDSNVNLSESVLPGVPHTQPTVLNMKSLDSKSNAAVAWSSKSNFDCRNTFGDPTFYPSSDYPCNATLVEESFETDMNLNLENLSVMALRILLLKVAGFNLLMTLRLWSS

SEQ ID NO: 31 - protein, 134 aa, Felix catus
IQNPDPAVYQLKSPESSNISVCLFTDFDSEVNVNPSTESNMIRLKSTSLDMKTMDSKSNGALAWGNSFDLGCNSTFNYTFHSSSEFPCDANVVEKGFETDMNLNFDNLTVIMLRIIFLKAVGFNLLMTLKLWSS

SEQ ID NO: 32 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 173 aa, Mus musculus
  misc_feature (1)..(1): Xaa can be any naturally occurring amino acid
XDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 34 - protein, 174 aa, Rattus norvegicus
EDLKTVTPPKVSLFEPSEAEIADKQKATLVCLARGFFPDHVELSWWVNGKEIRNGVSTDPQAYKESNNITYCLSSRLRVSAPFWHNPRNHFRCQVQFYGLTEEDNWSEDSPKPVTQNISAEAWGRADCGITSASYHQGVLSATILYEILLGKATLYAVLVSALVLMAMVKKKNS

SEQ ID NO: 35 - protein, 177 aa, Sus scrofa
EDLQQVRPPKVAVFEPSEAEISRTQKATLVCLATGFYPDHVELSWWVNGKQVQSGVSTDLQPYREDPSRNDSSYCLSSRLRVTAAFWHNPRNHFRCQVQFYGLTEDDEWEYNWTKPITQNISAEAWGKADCGFSSASYQQGVLSATLLYEILLGKATLYAVLVSALVLMATVKKKDS

SEQ ID NO: 36 - protein, 177 aa, Callithrix jacchus
DDLSKVFPPTVAVFEPSETEISRTQKATLVCLATGFYPDHVELSWWVNGKEVHSGVSTDPQPLKEQPALNDSKYCLSSRLRVSATFWHNPRNHFRCQVQFYGLSEDDEWTQPRAKPIDQNVSAEAWGRAGERASYQQGVLSATVLYEILLGKATLYAVLVSALVLMAMVRRRVGSDR

SEQ ID NO: 37 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 38 - protein, 173 aa, Mus musculus
MEHSGILASLILIAVLPQGSPFKIQVTEYEDKVFVTCNTSVMHLDGTVEGWFAKNKTLNLGKGVLDPRGIYLCNGTEQLAKVVSSVQVHYRMCQNCVELDSGTMAGVIFIDLIATLLLALGVYCFAGHETGRPSGAAEVQALLKNEQLYQPLRDREDTQYSRLGGNWPRNKKS

SEQ ID NO: 39 - protein, 171 aa, Sus scrofa
MEHSRFLSGLILAAFLSRVSPYEVEMEELEDKVFVSCNTSIIWLQGTEGELLSDKKIDLGKRILDPRGLYKCNAPKEQDSNSKIFLQVYYRMCQNCVELDSATLAGIIVTDIIATLLLALGVYCFAGHEMGRFSRAADTQDLLRNDQLYQPLRDRNDGQYSRLGENWARNK

SEQ ID NO: 40 - protein, 173 aa, Rattus norvegicus
MEHYGILVSLLLATVLPQGSPFKIEVVEYEDKVFVNCNTSIRHLDGSVERWLTKNKSLILGKGILDPRGMYMCNGTEELAKEVSTVQVYYRMCQNCVELDSATLAGVIITDLIATLLLALGVYCFAGHETGRLSGAVDTQVLLKNEQLYQPLRDRDDAQYSRLGGNWPRNKRS

SEQ ID NO: 41 - protein, 171 aa, Macaca fascicularis
MEHSTFLSGLVLATLLSQVSPFKIPVEELEDRVFVKCNTSVTWVEGTVGTLLTNNTRLDLGKRILDPRGIYRCNGTDIYKDKESAVQVHYRMCQNCVELDPATLAGIIVTDVIATLLLALGVFCFAGHETGRLSGAADTQALLRNDQVYQPLRDRDDAQYSRLGGNWARNK

SEQ ID NO: 42 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 43 - protein, 189 aa, Mus musculus
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 44 - protein, 196 aa, Sus scrofa
MPSGNLWKVLGLCLLSVGAWGQEDIERPDEDTQKTFKVSISGDKVELTCPEDPESEKMTWKRNDMQIYESYDNYMLLESFSEVENSGYYTCTVGEKTSHRLYLKARVCENCVEVDLMAVVTIIVVDICITLGLLMVVYYYSKSRKAKAMPVTRGAGAGGRPRGQNRERPPPVPNPDYEPIRKGQRDLYSGLNQRGR

SEQ ID NO: 45 - protein, 198 aa, Oryctolagus cuniculus
MRAGTLWRVLALWLLSVAAWGQEDDDHADDYTQKLFTVSISGTRVVLTCPVEAEGGDIHWERDEKSLPNTKKELDLTDFSEMEHSGYYSCYVGTKNKENEHILYLKARVCEACMEVDLTTVASIVVADVCVTLGLLLLVYYWSKNRKAKCKPVTRGAGAGGRPRGQNKERPPPVPNPDYEPIRKGQRDLYSGLNQRGI

SEQ ID NO: 46 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 47 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 48 - protein, 182 aa, Mus musculus
MEQRKGLAGLFLVISLLQGTVAQTNKAKNLVQVDGSRGDGSVLLTCGLTDKTIKWLKDGSIISPLNATKNTWNLGNNAKDPRGTYQCQGAKETSNPLQVYYRMCENCIELNIGTISGFIFAEVISIFFLALGVYLIAGQDGVRQSRASDKQTLLQNEQLYQPLKDREYDQYSHLQGNQLRKK

SEQ ID NO: 49 - protein, 182 aa, Sus scrofa
MEQGKHLAGLILAITLLQGTMAQLKEGKHSVLLDDNREDGSVLLTCGLPDQNIRWFKDGKEICSLNNSRSTCNLGSSSKDPRGIYWCEGSKENSKRLQVYYRMCQNCIELNSATVSGFIFTEIISLFFLAVGVYFIAGQDGVRQSRASDKQTLLSNDQLYQPLKDREDDQYSHLQGNNSRKN

SEQ ID NO: 50 - protein, 182 aa, Rattus norvegicus
MEQGKGLAGLFLVISLLQGTMAQQKEEKHLVKVDDSQGDGSVLLTCDFNEKTITWLKDGHRISPPNATKSTWNLGNGAKDPRGMYQCRGAKKKSQLLQVYYRLCENCIELNMGTVSGFIFAEIISIFFLAVGVYFIAGQDGVRQSRASDKQTLLQNEQVYQPLKDREYEQYSRLQGNQVRKK

SEQ ID NO: 51 - protein, 181 aa, Macaca fascicularis
MVQGKGLTGFILAIILLQGSLAQSFEENRKLNVYNQEDGSVLLTCHVKNTNITWFKEGKMIDILTAHKNKWNLGSNTKDPRGVYQCKGSKDKSKTLQVYYRMCQNCIELNAATILGFVFAEIISIFFLAVGVYFIAGQDGVRQSRASDKQTLLPNDQLYQPLKDREDDQYSHLQGNQLRMN

SEQ ID NO: 52 - protein, 455 aa, Homo sapiens
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFHALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 53 - protein, 410 aa, Homo sapiens [sequence omitted]

SEQ ID NO: 54 - protein, 93 aa, Homo sapiens
FFYGSISRAEAEEHLKLAGMADGLFLLRQCLRSLGGYVLSLVHDVRFHHFPIERQLNGTYAIAGGKAHCGPAELCEFYSRDPDGLPCNLRKPC

SEQ ID NO: 55 - protein, 92 aa, Homo sapiens
WYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 56 - protein, 651 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 57 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 58 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 59 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 60 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 61 - protein, 168 aa, Homo sapiens
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 62 - protein, 116 aa, Homo sapiens
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 63 - protein, 167 aa, Homo sapiens
RRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 64 - protein, 168 aa, Homo sapiens
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 65 - protein, 111 aa, Homo sapiens
RHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 66 - protein, 144 aa, Homo sapiens
TERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 67 - protein, 167 aa, Homo sapiens
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 68 - protein, 50 aa, Homo sapiens
LSGAADTQALLRNDVTYAQLHSLTLRREATEPPPSQEGPSPAVPSIYATL

SEQ ID NO: 69 - protein, 36 aa, Homo sapiens
VTYAQLHSLTLRREATEPPPSQEGPSPAVPSIYATL

SEQ ID NO: 70 - protein, 6 aa, Artificial Sequence
  LIR1 ITIM
NLYAAV

SEQ ID NO: 71 - protein, 6 aa, Artificial Sequence
  LIR1 ITIM
VTYAEV

SEQ ID NO: 72 - protein, 6 aa, Artificial Sequence
  LIR1 ITIM
VTYAQL

SEQ ID NO: 73 - protein, 6 aa, Artificial Sequence
  LIR1 ITIM
SIYATL

SEQ ID NO: 74 - protein, 95 aa, Homo sapiens
RAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 75 - protein, 85 aa, Artificial Sequence
  inhibitory domain
RVKFSRSADAPAYQQPDPQEVTYAQLDKRRGRDPEMGGKPRRKPDPQEVTYAQLGMKGERRRGKGHPDPQEVTYAQLHMQALPPR

SEQ ID NO: 76 - protein, 21 aa, Homo sapiens
LCYLLDGILFIYGVILTALFL

SEQ ID NO: 77 - protein, 21 aa, Artificial Sequence
  CD3 zeta transmembrande domain with D15K mutation
LCYLLKGILFIYGVILTALFL

SEQ ID NO: 78 - protein, 21 aa, Artificial Sequence
  CD3 zeta domain with D15R mutation
LCYLLRGILFIYGVILTALFL

SEQ ID NO: 79 - protein, 21 aa, Artificial Sequence
  CD3 gamma transmembrane domaine with E122K mutation
GFLFAKIVSIFVLAVGVYFIA

SEQ ID NO: 80 - protein, 23 aa, Artificial Sequence
  TCR alpha transmembrane domain with R288D mutation
VIGFDILLLKVAGFNLLMTLRLW

SEQ ID NO: 81 - protein, 215 aa, Artificial Sequence
  CD3 delta fused to CD28 stimulatory domain with GGS linker
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKGGSRSKRSRLLHSDYMNMTPRRPGPTRKHYQPYAPPRDFAAYRS

SEQ ID NO: 82 - DNA, 600 bp, Artificial Sequence
  CD3 delta fused to CD28 stimulatory domain with GGS linker
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagggcggaagcaggagcaagcggagcagactgctgcacagcgactacatgaacatgaccccccggaggcctggccccacccggaagcac

SEQ ID NO: 83 - protein, 251 aa, Artificial Sequence
  CD3 epsilon fused to CD28 stimulatory domain with GGS linker
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGGSRSKRSRLLHSDYMNMTPRRPGPTRKHYQPYAPPRDFAAYRS

SEQ ID NO: 84 - DNA, 720 bp, Artificial Sequence
  CD3 epsilon fused to CD28 stimulatory domain with GGS linker
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagcgcagaatcggcggaagcaggagcaagcggagcagactgctgcacagcgactacatgaacatgaccccccggaggcctggccccacccggaagcactaccagccctac

SEQ ID NO: 85 - protein, 226 aa, Artificial Sequence
  CD3 gamma fused to CD28 stimulatory domain with GGS linker
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNGGSRSKRSRLLHSDYMNMTPRRPGPTRKHYQPYAPPRDFAAYRS

SEQ ID NO: 86 - DNA, 660 bp, Artificial Sequence
  CD3 gamma fused to CD28 stimulatory domain with GGS linker
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatggcggaagcaggagcaagcggagcagactgctgcacagcgactacatgaacatgaccccccggaggcctggccccacccggaagcactaccagccctacgcccctcccagggat

SEQ ID NO: 87 - protein, 318 aa, Artificial Sequence
  NY-ESO1 1G4 extracellular domain, TCR alpha T48C extracellular and transmembrane domains, CD28 stimulatory domain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGGSRSKRSRLLHSDYMNMTPRRPGPTRKHYQPYAPPRDFAAYRS

SEQ ID NO: 88 - DNA, 957 bp, Artificial Sequence
  NY-ESO1 1G4 extracellular domain, TCR alpha T48C extracellular and transmembrane domains, CD28 stimulatory domain
atggagacactcttgggcctgcttatcctttggctgcagctgcaatgggtgagcagcaaacaggaggtgacgcagattcctgcagctctgagtgtcccagaaggagaaaacttggttctgaactgcagtttcactgatagcgctatttacaacctccagtggtttaggcaggaccctgggaaaggactcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctctacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 89 - protein, 354 aa, Artificial Sequence
  NY-ESO1 1G4 extracellular domain, TCR beta extracellular and transmembrane domains, CD28 stimulatory domain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSRSKRSRLLHSDYMNMTPRRPGPTRKHYQPYAPPRDFAAYR

SEQ ID NO: 90 - DNA, 1020 bp, Artificial Sequence
  NY-ESO1 1G4 extracellular domain, TCR beta extracellular and transmembrane domains, CD28 stimulatory domain
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcggcggaagcaggagcaagcggagcagactgctgcacagcgactacatgaacatgaccccccggaggcctggccccacccggaagcac

SEQ ID NO: 91 - RNA, 20 nt, Artificial Sequence
  CD3 delta guide RNA targeting sequence
gguccaggaugcguuuuccc

SEQ ID NO: 92 - RNA, 20 nt, Artificial Sequence
  CD3 delta guide RNA targeting sequence
caucacauggguagagggaa

SEQ ID NO: 93 - RNA, 20 nt, Artificial Sequence
  CD3 delta guide RNA targeting sequence
ccucuauagguaucuugaag

SEQ ID NO: 94 - RNA, 20 nt, Artificial Sequence
  CD3 epsilon guide RNA targeting sequence
uuucagauccaggauacuga

SEQ ID NO: 95 - RNA, 20 nt, Artificial Sequence
  CD3 epsilon guide RNA targeting sequence
gaugaggaugauaaaaacau

SEQ ID NO: 96 - RNA, 20 nt, Artificial Sequence
  CD3 epsilon guide RNA targeting sequence
uauuaugucugcuaccccag

SEQ ID NO: 97 - RNA, 20 nt, Artificial Sequence
  CD3 gamma guide RNA targeting sequence
ucucauuucaggaaaccacu

SEQ ID NO: 98 - RNA, 20 nt, Artificial Sequence
  CD3 gamma guide RNA targeting sequence
cagaagccaaaaauaucaca

SEQ ID NO: 99 - RNA, 20 nt, Artificial Sequence
  CD3 gamma guide RNA targeting sequence
auuuuuuuuuaucuucaguu

SEQ ID NO: 100 - RNA, 20 nt, Artificial Sequence
  CD3 zeta guide RNA targeting sequence
gaaacucuuuguguaugugu

SEQ ID NO: 101 - RNA, 20 nt, Artificial Sequence
  CD3 zeta guide RNA targeting sequence
acaguugccgauuacaggua

SEQ ID NO: 102 - RNA, 20 nt, Artificial Sequence
  CD3 zeta guide RNA targeting sequence
aaaggacaagaugaagugga

SEQ ID NO: 103 - protein, 23 aa, Artificial Sequence
  TCR alpha transmembrane domain with R253E mutation
VIGFEILLLKVAGFNLLMTLRLW

SEQ ID NO: 104 - protein, 23 aa, Artificial Sequence
  TCR beta transmembrane domain with K288D mutation
TILYEILLGDATLYAVLVSALVL

SEQ ID NO: 105 - protein, 23 aa, Artificial Sequence
  TCR beta transmembrane domain with K288E mutation
TILYEILLGEATLYAVLVSALVL

SEQ ID NO: 106 - protein, 21 aa, Homo sapiens
GIIVTDVIATLLLALGVFCFA

SEQ ID NO: 107 - protein, 21 aa, Artificial Sequence
  CD3D transmembrane domain with D137R mutation
GIIVTRVIATLLLALGVFCFA

SEQ ID NO: 108 - protein, 21 aa, Artificial Sequence
  CD3D transmembrane domain with D137K mutation
GIIVTKVIATLLLALGVFCFA

SEQ ID NO: 109 - DNA, 78 bp, Homo sapiens
gtgatgtcggtggccacaattgtcatagtggacatctgcatcactgggggcttgctgctgctggtttactactggagc

SEQ ID NO: 110 - protein, 26 aa, Artificial Sequence
  CD3E transmembrane domain with D137R mutation
VMSVATIVIVRICITGGLLLLVYYWS

SEQ ID NO: 111 - protein, 26 aa, Artificial Sequence
  CD3E transmembrane domain with D137K mutation
VMSVATIVIVKICITGGLLLLVYYWS

SEQ ID NO: 112 - protein, 21 aa, Homo sapiens
GFLFAEIVSIFVLAVGVYFIA

SEQ ID NO: 113 - protein, 21 aa, Artificial Sequence
  CD3G transmembrane domain E122R mutation
GFLFARIVSIFVLAVGVYFIA

SEQ ID NO: 114 - protein, 21 aa, Artificial Sequence
  CD3G transmembrane domain with E122K mutation
GFLFAKIVSIFVLAVGVYFIA

SEQ ID NO: 115 - protein, 6 aa, Artificial Sequence
  ITIM motif
  X (1)..(1): X can be S, I, V or L
  X (2)..(2): X can be any amino acid
  X (4)..(5): X can be any amino acid
  X (6)..(6): X can be I, V or L
XXYXXX

SEQ ID NO: 116 - protein, 6 aa, Artificial Sequence
  ITSM motif
  X (2)..(2): X can be any amino acid
  X (4)..(5): X can be any amino acid
  X (6)..(6): X can be V or I
TXYXXX

SEQ ID NO: 117 - protein, 20 aa, Homo sapiens
DSDVYITDKTVLDMRSMDFK

SEQ ID NO: 118 - protein, 249 aa, Artificial Sequence
  NY-ESO1 TCR alpha extracellular domain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLS

SEQ ID NO: 119 - protein, 277 aa, Artificial Sequence
  NY-ESO1 TCR beta extracellular domain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSA

SEQ ID NO: 120 - DNA, 63 bp, Homo sapiens
ggcatcattgtcactgatgtcattgccactctgctccttgctttgggagtcttctgctttgct

SEQ ID NO: 121 - DNA, 63 bp, Homo sapiens
ggctttctctttgctgaaatcgtcagcattttcgtccttgctgttggggtctacttcattgct

SEQ ID NO: 122 - DNA, 69 bp, Homo sapiens
gtgattgggttccgaatcctcctcctgaaagtggccgggtttaatctgctcatgacgctgcggctgtgg

SEQ ID NO: 123 - DNA, 69 bp, Homo sapiens
accatcctctatgagatcttgctagggaaggccaccttgtatgccgtgctggtcagtgccctcgtgctg

SEQ ID NO: 124 - protein, 90 aa, Homo sapiens
NKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 125 - protein, 4 aa, Artificial Sequence
  ITAM motif
  X (2)..(3): X can be any amino acid
  X (4)..(4): X can be L or I
YXXX

SEQ ID NO: 126 - DNA, 269 bp, Homo sapiens
ggacatgagactggaaggctgtctggggctgccgacacacaagctctgttgaggaatgaccaggtctatcagcccctccgagatcgagatgatgctcagtacagccaccttggaggaaactgggctcggaacaagggcggaagcaggagcaagcggagcagactgctgcacagcgactacatgaacatgaccccccggaggcctggccccacccggaagcactaccagccctacgcccctcccagggatttcgccgcctaccggagcta

SEQ ID NO: 127 - DNA, 300 bp, Homo sapiens
aagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagcgcagaatcggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 128 - DNA, 270 bp, Homo sapiens
ggacaggatggagttcgccagtcgagagcttcagacaagcagactctgttgcccaatgaccagctctaccagcccctcaaggatcgagaagatgaccagtacagccaccttcaaggaaaccagttgaggaggaatggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 129 - DNA, 30 bp, Homo sapiens
atggccatggtcaagagaaaggattccaga

SEQ ID NO: 130 - DNA, 123 bp, Homo sapiens
aggagcaagcggagcagactgctgcacagcgactacatgaacatgaccccccggaggcctggccccacccggaagcactaccagccctacgcccctcccagggatttcgccgcctaccggagc

SEQ ID NO: 131 - DNA, 126 bp, Homo sapiens
aaacggggcagaaagaaactcctgtatatattcaaacaaccatttatgaggccagtacaaactactcaagaggaagatggctgtagctgccgatttccagaagaagaagaaggaggatgtgaactg

SEQ ID NO: 132 - protein, 44 aa, Homo sapiens
QLRKTQLLLEVPPSTEDARSCQFPEEERGERSAEEKGRLGDLWV

SEQ ID NO: 133 - DNA, 132 bp, Homo sapiens
cagctgaggaagacacagctgctgctggaggtgccgccgtcgaccgaagacgccagaagctgccagttccccgaggaagagcggggcgagcgatcggcagaggagaaggggcggctgggggacctgtgggtg

SEQ ID NO: 134 - DNA, 123 bp, Homo sapiens
tgtgtcaggtgccggcaccgaaggcgccaagcagagcggatgtctcagatcaagagactcctcagtgagaagaagacctgccagtgtcctcaccggtttcagaagacatgtagccccatttag

SEQ ID NO: 135 - DNA, 99 bp, Homo sapiens
ctttactgcaaccacaggaaccgaagacgtgtttgcaaatgtccccggcctgtggtcaaatcgggagacaagcccagcctttcggcgagatacgtctag

SEQ ID NO: 136 - DNA, 1614 bp, Homo sapiens
atgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcgtcttctcatacggggaccttgcgtacgagaggaggaacaggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggtgaccatgtcaaacattataaaattcgcaaacttgacaatggtggatactacattaccacccgggcccagtttgaaacacttcagcagcttgtacaacattactcagagagagctgcaggactctgctgccgcctagtagttccctgtcacaaagggatgccaaggcttaccgatctgtctgtcaaaaccaaagatgtctgggaaatccctcgagaatccctgcagttgatcaagagactgggaaatgggcagtttggggaagtatggatgggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtag

SEQ ID NO: 137 - DNA, 1530 bp, Homo sapiens
atgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacactgaagctggtggagcggctgggggctggacagttcggggaggtgtggatggggtactacaacgggcacacgaaggtggcggtgaagagcctgaagcagggcagcatgtccccggacgccttcctggccgaggccaacctcatgaagcagctgcaacaccagcggctggttcggctctacgctgtggtcacccaggagcccatctacatcatcactgaatacatggagaatgggagtctagtggattttctcaagaccccttcaggcatcaagttgaccatcaacaaactcctggacatggcagcccaaattgcagaaggcatggcattcattgaagagcggaattatattcatcgtgaccttcgggctgccaacattctggtgtctgacaccctgagctgcaagattgcagactttggcctagcacgcctcattgaggacaacgagtacacagccagggagggggccaagtttcccattaagtggacagcgccagaagccattaactacgggacattcaccatcaagtcagatgtgtggtcttttgggatcctgctgacggaaattgtcacccacggccgcatcccttacccagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagccttag

SEQ ID NO: 138 - DNA, 1860 bp, Homo sapiens
atgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagatgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgactcatcctcagcgaagaatcgacaccctcaactcagatggatacacccctgagccagcacgcataacgtccccagacaaaccgcggccgatgcccatggacacgagcgtgtatgagagcccctacagcgacccagaggagctcaaggacaagaagctcttcctgaagcgcgataacctcctcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctggccagcaaggtggaagggcccccaggcagcacacagaaggctgaggctgcctgtgcctag

SEQ ID NO: 139 - protein, 365 aa, Homo sapiens
PNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 140 - protein, 263 aa, Homo sapiens
LIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 141 - DNA, 702 bp, Homo sapiens
atggaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgaggaaccagcctgtgaggatgcggatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtccctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaactag

SEQ ID NO: 142 - DNA, 1602 bp, Homo sapiens
atggcactgaggaatgtgccctttcgctcagaggtcctgggctgggaccccgacagccttgctgactatttcaagaagctcaactataaggactgtgagaaggcagtgaagaagtaccacatcgatggggctcgcttcttgaacctgacagaaaatgacatccagaagttccccaagctccgggtgccgattctcagtaagttaagtcaggaaatcaacaagaacgaagagaggaggagcatcttcacacgcaaaccccaagtcccgcggtttcctgaagagacagaaagccacgaagaggacaatgggggctggtcgtcctttgaagaagacgattatgaaagtcccaatgatgaccaggatggggaggatgatggagactatgagtcccccaatgaggaggaagaggcacccgtggaagatgacgcggattatgagccgccaccctccaatgacgaggaagctctgcagaactccatcctgcctgccaagcctttccccaactccaactccatgtacatcgaccggcccccctctgggaaaaccccccagcagcctcctgtgcccccccagaggccgatggccgccctcccgcccccaccagccggccggaatcactcgccactgcccccaccccagaccaaccacgaagaacccagcagaagcagaaaccacaaaacggcaaagctccctgctccttcaatagacagaagcacgaaacctcccctagatcgttcattagctccgtttgatagagaacccttcacactaggaaagaaaccaccattttctgacaagccctcgattccagcgggaaggtcactcggggagcatttacccaagattcaaaagcctcctttaccaccgaccacggaaagacatgaaaggagcagccccctgccagggaagaagccacctgtgccaaagcatggatggggaccagacagaagagagaatgatgaagatgatgtgcatcagaggcctttgccccagccagcactacttcctatgagctccaacactttcccttcaagatctactaagccaagtcccatgaaccctctcccatcctctcacatgcctggagcattctcagaaagtaacagcagttttccacagagtgcctccctgccaccatacttctctcaaggccctagcaacagaccacctatcagagccgaaggcagaaacttccccttgccacttccaaacaaacctcggcccccatcccccgcggaggaagagaattcattaaatgaagagtggtacgtttcttatattacccgaccagaggcagaagctgctcttagaaagataaaccaggatggcacatttctggtcagagacagctctaaaaaaacaacaaccaatccatatgtcctcatggtgttgtacaaagataaagtttacaacatccagatccgttatcagaaggaaagtcaagtttacttgttgggaactggactccgagggaaagaggactttctgtctgtgtcagatattattgactacttcaggaaaatgccacttctgctcattgatgggaaaaaccgaggttccagataccagtgcacattaacgcatgctgcagggtacccatag

SEQ ID NO: 143 - DNA, 270 bp, Homo sapiens
aacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttc

SEQ ID NO: 144 - protein, 215 aa, Artificial Sequence; identical to SEQ ID NO: 81
  CD3 delta fused to CD28 ICD with GGS linker

SEQ ID NO: 145 - DNA, 647 bp, Artificial Sequence
  CD3 delta fused to CD28 ICD with GGS linker
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagggcggaagcaggagcaagcggagcagactgctgcacagcgactacatgaacatgaccccccggaggcctggccccacccggaagcactaccagccctacgcccctcccagggatttcgccgcctaccggagcta

SEQ ID NO: 146 - protein, 251 aa, Artificial Sequence; identical to SEQ ID NO: 83
  CD3 epsilon fused to CD28 ICD with GGS linker

SEQ ID NO: 147 - DNA, 756 bp, Artificial Seqeunce
  CD3 epsilon fused to CD28 ICD with GGS linker
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagcgcagaatcggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 148 - protein, 226 aa, Artificial Sequence; identical to SEQ ID NO: 85
  CD3 gamma fused to CD28 ICD with GGS linker

SEQ ID NO: 149 - DNA, 681 bp, Artificial Sequence
  CD3 gamma fused to CD28 ICD with GGS linker
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 150 - protein, 318 aa, Artificial Sequence; identical to SEQ ID NO: 87
  NY-ESO1 1G4, TCR alpha T48C transmembrane domain, CD28 intracellular domain linked to TCR alpha with a GGS linker

SEQ ID NO: 151 - DNA, 957 bp, Artificial Sequence; identical to SEQ ID NO: 88
  NY-ESO1 1G4, TCR alpha T48C transmembrane domain, CD28 intracellular domain linked to TCR alpha with a GGS linker

SEQ ID NO: 152 - protein, 355 aa, Artificial Sequence
  NY-ESO1 1G4,TCR beta transmembrane domain, CD28 intracellular domain linked to TCR alpha with a GGS linker
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSRSKRSRLLHSDYMNMTPRRPGPTRKHYQPYAPPRDFAAYRS

SEQ ID NO: 153 - DNA, 1068 bp, Artificial Sequence
  NY-ESO1 1G4, TCR beta transmembrane domain, CD28 intracellular domain linked to TCR alpha with a GGS linker
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcggcggaagcaggagcaagcggagcagactgctgcacagcgactacatgaacatgaccccccggaggcctggccccacccggaagcactaccagccctacgcccctcccagggatttcgccgcctaccggagctag

SEQ ID NO: 154 - protein, 258 aa, Artificial Sequence
  Synthetic inhibitory CD3z
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGGSGGGGSRVKFSRSADAPAYQQPDPQEVTYAQLDKRRGRDPEMGGKPRRKPDPQEVTYAQLGMKGERRRGKGHPDPQEVTYAQLHMQALPPR

SEQ ID NO: 155 - protein, 340 aa, Artificial Sequence
  Synthetic inhibitory CD3z
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGGSGGGGSRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 156 - protein, 136 aa, Artificial Sequence
  Synthetic inhibitory CD3z
MKWKALFTAAILQAQLPITEAQSFGLLDPKLCYLLDGILFIYGVILTALFLRVKFSRSADAPAYQQPDPQEVTYAQLDKRRGRDPEMGGKPRRKPDPQEVTYAQLGMKGERRRGKGHPDPQEVTYAQLHMQALPPR

SEQ ID NO: 157 - protein, 389 aa, Artificial Sequence
  TCR alpha and PD1 intracellular domain fusion protein
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGGGGSGGGGSGGGGSGGGGSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 158 - protein, 384 aa, Artificial Sequence
  TCR alpha and KIRDL2 intracellular domain fusion protein
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGGGGSGGGGSGGGGSGGGGSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 159 - protein, 426 aa, Artificial Sequence
  TCR alpha and LIR1 intracellular domain fusion protein
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGGGGSGGGGSGGGGSGGGGSRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDSAIH

SEQ ID NO: 160 - protein, 379 aa, Artificial Sequence
  Synthetic inhibitory CD3 zeta
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGGGGSGGGGSGGGGSGGGGSRVKFSRSADAPAYQQPDPQEVTYAQLDKRRGRDPEMGGKPRRKPDPQEVTYAQLGMKGERRRGKGHPDPQEVTYAQLHMQALPPR

SEQ ID NO: 161 - protein, 421 aa, Artificial Sequence
  TCR beta and PD1 intracellular domain fusion protein
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGGGSGGGGSGGGGSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 162 - protein, 416 aa, Artificial Sequence
  TCR beta and KIR3DL2 intracellular domain fusion protein
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGGGSGGGGSGGGGSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 163 - protein, 493 aa, Artificial Sequence
  TCR beta and LIR1 intracellular domain fusion protein
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGGGSGGGGSGGGGSRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 164 - protein, 411 aa, Artificial Sequence
  Synthetic inhibitory CD3 zeta
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGGGSGGGGSGGGGSRVKFSRSADAPAYQQPDPQEVTYAQLDKRRGRDPEMGGKPRRKPDPQEVTYAQLGMKGERRRGKGHPDPQEVTYAQLHMQALPPR

SEQ ID NO: 165 - DNA, 1167 bp, Artificial Sequence
  TCR alpha and PD1 intracellular domain fusion protein
atggagacactcttgggcctgcttatcctttggctgcagctgcaatgggtgagcagcaaacaggaggtgacgcagattcctgcagctctgagtgtcccagaaggagaaaacttggttctgaactgcagtttcactgatagcgctatttacaacctccagtggtttaggcaggaccctgggaaaggactcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctctacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcggaggtggaagcggagggggaggatctggcggcggaggaagcggaggcggcgggtcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 166 - DNA, 1152 bp, Artificial Sequence
  TCR alpha and KIR3DL2 intracellular domain fusion protein
atggagacactcttgggcctgcttatcctttggctgcagctgcaatgggtgagcagcaaacaggaggtgacgcagattcctgcagctctgagtgtcccagaaggagaaaacttggttctgaactgcagtttcactgatagcgctatttacaacctccagtggtttaggcaggaccctgggaaaggactcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctctacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcggaggtggaagcggagggggaggatctggcggcggaggaagcggaggcggcgggtccaacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttc

SEQ ID NO: 167 - DNA, 1383 bp, Artificial Sequence
  TCR alpha and LIR1 intracellular domain fusion protein
atggagacactcttgggcctgcttatcctttggctgcagctgcaatgggtgagcagcaaacaggaggtgacgcagattcctgcagctctgagtgtcccagaaggagaaaacttggttctgaactgcagtttcactgatagcgctatttacaacctccagtggtttaggcaggaccctgggaaaggactcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctctacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcggaggtggaagcggagggggaggatctggcggcggaggaagcggaggcggcgggtcccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 168 - DNA, 1263 bp, Artificial Sequence
  TCR beta and PD1 intracellular domain fusion protein
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcggagggggaggatctggcggcggaggaagcggaggcggcgggtcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 169 - DNA, 1248 bp, Artificial Sequence
  TCR beta and KIR3DL2 intracellular domain fusion protein
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcggagggggaggatctggcggcggaggaagcggaggcggcgggtccaacaaaaagaatgctgctgtaatggaccaagagcctgcgggggacagaacagtgaataggcaggactctgatgaacaagaccctcaggaggtgacgtacgcacagttggatcactgcgttttcatacagagaaaaatcagtcgcccttctcagaggcccaagacacccctaacagataccagcgtgtacacggaacttccaaatgctgagcccagatccaaagttgtctcctgcccacgagcaccacagtcaggtcttgagggggttttc

SEQ ID NO: 170 - DNA, 1479 bp, Artificial Sequence
  TCR beta and LIR1 intracellular domain fusion protein
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcggagggggaggatctggcggcggaggaagcggaggcggcgggtcccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 171 - protein, 132 aa, Artificial Sequence
  CD3D with ITAM deletion
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGR

SEQ ID NO: 172 - protein, 160 aa, Artificial Sequence
  CD3E with ITAM deletion
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAK

SEQ ID NO: 173 - protein, 143 aa, Artificial Sequence
  CD3G with ITAM deletion
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVR

SEQ ID NO: 174 - protein, 59 aa, Artificial Sequence
  CD3Z with ITAM deletion
MKWKALFTAAILQAQLPITEAQSFGLLDPKLCYLLDGILFIYGVILTALFLRVKFSRSA

SEQ ID NO: 175 - DNA, 396 bp, Artificial Sequence
  CD3D with ITAM deletion
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaagg

SEQ ID NO: 176 - DNA, 480 bp, Artificial Sequence
  CD3E with ITAM deletion
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaag

SEQ ID NO: 177 - DNA, 429 bp, Artificial Sequence
  CD3G with ITAM deletion
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgc

SEQ ID NO: 178 - DNA, 177 bp, Artificial Sequence
  CD3Z with ITAM deletion
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgca

SEQ ID NO: 179 - protein, 276 aa, Artificial Sequence
  CD3D and PD1 intracellular domain fusion protein
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 180 - protein, 304 aa, Artificial Sequence
  CD3E and PD1 intracellular domain fusion protein
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 181 - protein, 287 aa, Artificial Sequence
  CD3G and PD1 intracellular domain fusion protein
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 182 - protein, 203 aa, Artificial Sequence
  CD3Z and PD1 intracellular domain fusion protein
MKWKALFTAAILQAQLPITEAQSFGLLDPKLCYLLDGILFIYGVILTALFLRVKFSRSATERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 183 - protein, 296 aa, Artificial Sequence
  CD3D and LIR1 ICD fusion protein
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHERHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 184 - protein, 322 aa, Artificial Sequence
  CD3E and LIR1 intracellular domain fusion protein
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 185 - protein, 305 aa, Artificial Sequence
  CD3G and LIR1 intracellular domain fusion protein
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 186 - protein, 222 aa, Artificial Sequence
  CD3Z and LIR1 intracellular domain fusion protein
MKWKALFTAAILQAQLPITEAQSFGLLDPKLCYLLDGILFIYGVILTALFLRVKFRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 187 - protein, 190 aa, Artificial Sequence
  CD3D LIR1 fusion protein
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDVTYAQLHSLTLRREATEPPPSQEGPSPAVPSIYATLGGNWARNK

SEQ ID NO: 188 - protein, 226 aa, Artificial Sequence
  CD3E LIR1 fusion protein
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNVTYAQLHSLTLRREATEPPPSQEGPSPAVPSIYATLNQRRI

SEQ ID NO: 189 - protein, 201 aa, Artificial Sequence
  CD3G LIR1 fusion protein
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDVTYAQLHSLTLRREATEPPPSQEGPSPAVPSIYATLQGNQLRRN

SEQ ID NO: 190 - protein, 219 aa, Artificial Sequence
  CD3Z LIR1 fusion protein
MKWKALFTAAILQAQLPITEAQSFGLLDPKLCYLLDGILFIYGVILTALFLRVKFSRSADAPAYQQGQNVTYAQLHSLTLRREATEPPPSQEGPSPAVPSIYATLDKRRGRDPEMGGKPRRKNPQEVTYAQLHSLTLRREATEPPPSQEGPSPAVPSIYATLGMKGERRRGKGHDVTYAQLHSLTLRREATEPPPSQEGPSPAVPSIYATLHMQALPPR

SEQ ID NO: 191 - DNA, 828 bp, Artificial Sequence
  CD3D and PD1 intracellular domain fusion protein
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 192 - skipped

SEQ ID NO: 193 - DNA, 912 bp, Artificial Sequence
  CD3E and PD1 intracellular domain fusion protein
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 194 - DNA, 861 bp, Artificial Sequence
  CD3G and PD1 intracellular domain fusion protein
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgcacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 195 - DNA, 609 bp, Artificial Sequence
  CD3Z and PD1 intracellular domain fusion protein
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcaacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 196 - DNA, 888 bp, Artificial Sequence
  CD3D and LIR1 intracellular domain fusion protein
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagcgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 197 - DNA, 966 bp, Artificial Sequence
  CD3E and LIR1 intracellular domain fusion protein
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagacgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 198 - DNA, 915 bp, Artificial Sequence
  CD3G and LIR1 intracellular domain fusion protein
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 199 - DNA, 666 bp, Artificial Sequence
  CD3Z and LIR1 intracellular domain fusion protein
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 200 - DNA, 570 bp, Artificial Sequence
  CD3D LIR1 fusion protein
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgacgtgacctacgcccagctgcacagcttgaccctcagacgggaggcaactgagcctcctccatcccaggaagggccctctccagctgtgcccagcatctacgccactctgggaggaaactgggctcggaacaag

SEQ ID NO: 201 - DNA, 678 bp, Artificial Sequence
  CD3E LIR1 fusion protein
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacgtgacctacgcccagctgcacagcttgaccctcagacgggaggcaactgagcctcctccatcccaggaagggccctctccagctgtgcccagcatctacgccactctgaatcagcgcagaatc

SEQ ID NO: 202 - DNA, 603 bp, Artificial Sequence
  CD3G LIR1 fusion protein
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgacgtgacctacgcccagctgcacagcttgaccctcagacgggaggcaactgagcctcctccatcccaggaagggccctctccagctgtgcccagcatctacgccactctgcaaggaaaccagttgaggaggaat

SEQ ID NO: 203 - DNA, 657 bp, Artificial Sequence
  CD3Z LIR1 fusion protein
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaacgtgacctacgcccagctgcacagcttgaccctcagacgggaggcaactgagcctcctccatcccaggaagggccctctccagctgtgcccagcatctacgccactctggacaagcgtagaggccgggaccctgagatggggggaaagccgagaaggaagaaccctcaggaagtgacctacgcccagctgcacagcttgaccctcagacgggaggcaactgagcctcctccatcccaggaagggccctctccagctgtgcccagcatctacgccactctggggatgaaaggcgagcgccggaggggcaaggggcacgatgtgacctacgcccagctgcacagcttgaccctcagacgggaggcaactgagcctcctccatcccaggaagggccctctccagctgtgcccagcatctacgccactctgcacatgcaggccctgccccctcgc

SEQ ID NO: 204 - protein, 219 aa, Artificial Sequence
  CD3Z LIR1 fusion protein with D15K mutation
MKWKALFTAAILQAQLPITEAQSFGLLDPKLCYLLKGILFIYGVILTALFLRVKFSRSADAPAYQQGQNVTYAQLHSLTLRREATEPPPSQEGPSPAVPSIYATLDKRRGRDPEMGGKPRRKNPQEVTYAQLHSLTLRREATEPPPSQEGPSPAVPSIYATLGMKGERRRGKGHDVTYAQLHSLTLRREATEPPPSQEGPSPAVPSIYATLHMQALPPR

SEQ ID NO: 205 - protein, 201 aa, Artificial Sequence
  CD3G LIR1 fusion protein with E122K mutation
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAKIVSIFVLAVGVYFIAGQDGVRQSRASDKQTLLPNDVTYAQLHSLTLRREATEPPPSQEGPSPAVPSIYATLQGNQLRRN

SEQ ID NO: 206 - protein, 274 aa, Artificial Sequence
  TCR alpha with R253D mutation
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFDILLLKVAGFNLLMTLRLWSS

SEQ ID NO: 207 - protein, 311 aa, Artificial Sequence
  TCR beta with K288D mutation
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGDATLYAVLVSALVLMAMVKRKDSRG

SEQ ID NO: 208 - protein, 163 aa, Artificial Sequence
  CD3Z with D15R mutation
MKWKALFTAAILQAQLPITEAQSFGLLDPKLCYLLRGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 209 - protein, 163 aa, Artificial Sequence
  CD3Z with D15K mutation
MKWKALFTAAILQAQLPITEAQSFGLLDPKLCYLLKGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 210 - protein, 163 aa, Artificial Sequence
  CD3Z D15R disulfide mutant
MKWKALFTAAILQAQLPITECQSFGLLDPKLGYLLRGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - protein, 163 aa, Artificial Sequence
  CD3Z D15K disulfide mutant
MKWKALFTAAILQAQLPITECQSFGLLDPKLGYLLKGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 212 - protein, 172 aa, Artificial Sequence
  CD3D with D137K mutation
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTKVIATLLLALGVFCFAGHETGRLSGAADTQALLRNDQVYQPLRDRDDAQYSHLGGNWARNKA

SEQ ID NO: 213 - protein, 171 aa, Artificial Sequence
  CD3D with D137R mutation
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTRVIATLLLALGVFCFAGHETGRLSGAADTQALLRNDQVYQPLRDRDDAQYSHLGGNWARNK

SEQ ID NO: 214 - protein, 207 aa, Artificial Sequence
  CD3E with D137R mutation
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVRICITGGLLLLVYYWSKNRKAKAKPVTRGAGAGGRQRGQNKERPPPVPNPDYEPIRKGQRDLYSGLNQRRI

SEQ ID NO: 215 - protein, 207 aa, Artificial Sequence
  CD3E with D137K mutation
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVKICITGGLLLLVYYWSKNRKAKAKPVTRGAGAGGRQRGQNKERPPPVPNPDYEPIRKGQRDLYSGLNQRRI

SEQ ID NO: 216 - protein, 182 aa, Artificial Sequence
  CD3G with E122R mutation
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFARIVSIFVLAVGVYFIAGQDGVRQSRASDKQTLLPNDQLYQPLKDREDDQYSHLQGNQLRRN

SEQ ID NO: 217 - protein, 182 aa, Artificial Sequence
  CD3G with E122K mutation
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAKIVSIFVLAVGVYFIAGQDGVRQSRASDKQTLLPNDQLYQPLKDREDDQYSHLQGNQLRRN

SEQ ID NO: 218 - DNA, 657 bp, Artificial Sequence
  CD3Z LIR1 fusion protein with D15K mutation
atgaagtggaaggcgcttttcaccgcggccatcctgcaggcacagttgccgattacagaggcacagagctttggcctgctggatcccaaactctgctacctgctgaaaggaatcctcttcatctatggtgtcattctcactgccttgttcctgagagtgaagttcagcaggagcgcagacgcccccgcgtaccagcagggccagaacgtgacctacgcacagctccatagtcttactcttagaagggaagctactgagccgccgcctagccaggagggcccttccccagcggtcccttctatctatgcgactttggacaaaagaaggggccgagaccctgaaatgggaggtaaaccgcgtcgaaagaatccccaagaagtaacatacgcgcaattgcatagcttgacactcaggcgcgaggcaacggagcctccccccagccaagagggtccgagtcctgccgttccgagtatatatgccactctcggtatgaaaggagaaaggcgcaggggcaaaggccatgacgtgacgtatgcgcaattgcacagtctgacgttgaggcgggaagcgactgagccaccaccatcccaagaagggccatctccggcggtgccgtccatatatgcaactcttcacatgcaagctctgccccctcgc

SEQ ID NO: 219 - DNA, 603 bp, Artificial Sequence
  CD3G LIR1 fusion protein with E122K mutation
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctaagatcgtcagcattttcgtccttgctgttggggtctacttcattgctggacaggatggagttcgccagtcgagagcttcagacaagcagactctgttgcccaatgacgtgacctacgcccagctgcacagcttgaccctcagacgggaggcaactgagcctcctccatcccaggaagggccctctccagctgtgcccagcatctacgccactctgcaaggaaaccagttgaggaggaat

SEQ ID NO: 220 - DNA, 822 bp, Artificial Sequence
  TCR alpha with R253D mutation
atggagacactcttgggcctgcttatcctttggctgcagctgcaatgggtgagcagcaaacaggaggtgacgcagattcctgcagctctgagtgtcccagaaggagaaaacttggttctgaactgcagtttcactgatagcgctatttacaacctccagtggtttaggcaggaccctgggaaaggactcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctctacggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttcgatatcctcctcctgaaagtggccgggtttaatctgctcatgacgctgcggctgtggtccagc

SEQ ID NO: 221 - DNA, 933 bp, Artificial Sequence
  TCR beta with K288D mutation
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctaggggacgccaccttgtatgccgtgctggtcagtgccctcgtgctgatggccatggtcaagagaaaggattccagaggc

SEQ ID NO: 222 - DNA, 489 bp, Artificial Sequence
  CD3Z with D15R mutation
atgaagtggaaggcgcttttcaccgcggccatcctgcaggcacagttgccgattacagaggcacagagctttggcctgctggatcccaaactctgctacctgctgcgtggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 223 - DNA, 489 bp, Artificial Sequence
  CD3Z with D15K mutation
atgaagtggaaggcgcttttcaccgcggccatcctgcaggcacagttgccgattacagaggcacagagctttggcctgctggatcccaaactctgctacctgctgaaaggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 224 - DNA, 489 bp, Artificial Sequence
  CD3Z with D15R disulfide mutant
atgaagtggaaggcgcttttcaccgcggccatcctgcaggcacagttgccgattacagagtgtcagagctttggcctgctggatcccaaactcggctacctgctgcgtggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 225 - DNA, 489 bp, Artificial Sequence
  CD3Z with D15K disulfide mutant
atgaagtggaaggcgcttttcaccgcggccatcctgcaggcacagttgccgattacagagtgtcagagctttggcctgctggatcccaaactcggctacctgctgaaaggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 226 - DNA, 513 bp, Artificial Sequence
  CD3D with D137K mutation
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactaaggtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaag

SEQ ID NO: 227 - DNA, 513 bp, Artificial Sequence
  CD3D with D137R mutation
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactagggtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaag

SEQ ID NO: 228 - DNA, 621 bp, Artificial Sequence
  CD3E with D137R mutation
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtgcgcatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagcgcagaatc

SEQ ID NO: 229 - DNA, 624 bp, Artificial Sequence
  CD3E with D137K mutation
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtgaagatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagcgcagaatctag

SEQ ID NO: 230 - DNA, 546 bp, Artificial Sequence
  CD3G with E122R mutation
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctagaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 231 - DNA, 546 bp, Artificial Sequence
  CD3G with E122K mutation
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctaagatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 232 - DNA, 501 bp, Homo sapiens
cgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 233 - DNA, 151 bp, Homo sapiens
ctgtctggggctgccgacacacaagctctgttgaggaatgacgtgacctacgcccagctgcacagcttgaccctcagacgggaggcaactgagcctcctccatcccaggaagggccctctccagctgtgcccagcatctacgccactctgg

SEQ ID NO: 234 - DNA, 108 bp, Homo sapiens
gtgacctacgcccagctgcacagcttgaccctcagacgggaggcaactgagcctcctccatcccaggaagggccctctccagctgtgcccagcatctacgccactctg

SEQ ID NO: 235 - DNA, 285 bp, Homo sapiens
cgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 236 - DNA, 432 bp, Homo sapiens
acagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 237 - protein, 14 aa, Artificial Sequence
  ITAM pair
  misc_feature (2)..(3): Xaa can be any naturally occurring amino acid
  X (4)..(4): X can be L or I
  X (5)..(10): X can be any amino acid
  X (12)..(13): X can be any amino acid
  X (14)..(14): X can be L or I
YXXXXXXXXXYXXX

SEQ ID NO: 238 - protein, 15 aa, Artificial Sequence
  ITAM pair
  misc_feature (2)..(3): Xaa can be any naturally occurring amino acid
  misc_feature (4)..(4): X can be L or I
  misc_feature (5)..(11): X can be any amino acid
  misc_feature (13)..(14): X can be any amino acid
  misc_feature (15)..(15): X can be L or I
YXXXXXXXXXXYXXX

SEQ ID NO: 239 - protein, 16 aa, Artificial Sequence
  ITAM pair
  X (2)..(3): X can be any amino acid
  X (4)..(4): X can be L or I
  X (5)..(12): X can be any amino acid
  X (14)..(15): X can be any amino acid
  X (16)..(16): X can be L or I
YXXXXXXXXXXXYXXX

SEQ ID NO: 240 - protein, 4 aa, Artificial Sequence
  linker
GGGS

(19) United States
(12) Patent Application Publication
(10) Pub. No.: US 2022/0389075 A1
(43) Pub. Date: Dec. 8, 2022

(54) ENGINEERED T CELL RECEPTORS AND USES THEREOF

(71) Applicant: A2 Biotherapeutics, Inc., Agoura Hills, CA (US)

(72) Inventors: Julyun OH, Woodland Hills, CA (US); Chang Yi, Camarillo, CA (US); Dora Toledo Warshaviak, Oak Park, CA (US); Jee Young Mock, North Hollywood, CA (US); Mark Daris, Newbury Park, CA (US); Agnes Hamburger, Newbury Park, CA (US); James Johnston, Agoura Hills, CA (US); Carl Alexander Kamb, Agoura Hills, CA (US)

(21) Appl. No.: 17/755,750

(22) PCT Filed: Nov. 10, 2020
(86) PCT No.: PCT/US2020/059856

§ 371 Date: May 6, 2022

Related U.S. Application Data
(60) Provisional application No. 62/934,419, filed on Nov. 12, 2019.
(60) Provisional application No. 63/111,501, filed on Nov. 9, 2020.

(51) Int. Cl. C07K 14/725; C07K 14/705; C12N 9/12; C12N 5/0783; C12N 15/85; A61K 35/17; C07K 14/73

(52) U.S. Cl. CPC C07K 14/7051; C07K 14/70503; C07K 14/70521; C12N 9/12; C12Y 207/10002; C12N 5/0636; C12N 15/85; A61K 35/17; C07K 14/70578; C07K 14/70514; C07K 2319/03; C07K 2319/02; C12N 2510/00

(57) ABSTRACT

Provided are engineered T-cell receptors comprising fusion proteins comprising a transmembrane domain and an intracellular domain capable of providing a stimulatory signal or an inhibitory signal, and immune cells comprising same.

### CROSS REFERENCE TO RELATED APPLICATIONS

This application claims priority to and the benefit of U.S. Provisional Patent Application Nos. 63/111,501, filed on Nov. 9, 2020, and 62/934,419, filed on Nov. 12, 2019, the contents of each of which are incorporated herein by reference in their entireties.

### INCORPORATION BY REFERENCE OF SEQUENCE LISTING

The contents of the text file submitted electronically herewith are incorporated herein by reference in their entirety: A computer readable format copy of the Sequence Listing (filename: A2BI_024_02WO_SeqList.txt, date recorded: Nov. 10, 2020, file size: 309 kilobytes).

### BACKGROUND

T-cell receptors (TCRs) are expressed on the surface of T-cells, and are responsible for recognizing peptide antigens bound to the major histocompatibility complex (MHC) on the surface of antigen presenting cells. When a T-cell receptor engages its cognate peptide-major histocompatibility complex (pMHC), the TCR elicits intracellular signals via the tyrosine kinases lymphocyte-specific tyrosine kinase (Lck) and zeta-associated protein 70 (ZAP70) and downstream signaling via linker for activation of T cells (LAT). This leads, together with a second co-stimulatory signal, to activation of the T-cell. However, in some cases, TCR signaling can be insufficient to activate T-cells. Recent reports (see, for example, Baeuerle et al., Nature Communications 10: 2087, 2019) suggest that one can alter the DNA encoding a TCR subunit, including TCR alpha, TCR beta, CD3 delta, CD3 epsilon or CD3 gamma, by attaching a separate binder to the external portion of the protein, for instance a single-chain variable fragment (scFv), and thus redirect the chimeric scFv-TCR to a new surface antigen/protein on the target cell. In some cases, the new surface antigen/protein is a tumor antigen with restricted expression on normal cells. Both conventional TCRs and chimeric TCRs have potential uses in medicine, in particular cancer. There remains, however, a need in the art for TCRs with altered activity. The disclosure provides compositions and methods for altering TCR activity.

### SUMMARY

The disclosure provides, in part, engineered T cell receptors (TCR) comprising one or more inhibitory signaling domain(s) fused to the intracellular portion of at least one TCR subunit. By coupling TCR alpha, TCR beta, CD3 delta, CD3 epsilon or CD3 gamma to one or more intracellular signaling domains, TCR-mediated signaling can be used to inhibit, rather than activate, immune cells.

The disclosure provides, in part, engineered T cell receptors (TCR) comprising one or more inhibitory signaling domain(s) fused to the intracellular portion of at least one TCR subunit. By coupling TCR alpha, TCR beta, CD3 delta, CD3 epsilon, CD3 gamma or CD3 zeta to one or more inhibitory intracellular signaling domains, TCR-mediated signaling can be inhibited in response to binding of the TCR complex to an antigen. Engineered TCRs comprising inhibitory signaling domains can specifically inhibit the function of immune cells expressing the engineered TCRs, for example inhibiting proliferation, cytokine production or cytotoxicity. Engineered TCRs of the disclosure may be provided within a T cell of the disclosure.

In some aspects, the disclosure provides a fusion protein that includes an extracellular domain, a transmembrane domain and a first intracellular domain capable of providing an inhibitory signal. In some embodiments, the fusion protein comprises a second intracellular domain, for example an intracellular domain isolated or derived from the TCR subunit to which the first intracellular domain is fused. The first intracellular domain capable of providing an inhibitory signal can comprise one or more motifs capable of providing the inhibitory signal, for example immunoreceptor tyrosine-based inhibitory motifs (ITIMs) and/or Src Homology 2 (SH2) domains. The ITIM can be natural, i.e. part of a domain isolated or derived from a protein, or non-naturally occurring or synthetic. In some aspects, the first intracellular domain comprises a programmed cell death 1 (PD-1) intracellular domain, a cytotoxic T-lymphocyte associated protein 4 (CTLA-4) intracellular domain, a killer cell immunoglobulin like receptor three Ig domains and long cytoplasmic tail 2 (KIR3DL2) intracellular domain, a killer cell immunoglobulin like receptor three Ig domains and long cytoplasmic tail 3 (KIR3DL3) intracellular domain, a zeta chain of T cell receptor associated protein kinase 70 (ZAP70) domain comprising a Src Homology 2 (SH2) domain, a ZAP70 inactivated kinase domain, a leukocyte immunoglobulin like receptor B1 (LIR1) intracellular domain, an Fc gamma receptor IIB (FcgRIIB) intracellular domain, or a killer cell lectin like receptor K1 (NKG2D) intracellular domain.

Other aspects of the disclosure include TCRs comprising a fusion protein as described herein, polynucleotides encoding fusion proteins, vectors for delivering polynucleotides encoding fusion proteins, and immune cells comprising polynucleotides encoding fusion proteins or expressing the fusion proteins, pharmaceutical compositions comprising such immune cells, methods of generating immune cells, methods of use, and kits.

Other features and advantages of the invention will be apparent from and encompassed by the following detailed description and claims.

### BRIEF DESCRIPTION OF DRAWINGS

The present application can be understood by reference to the following description taken in conjunction with the accompanying figures.

FIG. 1A is a series of plots showing T Cell Receptor TCR expression using fluorescence activated cell sorting (FACS). SSC-W: side scatter signal width.

FIG. 1B is a plot showing TCR-mediated signaling for CD28-tagged engineered TCRs where an alpha or beta, or both alpha and beta chains were fused to a CD28 domain.

FIG. 2A is a series of plots showing TCR expression using FACS.

FIG. 2B is a plot showing TCR-mediated signaling for CD28-tagged CD3 engineered TCRs where a CD3 gamma, delta, or epsilon subunit were fused to a CD28 domain.

FIG. 3A is a series of FACS plots showing TCR expression.

FIG. 3B is a plot showing TCR-mediated signaling for 4-1BB or 4-1BB and CD28-tagged engineered TCRs. The 4-1BB and/or CD28 intracellular domains were fused to TCR alpha or beta chains, or both, of the TCR.

FIG. 4A is a series of FACS plots showing TCR expression.

FIG. 4B is a plot showing TCR-mediated signaling for 4-1BB and/or CD28-tagged engineered TCRs. The 4-1BB and/or CD28 intracellular domains were fused to TCR alpha or beta chains, or both, of the TCR using a long (L) linker.

FIG. 5A is a series of FACS plots showing TCR expression.

FIG. 5B is a plot showing TCR-mediated signaling for CD4-tagged engineered TCRs. The CD4 intracellular domain was fused to the TCR alpha or beta chain, or both.

FIG. 6A is a series of FACS plots showing TCR expression.

FIG. 6B is a plot showing TCR-mediated signaling for CD8a or CD4 and CD8a-tagged engineered TCRs. The CD8a intracellular domain was fused to TCR alpha and/or beta chain of the TCR, or CD8a and CD4 domains were fused to both the alpha and beta chains of the TCR.

FIG. 7A is a series of FACS plots showing TCR expression.

FIG. 7B is a plot showing TCR-mediated signaling for CD4 or CD8-tagged engineered TCRs. CD4 or CD8 intracellular domains were fused to the beta chain of the TCR using a long (L) linker.

FIG. 8A is a series of FACS plots showing TCR expression.

FIG. 8B is a plot showing TCR-mediated signaling (FIG. 8B) for Lck-tagged engineered TCRs. The LCK intracellular domain was fused to the alpha chain, the beta chain, or both, of the TCR using a short (S) or long (L) linker.

FIG. 9A is a series of FACS plots showing TCR expression.

FIG. 9B is a plot showing TCR-mediated signaling (FIG. 9B) for Lck-tagged on beta chain of engineered TCRs. Lck intracellular domains of the indicated amino acid residues were fused to the beta chain of the TCR.

FIG. 10A is a series of FACS plots showing TCR expression.

FIG. 10B is a plot showing TCR-mediated signaling for Fyn-tagged engineered TCRs. The FYN intracellular domains were fused to the alpha or beta chain, or both, of the TCR using a short (S) or long (L) linker.

FIG. 11A is a series of FACS plots showing TCR expression.

FIG. 11B is a plot showing TCR-mediated signaling for ZAP70-tagged engineered TCRs. ZAP70(KD) or ZAP70(IB, KD) intracellular domains were fused to the alpha or beta chain, or both, of the TCR using a short (S) or long (L) linker.

FIG. 12A is a series of FACS plots showing TCR expression.

FIG. 12B is a plot showing TCR-mediated signaling for LAT-tagged engineered TCRs. The LAT intracellular domain was fused to the alpha or beta chain, or both, of the TCR, using a short (S) or long (L) linker.

FIG. 13A is a FACS plot showing TCR expression.

FIG. 13B is a plot showing TCR-mediated signaling (FIG. 13B) for SLP76-tagged engineered TCRs. The SLP76 intracellular domain was fused to the alpha or beta chain of the TCR using a short (S) or long (L) linker.

FIG. 14A is a FACS plot showing TCR expression.

FIG. 14B is a plot showing TCR-mediated signaling for LAT-FYN or LAT-LCK-tagged engineered TCRs. LAT-Fyn, or LAT-Lck intracellular domains were fused to the beta chain of the TCR using a short (S) or long (L) linker.

FIG. 15 shows a scatter plot summarizing results with engineered TCRs in the Jurkat-NFAT reporter system.

FIG. 16A shows plots comparing TCRs with both engineered alpha and engineered beta chains against with only alpha or beta chain engineered.

FIG. 16B shows plots comparing TCRs with short or long linkers.

FIG. 17A is a diagram showing an exemplary TCR with a heterologous domain fused to the intracellular tail of TCR alpha. Cell membranes are shown in gray, TCR complex subunits in white, linkers are represented as black lines, and domains capable of providing additional stimulatory signal are cross-hatched.

FIG. 17B shows a TCR with a heterologous domain fused to the intracellular tail of TCR beta.

FIG. 17C shows a TCR with heterologous domains fused to the intracellular tails of TCR alpha and TCR beta.

FIG. 17D shows a TCR with two heterologous domains fused to the intracellular tail of TCR beta in tandem.

FIG. 17E shows a TCR with two heterologous domains fused to the intracellular tail of TCR alpha in tandem.

FIG. 17F shows a TCR with a heterologous domain fused to the intracellular tail of CD3 gamma.

FIG. 17G shows a TCR with a heterologous domain fused to the intracellular tail of CD3 epsilon.

FIG. 17H shows a TCR with a heterologous domain fused to the intracellular tail of CD3 delta.

FIG. 18 shows a sequence of an exemplary fusion protein of the disclosure comprising a CD3 delta subunit fused to a CD28 stimulatory domain with a GGS linker. The DNA sequence of SEQ ID NO: 82 is shown at top, the amino acid sequence of SEQ ID NO: 81 is shown at bottom.

FIG. 19 shows a sequence of an exemplary fusion protein of the disclosure comprising a CD3 epsilon subunit fused to a CD28 stimulatory domain with a GGS linker. The DNA sequence of SEQ ID NO: 84 is shown at top, the amino acid sequence of SEQ ID NO: 83 is shown at bottom.

FIG. 20 shows a sequence of an exemplary fusion protein of the disclosure comprising a CD3 gamma subunit fused to a CD28 stimulatory domain with a GGS linker. The DNA sequence of SEQ ID NO: 86 is shown at top, the amino acid sequence of SEQ ID NO: 85 is shown at bottom.

FIG. 21 shows a sequence of an exemplary fusion protein of the disclosure comprising a an extracellular domain targeting NY-ESO1 1G4, TCR alpha T48C extracellular and transmembrane domains, and a CD28 stimulatory domain fused to the TCR alpha subunit with a GGS linker. The DNA sequence of SEQ ID NO: 88 is shown at top, the amino acid sequence of SEQ ID NO: 87 is shown at bottom.

FIG. 22 shows a sequence of an exemplary fusion protein of the disclosure comprising an extracellular domain targeting NY-ESO1 1G4, TCR beta extracellular and transmembrane domains, and a CD28 stimulatory domain fused to the TCR alpha subunit with a GGS linker. The DNA sequence of SEQ ID NO: 90 is shown at top, the amino acid sequence of SEQ ID NO: 89 is shown at bottom.

FIG. 23A illustrates an alignment of TCR alpha chain protein sequences. The box indicates the transmembrane domain. From top to bottom: human TCR alpha amino acids 1-140 (SEQ ID NO: 27), mouse TCR alpha amino acids 1-136 (SEQ ID NO: 28), monkey TCR alpha amino acids 1-140 (SEQ ID NO: 29), guinea pig TCR alpha amino acids 1-135 (SEQ ID NO: 30), cat TCR alpha amino acids 1-134 (SEQ ID NO: 31).

FIG. 23B illustrates an alignment of TCR beta chain protein sequences. The box indicates the transmembrane domain. From top to bottom: human TCR beta amino acids 1-176 (SEQ ID NO: 32), mouse TCR beta amino acids 1-173 (SEQ ID NO: 33), rat TCR beta amino acids 1-174 (SEQ ID NO: 34), pig TCR beta amino acids 1-177 (SEQ ID NO: 35), monkey TCR beta amino acids 1-177 (SEQ ID NO: 36).

FIG. 23C illustrates an alignment of CD3 delta protein sequences. The box indicates the transmembrane domain. From top to bottom: human CD3 delta amino acids 1-171 (SEQ ID NO: 37), mouse CD3 delta amino acids 1-173 (SEQ ID NO: 38), pig CD3 delta amino acids 1-171 (SEQ ID NO: 39), rat CD3 delta amino acids 1-173 (SEQ ID NO: 40), macaque CD3 delta amino acids 1-171 (SEQ ID NO: 41).

FIG. 23D illustrates an alignment of CD3 epsilon protein sequences. The box indicates the transmembrane domain. From top to bottom: human CD3 epsilon amino acids 1-207 (SEQ ID NO: 42), mouse CD3 epsilon amino acids 1-189 (SEQ ID NO: 43), pig CD3 epsilon amino acids 1-196 (SEQ ID NO: 44), rabbit CD3 epsilon amino acids 1-198 (SEQ ID NO: 45), macaque CD3 epsilon amino acids 1-198 (SEQ ID NO: 46).

FIG. 23E illustrates an alignment of CD3 gamma protein sequences. The box indicates the transmembrane domain. From top to bottom: human CD3 gamma amino acids 1-182 (SEQ ID NO: 47), mouse CD3 gamma amino acids 1-182 (SEQ ID NO: 48), pig CD3 gamma amino acids 1-182 (SEQ ID NO: 49), rat CD3 gamma amino acids 1-182 (SEQ ID NO: 50), macaque CD3 gamma amino acids 1-181 (SEQ ID NO: 51).

FIG. 24A illustrates an exemplary inhibitory TCR with an intracellular inhibitory domain fused to the TCR alpha subunit via a linker.

FIG. 24B illustrates an exemplary inhibitory TCR with an intracellular inhibitory domain fused to the TCR beta subunit via a linker.

FIG. 24C illustrates an exemplary inhibitory TCR with an intracellular inhibitory domain fused to the CD3 zeta subunits.

FIG. 25A shows a table containing illustrative engineered TCR fusion constructs.

FIG. 25B shows a table containing diagrams and domain descriptions for illustrative engineered TCR fusion constructs.

FIG. 26A is a series of diagrams (top) and a plot showing that C-terminal fusions to TCRα have negligible effect on acute TCR activity.

FIG. 26B is a series of diagrams (top) and a plot showing that C-terminal fusions to TCRβ have negligible effect on acute TCR activity.

FIG. 27A is a diagram (top) showing the generation of a CD3 knockout Jurkat-NFAT-Luciferase reporter line, and a series of plots (bottom) showing the expression of CD3E in transfected Jurkat reporter cells.

FIG. 27B is a plot showing the activity of CD3 knockout Jurkat-NFAT-Luciferase reporter cells which have been transfected with CD3 subunits with deleted ITAM domains specified at right (top), and a series of plots showing expression of TCR Vbeta 13.1 (labeled with phycoerythrin, or PE) and HLA-A*0201 NY-ESO-1 pMHC (labeled with allophycocyanin, or APC).

FIG. 27C is a diagram (top) showing the generation of a partial CD3 knockout Jurkat-NFAT-Luciferase reporter line, and a series of plots (bottom) showing expression of CD3E in transfected Jurkat reporter cells.

FIG. 27D is a plot showing the activity of CD3 partial knockout Jurkat-NFAT-Luciferase reporter cells which have been transfected with CD3 subunits with deleted ITAM domains specified at right (top), and a series of plots showing expression of TCR Vbeta 13.1 (PE) and HLA-A*0201 NY-ESO-1 pMHC (APC).

FIG. 28 is a table showing gRNAs that target CD3 delta, CD3 epsilon, CD3 gamma and CD3 zeta subunits which were used to make CD3 knockout Jurkat-NFAT-Luciferase reporter cells. gRNA sequences, from top to bottom: SEQ ID NOs: 91-102.

FIG. 29A is a pair of diagrams (top) and a plot (bottom) showing the reconstitution and activity of inhibitory (termed herein “inverter” or “inv”) TCRs in a CD3-null Jurkat NFAT luciferase line. A LIR1 intracellular inhibitory domain was fused to CD3 delta and epsilon, or CD3 delta, epsilon, gamma and zeta subunits of the TCR, and the ability of the inhibitory TCR to inhibit activation of Jurkat cells by a CD19 CAR was assayed.

FIG. 29B is a series of diagrams (top) and a plot (bottom) showing the reconstitution and activity of inhibitory TCRs in a CD3-null Jurkat NFAT luciferase line. A LIR1 intracellular domain was fused to the CD3 delta, epsilon, gamma and zeta subunits of the TCR, or the TCR alpha and beta subunits of the TCR, and the ability of inhibitory TCR or an inhibitory CAR (iCAR) to inhibit Jurkat cell activation by a CD19 CAR was assayed.

FIG. 29C is a series of diagrams (top) and a plot (bottom) showing the reconstitution and activity of inhibitory TCRs in a CD3-null Jurkat NFAT luciferase line. A LIR1 intracellular domain, or a PD1 intracellular domain was fused to the CD3 delta, CD3 epsilon, CD3 gamma, and CD3 zeta of the TCR, and the ability of inhibitory TCRs to inhibit Jurkat cell activation by a CD19 CAR was assayed. For LIR_34 (top, right diagram), the ITAM regions of the CD3 subunits were replaced by the third and fourth ITIM region of LIR1, and a few residues of the original CD3 subunits were preserved to maintain the overall backbone.

FIG. 30 is a diagram (top) and a pair of plots (bottom) showing that TCR transmembrane interactions can generate distinct activator and inhibitor TCR complexes. The positively charged K288 of TCRb interacts with corresponding negatively charged residues of CD3e (D137) and CD3g (E122). These charge pairs were charge swapped to generate distinct TCRb-CD3e/g complexes

FIG. 31 is a diagram (top) and a pair of plots (bottom) showing that TCR transmembrane interactions can generate distinct activator and inhibitor TCR complexes. The positively charged R253 of TCRa interacts with corresponding negatively charged residue of CD3ζ (D15). The charge pair was charge swapped to generate distinct TCRa-CD3ζ complexes.

FIG. 32 is a diagram (left) and a plot (right) showing that TCR transmembrane interactions can generate distinct activator and inhibitor TCR complexes. Charge-swapped transmembrane-containing TCRa and TCRb pairs well with corresponding mutant CD3z and CD3g to inhibit CD19 CAR.

### DETAILED DESCRIPTION

Chimeric antigen receptors (CAR) in which the intracellular CD3 zeta (also known as CD247 molecule) domain is fused to an intracellular signaling domain are known in the art as increasing the effectiveness of CAR T cell-based therapy. However, in comparison to CARs, TCRs have been thought to be a highly effective molecular machine, and thus not in need of further functional enhancement. As TCRs have ten-fold greater sensitivity than CARs, it has previously been thought that no intracellular signaling domain fusion was needed to achieve effective signaling. Further, the intracellular domains of T Cell Receptor Alpha (TCR alpha), T Cell Receptor Beta (TCR beta), CD3d molecule (CD3 delta), CD3e molecule (CD3 epsilon) and CD3g molecule (CD3 gamma) are strongly conserved in evolution. The highly conserved nature of the T cell subunits through evolution suggests that they may not tolerate protein engineering in those regions critical to signal transduction (i.e., the intracellular domains). Nor was it expected that adding the stimulatory domains described herein, such as LCK proto-oncogene, Src family tyrosine kinase (Lck), FYN proto-oncogene, Src family tyrosine kinase (Fyn), linker for activation of T cells (Lat), zeta chain of T cell receptor associated protein kinase 70 (Zap70) or lymphocyte cytosolic protein 2 (Slp76), would provide any benefit in enhancing TCR signaling, as such domains are already present in the TCR complex.

The present inventors have recognized that some antigens (such as sparsely expressed antigens or antigens with weak affinity for the TCR) may insufficiently trigger TCR-mediated signaling. Moreover, T cell activity is restrained in the tumor micro-environment (TME) of solid tumors due to decreased co-stimulation and increased co-inhibition. Therefore, to better access treatments directed against sparsely expressed antigens or weakly bound antigens, and to enhance functionality in the TME, the inventors have developed and tested fusion proteins for use as in engineered TCRs having one or more additional intracellular domains. Disclosed herein are fusions of intracellular domains of TCR alpha, beta and CD3 subunits with various signaling domains that enhance TCR function. Chimeric or engineered TCRs comprising these fusion proteins provide an advantage over naturally occurring TCRs in ligand sensitivity and cytolytic function.

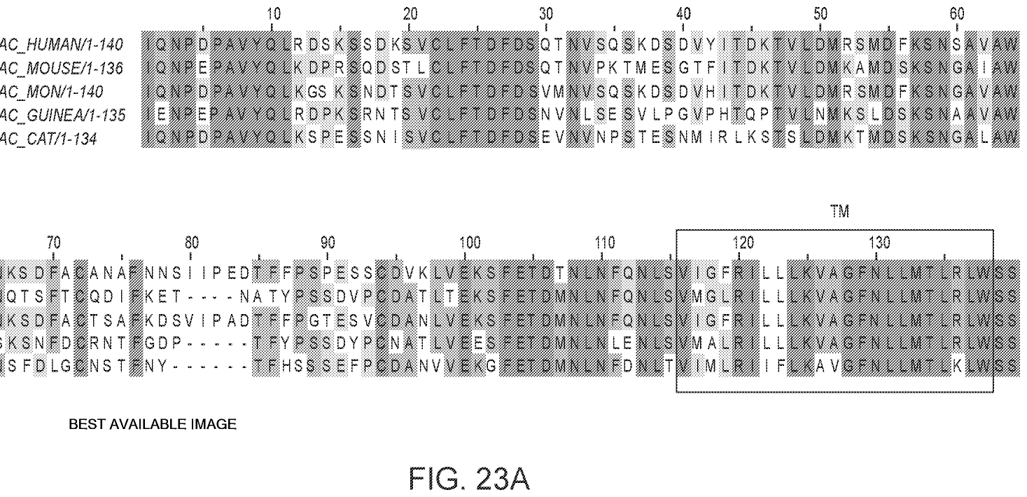

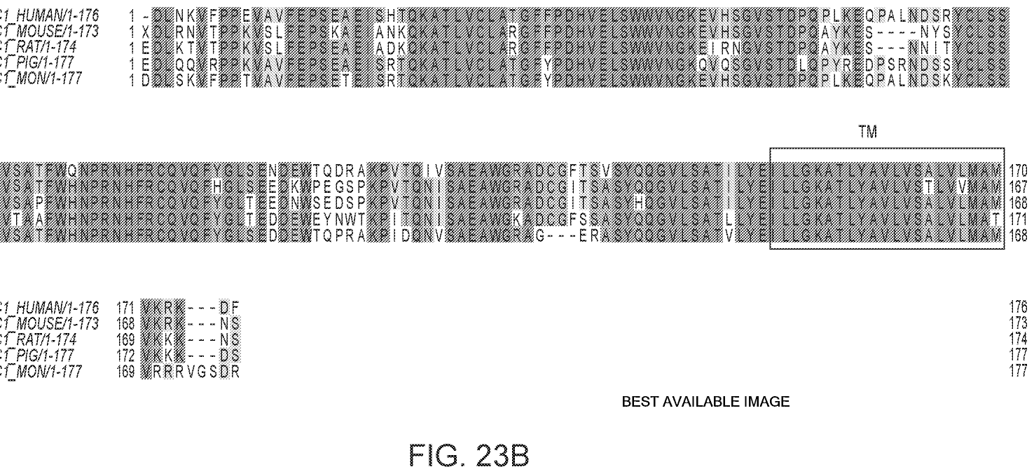

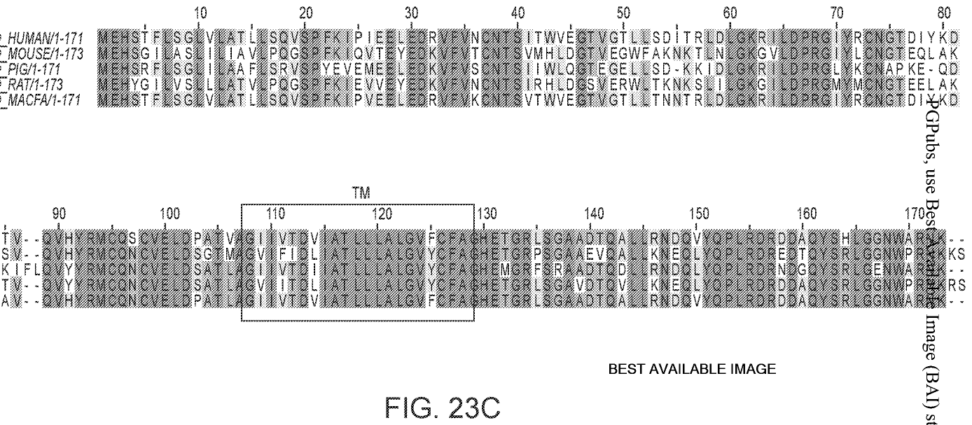

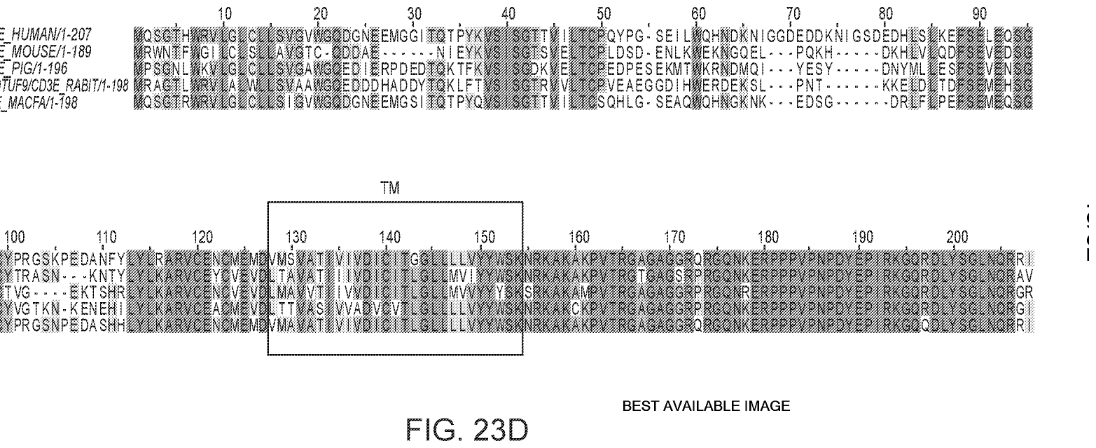

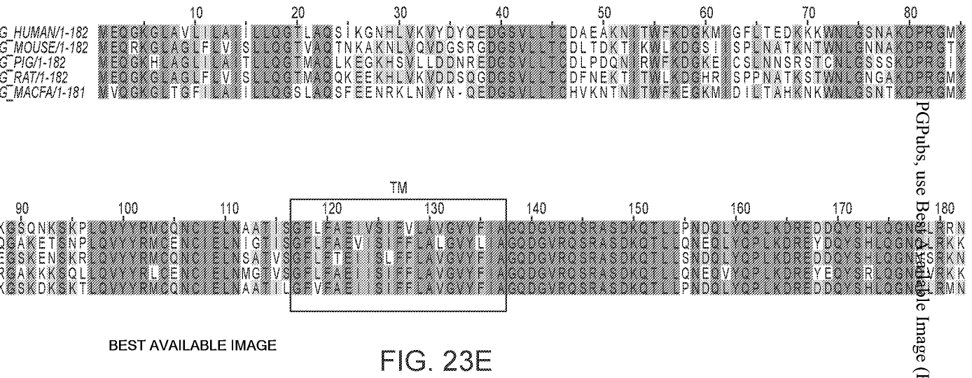

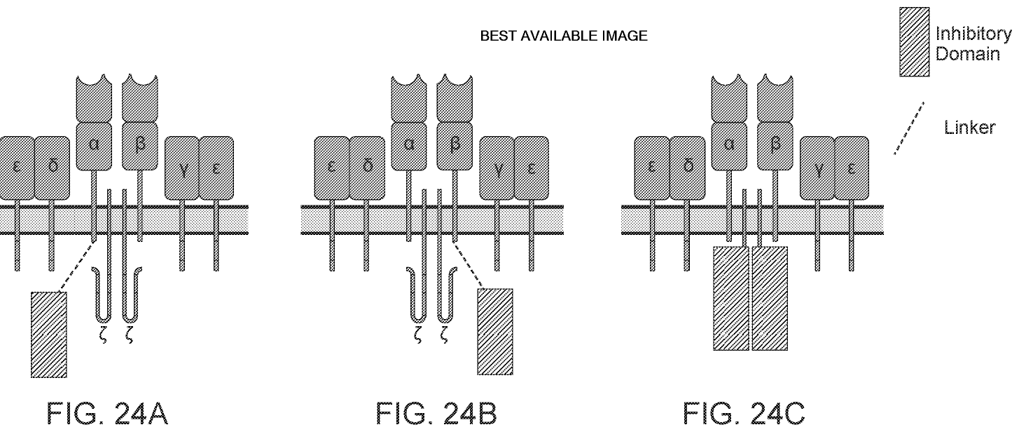

TCR subunits display high level of inter-species conservation in the C-terminal cytoplasmic domain, as shown in alignments provided in FIGS. 23A-23E. Without wishing to be bound by theory, it likely that this level of conservation is due to interaction of C terminal portions of the TCR subunit with other members of the TCR signaling pathway and other pathways. For example, the cytoplasmic domains of CD3 delta, gamma, epsilon and zeta all contain immunoreceptor tyrosine-based activation motifs (ITAMs) which can be phosphorylated by the Lck and Fyn kinases. These intracellular domains can bind to ZAP70. As a further example, the intracellular domain of CD3 epsilon contain not only an ITAM, but a basic-rich stretch (BRS) that facilitates biding of CD3 epsilon to acidic phospholipid second messengers such as Phosphatidylinositol 3-phosphate (PI(3)P), PI(4) and PI(5)P. Transgenic mice containing mutations of the BRS exhibited varying developmental defects, ranging from reduced thymic cellularity to a complete block in T cell development (DeFord-Watts, L. et. al. J. Immunol. 183:1055-1064 (2009)). CD3 epsilon also interacts with the NCK adaptor protein 1 (Nck). Therefore, given the role that the TCR subunit intracellular domains TCR signaling and the high level of conservation of these domains, one would expect that changes to these domains, including C-terminal fusions, to be deleterious to the function of the TCR.

The inventors have further recognized that engineered TCRs can be used to inhibit, or invert, endogenous TCR signaling by fusing inhibitory intracellular domains to subunits of the TCR complex. Use of such inhibitory TCRs can help treat adverse effects sometimes seen with adoptive cell therapies, such as “on-target, off-tumor” toxicities and graft versus host disease after donor cell infusion.

Immune cells (e.g., T cells) that include the disclosed engineered TCRs may have notable therapeutic benefits. In some embodiments, the engineered TCR inhibits a function of the immune cell—i.e. it may serve as an “inhibitor” TCR. The immune cell (e.g. T cell) may also include an endogenous or exogenous “activator” TCR or CAR—i.e. a TCR or CAR that activates a cellular response (T-cell response) in the response to an antigen.

In some embodiments, an immune cell of the disclosure may be an immune cell (e.g., a T cell) engineered to discriminate between tumor and non-tumor cells. In some embodiments, the immune cell may be engineered to respond to loss-of-heterozygosity (LoH) in tumor cells compared to non-tumor cells, where the subject has a heterozygous genotype at an allele encoding the target of the inhibitor TCR. For example, an engineered T cell according to the disclosure may include an activator TCR or CAR that recognizes an antigen on both tumor and non-tumor cells and an inhibitor TCR that recognizes an antigen present on non-tumor cells, but is lost in tumor cells due to the LoH event. When such an engineered T cell encounters a tumor cell, the activator TCR may bind the tumor cell, triggering an activity of the T cell (e.g., a cytotoxic response by the T cell) that may, for example, kill the tumor cell. When, in contrast, the engineered T cell encounters a non-tumor cell, though the activator TCR or CAR may bind to its antigen on the non-tumor cell, the inhibitor TCR binds its antigen on the non-tumor cell, which inhibits an activity of the T cell (e.g., the cytotoxic response), thereby protecting the non-tumor cell. Thus, engineered T cell receptors of the disclosure that include an inhibitory domain on the intracellular portion may provide significant clinical benefits when used to generate engineered T cells for therapeutic use that specifically kill tumor cells and protect non-tumor cells—for example, without limitation, in solid tumor and hematological malignancies characterized by LoH in the tumor cell.

Inhibitory TCRs can be generated by fusing heterologous intracellular domains from proteins that play a role in the inhibition of immune cell signaling pathways to any one of the TCR subunits. For example, inhibitory domains can be isolated from immune checkpoint inhibitors such as programmed cell death 1 (PD-1, also referred to as PD1) and cytotoxic T-lymphocyte associated protein 4 (CTLA-4, also referred to as CTLA4), whose intracellular domains contain motifs that mediate association with proteins that inhibit T cell signaling pathways. For example, the PD-1 intracellular domain contains an ITIM and an immunoreceptor tyrosine-based switch motif (ITSM) that mediate recruitment of SH2 domain containing phosphatases, thereby inhibiting PI3K/Akt signaling in T cells (Boussiotis et al. Cancer J. (2014) 20(4): 265-271). Similarly, the CTLA-4 intracellular domain interacts with the SH2 domain containing SHP2 protein, inhibiting Akt activation.

Inhibitory intracellular domains can also be isolated from the killer-cell immunoglobulin-like receptor (KIR) family of proteins. KIRs are a family of receptors that are expressed on the membrane of natural killer (NK) cells and some T cells. Most KIR family members have inhibitory activity. Recognition of MHC by the KIR protein suppresses cytotoxic NK cell activity. All inhibitory KIR family members have long cytoplasmic domains possessing ITIMs, which recruit protein tyrosine phosphatases that are critical for mediating inhibitory function. Exemplary KIRs whose intracellular domains can be used to generate engineered TCRs with inhibitory activity include killer cell immunoglobulin like receptor, three Ig domains and long cytoplasmic tail 2 (KIR3DL2) and killer cell immunoglobulin like receptor, three Ig domains and long cytoplasmic tail 3 (KIR3DL3).

Inhibitory intracellular domains also include domains that contain Src Homology 2 (SH2) domains. SH2 domains bind to phosphorylated tyrosine residues on proteins, for example proteins in the TCR signaling pathway, mediating downstream signaling events. Zeta-chain-associated protein kinase 70 (ZAP70, or ZAP-70) contains two SH2 domains (referred to as N and C terminal SH2 domains) which are engaged by phosphorylated ITAMs of CD3 zeta. This interaction positions ZAP70 to phosphorylate transmembrane protein linker of activated T cells (LAT). Phosphorylated LAT, in turn, serves as a docking site to which a number of signaling proteins bind, including SLP-76. Without wishing to be bound by theory, it is thought that fusing a ZAP70 domain containing one or more SH2 domains in the absence of the ZAP70 kinase domain can inhibit TCR signaling by preventing LAT phosphorylation. Similarly, kinase dead ZAP70 proteins or fragments thereof can also inhibit TCR signaling.

Inhibitory intracellular domains also include domains that are isolated or derived from members of the leukocyte immunoglobulin-like receptor (LIR) family, for example, members of the subfamily B class of LIR receptors. LIR family B receptors contain two or four extracellular immunoglobulin domains, a transmembrane domain and an intracellular domain with two to four ITIMs. These receptors are expressed on immune cells, and transduce a negative signal upon binding to MHC class I molecules. Exemplary LIR family B members include leukocyte immunoglobulin like receptor B1 (LIR1, also called LILRB1) and Leukocyte immunoglobulin-like receptor subfamily B member 3 (LILRB3).

Inhibitory domains also include domains isolated or derived from inhibitory receptors such as Fc gamma receptor IIB (FcgRIIB). FcgRIIB is a low affinity inhibitory receptor for the Fc region of immunoglobulin gamma (IgG), and inhibits the functions of activating Fc gamma receptors, such as phagocytosis and pro-inflammatory cytokine release. Phosphorylated ITIMs in the intracellular domain of FcgRIIB recruit the inositol phosphatases SHIP1 and SHIP2, which inhibit downstream signal transduction and negatively affect proliferation and survival.

Inhibitory domains also include domains isolated or derived from killer cell lectin like receptor K1 (KLRK1, or NKG2D). NKG2D is a transmembrane protein belonging to the CD94/NKG2 family of C-type lectin-like immune receptors, and is expressed on most NK cells and a subpopulation of T cells.

Unless defined otherwise, all technical and scientific terms used herein have the same meaning as commonly understood by one of ordinary skill in the art to which the invention pertains.

As used herein, “fusion protein” refers to a contiguous polypeptide or molecule containing multiple domains fused or joined together to form a novel protein or protein small molecule/carbohydrate compound. A “fusion protein” refers to a protein comprising a first polypeptide sequence and a second polypeptide sequence linked via a peptide bond or a polypeptide linker. The first or second polypeptide sequence may each individually or both be encoded by a naturally-occurring polynucleotide sequence, or a portion thereof. For example, “fusion protein” may refer to the product of transcription and translation from gene segments that have been fused together. The first or second polypeptide sequence may each individually or both be artificial polypeptide sequences and/or encoded by non-naturally occurring polynucleotide sequences. For example, the fusion protein may comprise one or more designed polypeptide sequences, heterologous linkers, or the like. Illustrative fusion proteins include an N-terminal fusion of a TCR subunit to an inhibitory intracellular domain from a different protein.

As used herein, “ligand sensitivity” refers to the amount of ligand needed to elicit a response from receptor. Receptors with increased ligand sensitivity will respond to lower amounts of ligand.

As used herein, “cytosolic function” refers to a function of a protein or protein complex that is carried out in the cytosol of a cell. For example, intracellular signal transduction cascades are cytosolic functions.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof that, when natively expressed by a T-cell, provides the primary cytoplasmic signaling sequence(s) that regulate activation of the TCR complex in a stimulatory way for at least some aspect of the T-cell signaling pathway. TCR alpha and/or TCR beta chains of wild type TCR complexes do not contain stimulatory domains and require association with CD3 subunits such as CD3 zeta to initiate signaling. In one aspect, the primary stimulatory signal is initiated by, for instance, binding of a TCR/CD3 complex with an a major histocompatibility complex (MIHC) bound to peptide, and which leads to mediation of a T-cell response, including, but not limited to, proliferation, activation, differentiation, and the like. One or more stimulatory domains, as described herein, can be fused to the intracellular portion of any one or more subunits of the TCR complex, including TCR alpha, TCR beta, CD3 delta, CD3 gamma and CD3 epsilon.

In some embodiments, a stimulatory domain can contain a primary cytoplasmic signaling sequence (also referred to as a “primary signaling domain”) that acts in a stimulatory manner, and may contain a signaling motif which is known as immunoreceptor tyrosine-based activation motif or “ITAM”. Examples of an ITAM containing primary cytoplasmic signaling sequence that is of particular use in the invention include, but are not limited to, those derived from CD3 zeta, FcR gamma, FcR beta, CD3 gamma, CD3 delta, CD3 epsilon, CD28 molecule (CD28), CD5 molecule (CD5), CD22 molecule (CD22), CD79a molecule (CD79a), CD79b molecule (CD79b), CD278 (also known as “ICOS”) and carcinoembryonic antigen related cell adhesion molecule 3 (CD66d).

In some embodiments, a stimulatory domain does not contain an ITAM signaling motif.

As used herein, a “domain capable of providing a stimulatory signal” refers to any domain that, either directly or indirectly, can provide a stimulatory signal that enhances or increases the effectiveness signaling mediated by the TCR complex to enhance at least some aspect of T-cell signaling. The domain capable of providing a stimulatory signal can provide this signal directly, for example with the domain capable of providing the stimulatory signal is a primary stimulatory domain or co-stimulatory domain. Alternatively, or in addition, the domain capable of providing the stimulatory signal can act indirectly. For example, the domain can be a scaffold that recruits stimulatory proteins to the TCR, or provide an enzymatic activity, such as kinase activity, that acts through downstream targets to provide a stimulatory signal.

An “extracellular domain”, as used herein, refers to the extracellular portion of a protein. For example, the TCR alpha and beta chains each comprise an extracellular domain, which comprise a constant and a variable region involved in peptide-MHC recognition. The “extracellular domain” can also comprise a fusion domain, for example of fusions between additional domains capable of binding to and targeting a specific antigen and the endogenous extracellular domain of the TCR subunit.

As used herein, a “domain capable of providing an inhibitory signal” refers to any domain that, either directly or indirectly, can provide an inhibitory signal that inhibits or decreases the effectiveness signaling mediated by the TCR complex. The domain capable of providing an inhibitory signal can reduce, or block, totally or partially, at least some aspect of T-cell signaling or function. The domain capable of providing an inhibitory signal can provide this signal directly, for example with the domain capable of providing the inhibitory signal provides a primary inhibitory signal. Alternatively, or in addition, the domain capable of providing the stimulatory signal can act indirectly. For example, the domain can recruit additional inhibitory proteins to the TCR, or can provide an enzymatic activity that acts through downstream targets to provide an inhibitory signal.

As used herein an “immunoreceptor tyrosine-based inhibitory motif” or “ITIM” refers to a conserved sequence of amino acids with a consensus sequence of S/I/V/LxYxxI/V/L (SEQ ID NO: 115) that is found in the cytoplasmic tails of many inhibitory receptors of the immune system. After ITIM-possessing inhibitory receptors interact with their ligand, the ITIM motif is phosphorylated, allowing the inhibitory receptor to recruit other enzymes, such as the phosphotyrosine phosphatases SHP-1 and SHP-2, or the inositol-phosphatase called SHIP.

As used herein, an “immunoreceptor tyrosine-based switch motif or “ITSM” refers to a conserved sequence of amino acids with a consensus sequence of TxYxx(V/I) (SEQ ID NO: 116) that is found in the intracellular domain of some immune receptors, such as SLAM/CD150 and related receptors. ITSM can be either activating or inhibitory, depending on context and the type of proteins that bind to the ITSM.

As used herein, a “Src Homology 2” or “SH2” domain refers to a structurally conserved protein domain contained within the Src oncoprotein and other intracellular signal-transducing proteins. SH2 domains allow the proteins that contain them to bind phosphorylated tyrosine residues on other proteins, and frequently aid in signal transduction in receptor tyrosine kinase signaling pathways.

In some embodiments, the domain capable of providing an inhibitory signal is selected from the intracellular domains of CTLA4, PD1, lymphocyte activating 3 (LAG3), hepatitis A virus cellular receptor 2 (HAVCR2, also referred to as TIM3), KIR2DL2, KIR3DL2, LILRB1 (LIR1), T cell immunoreceptor with Ig and ITIM domains (TIGIT), CEA cell adhesion molecule 1 (CEACAM1), colony stimulating factor 1 receptor (CSF1R), CD5, CD96 molecule (CD96), CD22 molecule (CD22) and leukocyte associated immunoglobulin like receptor 1 (LAIR1) or functional fragments thereof. Further inhibitory domains include those described in International Application Pub. No. WO2016075612A1. In some embodiments, the domain capable of providing an inhibitory signal is selected from the intracellular domain of a leukocyte immunoglobulin-like receptors (LIR) or a functional fragment thereof. In some embodiments, the domain is selected from the intracellular domain of LILRB1, LILRB2, LILRB3, LILRB4, and LILRB5, or functional fragments thereof. In some embodiments, the domain is selected from the intracellular domain of PIR-B or a functional fragment thereof. In some embodiments, the domain is the intracellular domain of LILRB1. In some embodiments, the domain is a functional fragment of the intracellular domain of LILRB1. In some embodiments, the domain capable of providing an inhibitory signal is selected from the intracellular domain of an inhibitory killer cell immunoglobulin like receptor (KIR) or a functional fragment thereof. In some embodiments, the domain is selected from the intracellular domain of KIR2DL1, KIR2DL2, KIR2DL3, KIR2DL4, KIR2DL5A, KIR2DL5B, KIR3DL1, KIR3DL2, and KIR3DL3, or functional fragments thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

As used herein, the term “chimeric antigen receptor (CAR)” refers to an artificial T Cell Receptor that grafts particular specificity onto an immune cell. Typically, CARs comprise an intracellular activation domain, a transmembrane domain, and an extracellular domain comprising an antigen binding region and optionally a hinge between the antigen binding domain and transmembrane domain. In particular aspects, CARs comprise fusions of single-chain variable fragments (scFv) derived from monoclonal antibodies, fused to CD3 a transmembrane domain and endodomain. CAR architectures will be known to persons of ordinary skill in the art.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

### T Cell Receptors (TCRs) and TCR Subunits

The disclosure provides fusion proteins comprising a subunit of a T-cell receptor (TCR), or a fragment or derivative thereof, fused to one or more intracellular domains that are capable of providing an inhibitory signal, thereby reducing or blocking TCR function. By fusing a one or more subunits of the TCR to intracellular domain(s), transmembrane domain(s), or extracellular domain(s) to an intracellular domain capable of providing an inhibitory signal, the fusion proteins of the disclosure are able to decrease or inhibit activity of the TCR complex. In some embodiments, the fusion proteins comprise the transmembrane domain of the TCR subunit fused to one or more domains capable of providing an inhibitory signal. In some embodiments, the fusion proteins comprise the extracellular domain and transmembrane domain of the TCR subunit fused to one or more domains capable of providing an inhibitory signal. In some embodiments, the fusion proteins comprise the extracellular domain, transmembrane domain and intracellular domain of the TCR subunit fused to one or more domains capable of providing an inhibitory signal. In some embodiments, the fusion proteins comprise the extracellular domain of the TCR subunit fused to a transmembrane domain and intracellular domain that are both isolated or derived from the same protein, and the intracellular domain is capable of providing an inhibitory signal (e.g., a transmembrane domain and intracellular domain from PD-1). In some embodiments, the fusion protein comprises a hinge region isolated or derived from the same protein as the intracellular domain and transmembrane domain, for example an ITIM containing protein. In some embodiments, the fusion protein comprises a intracellular cytoplasmic domain, a transmembrane domain, and an extracellular domain or a portion thereof isolated or derived from the same protein, for example an ITIM containing protein. In some embodiments, the fusion proteins further comprise an additional domain, for example an antigen binding domain, fused to the extracellular domain or transmembrane domain of the fusion protein.

The disclosure further provides fusion proteins comprising a subunit of a T-cell receptor (TCR), or a fragment or derivative thereof, fused to one or more intracellular domains that are capable of providing a stimulatory signal, thereby enhancing TCR function. By fusing a one or more subunits of the TCR to intracellular domain(s) capable of providing a stimulatory signal, the fusion proteins of the disclosure are able to increase activity of the TCR complex. In some embodiments, the fusion proteins comprise the transmembrane domain of the TCR subunit fused to one or more domains capable of providing a stimulatory signal. In some embodiments, the fusion proteins comprise the extracellular domain and transmembrane domain of the TCR subunit fused to one or more domains capable of providing a stimulatory signal. In some embodiments, the fusion proteins comprise the extracellular domain, transmembrane domain and intracellular domain of the TCR subunit fused to one or more domains capable of providing a stimulatory signal. In some embodiments, the fusion proteins further comprise an additional domain, for example an antigen binding domain, fused to the extracellular domain or transmembrane domain of the fusion protein.

TCR subunits include TCR alpha, TCR beta, CD3 zeta, CD3 delta, CD3 gamma and CD3 epsilon. Any one or more of TCR alpha, TCR beta chain, CD3 gamma, CD3 delta or CD3 epsilon, CD3 zeta, or fragments or derivative thereof, can be fused to one or more domains capable of providing a stimulatory or inhibitory signal of the disclosure, thereby enhancing or altering (e.g., inverting) TCR function and activity.

### Extracellular Domain

In some embodiments, the fusion proteins of the disclosure comprise an extracellular domain, a transmembrane domain and an intracellular domain. In some embodiments, the intracellular domain comprises one or more exogenous domains, for example as C-terminal fusions.

In some embodiments, the fusion protein comprises a TCR alpha or TCR beta extracellular domain.

In some embodiments, the fusion protein comprises a TCR alpha or TCR beta extracellular domain, transmembrane domain and first intracellular domain.

In some embodiments, the extracellular domain of TCR alpha and/or beta comprises an antigen binding domain isolated or derived from the TCR alpha and/or TCR beta subunit of a TCR. In some embodiments, the extracellular domain of TCR alpha and/or beta comprises a constant region and variable region. The extracellular domains of TCR alpha and TCR beta engage antigens using complementarity-determining regions (CDRs) in the variable region. TCR alpha and TCR beta each contain three complement determining regions (CDR1a, CDR2a, CDR3a and CDR1b, CDR2b and CDR3b, respectively).

Extracellular domains of TCR alpha and/or TCR beta may be derived either from a natural or from a recombinant source.

In some embodiments, for example in native TCRs, the TCR alpha and TCR beta CDRs are germ-line encoded in the case of CDR1 and CDR2, or the result of somatic rearrangement, in the case of CDR3. The TCRα gene locus contains variable (V) and joining (J) gene segments (Vβ and Jβ), whereas the TCRβ locus contains a D gene segment in addition to Vα and Jα segments. Accordingly, the α chain is generated from VJ recombination and the β chain is involved in VDJ recombination.

In some embodiments, the extracellular domain of TCR alpha and/or beta comprises a constant region and variable region. In some embodiments, the constant region comprises one or more mutations that stabilize a heterodimeric TCR α/β via a non-native disulfide bridge between the constant domains of TCR alpha and TCR beta. In some embodiments, the TCR alpha subunit comprises a mutation of threonine to cysteine a position of exon 1 according to the numbering described in Garboczi et al, Nature, 1996, 384(6605): 134-14. In some embodiments, the TCR alpha subunit comprises a mutation of T to C at position 10 in a sequence of DSDVYITDKTVLDMRSMDFK (SEQ ID NO: 117) or similar homologous sequence in the TCR alpha subunit. In some embodiments, the TCR alpha subunit comprises a mutation of T to C at position 10 in a sequence of DSDVYITDKTVLDMRSMDFK (SEQ ID NO: 117) in the TCR alpha subunit. Exemplary mutations that stabilize TCR α/β heterodimers are described in WO2004074322, the contents of which are incorporated herein by reference in their entirety. In some embodiments, for example in recombinant TCR alpha and/or beta extracellular domains, the extracellular domain can comprise a chimeric or exogenous extracellular domain. For example the extracellular domain of TCR alpha and/or beta may comprise CDRs specific to particular peptide-MHC complex.

In some embodiments, the extracellular domain of a subunit of the engineered TCR of the disclosure comprises a sequence of an antibody or an antibody fragment, for example an antigen binding domain of an antibody, or CDR sequences isolated or derived form an antibody, including single chain variable fragments (scFv). Antigen binding domains such as ScFv can be fused to one or more subunits of an engineered TCR described herein, including TCR alpha, TCR beta, CD3 delta, CD3 epsilon and CD3 zeta. Exemplary engineered TCRs comprising ScFv fused to the extracellular domain of a TCR subunit are described in U.S. Pat. No. 10,208,285, the contents of which are incorporated herein by reference in their entirety. In some embodiments, the extracellular domain of the engineered TCR that comprises a sequence of an antibody or an antibody fragment is a TCR alpha subunit or a TCR beta subunit. In some embodiments, both the TCR alpha subunit and the TCR beta subunit comprise a sequence of an antibody or an antibody fragment.

A TCR subunit, for example TCR alpha and/or beta comprising an antibody or antibody fragment thereof may exist in a variety of forms. For example, the antigen binding domain can be expressed as part of a contiguous polypeptide chain including, for example, a single domain antibody fragment (sdAb) or heavy chain antibodies HCAb, a single chain antibody (scFv) derived from a murine, humanized or human antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the antigen binding domain of a TCR of the disclosure comprises an antibody fragment. In further aspects, the TCR comprises an antibody fragment that comprises a scFv or an sdAb.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The term “antibody light chain,” refers to the smaller of the two types of polypeptide chains present in antibody molecules in their naturally occurring conformations. Kappa (“K”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

TCRs comprising all forms of antigen binding domains known in the art are envisaged as within the scope of the disclosure.

In some embodiments, the extracellular domain comprises a domain targeting an antigen fused to an extracellular domain of a TCR subunit. In some embodiments, the extracellular domain comprises a domain targeting an antigen fused to an extracellular domain of TCR alpha. In some embodiments, the extracellular domain comprises a domain targeting an antigen fused to an extracellular domain of TCR beta. In some embodiments, both the extracellular domain fused to TCR alpha and the extracellular domain fused to TCR beta target the same antigen. Exemplary extracellular domains of the disclosure comprise an extracellular domain targeting the NY-ESO-1 (1G4 clone) antigen fused to TCR alpha. In some embodiments, the extracellular domain fused to TCR alpha comprises a sequence of

```
(SEQ ID NO: 118)
    1 METLLGLLIL WLQLQWVSSK QEVTQIPAAL
        SVPEGENLVL NCSFTDSAIY NLQWFRQDPG
  61 KGLTSLLLIQ SSQREQTSGR LNASLDKSSG
        RSTLYIAASQ PGDSATYLCA VRPLYGGSYI
121 PTFGRGTSLI VHPYIQNPDP AVYQLRDSKS
        SDKSVCLFTD FDSQTNVSQS KDSDVYITDK
181 CVLDMRSMDF KSNSAVAWSN KSDFACANAF
        NNSIIPEDTF FPSPESSCDV KLVEKSFETD
241 TNLNFQNLS.
```

Exemplary extracellular domains of the disclosure comprise an extracellular domain targeting the NY-ESO-1 1G4 antigen fused to TCR beta. In some embodiments, the extracellular domain fused TCR beta comprises a sequence of

```
(SEQ ID NO: 119)
    1 MSIGLLCCAA LSLLWAGPVN AGVTQTPKFQ
        VLKTGQSMTL QCAQDMNHEY MSWYRQDPGM
  61 GLRLIHYSVG AGITDQGEVP NGYNVSRSTT
        EDFPLRLLSA APSQTSVYFC ASSYVGNTGE
121 LFFGEGSRLT VLEDLKNVFP PEVAVFEPSE
        AEISHTQKAT LVCLATGFYP DHVELSWWVN
181 GKEVHSGVCT DPQPLKEQPA LNDSRYCLSS
        RLRVSATFWQ NPRNHFRCQV QFYGLSENDE
241 WTQDRAKPVT QIVSAEAWGR ADCGFTSESY
        QQGVLSA.
```

### Transmembrane Domain

The disclosure provides fusion proteins comprising a transmembrane domain, and a first intracellular domain capable of providing a stimulatory or inhibitory signal. The disclosure provides fusion proteins comprising a transmembrane domain, and a first intracellular domain capable of providing an inhibitory signal. In some embodiments, the fusion protein comprises a second intracellular domain, for example from TCR alpha, TCR beta, CD3 delta, CD epsilon or CD3 gamma.

A “transmembrane domain”, as used herein, refers to a domain of a protein that spans membrane of the cell. Transmembrane domains typically consist predominantly of non-polar amino acids, and may traverse the lipid bilayer once or several times. Transmembrane domains usually comprise alpha helices, a configuration which maximizes internal hydrogen bonding.

Transmembrane domains isolated or derived from any source are envisaged as within the scope of the fusion proteins of the disclosure.

In some embodiments, the transmembrane domain is one that is associated with one of the other domains of the fusion protein, or isolated or derived from the same protein as one of the other domains of the fusion protein. In some embodiments, the transmembrane domain and the second intracellular domain are from the same protein, for example a TCR complex subunit such as TCR alpha, TCR beta, CD3 delta, CD3 epsilon, CD3 gamma or CD3 zeta. In some embodiments, the extracellular domain, the transmembrane domain and the second intracellular domain are from the same protein, for example a TCR complex subunit such as TCR alpha, TCR beta, CD3 delta, CD3 epsilon, CD3 gamma or CD3 zeta.

In some embodiments, a fusion protein can be designed to comprise a transmembrane domain that is heterologous to either an extracellular domain and/or one or more intracellular domains of the fusion protein.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region).

In some embodiments, the transmembrane domain can be selected or modified by amino acid substitution to avoid binding of such domains to the transmembrane domains of the same or different surface membrane proteins, e.g., to minimize interactions with other members of the receptor complex.

In some embodiments, the transmembrane domain is capable of homodimerization or heterodimerization with another protein on a cell surface. For example, a transmembrane domain of a fusion protein of disclosure may be able to form TCR alpha/beta heterodimers, CD3 epsilon/delta heterodimers or CD3 gamma/epsilon heterodimers in a cell.

In some embodiments, the transmembrane domain is mutated to favor the formation of specific TCR complexes, for example by altering the charge of specific amino acid residues in the transmembrane domains of TCR complex subunits. Exemplary amino acid residues that may be altered include K288 of TCR beta, D137 of CD3 epsilon, E122 of CD3 gamma, D15 of CD3 zeta, R253 of TCR alpha and E122 of CD3 gamma, which are described below.

In some embodiments, the transmembrane domain of a fusion protein of the disclosure forms a heterodimer with a native protein in a cell.

In some embodiments, the transmembrane domain of a fusion protein of the disclosure forms a heterodimer with a second fusion protein in a cell. In some embodiments, the amino acid sequence of the transmembrane domain may be modified or substituted so as to minimize interactions with the binding domains of the native binding partner present in the same cell.

The transmembrane domain may be derived either from a natural or from a recombinant source. Where the source is natural, the domain may be derived from any membrane-bound or transmembrane protein.

In some embodiments, the transmembrane domain is capable of signaling to the intracellular domain(s) whenever the TCR complex has bound to a target. A transmembrane domain of particular use in this invention may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the transmembrane domain can be attached to the extracellular region of the fusion protein, e.g., the antigen binding domain of the TCR alpha or beta chain, via a hinge, e.g., a hinge from a human protein. For example, in one embodiment, the hinge can be a human immunoglobulin (Ig) hinge, e.g., an IgG4 hinge, or a CD8a hinge.

In some embodiments, the hinge is isolated or derived from CD8a or CD28. In some embodiments, the CD8a hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 5). In some embodiments, the CD8a hinge comprises SEQ ID NO: 5. In some embodiments, the CD8a hinge consists essentially of SEQ ID NO: 5. In some embodiments, the CD8a hinge is encoded by a nucleotide sequence having at least 80% identity, at least 90% identity, at least 95% identity, at least 99% identity or is identical to a sequence of SEQ ID NO: 22. In some embodiments, the CD8a hinge is encoded by SEQ ID NO: 22.

In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP (SEQ ID NO: 23). In some embodiments, the CD28 hinge comprises or consists essentially of SEQ ID NO: 23. In some embodiments, the CD28 hinge is encoded by a nucleotide sequence having at least 80% identity, at least 90% identity, at least 95% identity, at least 99% identity or is identical to a sequence of SEQ ID NO: 24. In some embodiments, the CD28 hinge is encoded by SEQ ID NO: 24.

In some embodiments, the transmembrane comprises a CD3 delta transmembrane domain. In some embodiments, the CD3 delta transmembrane domain comprises an amino acid sequence having at least 85% identity, at least 90% identity, at least 95% identity, at least 96% identity, at least 97% identity, at least 98% identity, at least 99% identity or is identical to a sequence of: GIIVTDVIATLLLAGVFCFA (SEQ ID NO: 6). In some embodiments, the CD3 delta transmembrane domain comprises, or consists essentially of, GIIVTDVIATLLLAGVFCFA (SEQ ID NO: 6). In some embodiments, the CD3 delta transmembrane domain is encoded by a nucleotide sequence of SEQ ID NO: 120.

In some embodiments, the transmembrane comprises a CD3 epsilon transmembrane domain. In some embodiments, the CD3 epsilon transmembrane domain comprises an amino acid sequence having at least 85% identity, at least 90% identity, at least 95% identity, at least 96% identity, at least 97% identity, at least 98% identity, at least 99% identity or is identical to a sequence of: VMSVATIVIVDICITGGLLLLVYYWS (SEQ ID NO: 7). In some embodiments, the CD3 epsilon transmembrane domain comprises, or consists essentially of, SEQ ID NO: 7. In some embodiments, the CD3 epsilon transmembrane domain is encoded by a nucleotide sequence of SEQ ID NO: 109.

In some embodiments, the transmembrane comprises a CD3 gamma transmembrane domain. In some embodiments, the CD3 gamma transmembrane domain comprises an amino acid sequence having at least 85% identity, at least 90% identity, at least 95% identity, at least 96% identity, at least 97% identity, at least 98% identity, at least 99% identity or is identical to a sequence of: GFLFAEIVSIFVLAVGVYFIA (SEQ ID NO: 8). In some embodiments, the CD3 gamma transmembrane domain comprises, or consists essentially of, SEQ ID NO: 8. In some embodiments, the CD3 gamma transmembrane domain is encoded by a sequence of SEQ ID NO: 121.

In some embodiments, the transmembrane comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of: VIGFRILLLKVAGFNLLMTLRLW (SEQ ID NO: 9). In some embodiments, the TCR alpha transmembrane domain comprises, or consists essentially of, SEQ ID NO: 9. In some embodiments, the TCR alpha transmembrane domain is encoded by a sequence of SEQ ID NO: 122.

In some embodiments, the transmembrane comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of: TILYEILLGKATLYAVLVSALVL (SEQ ID NO: 10). In some embodiments, the TCR beta transmembrane domain comprises, or consists essentially of, SEQ ID NO: 10. In some embodiments, the TCR beta transmembrane domain is encoded by a sequence of SEQ ID NO: 123.

In some embodiments, for example those embodiments wherein the first intracellular domain is capable of providing an inhibitory signal, the transmembrane domain and the intracellular domain are both isolated or derived from the same protein. For example, the transmembrane domain and the first intracellular domain are both isolated or derived from the same inhibitory immunoreceptor.

As a further example, and in those embodiments where the first intracellular domain is capable of providing an inhibitory signal, the transmembrane domain is isolated or derived from the TCR subunit and the intracellular domain is derived from an heterologous protein (a protein not part of a wild type TCR), for example a protein with an ITIM-containing intracellular domain.

In some embodiments, both the transmembrane domain and first, inhibitory intracellular domain are isolated or derived from programmed cell death 1 (PD-1), cytotoxic T-lymphocyte associated protein 4 (CTLA-4), killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), zeta chain of T cell receptor associated protein kinase 70 (ZAP70), leukocyte immunoglobulin like receptor B1 (LIR1), Fc gamma receptor IIB (FcgRIIB), or killer cell lectin like receptor K1 (NKG2D). In some embodiments, both the transmembrane domain and first, inhibitory intracellular domain are isolated or derived from a leukocyte immunoglobulin-like receptors (LIR) or a functional fragment thereof. In some embodiments, both the transmembrane domain and first, inhibitory intracellular domain are isolated or derived from LILRB1, LILRB2, LILRB3, LILRB4, and LILRB5, or functional fragments thereof. In some embodiments, both the transmembrane domain and first, inhibitory intracellular domain are isolated or derived from PIR-B or a functional fragment thereof. In some embodiments, both the transmembrane domain and first, inhibitory intracellular domain are isolated or derived from LILRB1. In some embodiments, both the transmembrane domain and first, inhibitory intracellular domain are isolated or derived from a functional fragment of LILRB1. In some embodiments, both the transmembrane domain and first, inhibitory intracellular domain are isolated or derived from an inhibitory killer cell immunoglobulin like receptor (KIR) or a functional fragment thereof. In some embodiments, both the transmembrane domain and first, inhibitory intracellular domain are isolated or derived from KIR2DL1, KIR2DL2, KIR2DL3, KIR2DL4, KIR2DL5A, KIR2DL5B, KIR3DL1, KIR3DL2, and KIR3DL3, or functional fragments thereof.

In some embodiments, the transmembrane domain is isolated or derived from a TCR subunit such as TCR alpha, TCR beta, CD3 delta, CD3 epsilon, CD3 gamma or CD3 zeta, and the first, inhibitory intracellular domain is isolated or derived from PD-1, CTLA-4, KIR3DL2, KIR3DL3, ZAP70, LIR1, FcgRIIB, or NKG2D. In some embodiments, the transmembrane domain is isolated or derived from a TCR subunit, and the first, inhibitory intracellular domain is isolated or derived from a leukocyte immunoglobulin-like receptor (LIR), such as LIR1, or a functional fragment thereof. In some embodiments, the transmembrane domain is isolated or derived from a TCR subunit, and the first, inhibitory intracellular domain is isolated or derived from LIR1, PD-1, KIR3DL2, or a functional fragment thereof.

In some embodiments, the ZAP70 transmembrane domain and intracellular domain comprise a ZAP70 SH2 domain, for example the N terminal SH2 domain, the C terminal SH2 domain, or both. In some embodiments, the ZAP70 is kinase inactive. Any mutation inactivating ZAP70 kinase activity is envisaged as within the scope of the disclosure. Exemplary ZAP70 kinase inactive mutations include a substitution of alanine for lysine at position 369 of SEQ ID NO: 17.

In some embodiments, wherein the first intracellular domain is capable of providing an inhibitory signal, the transmembrane domain comprises a TCR alpha transmembrane domain, a TCR beta transmembrane domain, a CD3 delta transmembrane domain, a CD3 epsilon transmembrane domain, a CD3 gamma transmembrane domain, a CD3 zeta transmembrane domain or a functional derivative thereof.

### Intracellular Domain

The disclosure provides fusion proteins comprising an intracellular domain that can provide an inhibitory or stimulatory signal to an immune cell. An “intracellular domain,” as the term is used herein, refers to an intracellular portion of a protein.

In some embodiments, the intracellular domain comprises a C-terminal fusion of one or more domains capable of providing an inhibitory signal to a transmembrane domain. In some embodiments, the intracellular domain comprises a first intracellular domain capable of providing a inhibitory signal and a second intracellular domain. In some embodiments, the first intracellular domain capable of providing an inhibitory signal is selected from the group consisting of a PD-1, CTLA-4, KIR3DL2, KIR3DL3, ZAP70, LIR1, FcgRIIB, an NKG2D intracellular domain. In some embodiments, the first intracellular domain capable of providing an inhibitory signal comprises a LIR1 intracellular domain. In some embodiments, the second intracellular domain is selected from the group consisting of TCR alpha, TCR beta, CD3 delta, CD3 gamma, CD3 epsilon and CD3 zeta intracellular domains.

In some embodiments, the intracellular domain comprises a C-terminal fusion of one or more domains capable of providing a stimulatory signal to a transmembrane domain. In some embodiments, the intracellular domain comprises a first intracellular domain capable of providing a stimulatory signal and a second intracellular domain. In some embodiments, the first intracellular domain capable of providing a stimulatory signal is selected from the group consisting of a CD28 molecule (CD28) domain, a LCK proto-oncogene, Src family tyrosine kinase (Lck) domain, a TNF receptor superfamily member 9 (4-1BB) domain, a TNF receptor superfamily member 18 (GITR) domain, a CD4 molecule (CD4) domain, a CD8a molecule (CD8a) domain, a FYN proto-oncogene, Src family tyrosine kinase (Fyn) domain, a zeta chain of T cell receptor associated protein kinase 70 (ZAP70) domain, a linker for activation of T cells (LAT) domain, and a lymphocyte cytosolic protein 2 (SLP76) domain. In some embodiments, the second intracellular domain is selected from the group consisting of TCR alpha, TCR beta, CD3 delta, CD3 gamma and CD3 epsilon intracellular domains.

In some embodiments, an intracellular domain comprises at least one intracellular signaling domain, for example an intracellular signaling domain generates a signal that promotes a function a cell, for example an immune effector function of a TCR containing cell, e.g., a TCR-expressing T-cell, or inhibits the function of a TCR-expressing T-cell. In some embodiments, the intracellular domain of the fusion proteins of the disclosure includes at least one intracellular signaling domain. For example, the intracellular domains of CD3 gamma, delta or epsilon comprise signaling domains.

In some embodiments, the extracellular domain, transmembrane domain and a second intracellular domain are isolated or derived from the same protein, for example T-cell receptor (TCR) alpha, TCR beta, CD3 delta, CD3 gamma, CD3 epsilon or CD3 zeta.

Examples of intracellular domains for use in the fusion proteins of the disclosure include the cytoplasmic sequences of the TCR alpha, TCR beta, CD3 delta, CD3 gamma, CD3 epsilon and CD3 zeta, and the intracellular signaling co-receptors that act in concert to initiate signal transduction following antigen receptor engagement, as well as any derivative or variant of these sequences and any recombinant sequence that has the same functional capability.

In some embodiments, the intracellular signaling domain comprises a primary intracellular signaling domain. Exemplary primary intracellular signaling domains include those derived from the proteins responsible for primary stimulation, or antigen dependent stimulation.

An intracellular signaling domain can be responsible for activation of at least one of the normal effector functions of the immune cell in which the fusion protein has been introduced. The term “effector function” refers to a specialized function of a cell. Effector function of a T-cell, for example, may be cytolytic activity or helper activity including the secretion of cytokines. Thus the term “intracellular signaling domain” refers to the portion of a protein which transduces the effector function signal and directs the cell to perform a specialized function. Alternatively, or in addition, an intracellular domain, if inhibitory, can be responsible for inhibiting at least one of the normal effector functions of the immune cell in which the fusion protein has been introduced.

While in some cases the entire intracellular signaling domain can be employed, in many cases it is not necessary to use the entire intracellular signaling domain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the invention include those of CD3 zeta, FcR gamma, FcR beta, CD3 gamma, CD3 delta, CD3 epsilon, CD5, CD22, CD79a, CD79b, and CD66d. In one embodiment, a TFP of the invention comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs. An exemplary ITAM comprises a pair of YxxL/I (SEQ ID NO: 125) motifs separated by a defined interval, for example (YxxL/I-X6-8-YXXL/I, SEQ ID NOs: 237-239)).

Examples of primary intracellular signaling domains include, but are not limited to CD3 delta, CD3 gamma and CD3 epsilon intracellular domains.

In some embodiments, the intracellular domain of the fusion proteins of the disclosure does not include an intracellular signaling domain. For example, the intracellular domains of TCR alpha and TCR beta are generally lacking in a signaling domain.

In some embodiments, the intracellular domain of a fusion protein described herein comprises one or more domains capable of providing aa stimulatory signal.

For example, a fusion protein of the disclosure can comprise CD3 delta, CD3 gamma or CD3 epsilon intracellular domain fused to one or more additional signaling domains capable of providing a stimulatory signal as described herein. As a further example, a fusion protein of the disclosure can comprise a TCR alpha or TCR beta intracellular domain fused to one or more signaling domains. Alternatively, a fusion of the disclosure can comprise an intracellular domain fused to a TCR alpha or TCR beta transmembrane domain. The one or more intracellular domains capable of providing a stimulatory signal can be selected from the group consisting of CD28 domain, a Lck domain, a 4-1BB domain, a GITR domain, a CD4 domain, a CD8a domain, a Fyn domain, a ZAP70 domain, a LAT domain, and a SLP76 domain.

In some embodiments, an intracellular domain can be a minimal intracellular domain. For example, the intracellular domain of wild type TCR alpha consists of only two amino acids.

In some embodiments, the intracellular domain comprises a CD3 delta intracellular domain. In some embodiments, the CD3 delta intracellular domain comprises an amino acid sequence having at least 85% identity, at least 90% identity, at least 95% identity, at least 96% identity, at least 97% identity, at least 98% identity, at least 99% identity or is identical to a sequence of: GHETGRLSGAADTQALLRNDQVYQPLRDRDDAQYSHLGGNWARNKGGSRSKRSRLLH SDYMNMTPRRPGPTRKHYQPYAPPRDFAAYRS (SEQ ID NO: 1). In some embodiments, the CD3 delta intracellular domain comprises or consists essentially of, SEQ ID NO: 1. In some embodiments, the CD3 delta intracellular domain is encoded by a sequence of SEQ ID NO: 126.

In some embodiments, the intracellular domain comprises a CD3 epsilon intracellular domain. In some embodiments, the CD3 epsilon intracellular domain comprises an amino acid sequence having at least 85% identity, at least 90% identity, at least 95% identity, at least 96% identity, at least 97% identity, at least 98% identity, at least 99% identity or is identical to a sequence of: KNRKAKAKPVTRGAGAGGRQRGQNKERPPPVPNPDYEPIRKGQRDLYSGLNQRRIGGS RSKRSRLLHSDYMNMTPRRPGPTRKHYQPYAPPRDFAAYRS (SEQ ID NO: 2). In some embodiments, the CD3 epsilon intracellular domain comprises or consists essentially of, SEQ ID NO: 2. In some embodiments, the CD3 epsilon intracellular domain is encoded by a sequence of SEQ ID NO: 127.

In some embodiments, the intracellular domain comprises a CD3 gamma intracellular domain. In some embodiments, the CD3 gamma intracellular domain comprises an amino acid sequence having at least 85% identity, at least 90% identity, at least 95% identity, at least 96% identity, at least 97% identity, at least 98% identity, at least 99% identity or is identical to a sequence of: GQDGVRQSRASDKQTLLPNDQLYQPLKDREDDQYSHLQGNQLRRNGGSRSKRSRLLHS DYMNMTPRRPGPTRKHYQPYAPPRDFAAYRS (SEQ ID NO: 3). In some embodiments, the CD3 gamma intracellular domain comprises, or consists essentially of, SEQ ID NO: 3. In some embodiments, the CD3 gamma intracellular domain is encoded by a sequence of SEQ ID NO: 128.

In some embodiments, the intracellular domain comprises a TCR alpha intracellular domain. In some embodiments, a TCR alpha intracellular domain comprises Ser-Ser. In some embodiments, a TCR alpha intracellular domain is encoded by a sequence of TCCAGC.

In some embodiments, the intracellular domain comprises a TCR beta intracellular domain. In some embodiments, the TCR beta intracellular domain comprises an amino acid sequence having at least 80% identity, at least 90% identity, or is identical to a sequence of: MAMVKRKDSR (SEQ ID NO: 4). In some embodiments, the TCR beta intracellular domain comprises, or consists essentially of SEQ ID NO: 4. In some embodiments, the TCR beta intracellular domain is encoded by a sequence of SEQ ID NO: 129.

### Fusion Proteins

The inventors have found that fusing a domain capable of providing an inhibitory signal to one or more subunits of the TCR can change the TCR signal from an activator signal to an inhibitory signal when the fusion protein is introduced into an immune cell.

In some embodiments, the domain capable of providing an inhibitory signal is selected from the intracellular domains of CTLA4, LAG3, HAVCR2 (TIM3), KIR2DL2, KIR3DL2, LILRB1, TIGIT, CEACAM1, CSF1R, CD5, CD96, CD22 and LAIR1, or functional fragments thereof. Further inhibitory domains include those described in International Application Pub. No. WO2016075612A1. In some embodiments, the domain is selected from the intracellular domains of LIR1, PD1 and KIR3DL2 or functional fragments thereof. In some embodiments, the domain capable of providing an inhibitory signal is selected from the intracellular domain of a leukocyte immunoglobulin-like receptor (LIRs), such as LIR1 (LILRB1), or a functional fragment thereof. In some embodiments, the domain is selected from the intracellular domain of LILRB1, LILRB2, LILRB3, LILRB4, and LILRB5, or functional fragments thereof. In some embodiments, the domain is selected from the intracellular domain of PIR-B or a functional fragment thereof. In some embodiments, the inhibitory intracellular domain is a LILRB1 intracellular domain. In some embodiments, the inhibitory intracellular domain is a functional fragment of the intracellular domain of LILRB1. In some embodiments, the domain capable of providing an inhibitory signal is selected from the intracellular domain of an inhibitory killer cell immunoglobulin like receptors (KIRs) or a functional fragment thereof. In some embodiments, the domain is selected from the intracellular domain of KIR2DL1, KIR2DL2, KIR2DL3, KIR2DL4, KIR2DL5A, KIR2DL5B, KIR3DL1, KIR3DL2, and KIR3DL3, or functional fragments thereof.

In some embodiments, the fusion protein comprises a first and a second domain capable of providing a stimulatory signal, and first domain capable of providing a stimulatory signal is fused to the C terminus of an intracellular domain, for example a TCR alpha, TCR beta, CD3 gamma, CD3 delta, CD3 epsilon intracellular domain, and the second domain capable of providing a stimulatory signal is fused to the C-terminus of a first domain capable of providing a stimulatory signal.

TCRs provide potent signaling mechanisms. However, it is known that signals generated through the TCR alone are insufficient for full activation of naive T-cells and that a secondary and/or co-stimulatory signal is required. Productive T-cell activation thus requires a first signal provided by the interaction of antigenic peptide bound to major histocompatibility complex (pMHC) with the TCR and a second, antigen-independent co-signal or “co-stimulatory signal.” The two signals ensure proper coordination of T-cell activation, and the second signal is so important that it can be supplied in many molecular forms (e.g., CD80 and CD86). Thus, naïve T-cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

TCR-mediated activation without any co-stimulation results in antigen-specific unresponsiveness (termed T-cell anergy), rendering the T cells unable to respond to subsequent antigen and in this vein the absence of co-stimulation and the presence of immune checkpoints in the solid tumor microenvironment helps render the TCR unresponsive. This is a major contributor to reduced T cell responses in the tumor environment.

The inventors have discovered that altering the TCR intracellular domains with the addition of signaling intermediates creates a more sensitive TCR independent of co-stimulation and provides significant benefit by rendering the T cell more fit, functional and persistent. Further, adding specific intracellular signaling motifs enhances T cell receptor sensitivity and function. The inventors have altered TCR sensitivity and function by fusing signaling domains from stimulatory molecules, co-stimulatory molecules, co-receptors, kinases, and adaptors involved in the TCR signaling pathway to the intracellular domains or portions thereof of the TCR alpha, TCR beta, CD3 gamma, CD3 delta and/or CD3 epsilon subunits of the TCR complex.

Accordingly, the disclosure provides fusion proteins comprising a transmembrane domain, and an intracellular domain capable of providing additional stimulatory signals. In some embodiments, the fusion protein comprises a first second domain intracellular domain, for example a TCR alpha, TCR beta, CD3 delta, CD3 gamma or CD3 epsilon intracellular domain, and optionally a third intracellular domain capable of providing a stimulatory signal.

Fusion proteins comprising any one of TCR alpha, TCR beta, CD3 gamma, CD3 delta, CD3 epsilon, or derivatives or functional fragments thereof fused to one or more domains capable of providing stimulatory signals are provided herein.

In some embodiments, a fusion protein of the disclosure comprises an intracellular domain, for example TCR alpha, TCR beta, CD3 gamma, CD3 delta, or CD3 epsilon intracellular domain, a first domain capable of providing a stimulatory signal, and optionally, a second domain capable of providing a second stimulatory signal. All orders and orientations of the TCR-derived intracellular domain, and first and optionally second intracellular domains capable of providing a stimulatory signal, relative to the transmembrane domain, are envisaged as within the scope of the disclosure. In some embodiments, a fusion protein of the disclosure comprises an intracellular signaling domain capable of providing an inhibitory signal, as described herein.

In some embodiments, the domain capable of providing a stimulatory signal is fused to the C-terminus of a first intracellular domain, for example a TCR alpha, TCR beta, CD3 gamma, CD3 delta, or CD3 epsilon intracellular domain. In some embodiments, the domain capable of providing a stimulatory signal is fused to the C-terminus of the transmembrane domain, for example a TCR alpha, TCR beta, CD3 gamma, CD3 delta, or CD3 epsilon transmembrane domain.

### Stimulatory Molecules

In some embodiments, the one or more domains capable of providing stimulatory signals comprise stimulatory domains. In some embodiments, the one or more domains capable of providing stimulatory signals comprise co-stimulatory domains.

As used herein the stimulatory signaling domain refers to a portion of the fusion protein comprising the intracellular domain of a stimulatory molecule, or a molecule capable of recruiting a stimulatory molecule. Exemplary stimulatory molecules include co-stimulatory molecules, which can be cell surface proteins other than an antigen receptor or its ligands that are required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, a ligand that specifically binds with CD83, Lck, 4-1BB, CD4, CD8a, Fyn, ZAP70, LAT, SLP76 and the like. For example, CD27 co-stimulation has been demonstrated to enhance expansion, effector function, and survival of human TFP-T-cells in vitro and augments human T-cell persistence and antitumor activity in vivo (Song et al. Blood. 2012; 119(3):696-706).

In some embodiments, the intracellular signaling domain comprises at least one stimulatory intracellular domain. In some embodiments, the intracellular signaling domain comprises a primary intracellular signaling domain, such as a CD3 delta, CD3 gamma and CD3 epsilon intracellular domain, and one additional stimulatory intracellular domain, for example a co-stimulatory domain. In some embodiments, the intracellular signaling domain comprises a primary intracellular signaling domain, such as a CD3 delta, CD3 gamma and CD3 epsilon intracellular domain, and two additional stimulatory intracellular domains.

Exemplary co-stimulatory intracellular signaling domains include those derived from proteins responsible for co-stimulatory signals, or antigen independent stimulation.

The term “co-stimulatory molecule” refers to the cognate binding partner on a T-cell that specifically binds with a co-stimulatory ligand, thereby mediating a co-stimulatory response by the T-cell, such as, but not limited to, proliferation. Co-stimulatory molecules are cell surface molecules other than antigen receptors. Co-stimulatory molecules and their ligands are required for an efficient immune response. Co-stimulatory molecules include, but are not limited to an MHC class I molecule, BTLA, a Toll ligand receptor, as well as DAP10, DAP12, CD30, LIGHT, OX40, CD2, CD27, CDS, ICAM-1, LFA-1 (CD11a/CD18) 4-1BB (CD137, TNF receptor superfamily member 9), and CD28 molecule (CD28).

A “co-stimulatory domain”, sometimes referred to as “a co-stimulatory intracellular signaling domain” can be the intracellular portion of a co-stimulatory protein. A co-stimulatory domain can be a domain of a co-stimulatory protein that transduces the co-stimulatory signal. A co-stimulatory protein can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, CD4, and the like. The co-stimulatory domain can comprise the entire intracellular portion, or the entire native intracellular signaling domain, of the molecule from which it is derived, or a functional fragment thereof.

In some embodiments, the stimulatory domain comprises a co-stimulatory domain. In some embodiments, the co-stimulatory domain comprises a CD28 or 4-1BB co-stimulatory domain. CD28 and 4-1BB are well characterized co-stimulatory molecules required for full T cell activation and known to enhance T cell effector function. For example, CD28 and 4-1BB have been utilized in chimeric antigen receptors (CARs) to boost cytokine release, cytolytic function, and persistence over the first-generation CAR containing only the CD3 zeta signaling domain. Likewise, inclusion of co-stimulatory domains, for example CD28 and 4-1BB domains, in engineered TCR can increase T cell effector function and specifically allow co-stimulation in the absence of co-stimulatory ligand, which is typically down-regulated on the surface of tumor cells.

In some embodiments, the stimulatory domain comprises a CD28 intracellular domain. In some embodiments, the CD28 intracellular domain comprises an amino acid sequence having at least 85% identity, at least 90% identity, at least 95% identity, at least 96% identity, at least 97% identity, at least 98% identity, at least 99% identity or is identical to a sequence of: RSKRSRLLHSDYMNMTPRRPGPTRKHYQPYAPPRDFAAYRS (SEQ ID NO: 11). In some embodiments, the CD28 intracellular domain comprises, or consists essentially of, SEQ ID NO: 11. In some embodiments, a CD28 intracellular domain is encoded by a nucleotide sequence comprising: SEQ ID NO: 130.

In some embodiments, the stimulatory domain comprises a TNF receptor superfamily member 9 (4-1BB) intracellular domain. In some embodiments, the 4-1BB intracellular domain comprises an amino acid sequence having at least 85% identity, at least 90% identity, at least 95% identity, at least 96% identity, at least 97% identity, at least 98% identity, at least 99% identity or is identical to a sequence of: KRGRKKLLYIFKQPFMRPVQTTQEEDGCSCRFPEEEEGGCEL (SEQ ID NO: 12). In some embodiments, the 4-1BB intracellular domain comprises, or consists essentially of, KRGRKKLLYIFKQPFMRPVQTTQEEDGCSCRFPEEEEGGCEL (SEQ ID NO: 12). In some embodiments, a 4-1BB intracellular domain is encoded by a nucleotide sequence comprising SEQ ID NO: 131.

In some embodiments, the stimulatory domain comprises a TNF receptor superfamily member 18 (GITR) intracellular domain. In some embodiments, the GITR intracellular domain comprises an amino acid sequence having at least 85% identity, at least 90% identity, at least 95% identity, at least 96% identity, at least 97% identity, at least 98% identity, at least 99% identity or is identical to a sequence of: QLRKTQLLLEVPPSTEDARSCQFPEEERGERSAEEKGRLGDLWV (SEQ ID NO: 132). In some embodiments, a GITR intracellular domain is encoded by a nucleotide sequence comprising SEQ ID NO: 133.

### Co-Receptors

In some embodiments, the one or more domains capable of providing stimulatory signals comprises a domain from a co-receptor.

As used herein, a “co-receptor” refers to a cell surface receptor that binds a signaling molecule in addition to a primary receptor. Co-receptors can facilitate ligand recognition, ligand binding, and/or initiate, inhibit or change downstream signaling processes.

In some embodiments, the co-receptor is a TCR co-receptor. TCR co-receptors are associated with the TCR/CD3 complex upon TCR complex binding to pMHC and T cell activation. Their association with the TCR/CD3 complex amplifies or modulates the activation signal. In some embodiments, the presence of TCR co-receptors is required for productive signaling i.e. signaling that results in cell cycle progression and effector functions.

In some embodiments, the TCR co-receptor comprises a CD4 or CD8 co-receptor. CD4 and CD8 are known to recruit Lck to the TCR, thus increasing the local concentration of active Lck and lowering the threshold for TCR activation. Inclusion of Lck-binding domains of CD4 or CD8 in the engineered TCR is intended to enhance sensitivity to low levels of antigen.

In some embodiments, the stimulatory domain comprises a CD4 intracellular domain. In some embodiments, the CD4 intracellular domain comprises an amino acid sequence having at least 85% identity, at least 90% identity, at least 95% identity, at least 96% identity, at least 97% identity, at least 98% identity, at least 99% identity or is identical to a sequence of: CVRCRHRRRQAERMSQIKRLLSEKKTCQCPHRFQKTCSPI (SEQ ID NO: 13). In some embodiments, the CD4 intracellular domain comprises, or consists essentially of, SEQ ID NO: 13. In some embodiments, a CD4 intracellular domain is encoded by a nucleotide sequence comprising SEQ ID NO: 134.

In some embodiments, the stimulatory domain comprises a CD8 intracellular domain. In some embodiments, the CD8 intracellular domain comprises an amino acid sequence having at least 85% identity, at least 90% identity, at least 95% identity, at least 96% identity, at least 97% identity, at least 98% identity, at least 99% identity or is identical to a sequence of: LYCNHRNRRRVCKCPRPVVKSGDKPSLSARYV (SEQ ID NO: 14). In some embodiments, the CD8 intracellular domain comprises, or consists essentially of, SEQ ID NO: 14. In some embodiments, a CD8 intracellular domain is encoded by a nucleotide sequence comprising SEQ ID NO: 135.

### Kinases

In some embodiments, the one or more domains capable of providing stimulatory signals comprises a kinase or a functional fragment thereof. In some embodiments, the one or more domains capable of providing stimulatory signals comprises a kinase domain. As referred to herein, a “kinase” is an enzyme that catalyzes the transfer of a phosphate group from ATP to a specified molecule. A “kinase domain” is a structurally conserved domain that has kinase catalytic activity. Methods of identifying kinase domains, e.g. through sequence alignment, will be readily apparent to persons of ordinary skill in the art.

In some embodiments, the kinase is a SRC proto-oncogene, non-receptor tyrosine kinase (SRC) kinase domain, i.e. a kinase domain that has been isolated or derived from an SRC family kinase. SRC kinases are a family of non-receptor kinases, and include Src, Yes, Fyn, and Fgr (the SrcA subfamily) Lck, Hck, Blk, and Lyn (the SrcB subfamily) and Frk.

In some embodiments, the kinase is a Fyn kinase. Fyn and Lck are both part of the SRC kinase family and can compensate for each other in phosphorylating ITAM residues of the TCR. In some embodiments, the Fyn kinase comprises an amino acid sequence having at least 85% identity, at least 90% identity, at least 95% identity, at least 96% identity, at least 97% identity, at least 98% identity, at least 99% identity or is identical to a sequence of SEQ ID NO: 15. In some embodiments, the Fyn kinase comprises, or consists essentially of, SEQ ID NO: 15. In some embodiments, a Fyn kinase is encoded by a nucleotide sequence comprising SEQ ID NO: 136.

In some embodiments, the kinase is Lck kinase. Lck is the first molecule involved in the proximal signaling cascade after TCR binding to antigen. Basal Lck activation is known to set the threshold for TCR activation and is regulated by localization to membrane, and phosphorylation state of key tyrosine residues. Inclusion of Lck in the fusion proteins of the disclosure may lower the threshold for TCR activation, leading to enhanced sensitivity to low levels of antigen. In some embodiments, the Lck kinase comprises an amino acid sequence having at least 85% identity, at least 90% identity, at least 95% identity, at least 96% identity, at least 97% identity, at least 98% identity, at least 99% identity or is identical to a sequence of SEQ ID NO: 16. In some embodiments, the Lck kinase comprises, or consists essentially of, SEQ ID NO: 16. In some embodiments, the Lck kinase is encoded by a nucleotide sequence of SEQ ID NO: 137.

In some embodiments, the fusion protein comprises a full length Lck kinase.

In some embodiments, the fusion protein comprises a truncated Lck kinase. In some embodiments, the Lck domain comprises amino acids 245-498 of SEQ ID NO: 16, 2-509 of SEQ ID NO: 16, 6-509 of SEQ ID NO: 16, 73-509 of SEQ ID NO: 16, amino acids 122-509 of SEQ ID NO: 16, 225-509 of SEQ ID NO: 16 or 225-509 of SEQ ID NO: 16.

In some embodiments, the fusion protein comprises a mutated Lck kinase or functional fragment thereof. In some embodiments, the mutated Lck kinase comprises an Y505A substitution of SEQ ID NO: 16.

In some embodiments, the kinase is a ZAP70 kinase. ZAP70 is activated by Lck while bound to the ITAMs in the TCR complex and phosphorylates Tyrosine residues of two important adaptor molecules, LAT and SLP76. Whereas ZAP70 is thought to be a key mediator of TCR activation and downstream signaling, inclusion of ZAP70 in the engineered TCR can boost amplification of TCR signaling leading to more potent T cell activation. In some embodiments, the Zap70 domain comprises the Zap70 kinase domain, the Zap70 interdomain, or a combination thereof. In some embodiments, the ZAP70 kinase comprises an amino acid sequence having at least 85% identity, at least 90% identity, at least 95% identity, at least 96% identity, at least 97% identity, at least 98% identity, at least 99% identity or is identical to a sequence of SEQ ID NO: 17. In some embodiments, the ZAP70 kinase comprises, or consists essentially of, SEQ ID NO: 17. In some embodiments, the ZAP70 kinase is encoded by a nucleotide sequence of SEQ ID NO: 138.

In some embodiments, a Zap70 interdomain B and kinase domain comprises an amino acid sequence having at least 85% identity, at least 90% identity, at least 95% identity, at least 96% identity, at least 97% identity, at least 98% identity, at least 99% identity or is identical to a sequence of SEQ ID NO: 139. In some embodiments, a Zap70 kinase domain comprises an amino acid sequence having at least 85% identity, at least 90% identity, at least 95% identity, at least 96% identity, at least 97% identity, at least 98% identity, at least 99% identity or is identical to a sequence of: SEQ ID NO: 140.

### Adaptors

In some embodiments, the one or more domains capable of providing stimulatory signals comprises an adaptor. As used herein, an “adaptor” refers to a protein that influences signal transduction pathways by regulating cross-talk and specificity. Adaptor proteins contain protein-binding motifs, which facilitate interactions between protein-binding partners and the generation of bigger signaling complexes. Many adaptor proteins are required to link molecules within a signaling cascade to ensure the appropriate signaling response.

In some embodiments, the adaptor comprises a LAT protein or a functional fragment thereof. LAT is an important adaptor molecule which recruits multiple key enzymes and signaling molecules to the TCR complex to mediate downstream signaling after TCR ligation. Inclusion of LAT in the engineered TCR can enhance T cell activation. In some embodiments, LAT comprises an amino acid sequence having at least 85% identity, at least 90% identity, at least 95% identity, at least 96% identity, at least 97% identity, at least 98% identity, at least 99% identity or is identical to a sequence of SEQ ID NO: 25. In some embodiments, LAT comprises, or consists essentially of, SEQ ID NO: 25. In some embodiments, LAT comprises, or consists essentially of, a functional fragment of SEQ ID NO: 25. In some embodiments, LAT is encoded by a nucleotide sequence of SEQ ID NO: 141.

In some embodiments, the adaptor comprises a SLP76 protein or a functional fragment thereof. SLP76 is recruited to the LAT complex and activates key enzymes and signaling molecules involved in downstream signaling and cell adhesion. Inclusion of SLP76 in the engineered TCR can enhance T cell activation. In some embodiments, SLP76 comprises an amino acid sequence having at least 85% identity, at least 90% identity, at least 95% identity, at least 96% identity, at least 97% identity, at least 98% identity, at least 99% identity or is identical to a sequence of SEQ ID NO: 26. In some embodiments, SLP76 comprises, or consists essentially of, SEQ ID NO: 26. In some embodiments, SLP76 comprises, or consists essentially of, a functional fragment of SEQ ID NO: 26. In some embodiments, SLP76 is encoded by a nucleotide sequence of SEQ ID NO: 142.

### Inhibitory Intracellular Domains

In some embodiments, the intracellular domain comprises an intracellular domain capable of providing an inhibitory signal. The intracellular domain can comprise a C-terminal fusion of one or more domains capable of providing inhibitory signal to a transmembrane domain or an intracellular domain of a fusion protein of the disclosure.

Non-limiting examples of inhibitory domains are shown in Table 1 below:

TABLE 1
Proteins with Inhibitory Domains
Inhibitory Domains/ITIMS

huKIR3DL2
huKIR3DL3
huLIR1
huPD-1
huZAP70 SH2(N + C)
huZAP70 KI_K369A

In some embodiments, the intracellular domain comprises a first intracellular domain capable of providing an inhibitory signal and a second intracellular domain, for example an intracellular domain of a TCR subunit.

In some embodiments, the first intracellular domain capable of providing an inhibitory signal comprises an immunoreceptor tyrosine-based inhibitory motif (ITIM). In some embodiments, first intracellular domain capable of providing an inhibitory signal comprises an immunoreceptor tyrosine-based switch motif (ITSM). In some embodiments, first intracellular domain capable of providing an inhibitory signal comprises a Src Homology 2 (SH2) domain. In some embodiments, the first intracellular domain capable of providing an inhibitory signal comprises one or more of an ITIM, ITSM or SH2 domain.

In some embodiments, the inhibitory intracellular domain comprises an ITIM. In some embodiments, the inhibitory intracellular domain comprising an ITIM can be isolated or derived from an immune checkpoint inhibitor such as CTLA-4 or PD-1. CTLA-4 and PD-1 are immune inhibitory receptors expressed on the surface of T cells, and play a pivotal role in attenuating or terminating T cell responses. Further, exemplary proteins comprising ITIMs include, but are not limited to, KIR3DL2, KIR3DL3 and LIR. In some embodiments, the ITIM is non-naturally occurring or synthetic. For example an ITIM based on the ITIM consensus sequence of S/I/V/LxYxxI/V/L (SEQ ID NO: 115) is added to an intracellular domain. Without wishing to be bound by theory, it is thought that adding ITIMs to domains can confer inhibitory activity upon the domain, or increase the inhibitory activity of the domain.

In some embodiments, the first intracellular domain capable of providing an inhibitory signal is selected from the group consisting of a programmed cell death 1 (PD-1) intracellular domain, a cytotoxic T-lymphocyte associated protein 4 (CTLA-4) intracellular domain, a killer cell immunoglobulin like receptor, three Ig domains and long cytoplasmic tail 2 (KIR3DL2) intracellular domain, a killer cell immunoglobulin like receptor, three Ig domains and long cytoplasmic tail 3 (KIR3DL3) intracellular domain, a zeta chain of T cell receptor associated protein kinase 70 (ZAP70) domain comprising a Src Homology 2 (SH2) domain, a ZAP70 inactivated kinase domain, a leukocyte immunoglobulin like receptor B1 (LIR1) intracellular domain, an Fc gamma receptor JIB (FcgRIIB) intracellular domain, or a killer cell lectin like receptor K1 (NKG2D) intracellular domain.

Additional inhibitory domains can be isolated from human tumor necrosis factor related apoptosis inducing ligand (TRAIL) receptor and CD200 receptor 1. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as “helpless” CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the inhibitory domain is isolated or derived from a human protein. In some embodiments, the inhibitory domain is isolated or derived from a human TRAIL receptor, CD200 receptor 1, PD-1, CTLA-4, KIR3DL2, KIR3DL3, ZAP70, LIR1, FcgRIIB or NKG2D.

In some embodiments, the first intracellular domain capable of providing an inhibitory signal is selected from the group consisting of a PD-1 intracellular domain, a LIR1 intracellular domain, and a KIR3DL2 intracellular domain.

In some embodiments, the intracellular domain comprises a first, inhibitory intracellular domain, and a second intracellular domain. In some embodiments, the second intracellular domain is selected from a CD3D molecule (CD3 delta) intracellular domain (ICD), a CD3E molecule (CD3 epsilon) ICD, a CD3G molecule (CD3 gamma), a CD247 molecule (CD3Z or CD3 zeta) intracellular domain or fragments or functional derivatives thereof.

In some embodiments, the inhibitory domain comprises a KIR3DL2 intracellular domain or a functional fragment thereof. Inclusion of the KIR3DL2 domain in the engineered TCR can inhibit T cell activation. In some embodiments, KIR3DL2 comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 52)
    1 MSLTVVSMAC VGFFLLQGAW PLMGGQDKPF
        LSARPSTVVP RGGHVALQCH YRRGFNNFML
  61 YKEDRSHVPI FHGRIFQESF 1MGPVTPAHA
        GTYRCRGSRP HSLTGWSAPS NPLVIMVTGN
121 HRKPSLLAHP GPLLKSGETV ILQCWSDVMF
        EHFFLHREGI SEDPSRLVGQ IHDGVSKANF
181 SIGPLMPVLA GTYRCYGSVP HSPYQLSAPS
        DPLDIVITGL YEKPSLSAQP GPTVQAGENV
241 TLSCSSWSSY DIYHLSREGE AHERRLRAVP
        KVNRTFQADF PLGPATHGGT YRCFGSFHAL
301 PCVWSNSSDP LLVSVTGNPS SSWPSPTEPS
        SKSGICRHLH VLIGTSVVIF LFILLLFFLL
361 YRWCSNKKNA AVMDQEPAGD RTVNRQDSDE
        QDPQEVTYAQ LDHCVFIQRK ISRPSQRPKT
421 PLTDTSVYTE LPNAEPRSKV VSCPRAPQSG
        LEGVF.
```

In some embodiments, the KIR3DL2 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 52.

In some embodiments, the KIR3DL2 domain comprises an amino acid sequence having at least 85% identity, at least 90% identity, at least 95% identity, at least 96% identity, at least 97% identity, at least 98% identity, at least 99% identity or is identical to a sequence of NKKNAAVMDQEPAGDRTVNRQDSDEQDPQEVTYAQLDHCVFIQRKISRPSQRPKTPLTDTSVYTELPNAE PRSKVVSCPRAPQSGLEGVF (SEQ ID NO: 124). In some embodiments, the KIR3DL2 domain comprises or consists essentially of SEQ ID NO: 124.

In some embodiments, the KIR3DL2 domain is encoded by a sequence having at least 85% identity, at least 90% identity, at least 95% identity, at least 96% identity, at least 97% identity, at least 98% identity, at least 99% identity or is identical to a sequence of SEQ ID NO: 143. In some embodiments, the KIR3DL2 domain is encoded by a sequence comprising or consisting essentially of SEQ ID NO: 143.

In some embodiments, the inhibitory domain comprises a KIR3DL3 domain or a functional fragment thereof. Inclusion of the KIR3DL3 domain in the engineered TCR can inhibit T cell activation. In some embodiments, KIR3DL3 comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 53)
    1 MSLMVVSMAC VGFFLLEGPW PHVGGQDKPF
        LSAWPSTVVS EGQHVTLQCR SRLGFNEFSL
  61 SKEDGMPVPE LYNRIFRNSF LMGPVTPAHA
        GTYRCCSSHP HSPTGWSAPS NPVVIMVTGV
121 HRKPSLLAHP GPLVKSGETV ILQCWSDVRF
        ERFLLHREGI TEDPLRLVGQ LHDAGSQVNY
181 SMGPMTPALA GTYRCFGSVT HLPYELSAPS
        DPLDIVVVGL YGKPSLSAQP GPTVQAGENV
241 TLSCSSRSLF DIYHLSREAE AGELRLTAVL
        RVNGTFQANF PLGPVTHGGN YRCFGSFRAL
301 PHAWSDPSDP LPVSVTGNSR YLHALIGTSV
        VIIPFAILLF FLLHRWCANK KNAVVMDQEP
361 AGNRTVNRED SDEQDPQEVT YAQLNHCVFT
        QRKITRPSQR PKTPPTDTSV.
```

In some embodiments, the KIR3DL3 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 53.

In some embodiments, the inhibitory domain comprises ZAP70, a ZAP70 domain, or a functional fragment thereof. In some embodiments, the ZAP70 domain comprises one or more ZAP70 SH2 domains. The ZAP70 protein comprises two SH2 domains, referred to herein as the N and C terminal SH2 domains. In some embodiments, the ZAP70 N terminal SH2 domain comprises a sequence of FFYGSISRAEAEEHLKLAGMADGLFLLRQCLRSLGGYVLSLVHDVRFHHFPIERQLNGT YAIAGGKAHCGPAELCEFYSRDPDGLPCNLRKPC (SEQ ID NO: 54), or at least 85% identity, at least 90% identity, at least 95% identity, at least 96% identity, at least 97% identity, at least 98% identity, at least 99% identity thereto. In some embodiments, the ZAP70 C terminal SH2 domain comprises a sequence of WYHSSLTREEAERKLYSGAQTDGKFLLRPRKEQGTYALSLIYGKTVYHYLISQDKAGKY CIPEGTKFDTLWQLVEYLKLKADGLIYCLKEAC (SEQ ID NO: 55), or at least 85% identity, at least 90% identity, at least 95% identity, at least 96% identity, at least 97% identity, at least 98% identity, at least 99% identity thereto. In some embodiments, the ZAP70 domain comprises an amino acid sequence having at least 85% identity, at least 90% identity, at least 95% identity, at least 96% identity, at least 97% identity, at least 98% identity, at least 99% identity or is identical to a sequence or partial sequence of SEQ ID NO: 17. In some embodiments, the ZAP70 or a portion thereof is kinase inactive. In some embodiments, the kinase inactive ZAP70 comprises an amino acid substitution of Alanine for Lysine at position 369 of SEQ ID NO: 17.

In some embodiments, the inhibitory domain comprises a LIR1 domain or a functional fragment thereof. The LILRB1 intracellular domain includes 4 ITIMs, the sequences of which are The ITIMs of LILRB1 are NLYAAV (SEQ ID NO: 70), VTYAEV (SEQ ID NO: 71), VTYAQL (SEQ ID NO: 72), and SIYATL (SEQ ID NO: 73). LIR1 intracellular domains comprising any one or more of the LIR1 ITIMs can be used in the inhibitory TCRs described herein. Inclusion of the LIR1 domain, or a fragment thereof, in the engineered TCR can inhibit T cell activation. In some embodiments, LIR1 comprises an amino acid sequence having at least 85% identity, at least 90% identity, at least 95% identity, at least 96% identity, at least 97% identity, at least 980 identity, least 990 identity or is identical sequence of:

```
(SEQ ID NO: 56)
    1 MTPILTVLIC LGLSLGPRTH VQAGHLPKPT
        LWAEPGSVIT QGSPVTLRCQ GGQETQEYRL
  61 YREKKTALWI TRIPQELVKK GQFPIPSITW
        EHAGRYRCYY GSDTAGRSES SDPLELVVTG
121 AYIKPTLSAQ PSPVVNSGGN VILQCDSQVA
        FDGFSLCKEG EDEHPQCLNS QPHARGSSRA
181 IFSVGPVSPS RRWWYRCYAY DSNSPYEWSL
        PSDLLELLVL GVSKKPSLSV QPGPIVAPEE
241 TLTLQCGSDA GYNRFVLYKD GERDFLQLAG
        AQPQAGLSQA NFTLGPVSRS YGGQYRCYGA
301 HNLSSEWSAP SDPLDILIAG QFYDRVSLSV
        QPGPTVASGE NVTLLCQSQG WMQTFLLTKE
361 GAADDPWRLR STYQSQKYQA EFPMGPVTSA
        HAGTYRCYGS QSSKPYLLTH PSDPLELVVS
421 GPSGGPSSPT TGPTSTSAGP EDQPLTPTGS
        DPQSGLGRHL GVVIGILVAV ILLLLLLLLL
481 FLILRHRRQG KHWTSTQRKA DFQHPAGAVG
        PEPTDRGLQW RSSPAADAQE ENLYAAVKHT
541 QPEDGVEMDT RSPHDEDPQA VTYAEVKHSR
        PRREMASPPS PLSGEFLDTK DRQAEEDRQM
601 DTEAAASEAP QDVTYAQLHS LTLRREATEP
        PPSQEGPSPA VPSIYATLAI H.
```

In some embodiments, the LIR1 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 56.

In some embodiments, the inhibitory domain comprises a LILRB1 (also known as LIR1, or LIR-1) domain or a functional fragment thereof. Inclusion of the LILRB1 domain in the engineered TCR can inhibit T cell activation. In some embodiments, LILRB1 comprises an amino acid sequence having at least 85% identity, at least 90% identity, at least 95% identity, at least 96% identity, at least 97% identity, at least 98% identity, at least 99% identity or is identical to a sequence of: LRHRRQGKHWTSTQRKADFQHPAGAVGPEPTDRGLQWRSSPAADAQEENLYAAVKHT QPEDGVEMDTRSPHDEDPQAVTYAEVKHSRPRREMASPPSPLSGEFLDTKDRQAEEDR QMDTEAAASEAPQDVTYAQLHSLTLRREATEPPPSQEGPSPAVPSIYATLAIH (SEQ ID NO: 61). In some embodiments, the LILRB1 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 61. In some embodiments, the LILRB1 domain comprises or consists essentially of SEQ ID NO: 61.

In some embodiments, LILRB1 comprises an amino acid sequence having at least 85% identity, at least 90% identity, at least 95% identity, at least 96% identity, at least 97% identity, at least 98% identity, at least 99% identity or is identical to a sequence of: RHRRQGKHWTSTQRKADFQHPAGAVGPEPTDRGLQWRSSPAADAQEENLYAAVKHTQ PEDGVEMDTRSPHDEDPQAVTYAEVKHSRPRREMASPPSPLSGEFLDTKDRQAEEDRQ MDTEAAASEAPQDVTYAQLHSLTLRREATEPPPSQEGPSPAVPSIYATLAIH (SEQ ID NO: 67). In some embodiments, the LILRB1 domain comprises or consists essentially of SEQ ID NO: 67. In some embodiments, the LILRB1 domain is encoded by a nucleotide sequence having at least 85% identity, at least 90% identity, at least 95% identity or is identical to a sequence of SEQ ID NO: 232.

In some embodiments, LILRB1 comprises an amino acid sequence having at least 85% identity, at least 90% identity, at least 95% identity, at least 96% identity, at least 97% identity, at least 98% identity, at least 99% identity or is identical to a sequence of: LSGAADTQALLRNDVTYAQLHSLTLRREATEPPPSQEGPSPAVPSIYATL (SEQ ID NO: 68). In some embodiments, the LILRB1 domain comprises or consists essentially of SEQ ID NO: 68. In some embodiments, the LILRB1 domain is encoded by a nucleotide sequence having at least 85% identity, at least 90% identity, at least 95% identity or is identical to a sequence of SEQ ID NO: 233).

In some embodiments, LILRB1 comprises an amino acid sequence having at least 85% identity, at least 90% identity, at least 95% identity, at least 96% identity, at least 97% identity, at least 98% identity, at least 99% identity or is identical to a sequence of: VTYAQLHSLTLRREATEPPPSQEGPSPAVPSIYATL (SEQ ID NO: 69). In some embodiments, the LILRB1 domain comprises or consists essentially of SEQ ID NO: 69. In some embodiments, the LILRB1 domain is encoded by a nucleotide sequence having at least 85% identity, at least 90% identity, at least 95% identity or is identical to a sequence of SEQ ID NO: 234).

In some embodiments, the inhibitory domain comprises a LILRB2 domain or a functional fragment thereof. Inclusion of the LILRB2 domain in the engineered TCR can inhibit T cell activation. In some embodiments, LILRB2 comprises an amino acid sequence having at least 85% identity, at least 90% identity, at least 95% identity, at least 96% identity, at least 97% identity, at least 98% identity, at least 99% identity or is identical to a sequence of: LRHRRQGKHWTSTQRKADFQHPAGAVGPEPTDRGLQWRSSPAADAQEENLYAAVKDT QPEDGVEMDTRAAASEAPQDVTYAQLHSLTLRRKATEPPPSQEREPPAEPSIYATLAIH (SEQ ID NO: 62). In some embodiments, the LILRB2 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 62.

In some embodiments, the inhibitory domain comprises a LILRB3 domain or a functional fragment thereof. Inclusion of the LILRB3 domain in the engineered TCR can inhibit T cell activation. In some embodiments, LILRB3 comprises an amino acid sequence having at least 85% identity, at least 90% identity, at least 95% identity, at least 96% identity, at least 97% identity, at least 98% identity, at least 99% identity or is identical to a sequence of: RRQRHSKHRTSDQRKTDFQRPAGAAETEPKDRGLLRRSSPAADVQEENLYAAVKDTQS EDRVELDSQSPHDEDPQAVTYAPVKHSSPRREMASPPSSLSGEFLDTKDRQVEEDRQMD TEAAASEASQDVTYAQLHSLTLRRKATEPPPSQEGEPPAEPSIYATLAIH (SEQ ID NO: 63). In some embodiments, the LILRB3 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 63.

In some embodiments, the inhibitory domain comprises a LILRB4 domain or a functional fragment thereof. Inclusion of the LILRB4 domain in the engineered TCR can inhibit T cell activation. In some embodiments, LILRB4 comprises an amino acid sequence having at least 85% identity, at least 90% identity, at least 95% identity, at least 96% identity, at least 97% identity, at least 98% identity, at least 99% identity or is identical to a sequence of: QHWRQGKHRTLAQRQADFQRPPGAAEPEPKDGGLQRRSSPAADVQGENFCAAVKNTQ PEDGVEMDTRQSPHDEDPQAVTYAKVKHSRPRREMASPPSPLSGEFLDTKDRQAEEDR QMDTEAAASEAPQDVTYAQLHSFTLRQKATEPPPSQEGASPAEPSVYATLAIH (SEQ ID NO: 64). In some embodiments, the LILRB4 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 64.

In some embodiments, the inhibitory domain comprises a LILRB5 domain or a functional fragment thereof. Inclusion of the LILRB5 domain in the engineered TCR can inhibit T cell activation. In some embodiments, LILRB5 comprises an amino acid sequence having at least 85% identity, at least 90% identity, at least 95% identity, at least 96% identity, at least 97% identity, at least 98% identity, at least 99% identity or is identical to a sequence of: RHRHQSKHRTSAHFYRPAGAAGPEPKDQGLQKRASPVADIQEEILNAAVKDTQPKDGV EMDARAAASEAPQDVTYAQLHSLTLRREATEPPPSQEREPPAEPSIYAPLAIH (SEQ ID NO: 65). In some embodiments, the LILRB5 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 65.

In some embodiments, the inhibitory domain comprises a PD-1 (also referred to herein as PD1) domain or a functional fragment thereof. Inclusion of the PD-1 domain in the engineered TCR can inhibit T cell activation. In some embodiments, PD-1 comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 57)
    1 MQIPQAPWPV VWAVLQLGWR PGWFLDSPDR
        PWNPPTFSPA LLVVTEGDNA TFTCSFSNTS
  61 ESFVLNWYRM SPSNQTDKLA AFPEDRSQPG
        QDCRFRVTQL PNGRDFHMSV VRARRNDSGT
121 YLCGAISLAP KAQIKESLRA ELRVTERRAE
        VPTAHPSPSP RPAGQFQTLV VGWGGLLGS
181 LVLLVWVLAV ICSRAARGTI GARRTGQPLK
        EDPSAVPVFS VDYGELDFQW REKTPEPPVP
241 CVPEQTEYAT IVFPSGMGTS SPARRGSADG
        PRSAQPLRPE DGHCSWPL.
```

In some embodiments, the PD-1 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 57.

In some embodiments, the PD-1 intracellular domain comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 66)
    1 TERRAEVPTA HPSPSPRPAG QFQTLVVGVV
        GGLLGSLVLL VWVLAVICSR AARGTIGARR
  61 TGQPLKEDPS AVPVFSVDYG ELDFQWREKT
        PEPPVPCVPE QTEYATIVFP SGMGTSSPAR
121 RGSADGPRSA QPLRPEDGHC SWPL.
```

In some embodiments, the PD-1 intracellular domain comprises or consists essentially of SEQ ID NO: 66. In some embodiments, the PD-1 domain is encoded by a nucleotide sequence having at least 85% identity, at least 90% identity, at least 95% identity, at least 99% identity or is identical to a sequence of SEQ ID NO: 236.

In some embodiments, the PD-1 intracellular domain comprises an amino acid sequence having at least 85% identity, at least 90% identity, at least 95% identity, at least 96% identity, at least 97% identity, at least 98% identity, at least 99% identity or is identical to a sequence of: RAARGTIGARRTGQPLKEDPSAVPVFSVDYGELDFQWREKTPEPPVPCVPEQTEYATIVF PSGMGTSSPARRGSADGPRSAQPLRPEDGHCSWPL (SEQ ID NO: 74). In some embodiments, the PD-1 intracellular domain comprises or consists essentially of SEQ ID NO: 74. In some embodiments, the PD-1 domain is encoded by a nucleotide sequence having at least 85% identity, at least 90% identity, at least 95% identity or is identical to a sequence of SEQ ID NO: 235.

In some embodiments, the inhibitory domain comprises a CTLA-4 domain or a functional fragment thereof. Inclusion of the CTLA-4 domain in the engineered TCR can inhibit T cell activation. In some embodiments, CTLA-4 comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 58)
    1 MACLGFQRHK AQLNLATRTW PCTLLFFLLF
        IPVFCKAMHV AQPAVVLASS RGIASFVCEY
  61 ASPGKATEVR VTVLRQADSQ VTEVCAATYM
        MGNELTFLDD SICTGTSSGN QVNLTIQGLR
121 AMDTGLYICK VELMYPPPYY LGIGNGTQIY
        VIDPEPCPDS DFLLWILAAV SSGLFFYSFL
181 LTAVSLSKML KKRSPLTTGV YVKMPPTEPE
        CEKQFQPYFI PIN.
```

In some embodiments, the CTLA-4 domain comprises an amino acid sequence having at least 85% identity, at least 90% identity, at least 95% identity, at least 96% identity, at least 97% identity, at least 98% identity, at least 99% identity or is identical to a fragment of SEQ ID NO: 58.

In some embodiments, the inhibitory domain comprises a NKG2D domain or a functional fragment thereof. Inclusion of the NKG2D domain in the engineered TCR can inhibit T cell activation. In some embodiments, NKG2D comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 59)
    1 MGWIRGRRSR HSWEMSEFHN YNLDLKKSDF
        STRWQKQRCP VVKSKCRENA SPFFFCCFIA
  61 VAMGIRFIIM VAIWSAVFLN SLFNQEVQIP
        LTESYCGPCP KNWICYKNNC YQFFDESKNW
121 YESQASCMSQ NASLLKVYSK EDQDLLKLVK
        SYHWMGLVHI PTNGSWQWED GSILSPNLLT
181 IIEMQKGDCA LYASSFKGYI ENCSTPNTYI
        CMQRTV
```

In some embodiments, the NKG2D domain comprises an amino acid sequence having at least 85% identity, at least 90% identity, at least 95% identity, at least 96% identity, at least 97% identity, at least 98% identity, at least 99% identity or is identical to a fragment of SEQ ID NO: 59.

In some embodiments, the inhibitory domain comprises a FcgRIIB domain or a functional fragment thereof. Inclusion of the FcgRIIB domain in the engineered TCR can inhibit T cell activation. In some embodiments, FcgRIIB comprises an amino acid sequence having at least 85% identity, at least 90% identity, at least 95% identity, at least 96% identity, at least 97% identity, at least 98% identity, at least 99% identity or is identical to a sequence of:

```
(SEQ ID NO: 60)
    1 MGILSFLPVL ATESDWADCK SPQPWGHMLL
        WTAVLFLAPV AGTPAAPPKA VLKLEPQWIN
  61 VLQEDSVTLT CRGTHSPESD SIQWFHNGNL
        IPTHTQPSYR FKANNNDSGE YTCQTGQTSL
121 SDPVHLTVLS EWLVLQTPHL EFQEGETIVL
        RCHSWKDKPL VKVTFFQNGK SKKFSRSDPN
181 FSIPQANHSH SGDYHCTGNI GYTLYSSKPV
        TITVQAPSSS PMGIIVAVVT GIAVAAIVAA
241 VVALIYCRKK RISALPGYPE CREMGETLPE
        KPANPTNPDE ADKVGAENTI TYSLLMHPDA
301 LEEPDDQNRI.
```

In some embodiments, the FcgRIIB domain comprises an amino acid sequence having at least 85% identity, at least 90% identity, at least 95% identity, at least 96% identity, at least 97% identity, at least 98% identity, at least 99% identity or is identical to a fragment of SEQ ID NO: 60.

In some embodiments, the inhibitory intracellular domain comprises an amino acid sequence having at least 85% identity, at least 90% identity, at least 95% identity, at least 96% identity, at least 97% identity, at least 98% identity, at least 99% identity or is identical to a sequence of:

(SEQ ID NO: 75)
RVKFSRSADAPAYQQPDPQEVTYAQLDKRRGRDPE
MGGKPRRKPDPQEVTYAQLGMKGERRRGKGHPDPQ
EVTYAQLHMQALPPR.

In some embodiments, the inhibitory intracellular domain comprises or consists essentially of SEQ ID NO: 75.

### Linkers

In some embodiments, the fusion proteins described herein comprise two or more intracellular domains, which can be isolated or derived from separate sources. Provided herein are linkers that, in some embodiments, can be used link the domains of the fusion proteins described herein.

The terms “linker” and “flexible polypeptide linker” as used in the context of linking protein domains, for example intracellular domains or domains within an scFv, refers to a peptide linker that consists of amino acids such as glycine and/or serine residues used alone or in combination, to link two domains together. For example, a linker as described herein can be used to link variable heavy and variable light chain regions together, or two link two intracellular domains together.

In some embodiments, linkers can be used to link two intracellular domains in a fusion protein, an intracellular domain and a transmembrane domain, a transmembrane domain and a hinge or extracellular domain, or a multiple linkers may be used to link combinations of these domains.

The intracellular domains within the cytoplasmic portion of the fusion protein of the invention may be linked to each other in a random or specified order.

The intracellular domains within the cytoplasmic portion of the fusion protein of the invention may be linked to each other in any orientation of N to C terminus.

Optionally, a short oligo- or polypeptide linker, for example, between 2 and 40 amino acids (e.g., 2, 3, 4, 5, 6, 7, 8, 9, or 10 amino acids) in length may form the linkage between the domains.

In some embodiments, the polypeptide linker comprises at most 3, 4, 5, 6, 8, 9, 10, 12, 15, 16, 18 or 20 amino acids.

In some embodiments, the flexible polypeptide linker is a Gly/Ser linker and comprises the amino acid sequence (Gly-Gly-Ser), (Gly-Gly-Gly-Ser) (SEQ ID NO: 240), or (Gly-Gly-Gly-Gly-Ser) (SEQ ID NO: 18) which can be repeated n times, where n is a positive integer equal to or greater than 1. For example, n=1, n=2, n=3, n=4, n=5, n=6, n=7, n=8, n=9 and n=10. In some embodiments, the flexible polypeptide linkers include, but are not limited to, GGS, GGGGS (SEQ ID NO: 18), GGGGS GGGGS (SEQ ID NO: 19), GGGGS GGGGS GGGGS (SEQ ID NO: 20) or GGGGS GGGGS GGGGS GGGGS (SEQ ID NO: 21).

In some embodiments, the linkers include multiple repeats of (Gly Gly Ser), (Gly Ser) or (Gly Gly Gly Ser) (SEQ ID NO: 240). Also included within the scope of the invention are linkers described in WO2012/138475 (incorporated herein by reference).

In some embodiments, the linker sequence comprises a long linker (LL) sequence. In some embodiments, the long linker sequence comprises GGGGS, repeated four times. In some embodiments, a GGGGS GGGGS GGGGS GGGGS (SEQ ID NO: 21) is used to link intracellular domains in a TCR alpha fusion protein of the disclosure.

In some embodiments, the long linker sequence comprises GGGGS, repeated three times. In some embodiments, a GGGGS GGGGS GGGGS (SEQ ID NO: 20) is used to link intracellular domains in a TCR beta fusion protein of the disclosure.

In some embodiments, the linker sequence comprises a short linker (SL) sequence. In some embodiments, the short linker sequence comprises GGGGS (SEQ ID NO: 18).

In some embodiments, a glycine-serine doublet can be used as a suitable linker.

In some embodiments, domains are fused directly to each other via peptide bonds without use of a linker.

An exemplary CD3 subunit fusion protein of the disclosure comprises CD3 delta fused to a CD28 intracellular with a GGS linker. In some embodiments, CD3 delta fused to a CD28 intracellular domain comprises an amino acid sequence of SEQ ID NO: 144. In some embodiments, CD3 delta fused to a CD28 intracellular domain is encoded by the nucleotide sequence of SEQ ID NO: 145.

An exemplary CD3 subunit fusion protein of the disclosure comprises CD3 epsilon fused to a CD28 intracellular domain with a GGS linker. In some embodiments, CD3 epsilon fused a CD28 intracellular domain comprises an amino acid sequence of SEQ ID NO: 146. In some embodiments, CD3 epsilon fused a CD28 intracellular domain is encoded by the nucleotide sequence of SEQ ID NO: 147.

An exemplary CD3 subunit fusion protein of the disclosure comprises CD3 gamma fused to a CD28 intracellular domain with a GGS linker. In some embodiments, CD3 gamma fused to a CD28 intracellular domain comprises an amino acid sequence of SEQ ID NO: 148. In some embodiments, CD3 gamma fused to a CD28 intracellular domain is encoded by the nucleotide sequence of SEQ ID NO: 149.

An exemplary TCR alpha fusion protein of the disclosure comprises an extracellular domain targeting NY-ESO1 1G4, a TCR alpha T48C transmembrane domain, and a CD28 intracellular domain linked to TCR alpha with a GGS linker. In some embodiments, an extracellular domain targeting NY-ESO1 1G4, a TCR alpha T48C transmembrane domain, and a CD28 intracellular domain linked to TCR alpha with a GGS linker comprises an amino acid sequence of SEQ ID NO: 150.

In some embodiments, an extracellular domain targeting NY-ESO1 1G4, a TCR alpha T48C transmembrane domain, and a CD28 intracellular domain linked to TCR alpha with a GGS linker is encoded by nucleotide sequence of SEQ ID NO: 151.

An exemplary TCR beta fusion protein of the disclosure comprises an extracellular domain targeting NY-ESO1 1G4, a TCR beta transmembrane domain, and a CD28 intracellular domain linked to TCR alpha with a GGS linker. In some embodiments, an extracellular domain targeting NY-ESO1 1G4, a TCR beta transmembrane domain, and a CD28 intracellular domain linked to TCR alpha with a GGS linker comprises an amino acid sequence of SEQ ID NO: 152.

In some embodiments, an extracellular domain targeting NY-ESO1 1G4, a TCR beta transmembrane domain, and a CD28 intracellular domain linked to TCR alpha with a GGS linker is encoded by nucleotide sequence of SEQ ID NO: 153.

### Engineered TCRs

Provided herein are engineered TCRs comprising one or more of the fusion proteins of the instant disclosure.

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

As used herein, a “chimeric TCR” or “engineered TCR” refers to a TCR comprising at least one fusion protein comprising at least one domain capable of providing a stimulatory or inhibitory signal.

In some embodiments, a TCR comprises TCR alpha, TCR beta, CD3 zeta, CD3 delta, CD3 gamma and CD3 epsilon, and at least one of TCR alpha, TCR beta, CD3 delta, CD3 gamma and CD3 epsilon is a fusion protein comprising at least one domain capable of providing a stimulatory signal.

In some embodiments, a TCR comprises a TCR alpha polypeptide, a TCR beta polypeptide, two CD3 zeta polypeptides, a CD3 delta polypeptide, a CD3 gamma polypeptide and two CD3 epsilon polypeptides, and at least one of the TCR alpha polypeptide, the TCR beta polypeptide, the CD3 delta polypeptide, the CD3 gamma polypeptide and the two CD3 epsilon polypeptides is a fusion protein comprising at least one domain capable of providing a stimulatory or inhibitory signal.

### Inhibitory Engineered TCRs

Provided herein are engineered TCRs comprising fusion proteins comprising the signaling domains capable of providing an inhibitory signal described herein.

In some embodiments, the engineered TCR comprises a fusion protein comprising an inhibitory, intracellular signaling domain fused to a TCR alpha or TCR beta subunit. In some embodiments, the engineered TCR comprises a fusion protein comprising an inhibitory, intracellular signaling domain fused to the TCR alpha and TCR beta subunits. In some embodiments, the inhibitory, intracellular signaling domain is fused, N to C terminal, to the intracellular domain or transmembrane domain of TCR alpha or TCR beta. In some embodiments, the inhibitory, intracellular signaling domain is fused to TCR alpha or TCR beta using a linker as described herein.

In some embodiments, the engineered TCR comprises a fusion protein comprising an inhibitory, intracellular signaling domain fused to a CD3 delta, CD3 epsilon, CD3 gamma or CD3 zeta subunit. The inhibitory intracellular domain can be fused to the CD3 delta, CD3 epsilon, CD3 gamma or CD3 zeta intracellular domain or a fragment thereof, or the CD3 delta, CD3 epsilon, CD3 gamma or CD3 zeta transmembrane domain. In some embodiments, the inhibitory intracellular domain is fused to the CD3 delta, CD3 epsilon, CD3 gamma or CD3 zeta subunit using a linker as described herein.

In some embodiments, the engineered TCR comprises a fusion protein comprising an inhibitory, intracellular signaling domain fused to CD3 delta, CD3 epsilon, CD3 gamma and CD3 zeta subunits. In some embodiments, the engineered TCR comprises a fusion protein comprising an inhibitory, intracellular signaling domain fused to CD3 delta, CD3 epsilon, CD3 gamma, CD3 zeta, TCR alpha and TCR beta subunits.

In some embodiments, the engineered TCR comprises a fusion protein comprising a CD3 zeta transmembrane domain and an inhibitory intracellular domain. In some embodiments, the CD3 zeta transmembrane domain comprises a sequence of LCYLLDGILFIYGVILTALFL (SEQ ID NO: 76), or a sequence having at least 90%, at least 95%, at least 98% or 100% identity thereto. In some embodiments, the CD3 zeta transmembrane domain comprises or consists essentially of SEQ ID NO: 76. In some embodiments, the CD3 zeta transmembrane domain comprises a sequence of LCYLLKGILFIYGVILTALFL (SEQ ID NO: 77), the D15K mutation in the CD3 zeta transmembrane domain. In some embodiments, the CD3 zeta transmembrane domain comprises a sequence of LCYLLRGILFIYGVILTALFL (SEQ ID NO: 78), the D15R mutation in the CD3 zeta transmembrane domain. The D15 amino acid residue (the D15K and D15R mutations are substitutions of K or R for D at residue 6 of SEQ ID NO: 76) interacts with the R253 amino acid residue of TCR alpha (residue 5 of SEQ ID NO: 9), and the combination of specific positive and negatively charged amino acids at these positions can influence the formation of specific TCR complexes in the cell.

In some embodiments, the engineered TCR comprises a fusion protein comprising a CD3 gamma transmembrane domain and an inhibitory intracellular domain. In some embodiments, the CD3 gamma transmembrane domain comprises a sequence of GFLFAEIVSIFVLAVGVYFIA (SEQ ID NO: 8), or a sequence having at least 90%, at least 95%, at least 98% or 100% identity thereto. In some embodiments, the CD3 gamma transmembrane domain comprises or consists essentially of SEQ ID NO: 8. In some embodiments, the CD3 gamma transmembrane domain comprises a sequence of GFLFAKIVSIFVLAVGVYFIA (SEQ ID NO: 79), the E122K mutation of CD3 gamma. The E122 residue (E122K is a substitution of K for E at amino acid residue 6 of SEQ ID NO: 8) interacts with K288 of TCR beta and D137 of CD3 epsilon, and the combination of specific amino acids at these residues can influence the formation of specific TCR complexes in the cell via interaction of positively and negatively charged amino acids.

In some embodiments, the engineered TCR comprises a fusion protein comprising a TCR alpha transmembrane domain and an inhibitory intracellular domain. In some embodiments, the TCR alpha transmembrane domain comprises a sequence of VIGFRILLLKVAGFNLLMTLRLW (SEQ ID NO: 9), or a sequence having at least 90%, at least 95%, at least 98% or 100% identity thereto. In some embodiments, the TCR alpha transmembrane domain comprises or consists essentially of SEQ ID NO: 9. In some embodiments, the TCR alpha transmembrane domain comprises a substitution of D or E for R at position 253 (amino acid residue 5 of SEQ ID NO: 9). In some embodiments, the TCR alpha transmembrane domain comprises a sequence VIGFDILLLKVAGFNLLMTLRLW (SEQ ID NO: 80). In some embodiments, the TCR alpha transmembrane domain comprises a sequence VIGFEILLLKVAGFNLLMTLRLW (SEQ ID NO: 103).

In some embodiments, the engineered TCR comprises a fusion protein comprising a TCR beta transmembrane domain and an inhibitory intracellular domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of: TILYEILLGKATLYAVLVSALVL (SEQ ID NO: 10). In some embodiments, the TCR beta transmembrane domain comprises, or consists essentially of, SEQ ID NO: 10. In some embodiments, the TCR beta transmembrane domain comprises a substitution of D or E for K at position 288 (position 10 of SEQ ID NO: 10). In some embodiments, the TCR beta transmembrane domain comprises a sequence of TILYEILLGDATLYAVLVSALVL (SEQ ID NO: 104). In some embodiments, the TCR beta transmembrane domain comprises a sequence of TILYEILLGEATLYAVLVSALVL (SEQ ID NO: 105). TCR beta amino acid residue 288 interacts with residues 137 of CD3 epsilon and 122 of CD3 gamma, and the combination of specific positive and negatively charged amino acids at these positions can influence the formation of specific TCR complexes in the cell.

In some embodiments, the engineered TCR comprises a fusion protein comprising a CD3 delta transmembrane domain and an inhibitory intracellular domain. In some embodiments, the CD3 delta transmembrane domain comprises a sequence of GIIVTDVIATLLLALGVFCFA (SEQ ID NO: 106), GIIVTDVIATLLLAGVFCFA (SEQ ID NO: 6), or a sequence having at least 90%, at least 95%, at least 98% or 100% identity thereto. In some embodiments, the CD3 delta transmembrane domain comprises or consists essentially of SEQ ID NO: 106. In some embodiments, the CD3 delta transmembrane domain comprises a substitution at position 137 of CD3 delta (amino acid residue 6 of SEQ ID NO: 106). In some embodiments, the CD3 delta transmembrane domain comprises a substitution of R or K for D at position 6 of SEQ ID NO: 106. In some embodiments, the CD3 delta transmembrane domain comprises a sequence of GIIVTRVIATLLLALGVFCFA (SEQ ID NO: 107). In some embodiments, the CD3 delta transmembrane domain comprises a sequence of GIIVTKVIATLLLALGVFCFA (SEQ ID NO: 108).

In some embodiments, the engineered TCR comprises a fusion protein comprising a CD3 epsilon transmembrane domain and an inhibitory intracellular domain. In some embodiments, the CD3 epsilon transmembrane domain comprises a sequence of VMSVATIVIVDICITGGLLLLVYYWS (SEQ ID NO: 7), or a sequence having at least 90%, at least 95%, at least 98% or 100% identity thereto. In some embodiments, the CD3 epsilon transmembrane domain comprises or consists essentially of SEQ ID NO: 7. In some embodiments, the epsilon transmembrane domain comprises a substitution at position 137 of CD3 epsilon (position 11 of SEQ ID NO: 7). In some embodiments, the CD3 epsilon transmembrane domain comprises a substation of R or K for D at position 11 of SEQ ID NO: 110. In some embodiments, the CD3 epsilon transmembrane domain comprises a sequence of VMSVATIVIVRICITGGLLLLVYYWS (SEQ ID NO: 110). In some embodiments, the CD3 epsilon transmembrane domain comprises a sequence of VMSVATIVIVKICITGGLLLLVYYWS (SEQ ID NO: 111).

In some embodiments, the engineered TCR comprises a fusion protein comprising a CD3 gamma transmembrane domain and an inhibitory intracellular domain. In some embodiments, the CD3 gamma transmembrane domain comprises a sequence of GFLFAEIVSIFVLAVGVYFIA (SEQ ID NO: 112), or a sequence having at least 90%, at least 95%, at least 98% or 100% identity thereto. In some embodiments, the CD3 gamma transmembrane domain comprises or consists essentially of SEQ ID NO: 112. In some embodiments, the CD3 gamma transmembrane domain comprises a substitution at position 122 of CD3 gamma (position 6 of SEQ ID NO: 112). In some embodiments, the CD3 gamma transmembrane domain comprises a substitution of R or K for E at position 6 of SEQ ID NO: 112. In some embodiments, the CD3 gamma transmembrane domain comprises a sequence of GFLFARIVSIFVLAVGVYFIA (SEQ ID NO: 113). In some embodiments, the CD3 gamma transmembrane domain comprises a sequence of GFLFAKIVSIFVLAVGVYFIA (SEQ ID NO: 114).

In some embodiments, the engineered TCR comprises a fusion protein comprising a TCR beta transmembrane domain of SEQ ID NO: 104 or 105, a fusion protein comprising a CD3 epsilon transmembrane domain of SEQ ID NO: 110 or 111, and a fusion protein comprising a CD3 gamma transmembrane domain of SEQ ID NO: 113 or 114. In some embodiments, the engineered TCR comprises a fusion protein comprising a TCR beta transmembrane domain of SEQ ID NO: 10, a fusion protein comprising a CD3 epsilon transmembrane domain of SEQ ID NO: 7, and a fusion protein comprising a CD3 gamma transmembrane domain of SEQ ID NO: 112.

In some embodiments, the engineered TCR comprises a fusion protein comprising a TCR alpha transmembrane domain of SEQ ID NO: 9, and a fusion protein comprising a CD3 zeta transmembrane domain of SEQ ID NO: 76. In some embodiments, the engineered TCR comprises a fusion protein comprising a TCR alpha transmembrane domain of SEQ ID NO: 80 or 103, and a fusion protein comprising a CD3 zeta transmembrane domain of SEQ ID NO: 77 or 78.

In some embodiments, the engineered TCR comprises a fusion protein comprising a TCR beta transmembrane domain of SEQ ID NO: 104 and a fusion protein comprising a CD3 gamma transmembrane domain of SEQ ID NO: 113 or 114.

In some embodiments, the engineered TCR comprises a fusion protein comprising an inhibitory intracellular domain and transmembrane domain that are from the same inhibitory protein, for example, a TRAIL receptor, CD200 receptor 1, PD-1, CTLA-4, KIR3DL2, KIR3DL3, ZAP70, LIR1, FcgRIIB or NKG2D. In some embodiments, the intracellular domain and transmembrane domain from an inhibitory protein are fused to an extracellular domain of TCR alpha, TCR beta, CD3 delta, CD3 epsilon, CD3 gamma or CD3 zeta.

In some embodiments, the engineered TCR comprises an extracellular domain fused to one of the subunits of the TCR, for example an extracellular antigen binding domain or domains fused to one or more of TCR alpha, TCR beta, CD3 delta, CD3 epsilon, CD3 gamma or CD3 zeta. In some embodiments, the extracellular antigen binding domain comprises an antibody fragment, an ScFv, or a single domain antibody (sdAb). In some embodiments, the CD3 subunit comprises a CD3 delta, CD3 epsilon, CD3 gamma or CD3 zeta subunit with a deletion of one or more ITAMs.

In some embodiments, the CD3 delta subunit of a fusion protein of the disclosure comprises a deletion of an ITAM. In some embodiments, the sequence of the CD3 delta subunit comprises a sequence having at least 90%, at least 95%, at least 98% or 100% identity to a sequence of SEQ ID NO: 171. In some embodiments, the CD3 delta sequence is encoded by a nucleotide sequence having at least 90%, at least 95%, at least 98% or 100% identity to SEQ ID NO: 175.

In some embodiments, the CD3 epsilon subunit of a fusion protein of the disclosure comprises a deletion of an ITAM. In some embodiments, the sequence of the CD3 epsilon subunit comprises a sequence having at least 90%, at least 95%, at least 98% or 100% identity to a sequence of SEQ ID NO: 172. In some embodiments, the CD3 epsilon sequence is encoded by a nucleotide sequence having at least 90%, at least 95%, at least 98% or 100% identity to SEQ ID NO: 176.

In some embodiments, the CD3 gamma subunit of a fusion protein of the disclosure comprises a deletion of an ITAM. In some embodiments, the sequence of the CD3 gamma subunit comprises a sequence having at least 90%, at least 95%, at least 98% or 100% identity to a sequence of SEQ ID NO: 173. In some embodiments, the CD3 delta sequence is encoded by a nucleotide sequence having at least 90%, at least 95%, at least 98% or 100% identity to SEQ ID NO: 177.

In some embodiments, the CD3 zeta subunit of a fusion protein of the disclosure comprises a deletion of an ITAM. In some embodiments, the sequence of the CD3 zeta subunit comprises a sequence having at least 90%, at least 95%, at least 98% or 100% identity to a sequence of SEQ ID NO: 174. In some embodiments, the CD3 zeta sequence is encoded by a nucleotide sequence having at least 90%, at least 95%, at least 98% or 100% identity to SEQ ID NO: 178.

In exemplary embodiments, the engineered TCR comprises a fusion protein comprising a PD1 intracellular domain fused to a CD3 delta subunit. In some embodiments, the CD3 delta comprises a deletion of an ITAM sequence. In some embodiments, the fusion protein comprising a PD1 intracellular domain fused to CD3 delta comprises a sequence of SEQ ID NO: 179. In some embodiments, the fusion protein comprising a PD1 intracellular domain fused to CD3 delta is encoded by polynucleotide sequence of SEQ ID NO: 191.

In exemplary embodiments, the engineered TCR comprises a fusion protein comprising a PD1 intracellular domain fused to a CD3 epsilon subunit. In some embodiments, the CD3 epsilon subunit comprises a deletion of an ITAM sequence. In some embodiments, the fusion protein comprising a PD1 intracellular domain fused to CD3 epsilon comprises a sequence of SEQ ID NO: 180. In some embodiments, the fusion protein comprising a PD1 intracellular domain fused to CD3 epsilon is encoded by polynucleotide sequence of SEQ ID NO: 193.

In exemplary embodiments, the engineered TCR comprises a fusion protein comprising a PD1 intracellular domain fused to a CD3 gamma subunit. In some embodiments, the CD3 gamma subunit comprises a deletion of an ITAM sequence. In some embodiments, the fusion protein comprising a PD1 intracellular domain fused to CD3 gamma comprises a sequence of SEQ ID NO: 181. In some embodiments, the fusion protein comprising a PD1 intracellular domain fused to CD3 gamma is encoded by polynucleotide sequence of SEQ ID NO: 194.

In exemplary embodiments, the engineered TCR comprises a fusion protein comprising a PD1 intracellular domain fused to a CD3 zeta subunit. In some embodiments, the CD3 zeta subunit comprises a deletion of an ITAM sequence. In some embodiments, the fusion protein comprising a PD1 intracellular domain fused to CD3 zeta comprises a sequence of SEQ ID NO: 182. In some embodiments, the fusion protein comprising a PD1 intracellular domain fused to CD3 zeta is encoded by polynucleotide sequence of SEQ ID NO: 195.

In exemplary embodiments, the engineered TCR comprises a fusion protein comprising a LIR1 intracellular domain fused to a CD3 delta subunit. In some embodiments, the CD3 delta comprises a deletion of an ITAM sequence. In some embodiments, the fusion protein comprising a LIR1 intracellular domain fused to CD3 delta comprises a sequence of SEQ ID NO: 183. In some embodiments, the fusion protein comprising a LIR1 intracellular domain fused to CD3 delta is encoded by polynucleotide sequence of SEQ ID NO: 196. In some embodiments, the fusion protein comprising a LIR1 intracellular domain fused to CD3 delta comprises a sequence of SEQ ID NO: 187, and is encoded by a polynucleotide sequence of SEQ ID NO: 200.

In exemplary embodiments, the engineered TCR comprises a fusion protein comprising a LIR1 intracellular domain fused to a CD3 epsilon subunit. In some embodiments, the CD3 epsilon subunit comprises a deletion of an ITAM sequence. In some embodiments, the fusion protein comprising a LIR1 intracellular domain fused to CD3 epsilon comprises a sequence of SEQ ID NO: 184. In some embodiments, the fusion protein comprising a LIR1 intracellular domain fused to CD3 epsilon is encoded by polynucleotide sequence of SEQ ID NO: 197. In some embodiments, the fusion protein comprising a LIR1 intracellular domain fused to CD3 epsilon comprises a sequence of SEQ ID NO: 188, and is encoded by a polynucleotide sequence of SEQ ID NO: 201.

In exemplary embodiments, the engineered TCR comprises a fusion protein comprising a LIR1 intracellular domain fused to a CD3 gamma subunit. In some embodiments, the CD3 gamma subunit comprises a deletion of an ITAM sequence. In some embodiments, the fusion protein comprising a LIR1 intracellular domain fused to CD3 gamma comprises a sequence of SEQ ID NO: 185. In some embodiments, the fusion protein comprising a LIR1 intracellular domain fused to CD3 gamma is encoded by polynucleotide sequence of SEQ ID NO: 198. In some embodiments, the fusion protein comprising a LIR1 intracellular domain fused to CD3 gamma comprises a sequence of SEQ ID NO: 189, and is encoded by a polynucleotide sequence of SEQ ID NO: 202.

In exemplary embodiments, the engineered TCR comprises a fusion protein comprising a LIR1 intracellular domain fused to a CD3 zeta subunit. In some embodiments, the CD3 zeta subunit comprises a deletion of an ITAM sequence. In some embodiments, the fusion protein comprising a LIR1 intracellular domain fused to CD3 zeta comprises a sequence of SEQ ID NO: 186. In some embodiments, the fusion protein comprising a LIR1 intracellular domain fused to CD3 zeta is encoded by polynucleotide sequence of SEQ ID NO: 199. In some embodiments, the fusion protein comprising a LIR1 intracellular domain fused to CD3 zeta comprises a sequence of SEQ ID NO: 190, and is encoded by a polynucleotide sequence of SEQ ID NO: 202.

### Activator Engineered TCRs

In some embodiments, an engineered T-cell receptor comprises a first fusion protein and a second fusion protein. In some embodiments, the first fusion protein comprises a TCR alpha intracellular domain fused to at least one domain capable of providing a stimulatory signal, and the second fusion protein comprises a TCR beta intracellular domain fused to at least one domain capable of providing a stimulatory signal. In some embodiments, first fusion protein comprises a TCR alpha or beta intracellular domain fused to at least one domain capable of providing a stimulatory signal, and the second fusion protein comprises a CD3 delta, gamma or epsilon intracellular domain fused to at least one domain capable of providing a stimulatory signal. In some embodiments, first fusion protein comprises a TCR alpha or beta transmembrane domain fused to at least one domain capable of providing a stimulatory signal, and the second fusion protein comprises a CD3 delta, gamma or epsilon intracellular domain fused to at least one domain capable of providing a stimulatory signal. In some embodiments the domains capable of providing a stimulatory signal are the same domain (e.g., CD28 and CD28, Lck and Lck, etc). In some embodiments the domains capable of providing a stimulatory signal are not the same domain (e.g., 4-1BB and CD4, CD28 and Lck, etc).

In some embodiments, the first fusion protein comprises a TCR alpha intracellular domain fused to a first domain capable of providing a stimulatory signal and the second fusion protein comprises a TCR beta intracellular domain fused to a second domain capable of providing al stimulatory signal. In some embodiments, the first and/or second domains capable of providing a stimulatory signal is selected from CD28 domain, Lck domain, 4-1BB domain, CD4 domain, CD8a domain, Fyn domain, ZAP70 domain, LAT domain, and SLP76 domain. The first domain fused to TCR alpha, and the second domain fused to TCR beta, can be the same domain (e.g. both CD28) or different domains (e.g. CD28 fused to TCR alpha and 4-1BB fused to TCR beta or vice versa).

In some embodiments, the an engineered TCR comprises a first and second fusion protein, and the first fusion protein comprises a first domain capable of providing a stimulatory signal, and the second fusion protein comprises a second and a third domain each capable of providing a stimulatory signal. In some embodiments, the first, second and third domains capable of providing a stimulatory signal are selected from a CD28 domain, an Lck domain, a 4-1BB domain, a CD4 domain, a CD8a domain, a Fyn domain, a ZAP70 domain, a LAT domain, and a SLP76 domain. For example, an engineered TCR can comprise TCR alpha fused to CD28 and TCR beta fused to Lat and Fyn domains or fused to LAT and Lck domains.

In some embodiments, an engineered TCR comprises a fusion protein comprising a TCR alpha intracellular domain and a CD28 domain. In some embodiments, an engineered TCR comprises a fusion protein comprising a TCR beta intracellular domain and a CD28 domain. In some embodiments, an engineered TCR comprises a first fusion protein comprising a TCR alpha intracellular domain and a CD28 domain and a second fusion protein comprising a TCR beta intracellular domain and a CD28 domain.

In some embodiments, an engineered TCR comprises a fusion protein comprising a CD3 gamma intracellular domain and a CD28 domain. In some embodiments, an engineered TCR comprises a fusion protein comprising a CD3 delta intracellular domain and a CD28 domain. In some embodiments, an engineered TCR comprises a fusion protein comprising a CD3 epsilon intracellular domain and a CD28 domain.

In some embodiments, an engineered TCR comprises a fusion protein comprising a TCR alpha intracellular domain and a 4-1BB domain. In some embodiments, an engineered TCR comprises a fusion protein comprising a TCR beta intracellular domain and a 4-1BB domain. In some embodiments, an engineered TCR comprises a first fusion protein comprising a TCR alpha intracellular domain and a 4-1BB domain and a second fusion protein comprising a TCR beta intracellular domain and a 4-1BB domain. In some embodiments, an engineered TCR comprises a first fusion protein comprising a TCR alpha intracellular domain and a CD28 domain and a second fusion protein comprising a TCR beta intracellular domain and a 4-1BB domain. In some embodiments, an engineered TCR comprises a first fusion protein comprising a TCR alpha intracellular domain and a 4-1BB domain and a second fusion protein comprising a TCR beta intracellular domain and a CD28 domain. In some embodiments, the 4-1BB domain, the CD28 domain, or both are linked to the TCR alpha or TCR beta intracellular domain with a linker. In some embodiments, the linker comprises or consists essentially of a sequence of GGGGS GGGGS GGGGS (SEQ ID NO: 20). In some embodiments, the linker comprises or consists essentially of a sequence of GGGGS (SEQ ID NO: 18).

In some embodiments, an engineered TCR comprises a fusion protein comprising a TCR alpha intracellular domain and a CD4 domain. In some embodiments, an engineered TCR comprises a fusion protein comprising a TCR beta intracellular domain and a CD4 domain. In some embodiments, an engineered TCR comprises a first fusion protein comprising a TCR alpha intracellular domain and a CD4 domain and a second fusion protein comprising a TCR beta intracellular domain and a CD4 domain.

In some embodiments, an engineered TCR comprises a fusion protein comprising a TCR alpha intracellular domain and a CD8a domain. In some embodiments, an engineered TCR comprises a fusion protein comprising a TCR beta intracellular domain and a CD8a domain. In some embodiments, an engineered TCR comprises a first fusion protein comprising a TCR alpha intracellular domain and a CD8a domain and a second fusion protein comprising a TCR beta intracellular domain and a CD8a domain. In some embodiments, an engineered TCR comprises a first fusion protein comprising a TCR alpha intracellular domain and a CD4 domain and a second fusion protein comprising a TCR beta intracellular domain and a CD8a domain. In some embodiments, an engineered TCR comprises a first fusion protein comprising a TCR alpha intracellular domain and a CD8a domain and a second fusion protein comprising a TCR beta intracellular domain and a CD4 domain.

In some embodiments, an engineered TCR comprises a fusion protein comprising a TCR beta intracellular domain and a CD4 domain. In some embodiments, an engineered TCR comprises a fusion protein comprising a TCR beta intracellular domain and a CD8a domain. In some embodiments, the CD4 or CD8a domain is linked to the TCR beta intracellular domain with a linker. In some embodiments, the linker comprises or consists essentially of a sequence of SEQ ID NO: 20. In some embodiments, the linker comprises or consists essentially of a sequence of SEQ ID NO: 18.

In some embodiments, an engineered TCR comprises a fusion protein comprising a TCR alpha intracellular domain and Lck domain. In some embodiments, an engineered TCR comprises a fusion protein comprising a TCR beta intracellular domain and Lck domain. In some embodiments, an engineered TCR comprises a first fusion protein comprising a TCR alpha intracellular domain and a Lck domain and a second fusion protein comprising a TCR beta intracellular domain and a Lck domain. In some embodiments, the Lck domain is linked to the TCR alpha or beta intracellular domain with a linker. In some embodiments, the linker comprises a sequence of SEQ ID NO: 20. In some embodiments, the linker comprises or consists essentially of a sequence of SEQ ID NO: 18.

In some embodiments, an engineered TCR comprises a fusion protein comprising a TCR alpha intracellular domain and Fyn domain. In some embodiments, an engineered TCR comprises a fusion protein comprising a TCR beta intracellular domain and Fyn domain. In some embodiments, an engineered TCR comprises a first fusion protein comprising a TCR alpha intracellular domain and a Fyn domain and a second fusion protein comprising a TCR beta intracellular domain and a Fyn domain. In some embodiments, the Fyn domain is linked to the TCR alpha or beta intracellular domain with a linker. In some embodiments, the linker comprises a sequence of SEQ ID NO: 20. In some embodiments, the linker comprises or consists essentially of a sequence of SEQ ID NO: 18.

In some embodiments, an engineered TCR comprises a fusion protein comprising a TCR alpha intracellular domain and Zap70 domain. In some embodiments, an engineered TCR comprises a fusion protein comprising a TCR beta intracellular domain and Zap70 domain. In some embodiments, an engineered TCR comprises a first fusion protein comprising a TCR alpha intracellular domain and a Zap70 domain and a second fusion protein comprising a TCR beta intracellular domain and a Zap70 domain. In some embodiments, the Zap70 domain comprises the Zap70 interdomain B, the Zap70 kinase domain, or a combination thereof. In some embodiments, the Zap70 domain is linked to the TCR alpha or beta intracellular domain with a linker. In some embodiments, the linker comprises a sequence of SEQ ID NO: 20. In some embodiments, the linker comprises or consists essentially of a sequence of SEQ ID NO: 18.

In some embodiments, an engineered TCR comprises a fusion protein comprising a TCR alpha intracellular domain and Lat domain. In some embodiments, an engineered TCR comprises a fusion protein comprising a TCR beta intracellular domain and Lat domain. In some embodiments, an engineered TCR comprises a first fusion protein comprising a TCR alpha intracellular domain and a Lat domain and a second fusion protein comprising a TCR beta intracellular domain and a Lat domain. In some embodiments, the Lat domain is linked to the TCR alpha or beta intracellular domain with a linker. In some embodiments, the linker comprises a sequence of SEQ ID NO: 20. In some embodiments, the linker comprises or consists essentially of a sequence of SEQ ID NO: 18.

In some embodiments, an engineered TCR comprises a fusion protein comprising a TCR alpha intracellular domain and Slp76 domain. In some embodiments, an engineered TCR comprises a fusion protein comprising a TCR beta intracellular domain and Slp76 domain. In some embodiments, an engineered TCR comprises a first fusion protein comprising a TCR alpha intracellular domain and a Slp76 domain and a second fusion protein comprising a TCR beta intracellular domain and a Slp76 domain. In some embodiments, the Slp76 domain is linked to the TCR alpha or beta intracellular domain with a linker. In some embodiments, the linker comprises a sequence of SEQ ID NO: 20. In some embodiments, the linker comprises or consists essentially of a sequence of SEQ ID NO: 18.

In some embodiments, an engineered TCR comprises a fusion protein comprising a TCR alpha intracellular domain, a Lat domain and a Fyn domain. In some embodiments, an engineered TCR comprises a fusion protein comprising a TCR beta intracellular domain, a Lat domain, and a Fyn domain. In some embodiments, an engineered TCR comprises a fusion protein comprising a TCR alpha intracellular domain, a Lat domain, and a Lck domain. In some embodiments, an engineered TCR comprises a fusion protein comprising a TCR beta intracellular domain, a Lat domain, and a Lck domain.

### Antigens

The disclosure provides engineered TCRs, comprising one or more fusion proteins of the disclosure that are capable of recognizing an antigen.

The term “antigen” refers to a molecule that is capable of being bound specifically by an antibody, or being presented by a major histocompatibility complex and bound by a TCR, or otherwise provokes an immune response. This immune response may involve either antibody production, or the activation of specific immunologically-competent cells, or both.

As used herein, the term “sparse antigen” refers to that is present in the body of a subject in low amounts. For example, a sparse antigen may be derived from a protein with low levels of expression, or expressed in a rare cell type. T-cell activation by APCs is dependent in part on antigen concentration. Sparse antigens, because of their rarity, often fail to trigger a robust T cell activation. In contrast, “abundant antigens” are present in the body of a subject in high concentrations and are capable of triggering a robust (i.e., strong and reproducible) immune response.

In some embodiments, a sparse antigen is an antigen that is present in less than 1,000 copies per cell. In some embodiments, a sparse antigen is an antigen that is present in less than 5000 copies per cell, less than 4000 copies per cell, less than 3000 copies per cell, less than 2000 copies per cell, less than 1000 copies per cell, less than 900 copies per cell, less than 800 copies per cell, less than 700 copies per cell, less than 600 copies per cell, less than 500 copies per cell, less than 400 copies per cell, less than 300 copies per cell, less than 200 copies per cell, less than 100 copies per cell or less than 50 copies per cell.

The term “antigen presenting cell” or “APC” refers to an immune system cell such as an accessory cell (e.g., a B-cell, a dendritic cell, and the like) that displays a foreign antigen complexed with major histocompatibility complexes (MHCs) on its surface. T-cells may recognize these complexes using their T-cell receptors (TCRs). APCs process antigens and present them to T-cells.

In some embodiments, an antigen recognized by an engineered TCR of the disclosure is bound by a major histocompatibility complex (MHC). As used herein, “MHC” refers to a protein complex that binds to antigens and displays them on the cell surface for recognition by the appropriate T cell (e.g., via a TCR). Peptides are processed and presented by cells using two pathways, the MHC class I and MHC class II pathways, both of which are envisaged as within the scope of the disclosure. In MHC class II, phagocytes such as macrophages and immature dendritic cells take up exogenous material (e.g., pathogens, proteins) by phagocytosis into phagosomes or endosomes, which fuse with lysosomes whose acidic enzymes cleave the exogenous material protein into peptides. These peptides are loaded onto MHC class II molecules, which are trafficked to the cell surface and presented to immune cells. In MHC class I, nucleated cells present cytosolic peptides. These cytosolic peptides are mostly self peptides derived from protein turnover and defective ribosomal products. However, during infection with intracellular localization (e.g. by a virus or other microorganism), or a cancerous transformation, the proteins degraded in the proteasome include proteins from the infectious organism or cancer, and are also loaded onto MHC class I molecules and displayed on the cell surface. Thus, in some conditions Class I MHC can display cancer and non-self antigens.

In some embodiments, an engineered TCR of the disclosure recognizes a peptide antigen bound to MHC (pMHC), for example MHC class I or MHC class II.

In some embodiments, an engineered TCR of the disclosure recognizes an antigen that is not bound to MHC. For example, engineered TCRs whose antigen binding domain is derived from an ScFv, or VHH antigen binding domain can bind antigens that are not presented by MHC.

In some embodiments, the antigen is a non-self antigen, for example an antigen from a virus, bacteria, fungus or eukaryotic pathogen.

In some embodiments, the antigen is a self-antigen, for example a protein with a particular tissue-specific expression pattern.

In some embodiments, an engineered TCR of the disclosure recognizes a peptide antigen bound to MHC (pMHC). In some embodiments, the pMHC comprises a cancer antigen. In some embodiments, the pMHC comprises a neoantigen. As used herein, a “neoantigen” refers to an antigen not previously recognized by the immune system. Neoantigens can arise from altered tumor proteins, or from viral proteins, for example. In some embodiments, the neoantigen comprises KRAS G12D, KRAS G12V, p53 or a variant of phosphoinositide 3 kinase alpha (PI3K alpha). In some embodiments, the antigen comprises a viral antigen, such as a human papillomavirus (HPV) antigen. In some embodiments, the antigen comprises a testes or fetal antigen.

In some embodiments, the pMHC comprises a cancer antigen. In some embodiments, the cancer antigen comprises CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (RORI); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GMI; sialyl Lewis adhesion molecule (sLe); ganglioside GM3 (aNeu5Ac(2-3)bDG alp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEMI/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTi); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p⁵³ mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

In some embodiments, the engineered TCR comprises an antigen binding domain that has a binding affinity KD of 10⁻⁴ M to 10⁻⁹M, e.g., 10⁻⁵ M to 10⁻⁷ M, e.g., 10⁻⁶ M or 10⁻⁷ M, for its target antigen. The binding affinity of an antigen binding domain for an antigen can be determined by methods known in the art, e.g. by ELISA or surface plasmon resonance.

In some embodiments, for example those embodiments the engineered TCR is an inhibitory TCR, the antigen binding domain is specific to an antigen that is broadly, or ubiquitously expressed. Examples of broadly expressed antigens include human leukocyte antigens (for example, HLA-A, HLA-B or HLA-C), housekeeping genes and the like. In some embodiments, the antigen is lost through loss of heterozygosity in a cancer cell of a patient.

Exemplary antigen binding domains are described in PCT/US2020/045228, the contents of which are incorporated by reference herein in their entirety.

### Assays

Provided herein are assays that can be used to measure the activity of the engineered TCRs of the disclosure.

Engineered fusion proteins comprising a TCR subunit including (TCR alpha, TCR beta, CD3 delta, CD3 epsilon or CD3 gamma) and intracellular domains including various lengths, portions and combinations of the domains associated with inhibition, co-stimulation, co-activation, and downstream TCR signaling components were assembled using methods known in the art.

The activity of chimeric or engineered TCRs can be assayed using a cell line engineered to express a reporter of TCR activity such as a luciferase reporter. Exemplary cell lines include Jurkat T cells, although any suitable cell line known in the art may be used. For example, Jurkat cells expressing a luciferase reporter under the control of an NFAT promoter can be used as effector cells. Expression of luciferase by this cell line reflects TCR-mediated signaling.

The reporter cells can be transfected with each of the various fusion protein constructs, combinations of fusion protein constructs or controls described herein.

Expression of the fusion proteins in reporter cells can be was confirmed by using fluorescently labeled MHC tetramers, for example Alexa Fluor 647-labeled NY-ESO-1-MHC tetramer, to detect expression of the fusion protein.

To assay the activity of engineered TCRs, target cells are loaded with antigen prior to exposure to the effector cells comprising the report and the engineered TCR. For example, target cells can be loaded with antigen at least 12, 14, 16, 18, 20, 22 or 24 hours prior to exposure to effector cells. Exemplary target cells include A375 cells, although any suitable cells known in the art may be used. In some cases, target cells can be loaded with serially diluted concentrations of an antigen, such as NY-ESO-1 peptide. The effector cells can then be co-cultured with target cells for a suitable period of time, for example 6 hours. Luciferase is then measured by luminescence reading after co-culture. Luciferase luminescence can be normalized to maximum and minimum intensity to allow comparison of activating peptide concentrations for each TCR construct.

Provided herein are methods of determining the relative EC50 of engineered TCRs of the disclosure. As used herein, “EC50” refers to the concentration of an inhibitor or agent where the response (or binding) is reduced by half. EC50s of engineered TCRs of the disclosure refer to concentration of antigen where binding of the engineered TCR to the antigen is reduced by half. Binding of the antigen, or probe to the TCR can be measured by staining with labeled peptide or labeled peptide-MHC complex, for example MHC:NY-ESO-1 pMHC complex conjugated with fluorophore. EC50 can be obtained by nonlinear regression curve fitting of reporter signal with peptide titration. Probe binding and EC50 can be normalized to the levels of benchmark TCR without a fusion protein, e.g. NY-ESO-1 (clone 1G4).

In some embodiments, an engineered TCR comprising a fusion protein has a relative EC50 of greater than or equal to 0.2, 0.3, 0.4, 0.5, 0.9, 1.0, 1.1, 1.2, 1.3, 1.4, 1.5, 1.6, 1.8, 1.9 or 2.0 relative to a TCR that does not comprise a fusion protein.

In some embodiments, an engineered TCR comprising a fusion protein has a relative EC50 of about 0.2-2.0, about 0.5-2.0, about 0.9-2.0, about 1.0-2.0, about 1.1-2.0, about 1.2-2.0, about 0.9-1.7, about 1.0-1.7, or about 1.2-1.7 relative to a TCR that does not comprise a fusion protein

### Polynucleotides

The disclosure provides polynucleotides encoding the sequence of the fusion proteins described herein. In some embodiments, the sequence of the fusion protein is operably linked to a promoter.

The disclosure provides vectors comprising the polynucleotides herein.

The disclosure provides vectors encoding the coding sequence of any of the fusion proteins described herein. In some embodiments, the sequence of the fusion protein is operably linked to a promoter.

In some embodiments, the polynucleotide or vector comprises a sequence encoding a TCR alpha intracellular domain fused to at least one domain capable of providing a co-stimulatory signal. In some embodiments, the polynucleotide or vector comprises a sequence encoding a TCR beta intracellular domain fused to at least one domain capable of providing a stimulatory signal. In some embodiments, the polynucleotide or vector comprises a sequence encoding a CD3 delta, gamma or epsilon intracellular domain fused to at least one domain capable of providing a stimulatory signal. In some embodiments, the at least one domain capable of providing a stimulatory signal is selected from a CD28 domain, Lck domain, 4-1BB domain, CD4 domain, GITR domain, CD8a domain, Fyn domain, ZAP70 domain, LAT domain, and SLP76 domain. In some embodiments, the at least one domain capable of providing a stimulatory signal is fused to the C terminus of the intracellular domain. In some embodiments, the at least one domain capable of providing a stimulatory signal is fused to the C terminus of the intracellular domain with a linker. In some embodiments, the linker comprises, or consists essentially of GGS, SEQ ID NO: 240, SEQ ID NO: 18, SEQ ID NO: 19, SEQ ID NO: 20 or SEQ ID NO: 21. In some embodiments, the vector or polynucleotide comprises a sequence encoding a sequence of GGS, SEQ ID NO: 240, SEQ ID NO: 18, SEQ ID NO: 19, SEQ ID NO: 20 or SEQ ID NO: 21.

In some embodiments, the fusion protein comprises a CD3 delta, CD3 epsilon, CD3 gamma or CD3 zeta subunit fused to an inhibitory intracellular domain, and the vector comprises a sequence encoding the CD3 subunit fused to the inhibitory intracellular domain. In some embodiments the CD3 delta, CD3 epsilon, CD3 gamma or CD3 zeta subunit comprises a deletion of at least one ITAM. In some embodiments, the vector encoding the CD3 delta, CD3 epsilon, CD3 gamma comprises a sequence of SEQ ID NOs: 175-178. In some embodiments, the CD3 delta, CD3 epsilon, CD3 gamma or CD3 zeta subunit is fused to a LIR1 intracellular domain and the vector comprises a sequence of SEQ ID NOs: 196-203. In some embodiments, the CD3 delta, CD3 epsilon, CD3 gamma or CD3 zeta subunit is fused to a PD-1 intracellular domain and the vector comprises a sequence of SEQ ID NOs: 191-194

In some embodiments, the fusion protein comprises a TCR alpha or TCR beta subunit fused to an inhibitory intracellular domain, and the vector comprises a sequence encoding the TCR alpha or TCR beta subunit fused to an inhibitory intracellular domain.

In some embodiments, the vector is an expression vector, i.e. for the expression of the fusion protein in a suitable cell.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The expression of natural or synthetic nucleic acids encoding fusion proteins is typically achieved by operably linking a nucleic acid encoding the fusion protein or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The polynucleotides encoding the fusion proteins can be cloned into a number of types of vectors. For example, the polynucleotides can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a fusion protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Pharmaceutical Compositions

The disclosure provides pharmaceutical compositions comprising immune cells comprising the engineered TCRs of the disclosure and a pharmaceutically acceptable diluent, carrier or excipient.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

### Immune Cells

Provided herein are immune cells comprising the polynucleotides, vectors, fusion proteins and engineered TCRs described herein.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymophonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells which develop upon migration to the thymus, which include, helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

In some embodiments, the engineered immune cell comprising the TCR of the disclosure is a T cell. In some embodiments, the T cell comprises a deletion in one or more endogenous TCR subunits (i.e., TCR subunits encoded by the immune cell genome). Deletions in TCR subunits prevent the expression of a functional TCR subunit protein. In some embodiments, the T cell comprises deletions in the genomic sequences encoding CD3 delta, CD3 epsilon and/or CD3 gamma. In some embodiments, the T cell comprises deletions the genomic sequences encoding CD3 delta, CD3 epsilon, CD3 gamma and/or CD3 zeta. In some embodiments, the T cell comprises deletions in the genomic sequences TCR alpha, TCR beta, CD3 delta, CD3 epsilon, CD3 gamma and/or CD3 zeta. Deletions may be made by any methods known in the art, including, but not limited to CRISPR/Cas mediated genetic engineering using Cas9 and appropriate guide RNAs (gRNAs), where the gRNA are specific to exonic sequences in the TCR subunits. The Cas9-gRNA complex can create a double-stranded break at a specific location, which, when repaired by non-homologous end joining, produces a deletion at that location. Suitable additional methods creating deletions will be known to persons of ordinary skill in the art.

In some embodiments, the engineered immune cell comprising the TCR of the disclosure is a T cell. In some embodiments, the T cell is an effector T cell or a regulatory T cell.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Methods of activating and culturing populations of T cells comprising the engineered TCRs, fusion proteins or vectors encoding the fusion proteins of the instant disclosure, will be readily apparent to the person of ordinary skill in the art.

Whether prior to or after genetic modification of T cells to express an engineered TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10040846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% C02).

In some embodiments, the T cells comprising engineered TCRs of the disclosure are autologous. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Methods of Use

T Cells Comprising Engineered TCRs with Inhibitory Domains

Provided herein are methods of reducing or inhibiting the activity of immune cells comprising the inhibitory engineered TCRs of the disclosure. For example, immune cells comprising inhibitory TCRs can be used to reduce the “on target, off tumor” effects of adoptive cell therapies, and graft versus host disease, by inhibiting the activity of T cells comprising the inhibitory engineered upon contact with a specific antigen recognized by the engineered TCR.

The inhibitory engineered TCRs of the disclosure, by reducing or blocking TCR signaling upon activation of the TCR, can reduce T cell proliferation, survival, cytokine production or cytotoxicity, or a combination thereof. The term “effective amount” or “therapeutically effective amount” are used interchangeably herein, and refer to an amount of a composition, such as a composition comprising engineered TCRs or a composition comprising immune cells comprising engineered TCRs, as described herein, effective to achieve a particular biological or therapeutic result. In a subject with cancer, a therapeutically effective amount of the composition of immune cells comprising the engineered TCRs comprising a stimulatory domain reduces a sign or a symptom of the cancer. For example, a therapeutically effective amount of the composition reduces tumor number, volume, or size, or slows tumor growth, or a combination thereof. As a further example, in a subject undergoing a potentially toxic adoptive cell therapy, a therapeutically effective amount of a composition of immune cells comprising engineered TCRs comprising an inhibitory domain reduces on target, off tumor effects of the adoptive cell therapy.

Engineered TCRs with inhibitory domains, as described herein, can be used as part of a two-receptor system expressed by immune cells to increase the specificity of adoptive cell therapies, and reduce targeting of healthy cells by adoptive immune cells. For example, immune cells expressing an activator receptor, such as a CAR of TCR, may attack not only diseased cells (target cells) that express the target ligand, but also healthy cells (non-target cells) that also express the target ligand. Expressing a second, inhibitory receptor that inhibits immune cell activation in the presence of a second, non-target, ligand expressed by healthy cells but that is not expressed by diseased cells, the specificity of the adoptive cell therapy is increased and potentially harmful side effects are decreased. The second inhibitory receptor can be an inhibitory TCR, as described herein. As an example, if the disease is cancer, the non-target ligand may be lost in cancer cells through loss of heterozygosity. In various embodiments, the compositions and methods of the present disclosure are used in combination with the compositions and methods described in Int'l Pat. Appl. No. PCT/US2020/045228 and Int'l Pat. Appl. Pub Nos. WO 2018/061012, WO 2019/068007 and WO 2020/065406, the contents of which are incorporated by reference in their entireties. In particular, an inverter TCR may be generated for any of the negative or blocker targets disclosed therein, such that an inverter TCR supplements or takes the place of an inhibitor CAR. Additional receptors for use in the methods described herein can be found in PCT/US2020/045373, PCT/US2020/045250, PCT/CA2016/051421, the contents of each of which are incorporated by reference in their entireties.

T Cells Comprising Engineered TCRs with Stimulatory Domains

Provided herein are methods of promoting cell death of a target cell, comprising contacting the target cell with a T cell comprising the engineered TCR comprising intracellular domains capable of providing a stimulatory signal of the disclosure. For example, the target cell expresses an antigen, and the engineered TCR comprises an antigen binding domain that binds to the antigen expressed by the target cell. In some embodiments, the T cell exhibits a cytolytic effect on the target cell at an EC50 of less than 10⁻¹, 10⁻², or 10⁻³ μM peptide antigen. In some embodiments, the T cell exhibits a cytolytic effect when the target cell expresses a sparse antigen recognized by the engineered TCR.

Provided herein are methods of preventing T cell exhaustion, for example preventing exhaustion of T cells comprising the engineered TCRs of the instant disclosure that are used in the treatment of cancer or chronic viral infection.

Provided herein are methods of inducing T cell responses to overcome chronic infection. Chronic infections include chronic viral, bacterial and fungal infections. The methods described herein can be used to induce plastic T cell responses in T cells expressing the engineered TCRs of the disclosure. These T cells comprising the engineered TCRs described herein can be administered to a subject, to treat a chronic infection, such as an intracellular bacterial infection. Exemplary intracellular infections include infections that can form granulomas, such as include tuberculosis.

Provided herein are methods of treating an inflammatory disease, comprising administering to a subject the engineered TCRs described herein. Exemplary inflammatory diseases include, but are not limited to, asthma, rheumatoid arthritis, ulcerative colitis, Crohn's disease, psoriatic arthritis, inflammatory bowel disease and chronic obstructive pulmonary disease.

Provided herein are methods of treating an autoimmune disease, comprising administering to a subject the engineered TCRs described herein. Exemplary autoimmune diseases include, but are not limited to, systemic lupus erythematous, type-1 diabetes mellitus, multiple sclerosis, rheumatoid arthritis, psoriasis, chronic inflammatory demylelinating polyneuropathy and vasculitis.

Provided herein are methods of treating a subject in need thereof, comprising administering to the subject a therapeutically effective amount of a composition comprising immune cells comprising the engineered TCRs of the disclosure.

In some embodiments, the subject in need thereof has cancer. Cancer is a disease in which abnormal cells divide without control and spread to nearby tissue. In some embodiments, the cancer comprises a liquid tumor, or a solid tumor. Exemplary liquid tumors include leukemias and lymphomas. Exemplary solid tumors include sarcomas and carcinomas. Cancers can arise in virtually an organ in the body, including blood, bone marrow, lung, breast, colon, bone, central nervous system, pancreas, prostate and ovary.

In some embodiments, the subject in need thereof has an infection. Infections include viral, bacterial and fungal infections, as well as infection with eukaryotic parasites.

In some embodiments, the subject in need thereof has an autoimmune disease, allergic disease, or inflammatory disease.

In some embodiments, the autoimmune disease, allergic disease, or inflammatory disease is selected from the group consisting of systemic lupus erythematosus, rheumatoid arthritis, psoriatic arthritis, scleroderma, asthma, atopic dermatitis, and allergic rhinitis.

In some embodiments, the autoimmune disease, allergic disease, or inflammatory disease is an organ-specific inflammatory disease.

In some embodiments, the organ-specific inflammatory disease is selected from the group consisting of kidney disease and lung disease. In some embodiments, the organ-specific inflammatory disease is a disease of any other organ or group of organs in the body (for example, liver, brain, heart intestines, lymph nodes, circulatory system, stomach, spleen etc.).

In some embodiments, the autoimmune disease, allergic disease, or inflammatory disease is transplant rejection. In some embodiments, the transplant rejection occurs in response to transplanted blood, bone marrow, bone, skin, heart, kidney, lung, muscle, heart or liver. In some embodiments, the transplant rejection is hyperacute rejection. In some embodiments, the transplant rejection is acute rejection. In some embodiments, the transplant rejection is chronic rejection.

### Kits and Articles of Manufacture

The disclosure provides kits and articles of manufacture comprising the polynucleotides and vectors encoding the fusion proteins described herein, and immune cells comprising the engineered TCRs described herein. In some embodiments, the kit comprises articles such as vials, syringes and instructions for use.

In some embodiments, the kit comprises a polynucleotide or vector comprising a sequence encoding one or more fusion proteins of the disclosure. For example, the polynucleotide or vector comprises a sequence encoding TCR alpha, TCR beta, CD3 epsilon, CD3 delta and/or CD3 gamma and one or more domains capable of providing an inhibitory signal, as described herein.

In some embodiments, the kit comprises a plurality of immune cells comprising an engineered TCR as described herein, for example a TCR comprising an alpha and/or beta chain fused to a domain capable of providing a inhibitory signal. In some embodiments, the plurality of immune cells comprises a plurality of T cells.

The present description sets forth numerous exemplary configurations, methods, parameters, and the like. It should be recognized, however, that such description is not intended as a limitation on the scope of the present disclosure, but is instead provided as a description of exemplary embodiments.

### ENUMERATED EMBODIMENTS

The invention can be understood with reference to the following enumerated embodiments:

1. A fusion protein, comprising an extracellular domain, a transmembrane domain and a first intracellular domain capable of providing an inhibitory signal.

2. The fusion protein of embodiment 1, comprising a second intracellular domain.

3. The fusion protein of embodiment 1 or 2, wherein the first intracellular domain comprises an immunoreceptor tyrosine-based inhibitory motif (ITIM).

4. The fusion protein of embodiment 3, wherein the ITIM is synthetic or non-naturally occurring.

5. The fusion protein of embodiment 2 or 3, wherein the first intracellular domain comprises a programmed cell death 1 (PD-1) intracellular domain, a cytotoxic T-lymphocyte associated protein 4 (CTLA-4) intracellular domain, a killer cell immunoglobulin like receptor, three Ig domains and long cytoplasmic tail 2 (KIR3DL2) intracellular domain, a killer cell immunoglobulin like receptor, three Ig domains and long cytoplasmic tail 3 (KIR3DL3) intracellular domain, a zeta chain of T cell receptor associated protein kinase 70 (ZAP70) domain comprising a Src Homology 2 (SH2) domain, a ZAP70 inactivated kinase domain, a leukocyte immunoglobulin like receptor B1 (LIR1) intracellular domain, an Fc gamma receptor IIB (FcgRIIB) intracellular domain, or a killer cell lectin like receptor K1 (NKG2D) intracellular domain.

6. The fusion protein of embodiment 5, wherein the first intracellular domain comprises a LIR1 intracellular domain, a PD-1 intracellular domain or a KIR3DL2 intracellular domain.

7. The fusion protein of embodiment 5 or 6, wherein the LIR intracellular domain comprises a sequence of SEQ ID NOs: 61 or 67-69.

8. The fusion protein of embodiment 5 or 6, wherein the PD-1 intracellular domain comprises a sequence of SEQ ID NOs: 57, 66 or 74.

9. The fusion protein of embodiment 5 or 6, wherein the KIR3DL2 intracellular domain comprises a sequence of SEQ ID NO: 52.

10. The fusion protein of embodiment 5, wherein the ZAP70 domain comprising an SH2 domain comprises the N terminal ZAP70 SH2 domain, the C terminal ZAP70 SH2, or both the N and C terminal ZAP70 SH2 domains.

11. The fusion protein of embodiment 5, wherein the ZAP70 inactivated kinase comprises a substitution of alanine for lysine at position 369 of SEQ ID NO: 17.

12. The fusion protein of any one of embodiments 2-11, wherein the second intracellular domain is selected from a CD3D molecule (CD3 delta) intracellular domain (ICD), a CD3E molecule (CD3 epsilon) ICD, a CD3G molecule (CD3 gamma), and a CD247 molecule (CD3Z or CD3 zeta) intracellular domain, or fragments or functional derivatives thereof.

13. The fusion protein of embodiment 12, wherein the second intracellular domain comprises a deletion of at least one ITAM.

14. The fusion protein of embodiment 13, wherein the fusion protein comprises a CD3 delta extracellular domain, transmembrane domain and second intracellular domain.

15. The fusion protein of embodiment 14, comprising a sequence of SEQ ID NO: 171.

16. The fusion protein of embodiment 13, wherein the fusion protein comprises a CD3 epsilon extracellular domain, transmembrane domain and second intracellular domain.

17. The fusion protein of embodiment 16, comprising a sequence of SEQ ID NO: 172.

18. The fusion protein of embodiment 13, wherein the fusion protein comprises a CD3 gamma extracellular domain, transmembrane domain and second intracellular domain.

19. The fusion protein of embodiment 18, comprising a sequence of SEQ ID NO: 173.

20. The fusion protein of embodiment 13, wherein the fusion protein comprises a CD3 zeta extracellular domain, transmembrane domain and second intracellular domain.

21. The fusion protein of embodiment 20, comprising a sequence of SEQ ID NO: 174.

22. The fusion protein of any one of embodiments 2-12, wherein the second intracellular domain is selected from a T-cell receptor (TCR) alpha intracellular domain (ICD), TCR beta ICD, or functional derivatives thereof.

23. The fusion protein of any one of embodiments 1-22, wherein the transmembrane domain is selected from a TCR alpha transmembrane domain (TM), a TCR beta TM, a CD3 delta TM, a CD3 epsilon TM, a CD3 gamma TM, a CD3 zeta TM or functional derivatives thereof.

24. The fusion protein of embodiment 23, wherein the transmembrane domain is a TCR alpha transmembrane domain comprising a sequence of SEQ ID NOs: 9, 80 or 103.

25. The fusion protein of embodiment 23, wherein the transmembrane domain is a TCR beta transmembrane domain comprising a sequence of SEQ ID NOs: 10, 104 or 105.

26. The fusion protein of embodiment 23, wherein the transmembrane domain is a CD3 delta transmembrane domain comprising a sequence of SEQ ID NOs: 106-108.

27. The fusion protein of embodiment 23, wherein the transmembrane domain is a CD3 epsilon transmembrane domain comprising a sequence of SEQ ID NOs 7, 110 or 111.

28. The fusion protein of embodiment 23, wherein the transmembrane domain comprises a CD3 gamma transmembrane domain comprising a sequence of SEQ ID NO: 8 or 79.

29. The fusion protein of embodiment 23, wherein the transmembrane domain is a CD3 zeta transmembrane comprising a sequence of SEQ ID NOs: 76-78.

30. The fusion protein of any one of embodiments 2-12, wherein the first intracellular domain, optionally the second intracellular domain, and the transmembrane domain, comprise domains that are isolated or derived from PD-1, CTLA-4, KIR3DL2, KIR3DL3, ZAP70, LIR1, FcgRIIB or NKG2D.

31. The fusion protein of any one of embodiments 1-30, wherein the transmembrane domain and the first intracellular domain are connected by a polypeptide linker.

32. The fusion protein of any one of embodiments 2-31, wherein the first intracellular domain and the second intracellular domain are connected by polypeptide linker.

33. The fusion protein of embodiment 31 or 32, wherein the polypeptide linker comprises at most 3, 4, 5, 6, 8, 9, 10, 12, 15, 16, 18 or 20 amino acids.

34. The fusion protein of embodiment 33, wherein the polypeptide linker comprises a sequence of GS, GGS, SEQ ID NO: 240, SEQ ID NO: 18, SEQ ID NO: 19, SEQ ID NO: 20, or SEQ ID NO: 21.

35. The fusion protein of any one of embodiments 1-34, wherein the domains are arranged in an order of: extracellular domain, transmembrane domain, first intracellular domain, from N to C terminus.

36. The fusion protein of any one of embodiments 2-34, wherein the domains are arranged in an order from N to C terminus of: extracellular domain, transmembrane domain, first intracellular domain.

37. The fusion protein of any one of embodiments 2-34, wherein the domains, are arranged in an order from N to C terminus of: extracellular domain, transmembrane domain, second intracellular domain, first intracellular domain.

38. The fusion protein of any one of embodiments 2-34, wherein the domains, are arranged in an order from N to C terminus of: extracellular domain, transmembrane domain, first intracellular domain, second intracellular domain.

39. An engineered T-cell receptor comprising the fusion protein of any one of embodiments 1-38.

40. The engineered T-cell receptor of embodiment 39, comprising an extracellular antigen binding domain fused to an extracellular domain of a subunit of the TCR.

41. The engineered T-cell receptor of embodiment 40, wherein the extracellular antigen binding domain comprises an antibody fragment, a single chain variable fragment (ScFv), or a single domain antibody (sdAb).

42. The engineered T-cell receptor of embodiment 40, wherein the extracellular antigen binding domain comprises an extracellular antigen binding domain isolated or derived from a T cell receptor.

43. The engineered T-cell receptor of embodiment 42, wherein the extracellular antigen binding domain comprises a TCR alpha extracellular domain and a TCR beta extracellular domain.

44. The engineered T-cell receptor of embodiment 39-43, wherein the subunit of the TCR comprises TCR alpha, TCR beta, CD3 delta, CD3 epsilon, CD3 gamma or CD3 zeta.

45. The engineered T-cell receptor of any one of embodiments 39-44, wherein the engineered T-cell receptor comprises a first fusion protein comprising a TCR alpha transmembrane domain and a LIR1 intracellular domain, and a second fusion protein comprising a TCR beta transmembrane domain and a LIR1 intracellular domain.

46. The engineered T-cell receptor of any one of embodiments 39-44, wherein the engineered T-cell receptor comprises a first fusion protein comprising a CD delta transmembrane domain and a LIR1 intracellular domain, a second fusion protein comprising a CD3 epsilon transmembrane domain and a LIR1 inhibitory domain and a third fusion protein comprising a CD3 gamma transmembrane domain and a LIR1 inhibitory domain.

47. The engineered T-cell receptor of embodiment 46, wherein the first fusion protein comprises SEQ ID NO: 183 or 187, the second fusion protein comprises SEQ ID NO: 184 or 188, and the third fusion protein comprises 185 or 189.

48. The engineered T-cell receptor of embodiment 46 or 47, further comprising a fifth fusion protein comprising a CD3 zeta transmembrane domain and a LIR1 inhibitory domain.

49. The engineered T-cell receptor of embodiment 48, wherein the fifth fusion protein comprises a sequence of 186 or 190.

50. A polynucleotide comprising a coding sequence encoding the fusion protein of any one of embodiments 1-38.

51. A vector comprising a coding sequence encoding any of the fusion protein of any one of embodiments 1-38, wherein the coding sequence is operatively linked to a promoter.

52. A pharmaceutical composition comprising the polynucleotide of embodiment 50 or the vector of embodiment 51.

53. An immune cell, comprising a polynucleotide sequence encoding any of the fusion protein of any one of embodiments 1-39, wherein the coding sequence operatively linked to a promoter.

54. An immune cell, comprising the engineered TCR of any one of embodiments 39-49.

55. The immune cell of embodiment 54, wherein the immune cell is a T cell.

56. The immune cell of any one of embodiments 53-55, wherein an activity of the immune cell is inhibited when the immune cell is contacted with a target cell expressing an antigen recognized by the engineered TCR.

57. The immune cell of embodiment 56 wherein the activity that is inhibited comprises proliferation, cytokine production, cytotoxicity or a combination thereof.

58. The immune cell of any one of embodiments 53-57, wherein the immune cell a deletion of one or more subunits of the endogenous T cell receptor.

59. The immune cell of embodiment 58, wherein the immune cell comprises deletions in CD3 delta, CD3 epsilon and CD3 gamma.

60. The immune cell of embodiment 58, wherein the immune cell comprises deletions in CD3 delta, CD3 epsilon, CD3 gamma and CD3 zeta.

61. The immune cell of any one of embodiments 58-60, wherein the immune cell does not express a functional endogenous TCR.

62. The immune cell of any one of embodiments 54-61, further comprising an activator receptor.

63. The immune cell of embodiment 62, wherein the activator receptor is a TCR or a chimeric antigen receptor (CAR).

64. A pharmaceutical composition comprising the immune cell of any one of embodiments 53-61.

65. A kit comprising the polynucleotide of embodiment 50 or the vector of embodiment 51.

66. A kit comprising the immune cell of any one of embodiments 53-63.

67. A method of generating immune cells comprising an engineered T-cell receptor, comprising:

a. providing a plurality of immune cells;

b. transforming the plurality of immune cells with the vector of embodiment 51 to generate a plurality of transformed immune cells; and

c. culturing the plurality of transformed immune cells under conditions sufficient to express the engineered TCR from vector;

thereby generating immune cells comprising an engineered TCR.

68. The method of embodiment 67, wherein the immune cells are T cells.

69. The method of embodiment 67 or 68, wherein the T cell are autologous or allogeneic.

70. The method of any one of embodiments 67-69, comprising (d) activating the plurality of immune cells.

71. A method of inhibiting the activity of a T cell by expressing the fusion protein of any one of embodiments 1-38 in the T cell.

72. A method of inhibiting the activity of a T cell by expressing the engineered TCR of any one of embodiments 39-49 in the T cell.

73. The method of embodiment 71 or 72, wherein the activity of the T cell comprises TCR-mediated signaling in response to a cognate antigen.

74. The method of embodiment 73, wherein the TCR-mediated signaling comprises activation of one or more genes operatively linked to an NFAT promoter.

### EXAMPLES

### Methods

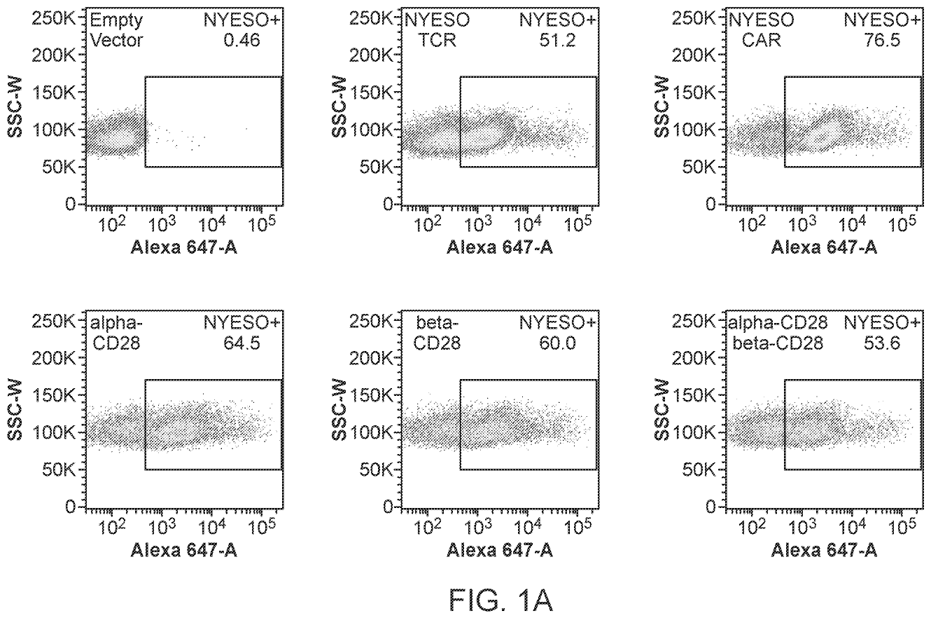

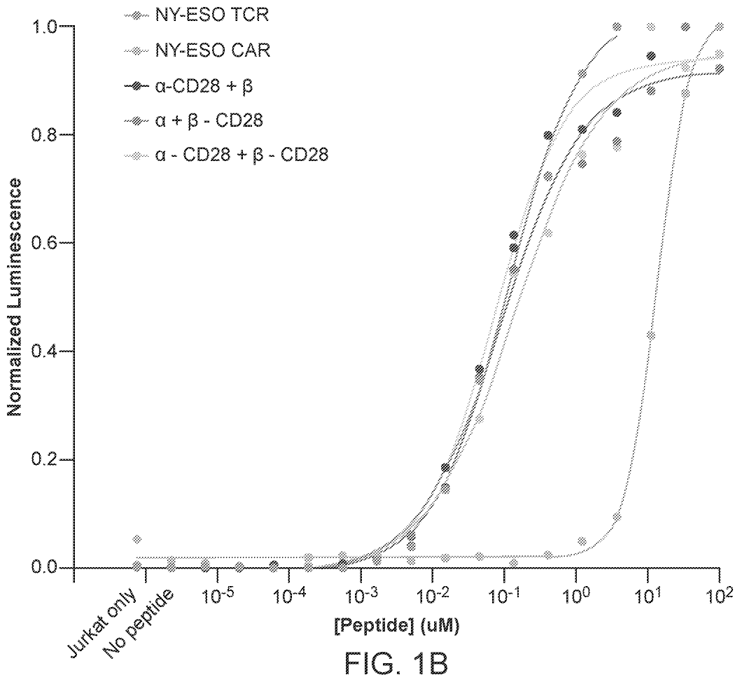

Jurkat T cells engineered to include an NFAT promoter-driven luciferase reporting gene were used as effector cells. Expression of luciferase by this cell line reflects TCR-mediated signaling. The reporter cells were transfected with each of the various engineered TCR constructs or control constructs in Tables 2A-2L. In each experiment, expression of the TCR was confirmed by flow cytometry using Alexa Fluor 647-labeled NY-ESO-1-MHC tetramer to detect expression of the TCR (panel A in FIGS. 1A-14B). As target cells, 16 hours prior to exposure to effector cells, A375 cells were loaded with serially diluted concentrations of NY-ESO-1 peptide. The effector cells were then co-cultured with target cells. Luciferase was measured by luminescence reading after 6 hours of co-culture. Luciferase luminescence was normalized to maximum and minimum intensity to allow comparison of activating peptide concentrations for each engineered TCR construct.

Fusions to TCR alpha and beta were made in two forms, short and long. Short engineered TCRs consist of a (G4S)×1 linker for TCR alpha and no linker for TCR beta, and long engineered TCRs consist of a (G4S)×4 linker for TCR alpha and (G4S)×3 linker for TCR beta. Signaling domains were fused to either TCR alpha, beta or both alpha and beta in either the short or long format.

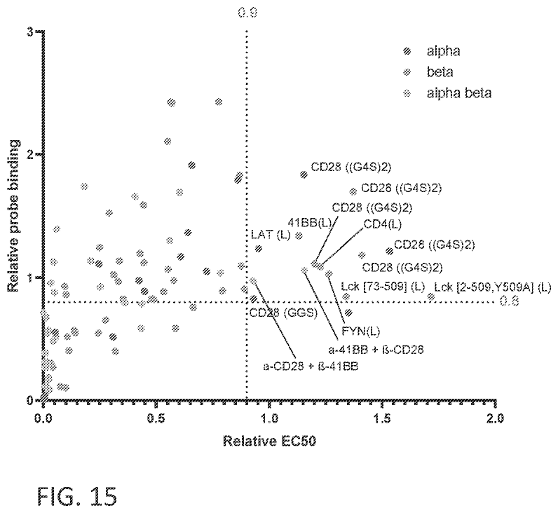

Examples 1-15 demonstrate the efficacy of TCR complexes comprising fusions to additional intracellular signaling domains. The results are summarized in FIG. 15, which shows EC50 and relative probe binding relative to the parental NY-ESO-1 TCR without the additional intracellular signaling domain.

### Example 1: CD28 and TCR Alpha/Beta Fusions

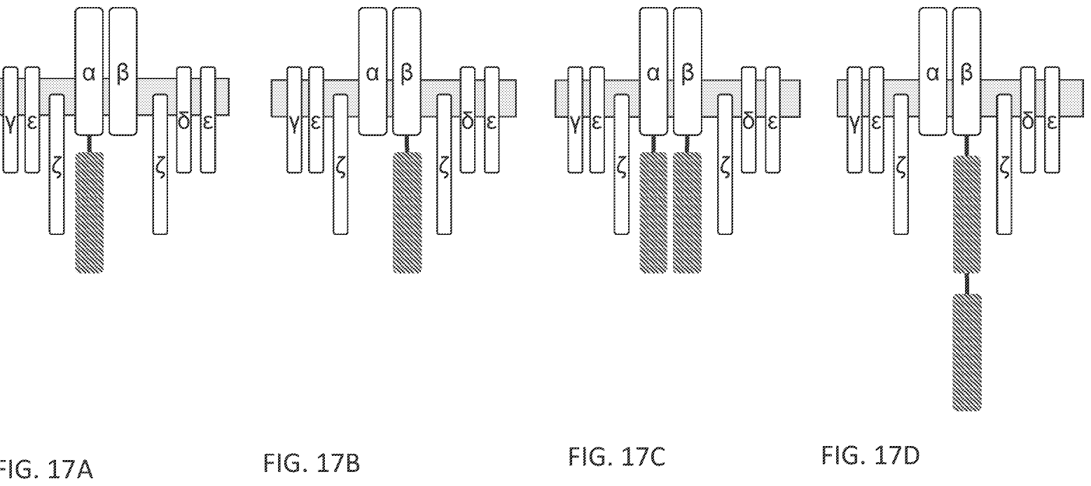

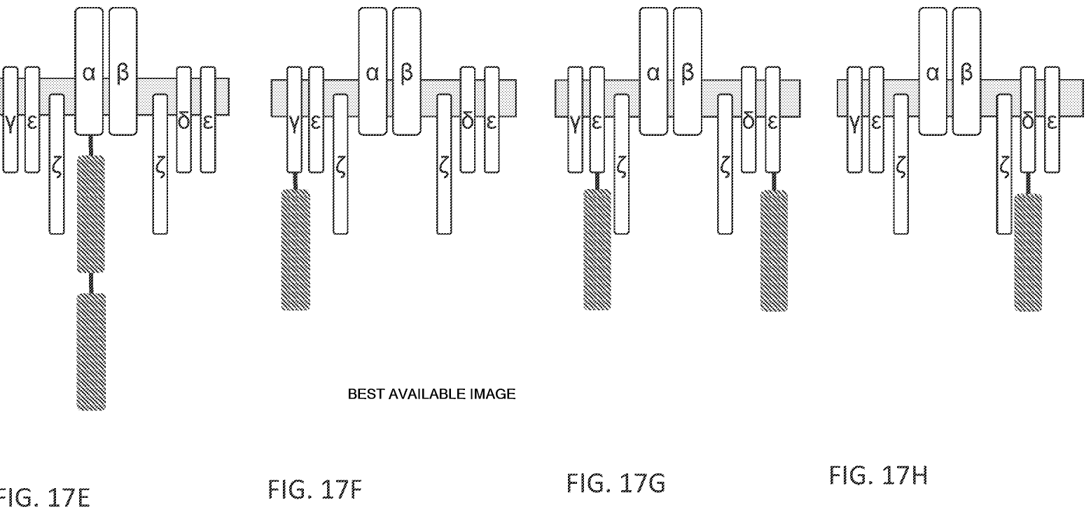

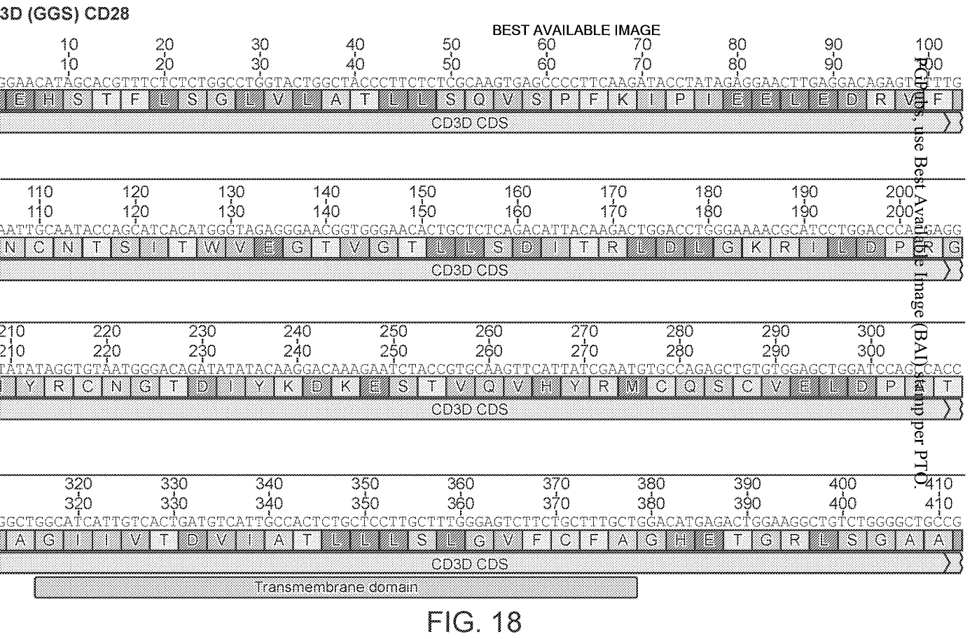

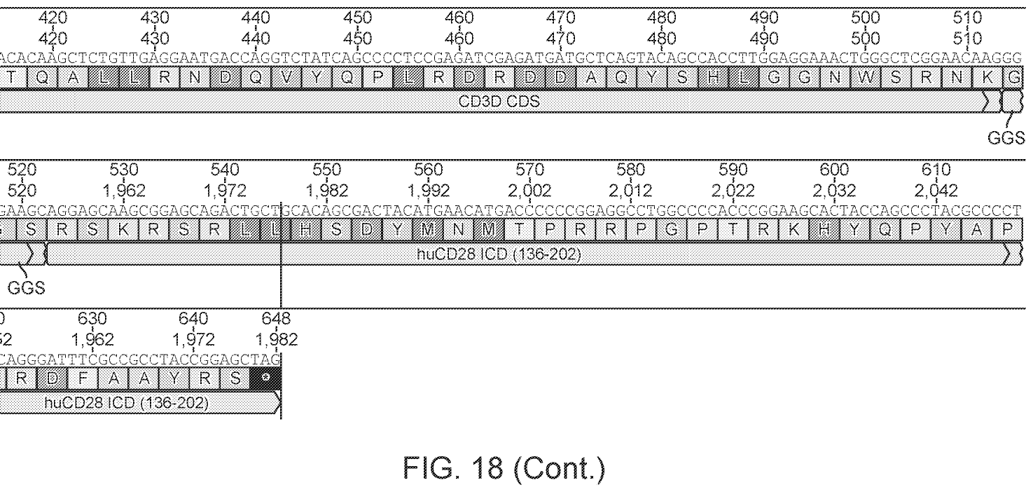

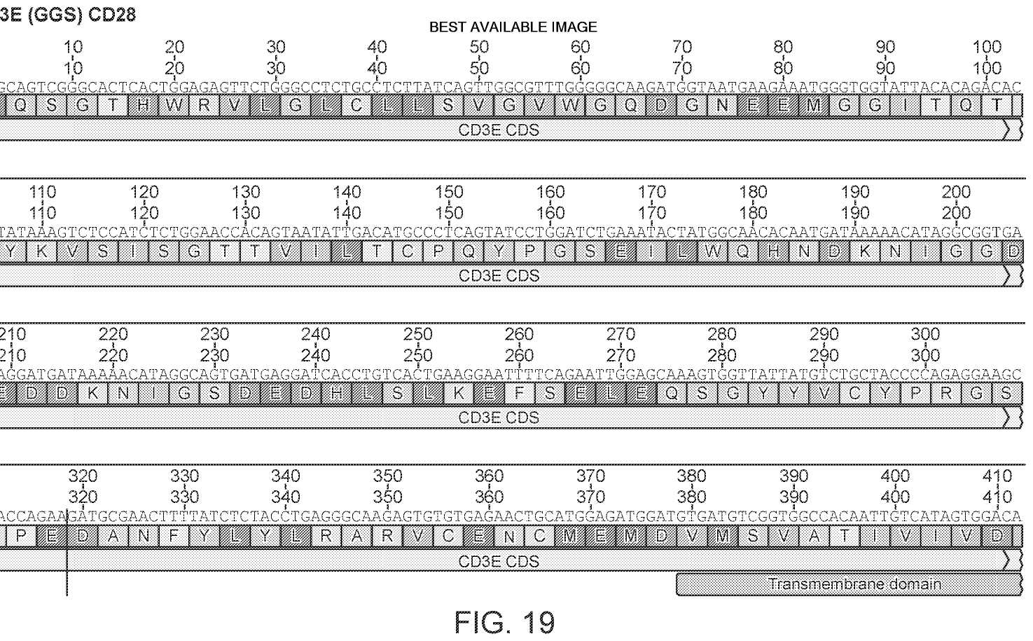

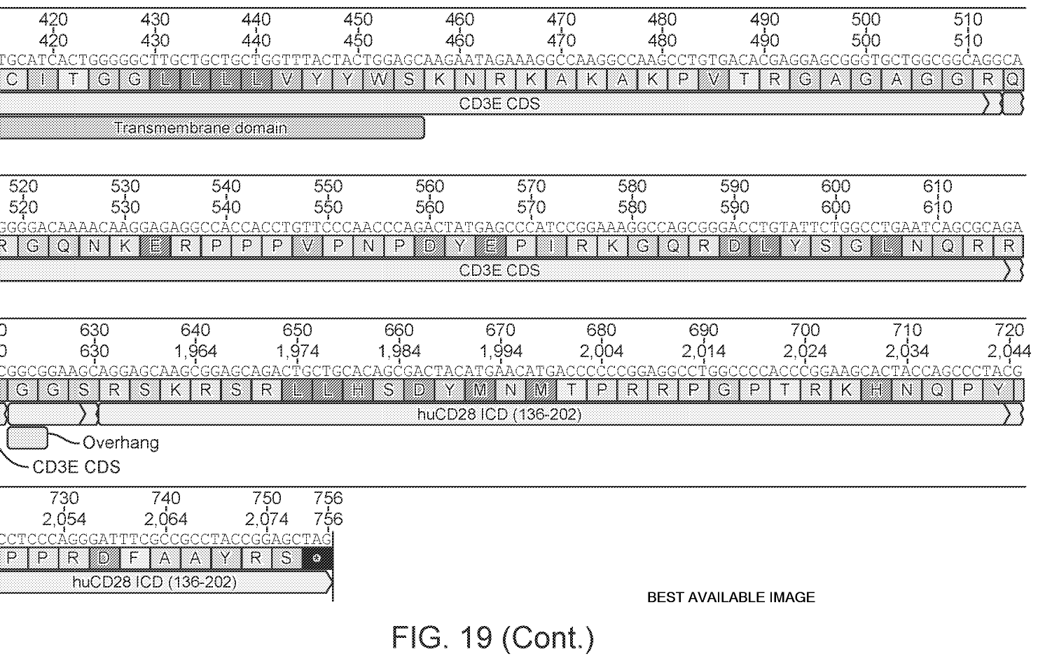

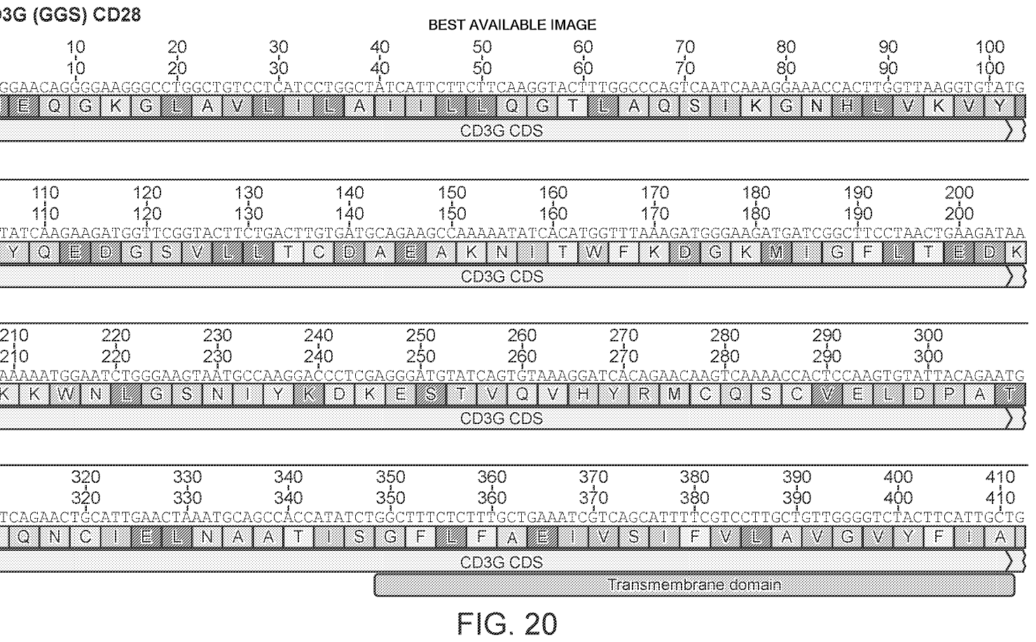

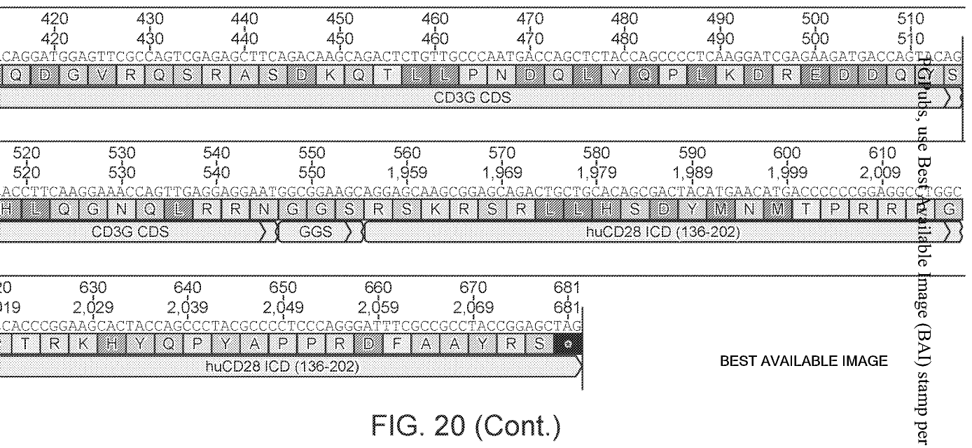

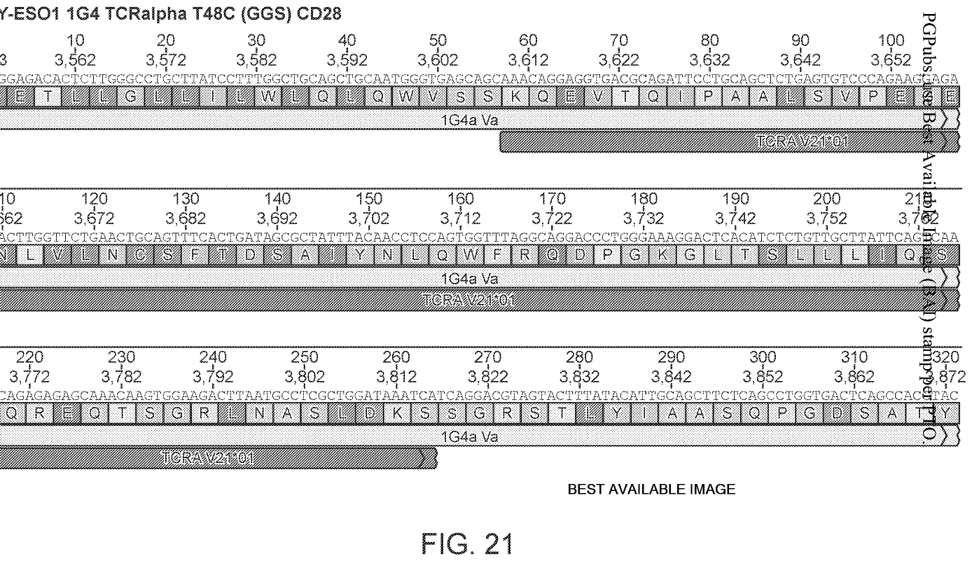

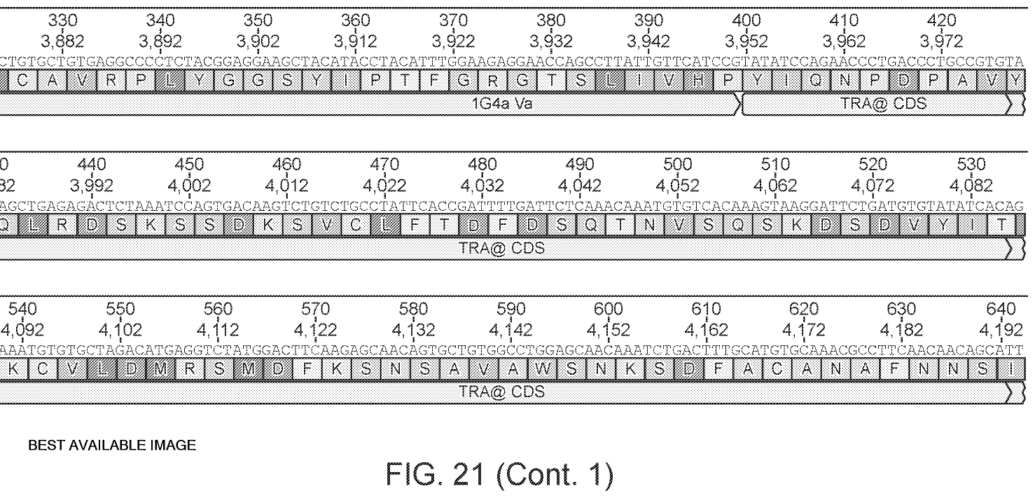

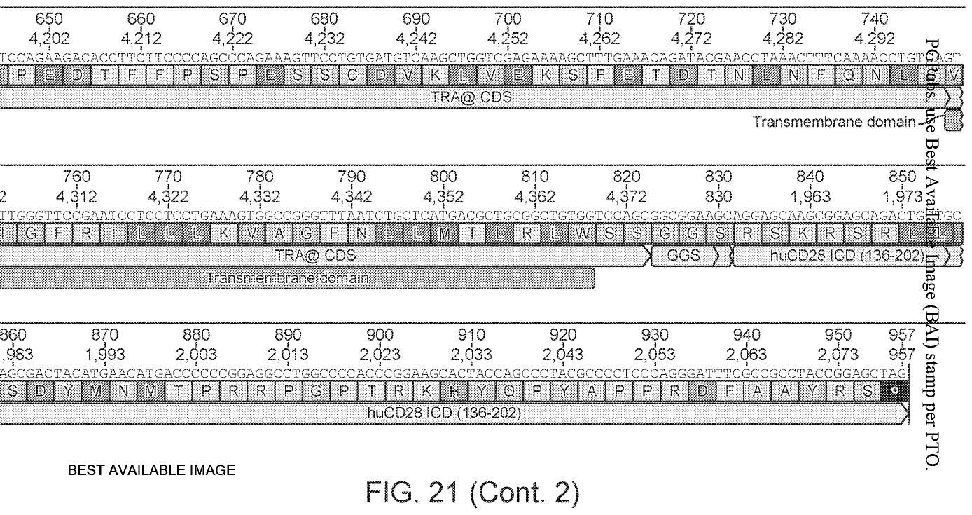

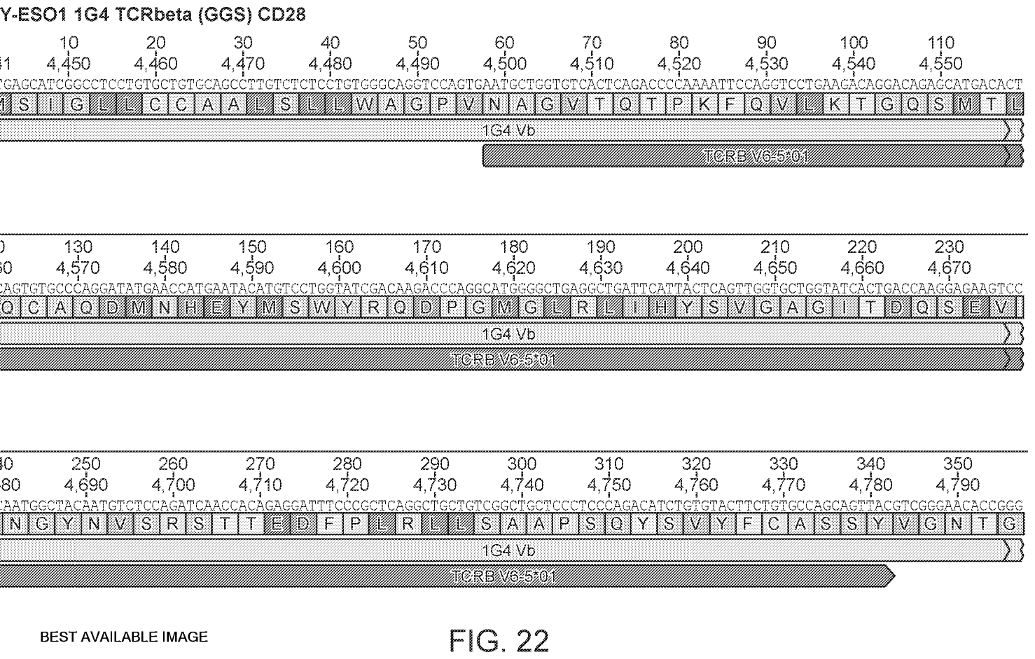

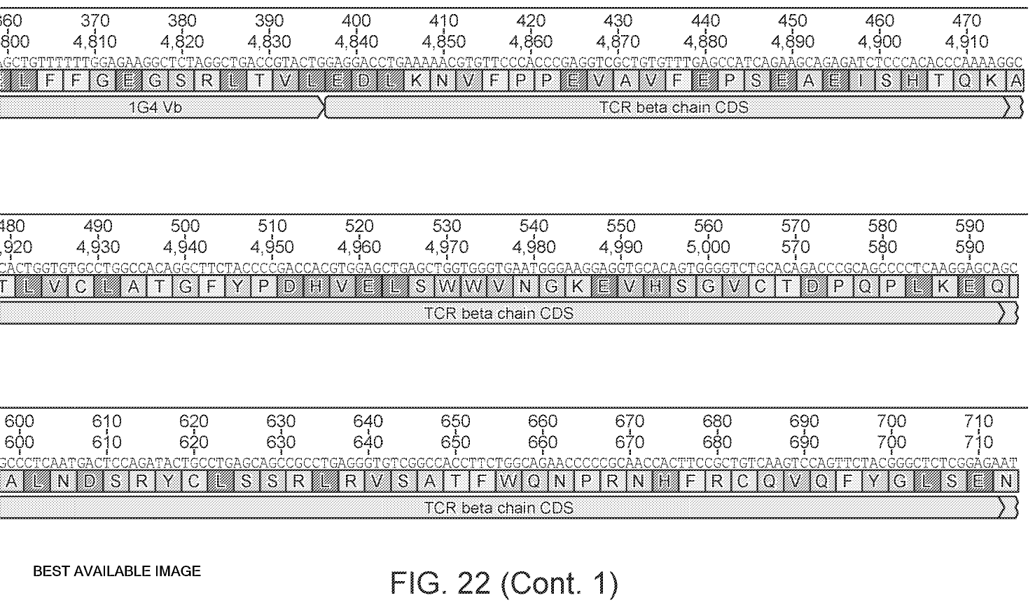

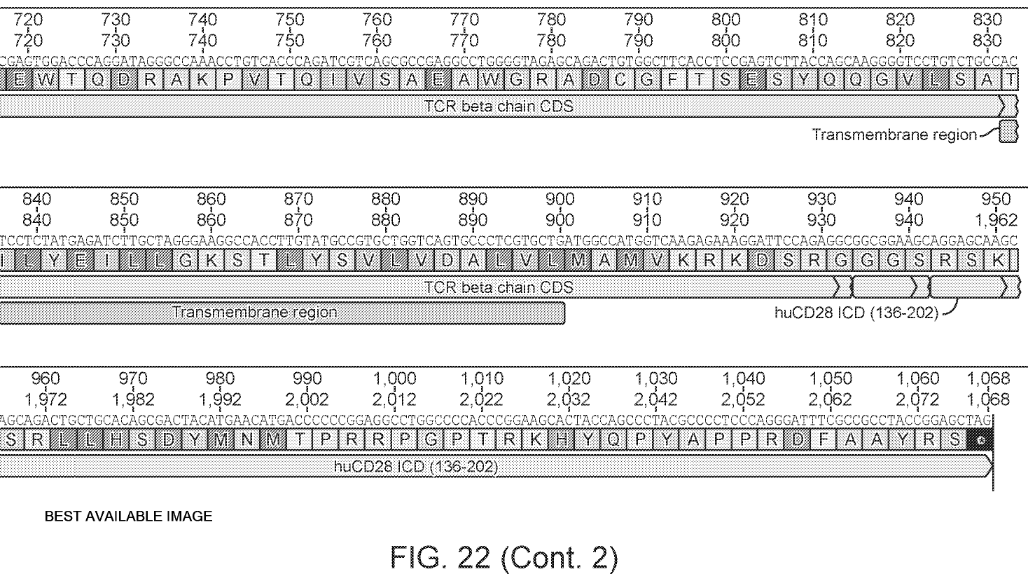

Jurkat T cells engineered with NFAT promoter-driven luciferase gene were transfected with engineered TCR constructs containing the intracellular domain of CD28 fused to the C-Terminus of alpha, beta or both alpha and beta chains of the NY-ESO TCR. CD28-engineered TCR surface expression was confirmed using Alexa Fluor 647-labeled NY-ESO-1-MIHC tetramer. Jurkat cell activation, as assayed by increase in luciferase expression, was dependent on co-culture with A375 cells loaded with activating concentration of NY-ESO-1 peptide. Luciferase luminescence was normalized to maximum and minimum intensity to allow comparison of activating peptide concentrations for each engineered TCR construct. NY-ESO TCR consistently showed higher sensitivity than the NY-ESO CAR, as NY-ESO TCR-transfected Jurkat cells became activated at a lower concentration of loaded NY-ESO-1 peptide compared to NY-ESO CAR-transfected cells. Moreover, CD28-engineered TCR-transfected Jurkat T cells showed similar sensitivity to antigen as the parental NY-ESO TCR. This result showed that the intracellular tail of TCR alpha and beta chains could be tagged with additional domains without disrupting the TCR signaling pathways leading to T cell activation. Results are shown in FIGS. 1A-1B. Examples of these engineered TCR architectures are shown in FIG. 17A (TCR alpha fusion), FIG. 17B (TCR beta fusion) and FIG. 17C (TCR alpha and TCR beta fusions).

### Example 2: CD28 and CD3 Fusions

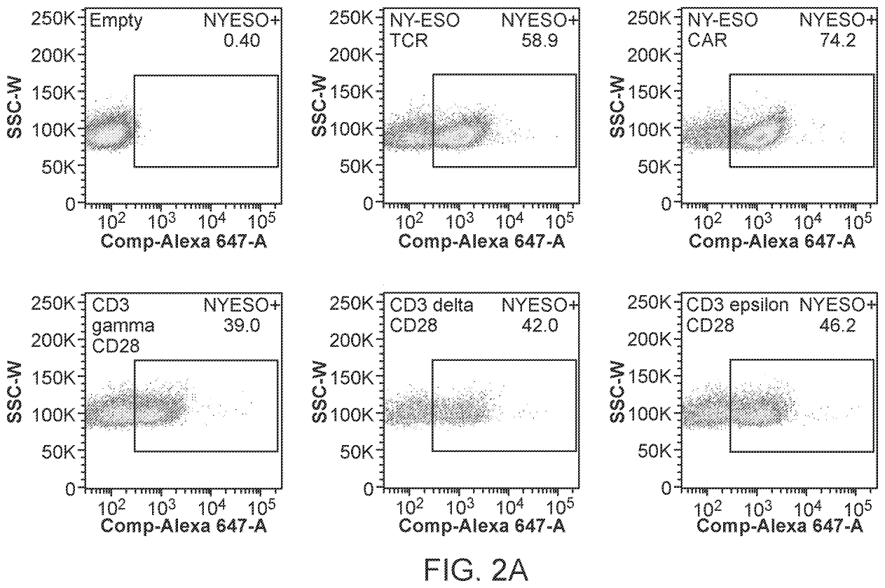

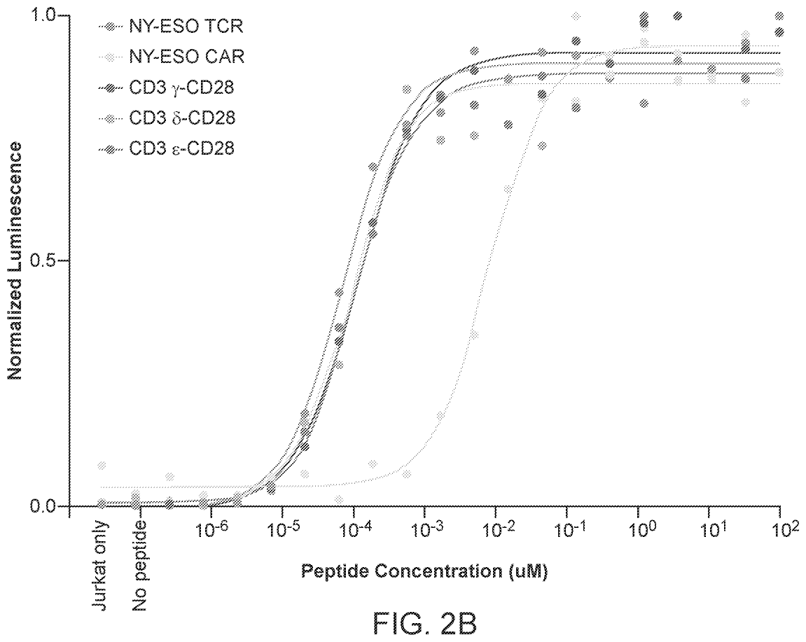

Jurkat T cells engineered with NFAT promoter-driven luciferase gene were transfected with engineered TCR constructs containing the intracellular domain of CD28 fused to the C-Terminus of CD3 gamma, delta, or epsilon and co-expressed with the NY-ESO TCR. NY-ESO TCR surface expression was confirmed using Alexa Fluor 647-labeled NY-ESO-1-MHC tetramer. Jurkat cells transfected with CD28 on CD3 subunits showed similar sensitivity to antigen as the parental NY-ESO TCR, indicating that the intracellular tails of CD3 subunits could be modified without disrupting TCR activation. Results are shown in FIGS. 2A-2B. Examples of these engineered TCR architectures are shown in FIG. 17F (CD3 gamma fusion), FIG. 17G (CD3 epsilon fusion) and FIG. 17H (CD3 delta fusion).

### Example 3: 4-1BB Fusions

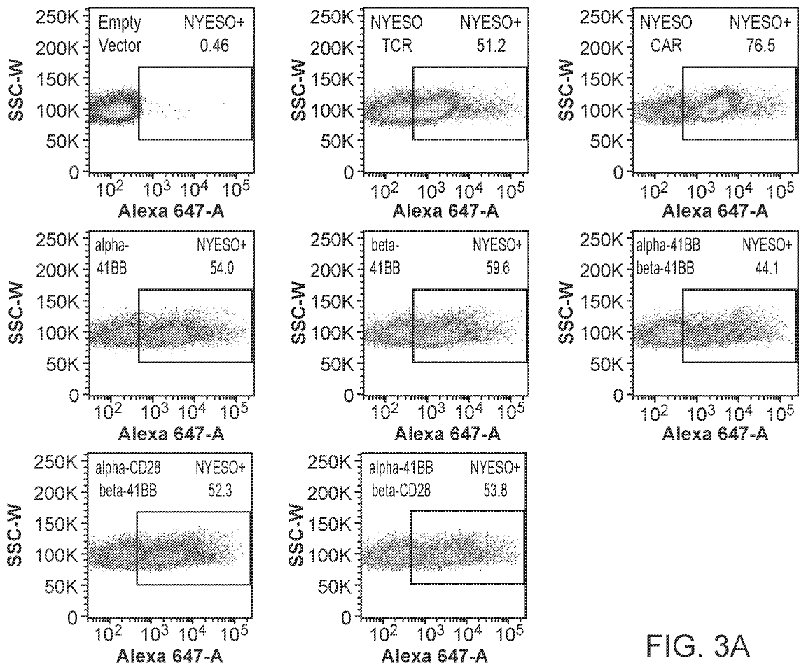

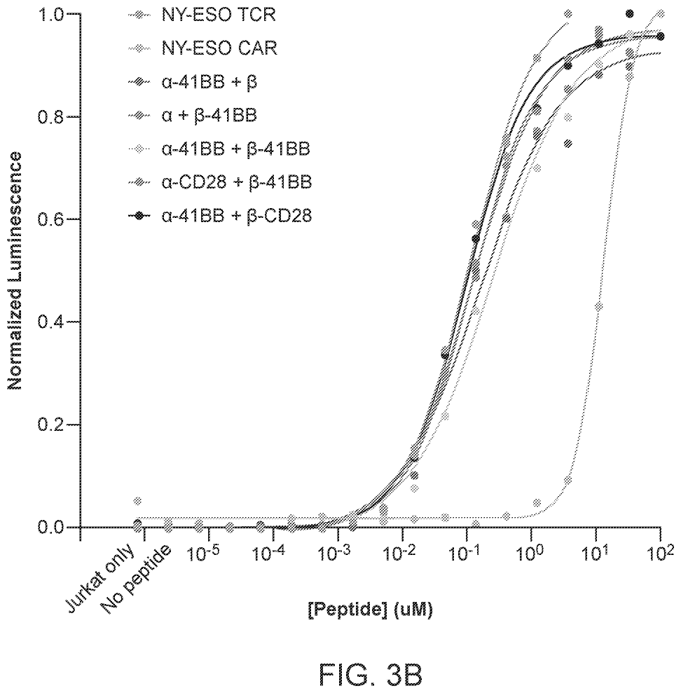

Jurkat T cells engineered with NFAT promoter-driven luciferase gene were transfected with engineered TCR constructs containing the intracellular domain of 4-1BB fused to the C-Terminus of alpha, beta or both alpha and beta chains of the NY-ESO TCR. 4-1BB-engineered TCR surface expression was confirmed using Alexa Fluor 647-labeled NY-ESO-1-MHC tetramer. Jurkat cells transfected with 4-1BB fusion to TCR beta or combinations of CD28 and 4-1BB on alpha and beta chains showed similar sensitivity to antigen as the parental NY-ESO TCR, indicating that the intracellular tails of TCR alpha and beta chains could be modified without disrupting TCR activation. However, fusion of 4-1BB signaling domain to the alpha chain or to both alpha and beta chains decreased sensitivity to antigen, as transfected cells were activated at higher peptide concentrations compared to NY-ESO TCR. This result suggested that certain modifications to the TCR could disrupt TCR signaling. Results are shown in FIGS. 3A-3B. Examples of these engineered TCR architectures are shown in FIG. 17A (TCR alpha fusion), FIG. 17B (TCR beta fusion) and FIG. 17C (TCR alpha and TCR beta fusions).

### Example 4: CD28 and 4-1BB Fusions

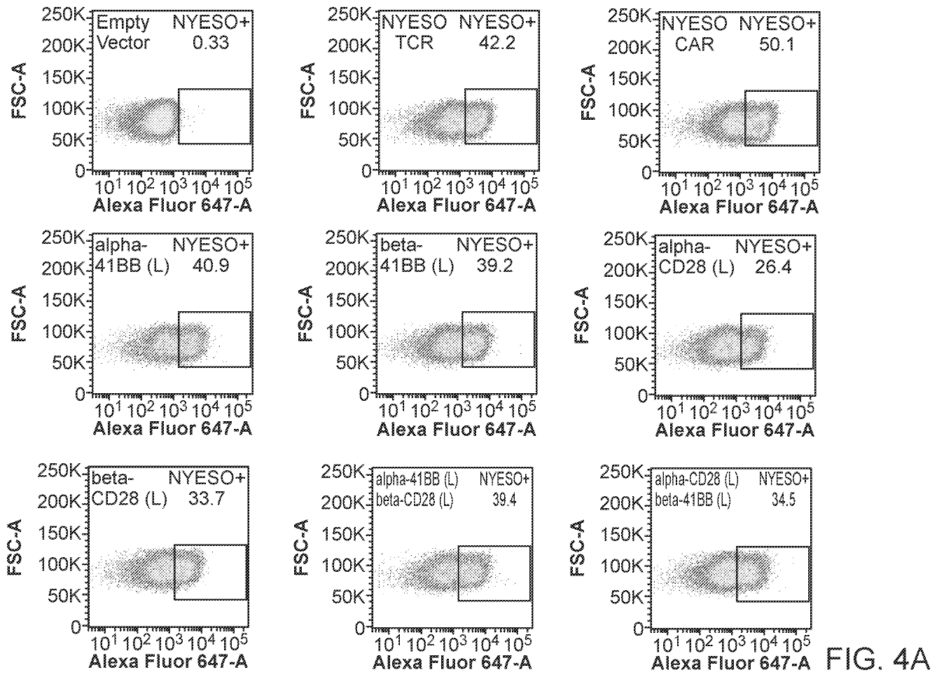

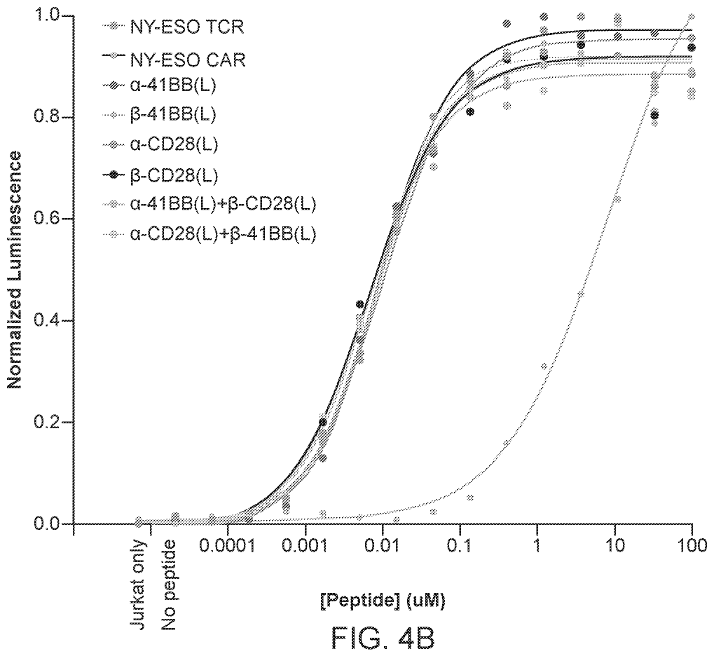

Jurkat T cells engineered with NFAT promoter-driven luciferase gene were transfected with engineered TCR constructs containing the intracellular domain of CD28 or 4-1BB fused to the C-Terminus of alpha, beta or both alpha and beta chains of the NY-ESO TCR. In contrast to the engineered TCR constructs in FIG. 2, long linkers were used to link the signaling domains to the TCR alpha and beta chains. These constructs showed equivalent sensitivity to peptide as the parent NY-ESO TCR. Importantly, decrease in TCR sensitivity observed in FIG. 2 with fusion of 4-1BB domain to the alpha chain was not observed with the longer linker, indicating that linker length affects sensitivity of engineered TCRs. Results are shown in FIGS. 4A-4B. Examples of these engineered TCR architectures are shown in FIG. 17A (TCR alpha fusion), FIG. 17B (TCR beta fusion) and FIG. 17C (TCR alpha and TCR beta fusions).

### Example 5: CD4 Fusions

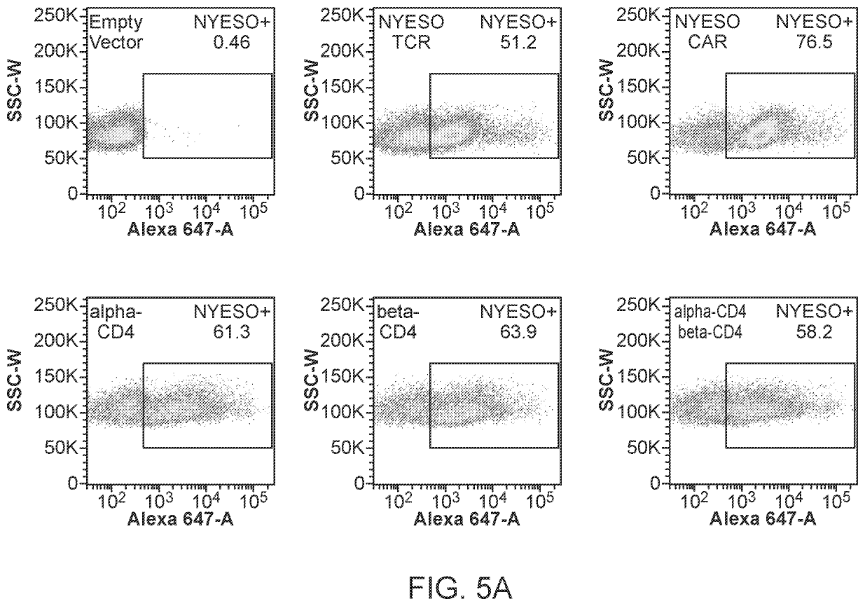

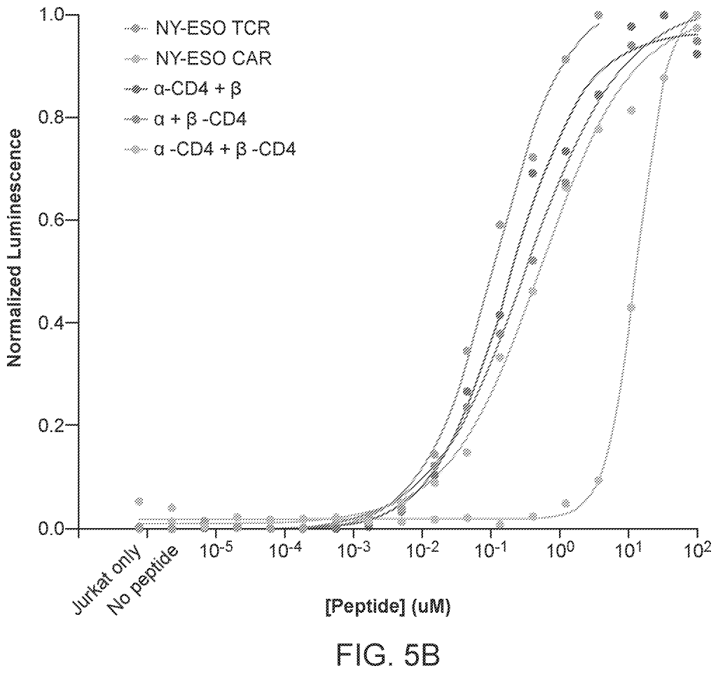

Jurkat T cells engineered with NFAT promoter-driven luciferase gene were transfected with engineered TCR constructs containing the intracellular domain of CD4 fused to the C-Terminus of alpha, beta or both alpha and beta chains of the NY-ESO TCR using a short GGS linker. Jurkat cells transfected with CD4-engineered TCR showed lower sensitivity to antigen than the parental NY-ESO TCR, indicating that addition of CD4 cytosolic domain to the TCR using a short linker disrupts TCR signaling. Results are shown in FIGS. 5A-5B. Examples of these engineered TCR architectures are shown in FIG. 17A (TCR alpha fusion), FIG. 17B (TCR beta fusion) and FIG. 17C (TCR alpha and TCR beta fusions).

### Example 6: CD8 Fusions

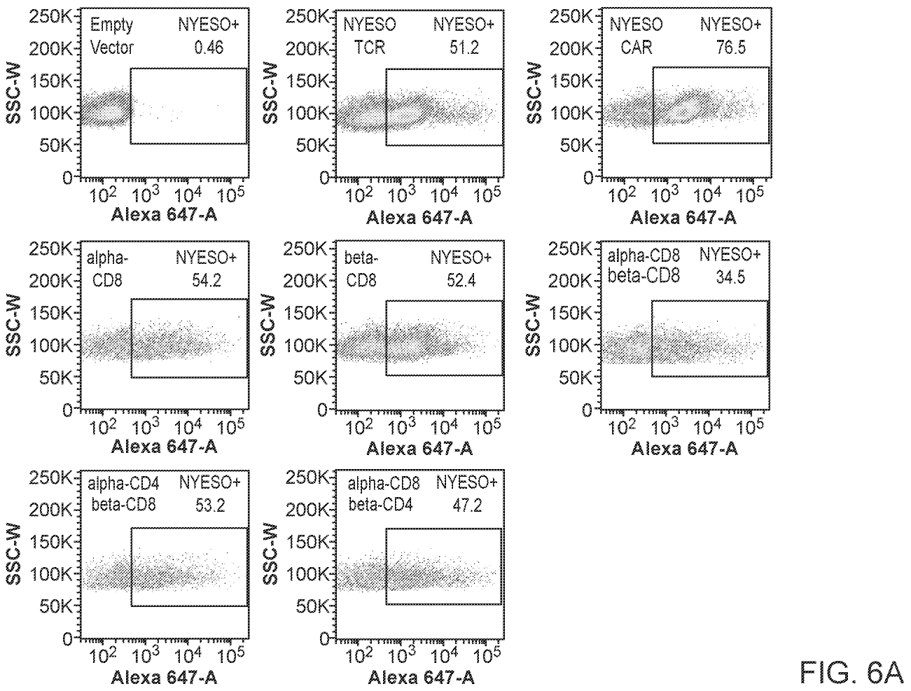

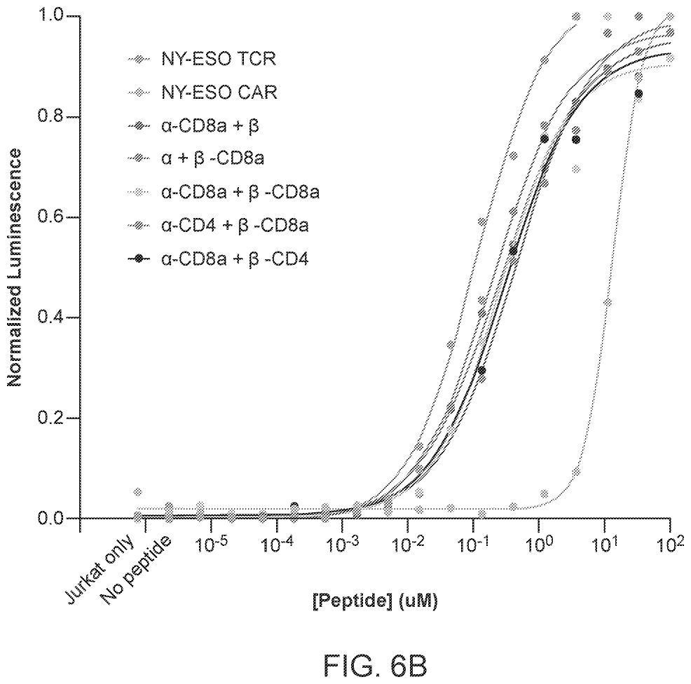

Jurkat T cells engineered with NFAT promoter-driven luciferase gene were transfected with engineered TCR constructs containing the intracellular domain of CD8 fused to the C-Terminus of alpha, beta or both alpha and beta chains of the NY-ESO TCR using a short GGS linker. Jurkat cells transfected with CD8-engineered TCR showed lower sensitivity to antigen than the parental NY-ESO TCR, indicating that addition of CD8 cytosolic domain to the TCR using a short linker disrupts TCR signaling. Results are shown in FIGS. 6A-6B. Examples of these engineered TCR architectures are shown in FIG. 17A (TCR alpha fusion), FIG. 17B (TCR beta fusion) and FIG. 17C (TCR alpha and TCR beta fusions).

### Example 7: CD4 and CD8 Fusions

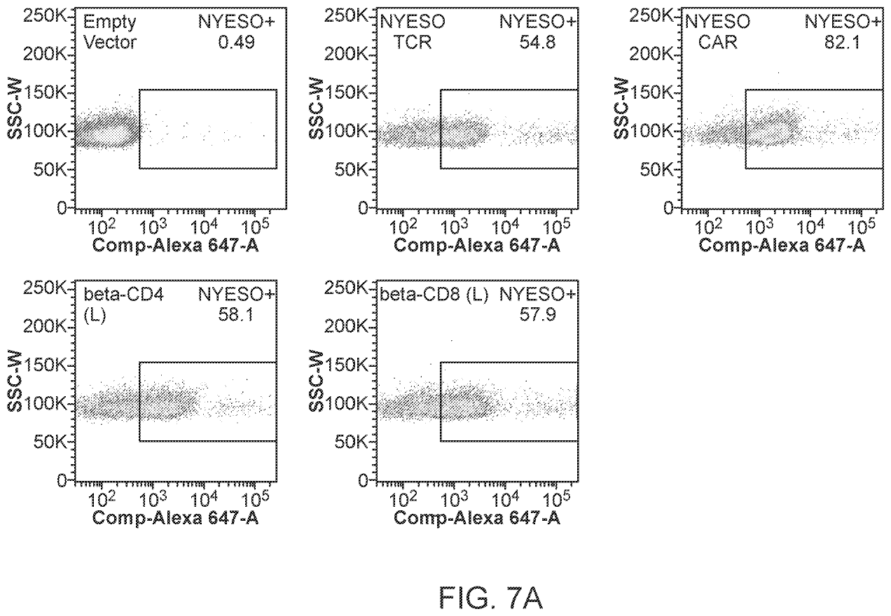

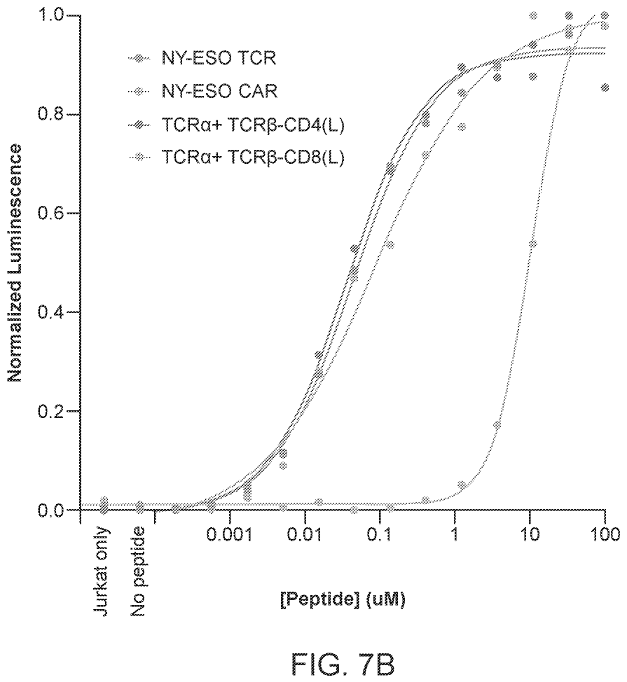

Jurkat T cells engineered with NFAT promoter-driven luciferase gene were transfected with engineered TCR constructs containing the intracellular domain of CD4 or CD8 fused to the C-Terminus of the beta chain of the NY-ESO TCR using a long (G4S)×3 linker. Jurkat cells transfected with CD4-engineered TCR showed slightly improved sensitivity to antigen than the parental NY-ESO TCR, indicating that addition of CD4 cytosolic domain to the TCR using a long linker enhances TCR signaling. However, Jurkat cells transfected with CD8-engineered TCR showed decreased sensitivity, indicating that addition of CD8 cytosolic domain to the TCR using a short or long linker disrupts TCR signaling. Results are shown in FIGS. 7A-7B. An example of this engineered TCR architectures is shown in FIG. 17B (TCR beta fusion).

### Example 8: Lck Fusions

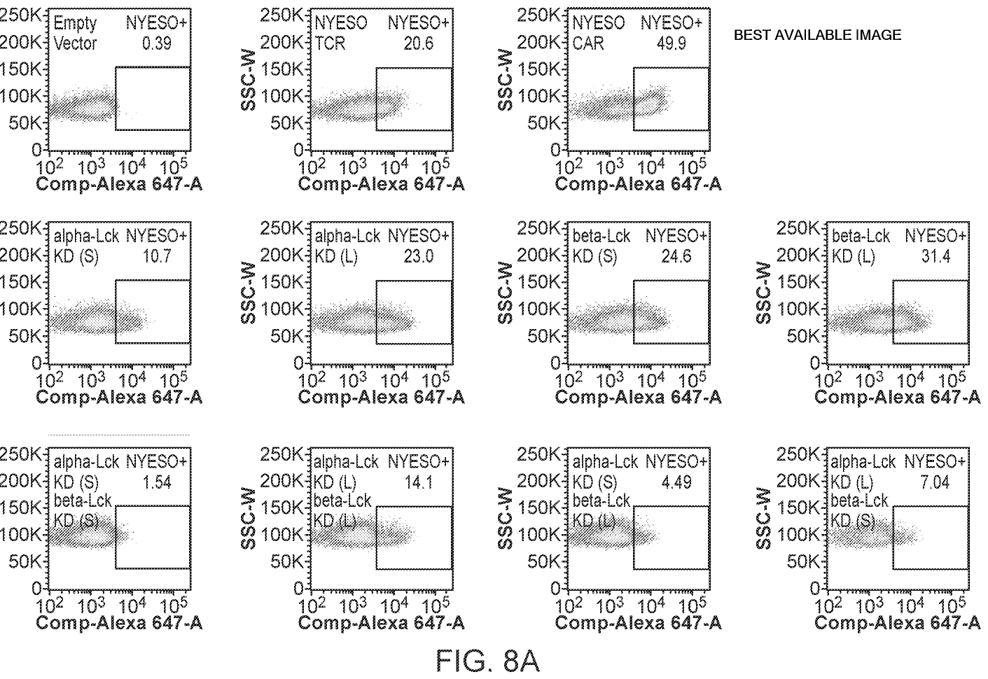

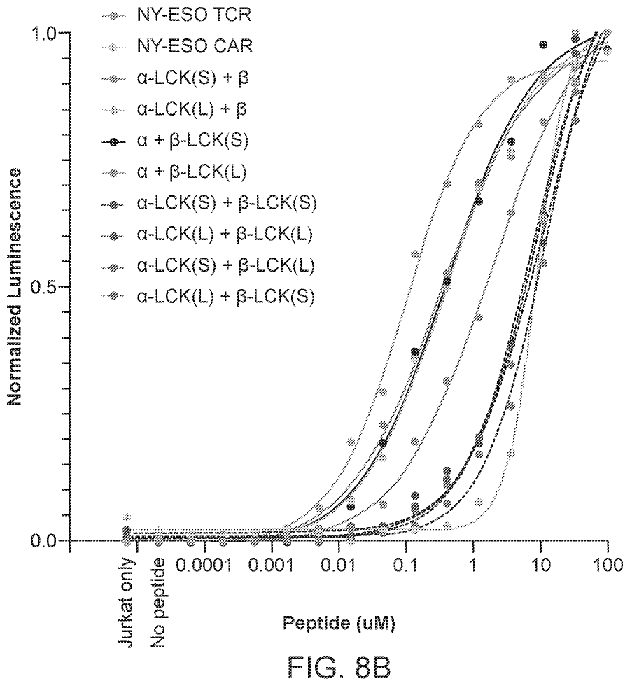

Jurkat T cells engineered with NFAT promoter-driven luciferase gene were transfected with engineered TCR constructs containing the Lck kinase domain (KD) fused to the C-Terminus of alpha, beta or both alpha and beta chains of the NY-ESO TCR using short or long linkers. These constructs showed decreased sensitivity to peptide compared to the parental NY-ESO TCR, indicating that addition of the globular Lck KD disrupts TCR signaling. In particular, fusion of Lck KD to the TCR alpha chain using the short linker resulted in lowered surface expression and decreased sensitivity to antigen compared to TCR alpha fusion using the long linker and all fusions to the beta chain. Lck KD fusions to both TCR alpha and beta chains resulted in the lowest sensitivity to peptide, and combined alpha and beta fusions containing a short linker resulted in the lowest surface expression, indicating that fusions of globular domains to both alpha and beta, or to alpha using the short linker is not well tolerated by Jurkat T cells. Results are shown in FIGS. 8A-8B. Examples of these engineered TCR architectures are shown in FIG. 17A (TCR alpha fusion), FIG. 17B (TCR beta fusion) and FIG. 17C (TCR alpha and TCR beta fusions).

### Example 9: Lck Fusions

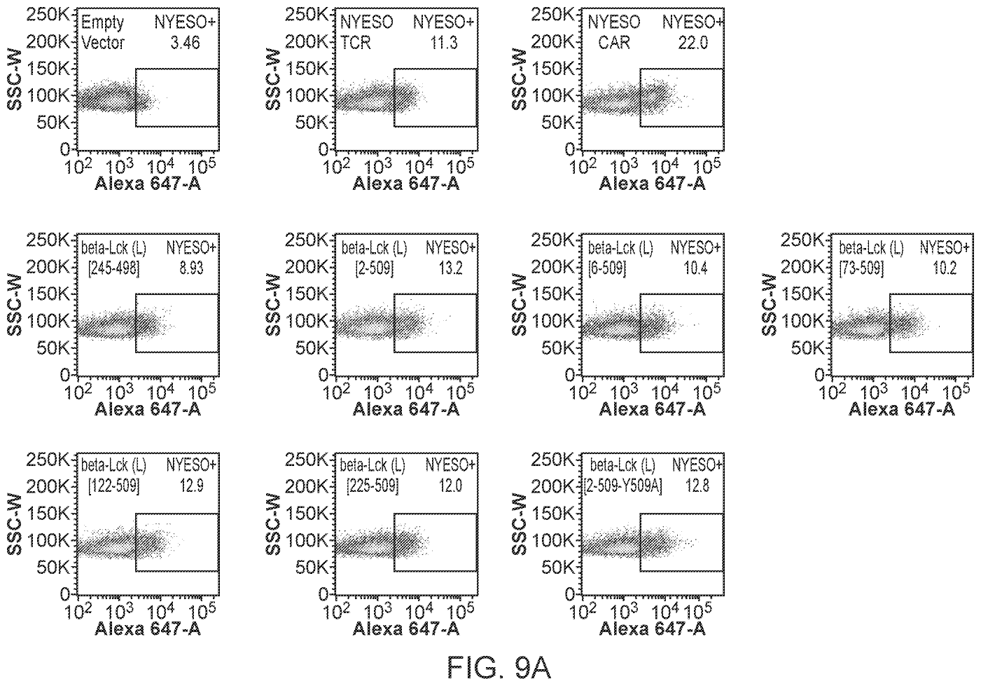

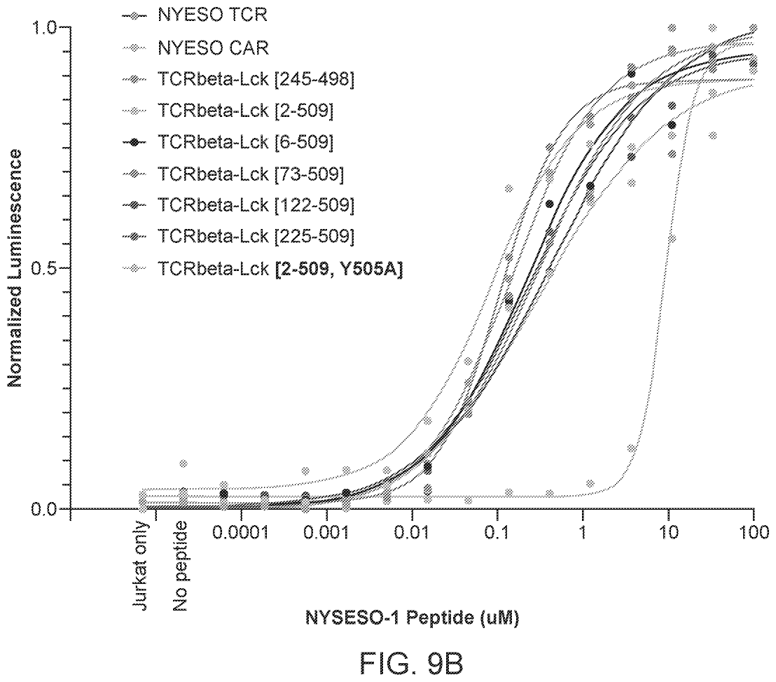

Jurkat T cells engineered with NFAT promoter-driven luciferase gene were transfected with engineered TCR constructs containing truncations or mutations of Lck fused to the C-Terminus of the beta chain of the NY-ESO TCR using a long (G4S)×3 linker. Engineered TCR containing the full length Lck with an Y505A mutation showed the highest sensitivity to antigen compared to the Lck-engineered TCR constructs, and showed an improvement in sensitivity over the parental NY-ESO TCR. Results are shown in FIGS. 9A-9B. An example of this engineered TCR architectures is shown in FIG. 17B (TCR beta fusion).

### Example 10: Fyn Fusions

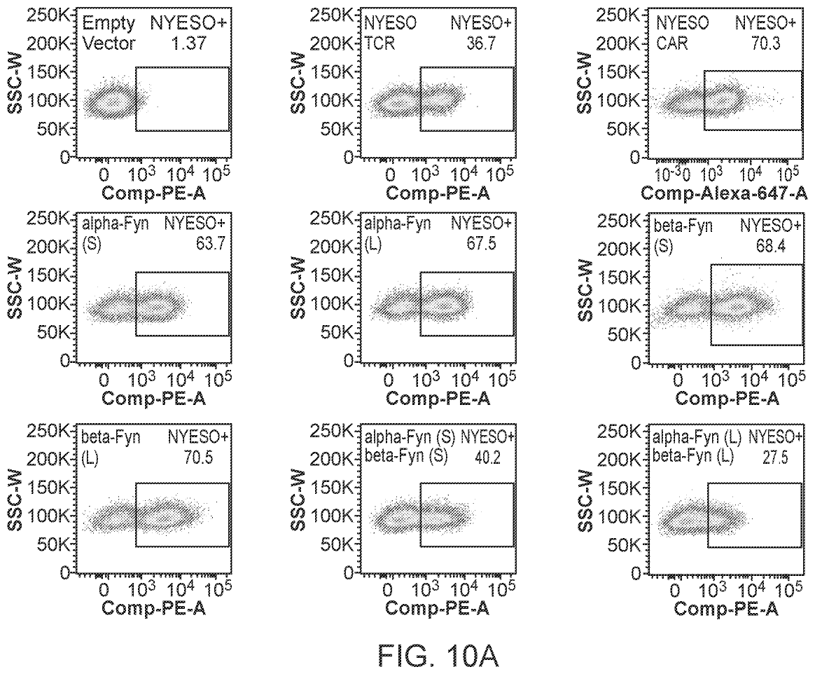

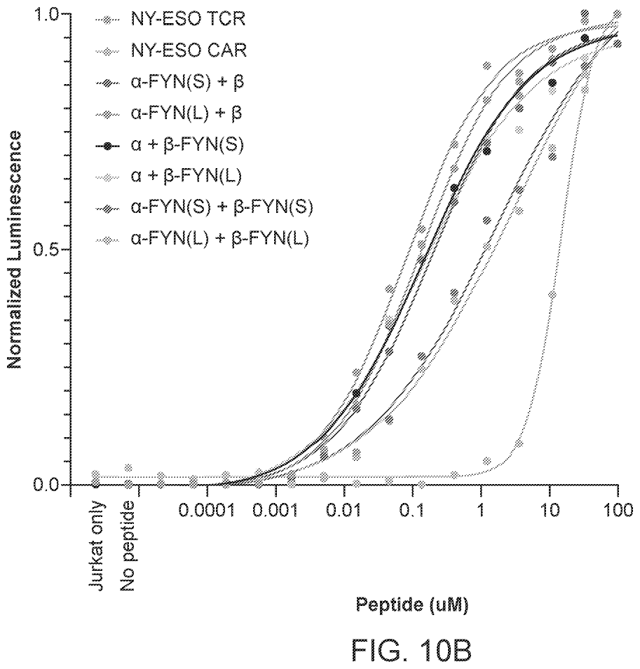

Jurkat T cells engineered with NFAT promoter-driven luciferase gene were transfected with engineered TCR constructs containing the Fyn KD fused to the C-Terminus of alpha, beta or both alpha and beta chains of the NY-ESO TCR using short or long linkers. These constructs showed decreased sensitivity to peptide compared to the parental NY-ESO TCR, indicating that addition of the globular Fyn KD disrupts TCR signaling. Results are shown in FIGS. 10A-10B. Examples of these engineered TCR architectures are shown in FIG. 17A (TCR alpha fusion), FIG. 17B (TCR beta fusion) and FIG. 17C (TCR alpha and TCR beta fusions).

### Example 11: ZAP70 Fusions

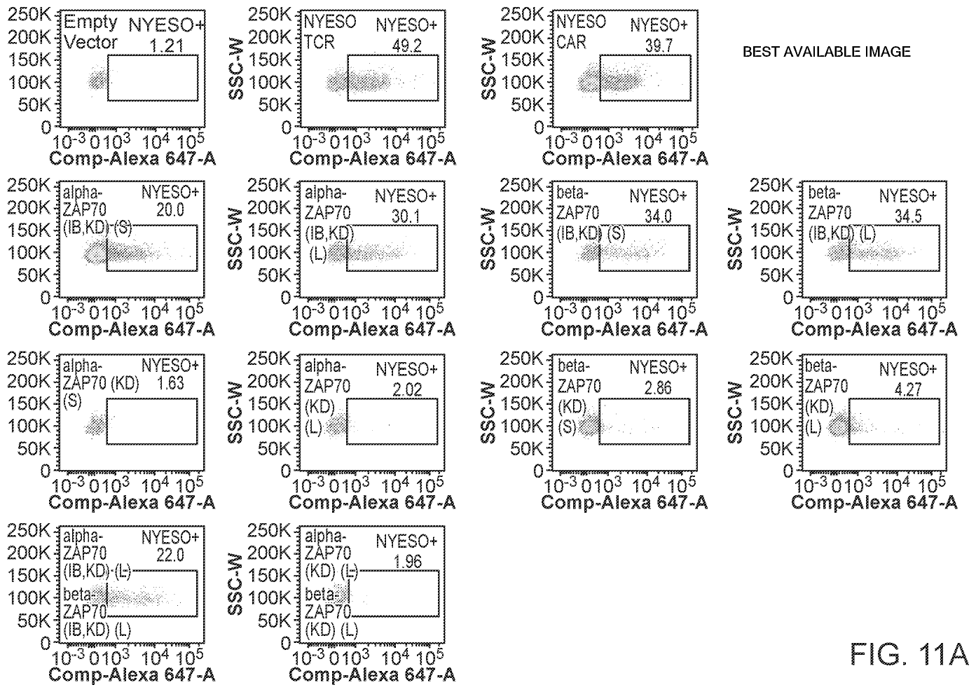

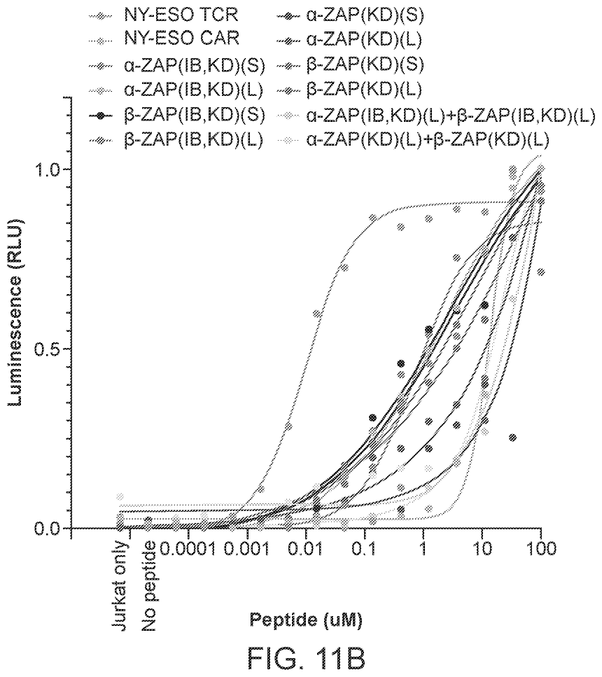

Jurkat T cells engineered with NFAT promoter-driven luciferase gene were transfected with engineered TCR constructs containing the Zap70 IB, KD or ZAP70 KD fused to the C-terminus of alpha, beta or alpha and beta chains of the NY-ESO TCR with short or long linkers. All of these constructs show significantly decreased sensitivity to peptide compared to the parental TCR. Expression of Zap70 IB, KD fusion constructs are reduced compared to the parental TCR while Zap70 KD fusions do not seem to be expressed at all. This suggests that the globular Zap70 KD reduces TCR stability and the addition of the IB domain may be acting as a linker to relieve the disruption. Results are shown in FIGS. 11A-11B. Examples of these engineered TCR architectures are shown in FIG. 17A (TCR alpha fusion), FIG. 17B (TCR beta fusion) and FIG. 17C (TCR alpha and TCR beta fusions).

### Example 12: Lat Fusions

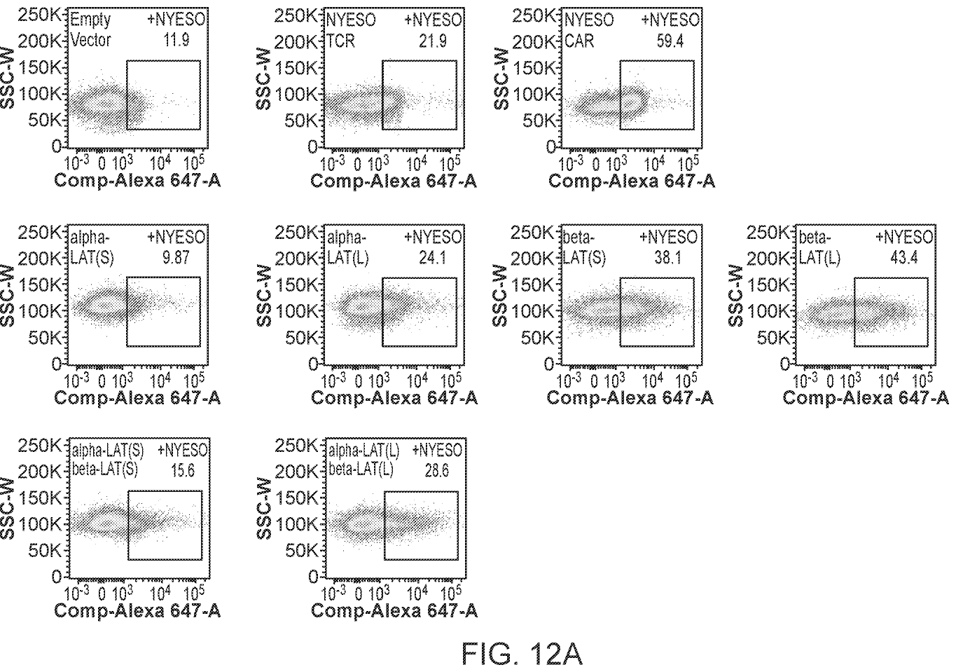

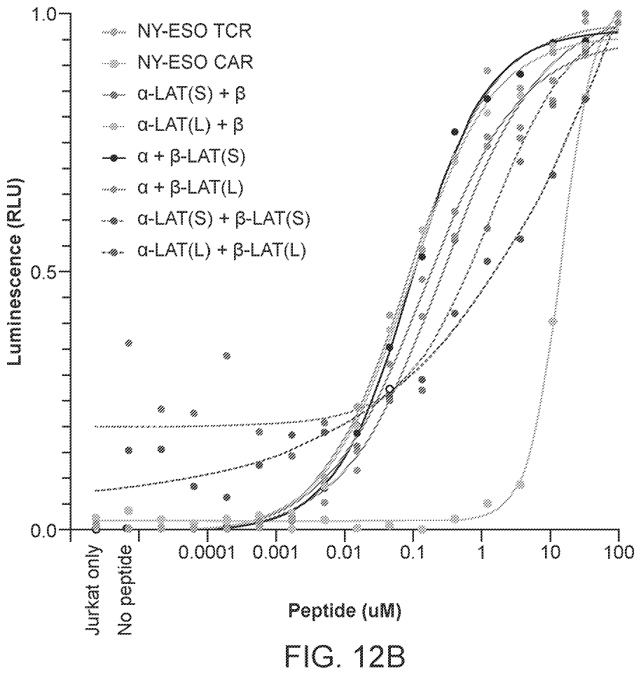

Jurkat T cells engineered with NFAT promoter-driven luciferase gene were transfected with engineered TCR constructs containing LAT fused to the C-terminus of alpha, beta or alpha and beta chains of the NY-ESO TCR with short or long linkers. A slight reduction of sensitivity is observed with LAT fused to the beta with a long linker and LAT fused to the alpha with a short linker. Combinations of LAT-fused alpha and beta TCR significantly increases background signal and reduces TCR sensitivity. The high background signal observed in Jurkat only or in co-culture with no peptide loaded target cells indicate nonspecific activation of the engineered TCR. Results are shown in FIGS. 12A-12B. Examples of these engineered TCR architectures are shown in FIG. 17A (TCR alpha fusion), FIG. 17B (TCR beta fusion) and FIG. 17C (TCR alpha and TCR beta fusions).

### Example 13: SLP76 Fusions

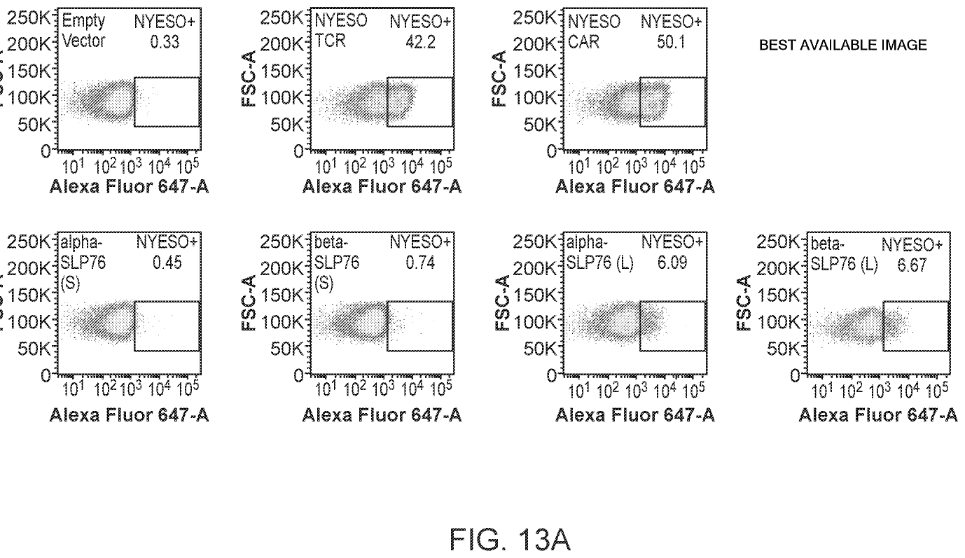

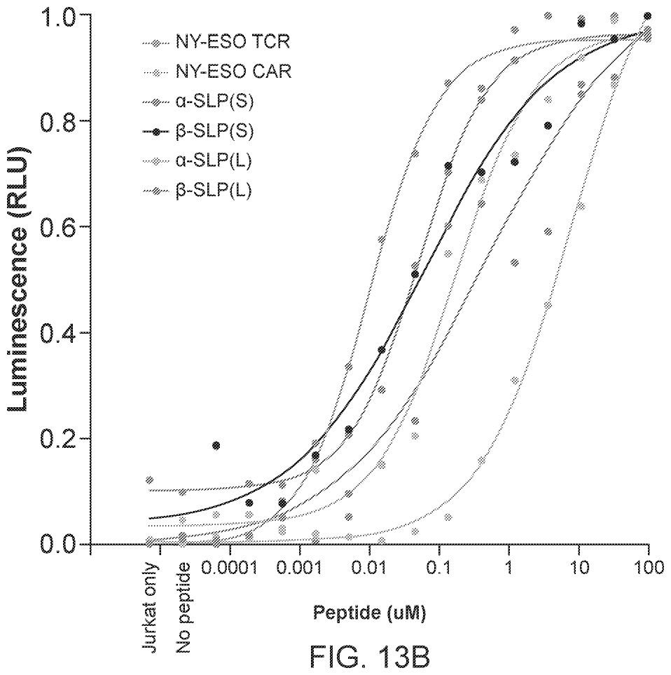

Jurkat T cells engineered with NFAT promoter-driven luciferase gene were transfected with engineered TCR constructs containing SLP76 fused to the C-terminus of alpha, beta or alpha and beta chains of the NY-ESO TCR with short or long linkers. SLP76 fusions with short linker show no expression and fusions with long linkers show significantly reduced expression level. Consistently, sensitivity all SLP76-fused engineered TCRs are reduced compared to the parental NY-ESO TCR. Results are shown in FIGS. 13A-13B. Examples of these engineered TCR architectures are shown in FIG. 17A (TCR alpha fusion) and FIG. 17B (TCR beta fusion).

### Example 14: SLP76 and Lat-Lck Fusions

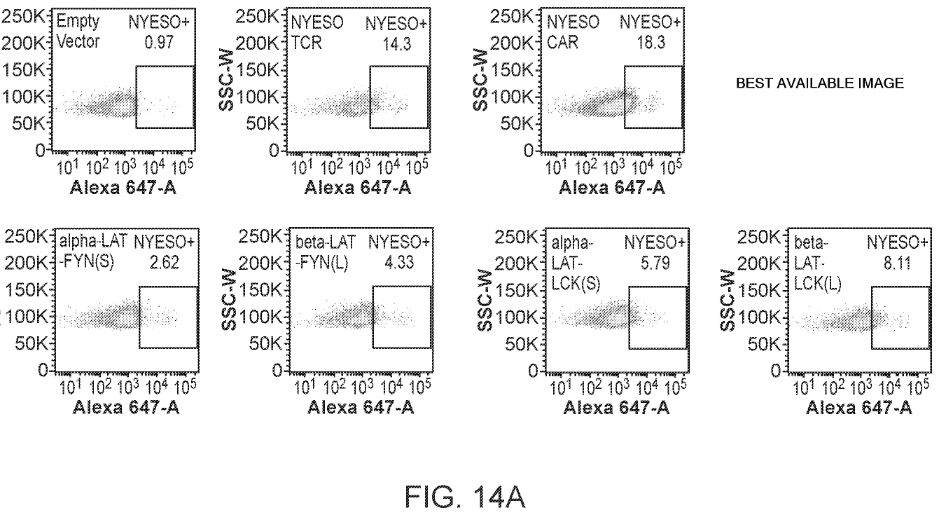

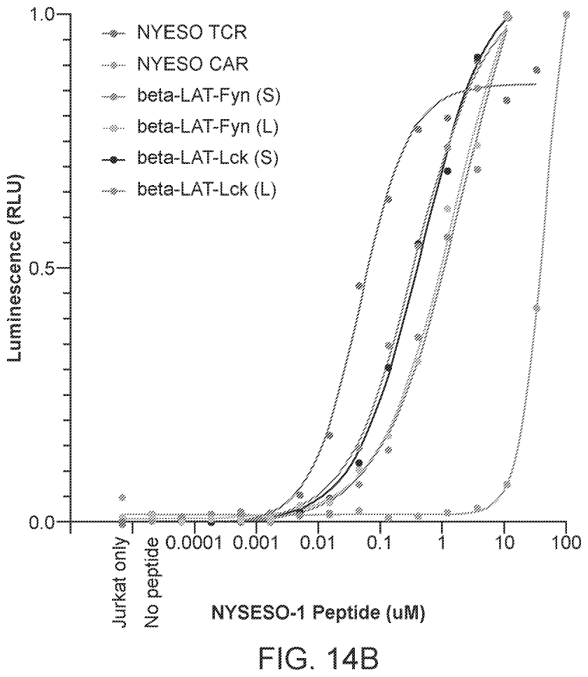

Jurkat T cells engineered with NFAT promoter-driven luciferase gene were transfected with engineered TCR constructs containing SLP76 fused to the C-terminus of alpha, beta or alpha and beta chains of the NY-ESO TCR with short or long linkers. These results show that addition of LAT-LCK fusion reduces TCR sensitivity and addition of LAT-FYN further reduces sensitivity. Results are shown in FIGS. 14A-14B. An example of this engineered TCR architectures is shown in FIG. 17D (TCR beta fusion with two domains).

### Example 15: Probe Binding and EC50 of Fusion Constructions

Tables 2A-2L summarize the results of Examples 2-14. For each experiment, relative percentage of cells expressing the TCR and relative EC50 are calculated by normalizing to NY-ESO TCR.

TABLE 2A

| TCR | | | % | Relative % | | relative |
|---|---|---|---|---|---|---|
| fusion | Linker | Construct | Expression | Expression | EC50 | EC50 |
| | | NY-ESO TCR | 21 | 1.00 | 6.25E−03 | 1.00 |
| α | GGS | α-CD4 + β | 37 | 1.80 | 7.25E−03 | 0.86 |
| α | GGS | α-41BB + β | 39 | 1.91 | 9.51E−03 | 0.66 |
| β | GGS | α + β-CD4 | 50 | 2.43 | 8.05E−03 | 0.78 |
| β | GGS | α + β-CD8 | 33 | 1.59 | 1.41E−02 | 0.44 |
| α, β | GGS | α-CD4 + β-CD4 | 36 | 1.74 | 3.43E−02 | 0.18 |
| α, β | GGS | α-41BB + β-CD8 | 27 | 1.30 | 1.12E−02 | 0.56 |
| α, β | GGS | α-CD4 + β-CD8 | 34 | 1.66 | 1.54E−02 | 0.41 |
| α, β | GGS | α-41BB + β-CD4 | 35 | 1.69 | 1.04E−02 | 0.60 |
| | | NY-ESO CAR | 0 | 0.00 | 3.14E+00 | 0.00 |

TABLE 2B

| TCR | | | % | Relative % | | relative |
|---|---|---|---|---|---|---|
| fusion | Linker | Construct | Expression | Expression | EC50 | EC50 |
| | | NY-ESO TCR | 50 | 1.00 | 1.13E−01 | 1.00 |
| α | GGS | α-CD4 + β | 58 | 1.17 | 1.86E−01 | 0.61 |
| α | GGS | α-CD8a + β | 49 | 0.98 | 2.66E−01 | 0.42 |
| β | GGS | α + β-CD4 | 57 | 1.13 | 3.35E−01 | 0.34 |
| β | GGS | α + β-CD8a | 44 | 0.89 | 2.12E−01 | 0.53 |
| α, β | GGS | α-CD4 + β-CD4 | 57 | 1.13 | 5.38E−01 | 0.21 |
| α, β | GGS | α-CD8a + β-CD8a | 29 | 0.59 | 2.61E−01 | 0.43 |
| α, β | GGS | α-CD4 + β-CD8a | 46 | 0.92 | 4.47E−01 | 0.25 |
| α, β | GGS | α-CD8a + β-CD4 | 40 | 0.80 | 3.11E−01 | 0.36 |
| α | GGS | α-CD28 + β | 61 | 1.21 | 7.36E−02 | 1.53 |
| β | GGS | α + β-CD28 | 55 | 1.11 | 9.40E−02 | 1.20 |
| α, β | GGS | α-CD28 + β-CD28 | 52 | 1.04 | 1.44E−01 | 0.78 |
| α | GGS | α-41BB + β | 52 | 1.05 | 1.56E−01 | 0.72 |
| β | GGS | α + β-41BB | 55 | 1.09 | 1.29E−01 | 0.88 |
| α, β | GGS | α-41BB + β-41BB | 39 | 0.79 | 2.58E−01 | 0.44 |
| α, β | GGS | α-CD28 + β-41BB | 49 | 0.97 | 1.22E−01 | 0.93 |
| α, β | GGS | α-41BB + β-CD28 | 53 | 1.06 | 9.77E−02 | 1.16 |
| | | NY-ESO CAR | 0 | 0.00 | 1.34E+01 | 0.00 |

TABLE 2C

| TCR | | | % | Relative % | | relative |
|---|---|---|---|---|---|---|
| fusion | Linker | Construct | Expression | Expression | EC50 | EC50 |
| | | NY-ESO TCR | 26 | 1.00 | 9.89E−02 | 1.00 |
| α | G4S | α-LCK (KD) + β | 14 | 0.55 | 1.86E+00 | 0.05 |
| α | (G4S) × 4 | α-LCK (KD) + β | 29 | 1.11 | 4.02E−01 | 0.25 |
| β | S | α + β-LCK (KD) | 32 | 1.24 | 3.96E−01 | 0.25 |
| β | (G4S) × 3 | α + β-LCK (KD) | 39 | 1.53 | 3.39E−01 | 0.29 |
| α, β | G4S, S | α-LCK (KD) + | 3 | 0.11 | 9.18E+00 | 0.01 |
| | | β-LCK (KD) | | | | |
| α, β | (G4S) × 4, | α-LCK (KD) + | 17 | 0.67 | 7.54E+00 | 0.01 |
| | (G4S) × 3 | β-LCK (KD) | | | | |
| α, β | G4S, | α-LCK (KD) + | 7 | 0.27 | 1.04E+01 | 0.01 |
| | (G4S) × 3 | β-LCK (KD) | | | | |
| α, β | (G4S) × 4, S | α-LCK (KD) + | 10 | 0.39 | 1.18E+01 | 0.01 |
| | | β-LCK (KD) | | | | |
| | | NY-ESO CAR | 0 | 0.00 | 8.63E+00 | 0.00 |

TABLE 2D

| TCR | | | % | Relative % | | relative |
|---|---|---|---|---|---|---|
| fusion | Linker | Construct | Expression | Expression | EC50 | EC50 |
| | | NY-ESO TCR | 21 | 1.00 | 8.23E−02 | 1.00 |
| α | G4S | α-LAT + β | 11 | 0.52 | 2.66E−01 | 0.31 |
| α | (G4S) × 4 | α-LAT + β | 26 | 1.23 | 8.64E−02 | 0.95 |
| β | S | α + β-LAT | 39 | 1.83 | 9.47E−02 | 0.87 |
| β | (G4S) × 3 | α + β-LAT | 45 | 2.11 | 1.50E−01 | 0.55 |
| α, β | G4S, S | α-LAT + β-LAT | 15 | 0.71 | 3.38E+02 | 0.00 |
| α, β | (G4S) × 4, | α-LAT + β-LAT | 30 | 1.39 | 1.38E+00 | 0.06 |
| | (G4S) × 3 | | | | | |
| α | G4S | α-FYN + β | 19 | 0.89 | 1.84E−01 | 0.45 |
| α | (G4S) × 4 | α-FYN + β | 29 | 1.37 | 1.29E−01 | 0.64 |
| β | S | α + β-FYN | 52 | 2.42 | 1.45E−01 | 0.57 |
| β | (G4S) × 3 | α + β-FYN | 52 | 2.43 | 1.46E−01 | 0.56 |
| α, β | G4S, S | α-FYN + β-FYN | 10 | 0.48 | 1.81E+00 | 0.05 |
| α, β | (G4S) × 4, | α-FYN + β-FYN | 6 | 0.29 | 3.18E+00 | 0.03 |
| | (G4S) × 3 | | | | | |
| | | NY-ESO CAR | 0 | 0.00 | 1.47E+01 | 0.00 |

TABLE 2E

| TCR | | | % | Relative % | | relative |
|---|---|---|---|---|---|---|
| fusion | Linker | Construct | Expression | Expression | EC50 | EC50 |
| | | NY-ESO TCR | 21 | 1.00 | 1.34E−02 | 1.00 |
| α, β | G4S, S | α-LAT + β-Fyn | 2 | 0.11 | 1.04E+00 | 0.01 |
| α, β | (G4S) × 4, | α-LAT + β-Fyn | 11 | 0.52 | 2.43E−01 | 0.06 |
| | (G4S) × 3 | | | | | |
| α, β | G4S, | α-LAT + β-Fyn | 3 | 0.17 | 5.92E−01 | 0.02 |
| | (G4S) × 3 | | | | | |
| α, β | (G4S) × 4, S | α-LAT + β-Fyn | 18 | 0.88 | 2.76E−01 | 0.05 |
| α, β | G4S, S | α-FYN + β-LAT | 11 | 0.55 | 6.73E−01 | 0.02 |
| α, β | (G4S) × 4, | α-FYN + β-LAT | 23 | 1.13 | 2.91E−01 | 0.05 |
| | (G4S) × 3 | | | | | |
| α, β | G4S, | α-FYN + β-LAT | 12 | 0.57 | 6.67E−01 | 0.02 |
| | (G4S) × 3 | | | | | |
| α, β | (G4S) × 4, S | α-FYN + β-LAT | 20 | 0.96 | 4.07E−01 | 0.03 |
| | | NY-ESO CAR | 0 | 0.00 | 5.86E+00 | 0.00 |

TABLE 2F

| TCR | | | % | Relative % | | relative |
|---|---|---|---|---|---|---|
| fusion | Linker | Construct | Expression | Expression | EC50 | EC50 |
| | | NY-ESO TCR | 54 | 1.00 | 4.29E−02 | 1.00 |
| β | (G4S) × 3 | α + β-CD4 | 59 | 1.09 | 3.50E−02 | 1.23 |
| β | (G4S) × 3 | α + β-CD8 | 58 | 1.07 | 7.77E−02 | 0.55 |
| β | (G4S) × 3 | α + β-CD28 | 64 | 1.18 | 3.04E−02 | 1.41 |
| β | (G4S) × 3 | α + β-41BB | 73 | 1.34 | 3.79E−02 | 1.13 |
| β | (G4S) × 3 | α + β-LCK | 45 | 0.83 | 8.92E−02 | 0.48 |
| β | (G4S) × 3 | α + β-FYN | 56 | 1.03 | 3.39E−02 | 1.26 |
| β | (G4S) × 3 | α + β-LAT | 32 | 0.59 | 7.33E−02 | 0.59 |
| | | NY-ESO CAR | 0 | 0.00 | 1.04E+01 | 0.00 |

TABLE 2G

| TCR | | | % | Relative % | | relative |
|---|---|---|---|---|---|---|
| fusion | Linker | Construct | Expression | Expression | EC50 | EC50 |
| | | NY-ESO TCR | 12 | 1.00 | 1.47E−01 | 1.00 |
| β | (G4S) × 3 | β-Lck [245-498] | 10 | 0.82 | 3.00E−01 | 0.49 |
| β | (G4S) × 3 | β-Lck [2-509] | 14 | 1.12 | 3.29E−01 | 0.45 |
| β | (G4S) × 3 | β-Lck [6-509] | 9 | 0.76 | 2.21E−01 | 0.66 |
| β | (G4S) × 3 | β-Lck [73-509] | 10 | 0.85 | 1.09E−01 | 1.34 |
| β | (G4S) × 3 | β-Lck [122-509] | 12 | 0.97 | 4.41E−01 | 0.33 |
| β | (G4S) × 3 | β-Lck [225-509] | 12 | 0.98 | 2.54E−01 | 0.58 |
| β | (G4S) × 3 | β-Lck [2-509, Y505A] | 10 | 0.85 | 8.53E−02 | 1.72 |
| | | NY-ESO CAR | 0 | 0.00 | 9.67E+00 | 0.00 |

TABLE 2H

| TCR | | | % | Relative % | | relative |
|---|---|---|---|---|---|---|
| fusion | Linker | Construct | Expression | Expression | EC50 | EC50 |
| | | NY-ESO TCR | 32 | 1.00 | 5.28E−02 | 1.00 |
| α | G4S | α-LAT-ZAP (KD) + β | 0 | 0.01 | 9.00E+01 | 0.00 |
| α | (G4S) × 4 | α-LAT-ZAP (KD) + β | 0 | 0.01 | 4.05E+02 | 0.00 |
| β | S | α + β-LAT-ZAP (KD) | 3 | 0.10 | 5.35E−01 | 0.10 |
| β | (G4S) × 3 | α + β-LAT-ZAP (KD) | 4 | 0.11 | 6.93E−01 | 0.08 |
| α | G4S | α-LAT-ZAP (IB.5 + KD) + β | 0 | 0.01 | 2.54E+01 | 0.00 |
| α | (G4S) × 4 | α-LAT-ZAP (IB.5 + KD) + β | 1 | 0.02 | 2.27E+01 | 0.00 |
| β | S | α + β-LAT-ZAP (IB.5 + KD) | 16 | 0.52 | 4.98E−01 | 0.11 |
| β | (G4S) × 3 | α + β-LAT-ZAP (IB.5 + KD) | 18 | 0.55 | 3.75E−01 | 0.14 |
| α | G4S | α-LAT-ZAP (IB + KD) + β | 1 | 0.04 | 6.87E+00 | 0.01 |
| α | (G4S) × 4 | α-LAT-ZAP (IB + KD) + β | 1 | 0.04 | 1.34E+01 | 0.00 |
| β | S | α + β-LAT-ZAP (IB + KD) | 29 | 0.93 | 5.58E−01 | 0.09 |
| β | (G4S) × 3 | α + β-LAT-ZAP (IB + KD) | 27 | 0.86 | 5.20E−01 | 0.10 |
| | | NY-ESO CAR | 0 | 0.00 | 1.96E+01 | 0.00 |

TABLE 21

| TCR | | | % | Relative % | | relative |
|---|---|---|---|---|---|---|
| fusion | Linker | Construct | Expression | Expression | EC50 | EC50 |
| | | NY-ESO TCR | 25 | 1.00 | 8.70E−02 | 1.00 |
| α, β | G4S, S | α-LAT + β-LAT | 1 | 0.06 | 0.00E+00 | 0.00 |
| α, β | (G4S) × 4, | α-LAT + β-LAT | 8 | 0.32 | 8.91E+00 | 0.01 |
| | (G4S) × 3 | | | | | |
| α, β | G4S, | α-LAT + β-LAT | 2 | 0.08 | 3.27E+00 | 0.03 |
| | (G4S) × 3 | | | | | |
| α, β | (G4S) × 4, S | α-LAT + β-LAT | 9 | 0.35 | 0.00E+00 | 0.00 |
| α, β | G4S, S | α-LAT-ZAP [IB.5 + KD] + | 0 | 0.01 | 0.00E+00 | 0.00 |
| | | β-LAT-ZAP [IB.5 + KD] | | | | |
| α, β | (G4S) × 4, | α-LAT-ZAP [IB.5 + KD] + | 0 | 0.01 | 0.00E+00 | 0.00 |
| | (G4S) × 3 | β-LAT-ZAP [IB.5 + KD] | | | | |
| α, β | G4S, | α-LAT-ZAP [IB.5 + KD] + | 0 | 0.01 | 3.12E+01 | 0.00 |
| | (G4S) × 3 | β-LAT-ZAP [IB.5 + KD] | | | | |
| α, β | (G4S) × 4, S | α-LAT-ZAP [IB.5 + KD] + | 0 | 0.01 | 0.00E+00 | 0.00 |
| | | β-LAT-ZAP [IB.5 + KD] | | | | |
| β | (G4S) × 3 | α + β-LAT | 26 | 1.02 | 2.79E−01 | 0.31 |
| β | (G4S) × 3 | α + β-LAT-ZAP [IB.5 + KD] | 7 | 0.27 | 2.02E+00 | 0.04 |
| | | NY-ESO CAR | 0 | 0.00 | 3.79E+01 | 0.00 |

TABLE 2J

| TCR | | | % | Relative % | | relative |
|---|---|---|---|---|---|---|
| fusion | Linker | Construct | Expression | Expression | EC50 | EC50 |
| | | NYESO TCR | 14 | 1.00 | 5.19E−02 | 1.00 |
| β | (G4S) × 3 | β-LAT | 6 | 0.40 | 1.62E−01 | 0.32 |
| β | S | β-LAT-Fyn | 3 | 0.18 | 2.33E+00 | 0.02 |
| β | (G4S) × 3 | β-LAT-Fyn | 4 | 0.30 | 1.59E+00 | 0.03 |
| β | S | β-LAT-Lck | 6 | 0.40 | 4.54E−01 | 0.11 |
| β | (G4S) × 3 | β-LAT-Lck | 8 | 0.57 | 3.77E−01 | 0.14 |
| β | (G4S) × 4 | β-LAT | 9 | 0.65 | 1.88E−01 | 0.28 |
| β | S | β-LAT-2KR | 8 | 0.55 | 2.13E−01 | 0.24 |
| β | (G4S) × 3 | β-LAT-2KR | 12 | 0.83 | 1.47E−01 | 0.35 |
| β | (G4S) × 4 | β-LAT-2KR | 17 | 1.20 | 1.21E−01 | 0.43 |
| | | NY-ESO CAR | 0 | 0.00 | 4.64E+01 | 0.00 |

TABLE 2K

| TCR | | | % | Relative % | | relative |
|---|---|---|---|---|---|---|
| fusion | Linker | Construct | Expression | Expression | EC50 | EC50 |
| | | NYESO TCR | 45 | 1.00 | 9.69E−03 | 1.00 |
| α | G4S | α-ZAP (IB, KD) | 17 | 0.38 | 7.43E−01 | 0.01 |
| α | (G4S) × 4 | α-ZAP (IB, KD) | 27 | 0.59 | 2.28E+00 | 0.00 |
| β | S | β-ZAP (IB, KD) | 30 | 0.66 | 3.55E+00 | 0.00 |
| β | (G4S) × 3 | β-ZAP (IB, KD) | 30 | 0.67 | 4.37E+00 | 0.00 |
| α | G4S | α-ZAP (KD) | 0 | 0.01 | 0.00E+00 | 0.00 |
| α | (G4S) × 4 | α-ZAP (KD) | 1 | 0.01 | 0.00E+00 | 0.00 |
| β | S | β-ZAP (KD) | 1 | 0.03 | 1.33E+01 | 0.00 |
| β | (G4S) × 3 | β-ZAP (KD) | 3 | 0.06 | 3.36E+03 | 0.00 |
| α, β | (G4S) × 4, | α-ZAP (IB, KD) + | 19 | 0.42 | 1.41E+03 | 0.00 |
| | (G4S) × 3 | β-ZAP (IB, KD) | | | | |
| α, β | (G4S) × 4, | α-ZAP (KD) + | 0 | 0.00 | 2.12E+01 | 0.00 |
| | (G4S) × 3 | β-ZAP (KD) | | | | |
| | | NY-ESO CAR | 0 | 0.00 | 1.38E+01 | 0.00 |

TABLE 2L

| TCR | | | % | Relative % | | relative |
|---|---|---|---|---|---|---|
| fusion | Linker | Construct | Expression | Expression | EC50 | EC50 |
| | | NYESO TCR | 29 | 1.00 | 4.11E−01 | 1.00 |
| β | (G4S) × 3 | β-Fyn [271-524] | 33 | 1.12 | 5.46E−01 | 0.75 |
| β | (G4S) × 3 | β-Fyn [2-537] | 21 | 0.73 | 1.69E+00 | 0.24 |
| β | (G4S) × 3 | β-Fyn [7-537] | 23 | 0.79 | 1.63E+00 | 0.25 |
| β | (G4S) × 3 | β-Fyn [144-537] | 28 | 0.97 | 2.06E+00 | 0.20 |
| β | (G4S) × 3 | β-Fyn [247-537] | 34 | 1.16 | 1.16E+00 | 0.35 |
| β | (G4S) × 3 | β-Fyn [2-537; Y531A] | 21 | 0.72 | 2.44E+00 | 0.17 |
| β | (G4S) × 3 | β-Fyn [82-537] | 32 | 1.10 | 7.80E−01 | 0.53 |
| | | NY-ESO CAR | 0 | 0.00 | 1.05E+01 | 0.00 |

FIG. 15 is a scatter plot summarizing the engineered TCR screened by the Jurkat-NFAT reporter assay. Relative EC50 and relative probe binding to the parental NY-ESO TCR are determined and plotted as a scatter. Top right quadrant shows the top engineered TCRs that show good expression and similar or better sensitivity to the parental TCR. Out of the 98 engineered TCRs screened, the top eight candidates are Lck[2-509, Y505A] fused to beta with long linker, Lck[73-509] fused to beta with long linker, CD28 fused to alpha or beta with long linker, CD4 fused to beta with long linker, 4-1BB fused to beta with long linker, and combination of 4-1BB on alpha and CD28 on beta.

TCR expression (measured as percentage of cells that bind probe) was measured by staining with biotin-labeled MHC:NY-ESO complex conjugated with fluorophore. EC₅₀ was obtained by nonlinear regression curve fitting of NFAT signal with peptide titration. Probe binding and EC₅₀ were normalized to the levels of benchmark NY-ESO-1 (clone 1G4). Color codes which subunit(s) of the TCR complex is engineered. Dotted lines are the cut off points for determining top engineered TCR constructs in terms of expression and function. The top 14 engineered TCR constructs are labeled with linker information. (L) refers to (G4S)4 on alpha and (G4S)3 on beta. (S) refers to G4S on alpha and no linker on beta.

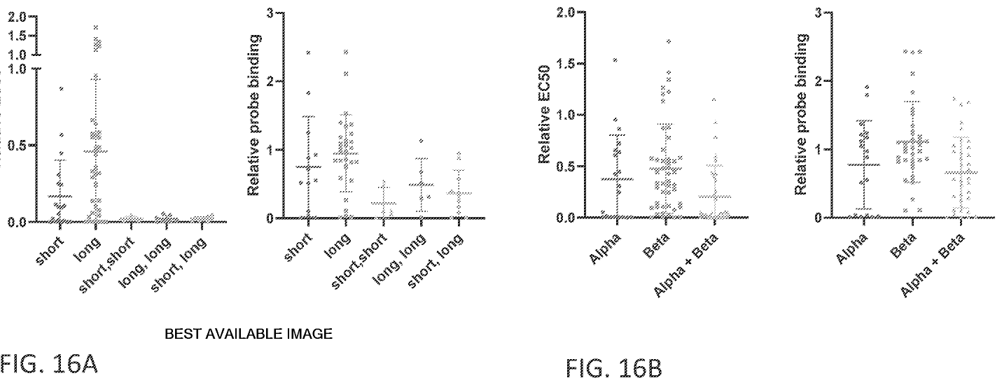

FIG. 16A shows Relative EC50 and relative probe binding of alpha, beta, or alpha and beta engineered TCRs are plotted in groups. Beta chain fusions are more sensitive and expressive on average. FIG. 16B shows relative EC50 and relative probe binding are plotted grouped according to the linker lengths. Engineered TCRs with long linkers are more sensitive and expressive on average.

Fusing intercellular signaling domains at the beta chain showed better sensitivity and expression on average than alpha-, gamma-, delta-, or epsilon-chain fusions. Individual fusions to TCR alpha or TCR beta chains resulted in greater sensitivity than engineered TCRs with both alpha- and beta-chain fusions. Engineered TCRs with long linkers were generally more sensitive and had more stable surface expression compared to engineered TCRs with short linkers. Attaching CD28 to any CD3 (TCR) subunit including TCR alpha, beta, gamma, delta and epsilon enabled signaling and did not disrupt function as measured by NFAT activation in Jurkat cells. The intracellular tail of TCR alpha and beta chains could be tagged with additional domains without disrupting the TCR signaling pathways leading to T cell activation. Using longer linker lengths affected sensitivity of engineered TCRs. In particular, higher TCR sensitivity was observed (e.g., in FIGS. 2A-2B) with fusions of 4-1BB domain to the alpha chain with a long linker than with a short linker. Various fusions resulted in improved sensitivity (e.g., CD28, 4-1BB, CD4, portions of Lck and combinations thereof). In general, fusions to TCR beta were expressed better and yielded improved sensitivity compared to TCR alpha fusions. Of all the chimeras screened, eight showed similar or better sensitivity compared to the parental TCR. These include Lck[2-509, Y505A] fused to beta chain with long linker, Lck[73-509] fused to beta with long linker, CD28 fused to alpha or beta with long linker, CD4 fused to beta with long linker, 4-1BB fused to beta with long linker, and the combination of 4-1BB on alpha and CD28 on beta.

### Example 16: Effect of C-Terminal Fusion of Inhibitory Domains on Acute TCR Activity

Jurkat cells were transiently transfected via 100 uL format Neon electroporation system (Thermo Fisher Scientific) according to manufacturer's protocol using the following settings: 3 pulses, 1500V, 10 msec. Cotransfection was performed with 0.5-1 ug of activator CAR construct and 1 ug of inhibitory TCR receptor constructs or empty vector per 1e6 cells and recovered in RPMI media supplemented with 20% heat-inactivated FBS and 0.1% Pen/Strep.

Jurkat cells were co-cultured with T2 cells loaded with titrating amounts of NY-ESO-1 peptide (ESO; SLLMWITQV) for 6 hours. To load the T2 cells, 10 uM NY-ESO-1 peptide was serially diluted 1:3 20×, and subsequently diluted and loaded onto 1e4 T2 cells in 15 uL of RPMI supplemented with 1% BSA and 0.1% Pen/Strep and incubated in Corning® 384-well Low Flange White Flat Bottom Polystyrene TC-treated Microplates. The following day, 1 e4 Jurkat cells were resuspended in 15 uL of RPMI supplemented with 10% heat-inactivated FBS and 0.1% Pen/Strep, added to the peptide-loaded T2 cells and cocultured for 6 hours. ONE-Step Luciferase Assay System (BPS Bioscience) was used to evaluate Jurkat luminescence.

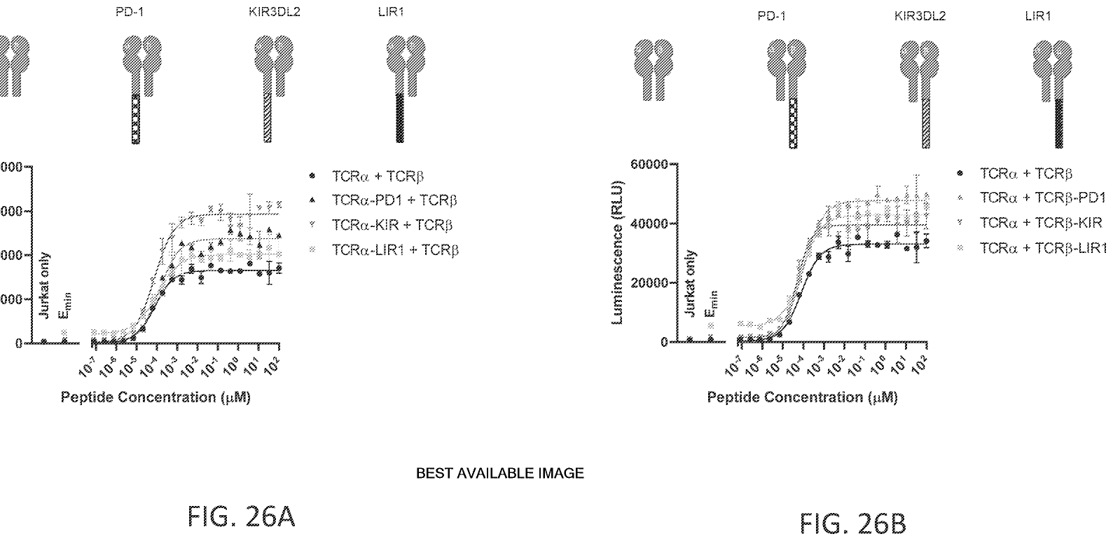

Jurkat NFAT luciferase cells were transfected with either TCRα (FIG. 26A) or TCRβ (FIG. 26B) fused to the intracellular domain of PD1, KIR3DL2 or LIR1 paired with wild type TCRβ or TCRα, respectively. ˜18-24 hours post-transfection, transfected Jurkat cells were co-cultured with T2 cells loaded with titrating amounts of NY-ESO-1(v) peptide for 6 hours. When ITIM domains were C-terminally fused to the ligand binding domain of a wild type TCR, no changes in TCR sensitivity were observed. EC₅₀ values for the various TCRα and TCRβ fusion constructs is shown in Table 3 below. Sequences of inhibitory TCR constructs are provided in Table 3 below.

TABLE 3
EC50 of TCRs with inhibitory domains
fused to the TCRα or TCRβ subunit.

| Constructs | EC50 |
|---|---|
| TCRα + TCRβ | 0.07951 |
| TCRα-PD1 + TCRβ | 0.1302 |
| TCRα-KIR + TCRβ | 0.07611 |
| TCRα-LIR1 + TCRβ | 0.1045 |
| TCRα + TCRβ-PD1 | 0.0905 |
| TCRα + TCRβ-KIR3DL2 | 0.05063 |
| TCRα + TCRβ-LIR1 | 0.08469 |

TABLE 4
Inhibitory TCR sequences

| | | Nucleotide |
|---|---|---|
| Construct | Amino Acid Sequence | Sequence |
| Synthetic | MKWKALFTAAILQAQLPITEAQSFGLLDPKLCYLLDGILFIYGVILTALF | ND |
| invCD3z | LRVKFSRSADAPAYQQGQNQLYNELNLGRREEYDVLDKRRGRDPEMGGKP | |
| (C2063) | RRKNPQEGLYNELQKDKMAEAYSEIGMKGERRRGKGHDGLYQGLSTATKD | |
| | TYDALHMQALPPRGGGGSGGGGSRVKFSRSADAPAYQQPDPQEVTYAQLD | |
| | KRRGRDPEMGGKPRRKPDPQEVTYAQLGMKGERRRGKGHPDPQEVTYAQL | |
| | HMQALPPR (SEQ ID NO: 154) | |
| Synthetic | MKWKALFTAAILQAQLPITEAQSFGLLDPKLCYLLDGILFIYGVILTALF | ND |
| invCD3z | LRVKFSRSADAPAYQQGQNQLYNELNLGRREEYDVLDKRRGRDPEMGGKP | |
| (C2064) | RRKNPQEGLYNELQKDKMAEAYSEIGMKGERRRGKGHDGLYQGLSTATKD | |
| | TYDALHMQALPPRGGGGSGGGGSRHRRQGKHWTSTQRKADFQHPAGAVGP | |
| | EPTDRGLQWRSSPAADAQEENLYAAVKHTQPEDGVEMDTRSPHDEDPQAV | |
| | TYAEVKHSRPRREMASPPSPLSGEFLDTKDRQAEEDRQMDTEAAASEAPQ | |
| | DVTYAQLHSLTLRREATEPPPSQEGPSPAVPSIYATLAIH (SEQ ID | |
| | NO: 155) | |
| Synthetic | MKWKALFTAAILQAQLPITEAQSFGLLDPKLCYLLDGILFIYGVILTALF | ND |
| invCD3z | LRVKFSRSADAPAYQQPDPQEVTYAQLDKRRGRDPEMGGKPRRKPDPQEV | |
| (C2065) | TYAQLGMKGERRRGKGHPDPQEVTYAQLHMQALPPR (SEQ ID NO: | |
| | 156) | |
| TCRα- | METLLGLLILWLQLQWVSSKQEVTQIPAALSVPEGENLVLNCSFTDSAIY | SEQ ID |
| PD1 | NLQWFRQDPGKGLTSLLLIQSSQREQTSGRLNASLDKSSGRSTLYIAASQ | NO: 165 |
| (C2066) | PGDSATYLCAVRPLYGGSYIPTFGRGTSLIVHPYIQNPDPAVYQLRDSKS | |
| | SDKSVCLFTDFDSQTNVSQSKDSDVYITDKCVLDMRSMDFKSNSAVAWSN | |
| | KSDFACANAFNNSIIPEDTFFPSPESSCDVKLVEKSFETDTNLNFQNLSV | |
| | IGFRILLLKVAGFNLLMTLRLWSSGGGGSGGGGSGGGGSGGGGSRAARGT | |
| | IGARRTGQPLKEDPSAVPVFSVDYGELDFQWREKTPEPPVPCVPEQTEYA | |
| | TIVFPSGMGTSSPARRGSADGPRSAQPLRPEDGHCSWPL (SEQ ID | |
| | NO: 157) | |
| TCRα- | METLLGLLILWLQLQWVSSKQEVTQIPAALSVPEGENLVLNCSFTDSAIY | SEQ ID |
| (C2067) | NLQWFRQDPGKGLTSLLLIQSSQREQTSGRLNASLDKSSGRSTLYIAASQ | NO: 166 |
| KIR3DL | PGDSATYLCAVRPLYGGSYIPTFGRGTSLIVHPYIQNPDPAVYQLRDSKS | |
| 2 | SDKSVCLFTDFDSQTNVSQSKDSDVYITDKCVLDMRSMDFKSNSAVAWSN | |
| | KSDFACANAFNNSIIPEDTFFPSPESSCDVKLVEKSFETDTNLNFQNLSV | |
| | IGFRILLLKVAGFNLLMTLRLWSSGGGGSGGGGSGGGGSGGGGSNKKNAA | |
| | VMDQEPAGDRTVNRQDSDEQDPQEVTYAQLDHCVFIQRKISRPSQRPKTP | |
| | LTDTSVYTELPNAEPRSKVVSCPRAPQSGLEGVF (SEQ ID NO: | |
| | 158) | |
| TCRα- | METLLGLLILWLQLQWVSSKQEVTQIPAALSVPEGENLVLNCSFTDSAIY | SEQ ID |
| LIR1 | NLQWFRQDPGKGLTSLLLIQSSQREQTSGRLNASLDKSSGRSTLYIAASQ | NO: 167 |
| (C2068) | PGDSATYLCAVRPLYGGSYIPTFGRGTSLIVHPYIQNPDPAVYQLRDSKS | |
| | SDKSVCLFTDFDSQTNVSQSKDSDVYITDKCVLDMRSMDFKSNSAVAWSN | |
| | KSDFACANAFNNSIIPEDTFFPSPESSCDVKLVEKSFETDTNLNFQNLSV | |
| | IGFRILLLKVAGFNLLMTLRLWSSGGGGSGGGGSGGGGSGGGGSRHRRQG | |
| | KHWTSTQRKADFQHPAGAVGPEPTDRGLQWRSSPAADAQEENLYAAVKHT | |
| | QPEDGVEMDTRSPHDEDPQAVTYAEVKHSRPRREMASPPSPLSGEFLDTK | |
| | DRQAEEDRQMDTEAAASEAPQDSAIH (SEQ ID NO: 159) | |
| Synthetic | METLLGLLILWLQLQWVSSKQEVTQIPAALSVPEGENLVLNCSFTDSAIY | ND |
| (C2069) | NLQWFRQDPGKGLTSLLLIQSSQREQTSGRLNASLDKSSGRSTLYIAASQ | |
| invCD3z | PGDSATYLCAVRPLYGGSYIPTFGRGTSLIVHPYIQNPDPAVYQLRDSKS | |
| | SDKSVCLFTDFDSQTNVSQSKDSDVYITDKCVLDMRSMDFKSNSAVAWSN | |
| | KSDFACANAFNNSIIPEDTFFPSPESSCDVKLVEKSFETDTNLNFQNLSV | |
| | IGFRILLLKVAGFNLLMTLRLWSSGGGGSGGGGSGGGGSGGGGSRVKFSR | |
| | SADAPAYQQPDPQEVTYAQLDKRRGRDPEMGGKPRRKPDPQEVTYAQLGM | |
| | KGERRRGKGHPDPQEVTYAQLHMQALPPR (SEQ ID NO: 160) | |
| TCRβ- | MSIGLLCCAALSLLWAGPVNAGVTQTPKFQVLKTGQSMTLQCAQDMNHEY | SEQ ID |
| (C2070) | MSWYRQDPGMGLRLIHYSVGAGITDQGEVPNGYNVSRSTTEDFPLRLLSA | NO: 168 |
| PD1 | APSQTSVYFCASSYVGNTGELFFGEGSRLTVLEDLKNVFPPEVAVFEPSE | |
| | AEISHTQKATLVCLATGFYPDHVELSWWVNGKEVHSGVCTDPQPLKEQPA | |
| | LNDSRYCLSSRLRVSATFWQNPRNHFRCQVQFYGLSENDEWTQDRAKPVT | |
| | QIVSAEAWGRADCGFTSESYQQGVLSATILYEILLGKATLYAVLVSALVL | |
| | MAMVKRKDSRGGGGGSGGGGSGGGGSRAARGTIGARRTGQPLKEDPSAVP | |
| | VFSVDYGELDFQWREKTPEPPVPCVPEQTEYATIVFPSGMGTSSPARRGS | |
| | ADGPRSAQPLRPEDGHCSWPL (SEQ ID NO: 161) | |
| TCRβ- | MSIGLLCCAALSLLWAGPVNAGVTQTPKFQVLKTGQSMTLQCAQDMNHEY | SEQ ID |
| (C2071) | MSWYRQDPGMGLRLIHYSVGAGITDQGEVPNGYNVSRSTTEDFPLRLLSA | NO: 169 |
| KIR3DL | APSQTSVYFCASSYVGNTGELFFGEGSRLTVLEDLKNVFPPEVAVFEPSE | |
| 2 | AEISHTQKATLVCLATGFYPDHVELSWWVNGKEVHSGVCTDPQPLKEQPA | |
| | LNDSRYCLSSRLRVSATFWQNPRNHFRCQVQFYGLSENDEWTQDRAKPVT | |
| | QIVSAEAWGRADCGFTSESYQQGVLSATILYEILLGKATLYAVLVSALVL | |
| | MAMVKRKDSRGGGGGSGGGGSGGGGSNKKNAAVMDQEPAGDRTVNRQDSD | |
| | EQDPQEVTYAQLDHCVFIQRKISRPSQRPKTPLTDTSVYTELPNAEPRSK | |
| | VVSCPRAPQSGLEGVF (SEQ ID NO: 162) | |
| TCRβ- | MSIGLLCCAALSLLWAGPVNAGVTQTPKFQVLKTGQSMTLQCAQDMNHEY | SEQ ID |
| LIR1 | MSWYRQDPGMGLRLIHYSVGAGITDQGEVPNGYNVSRSTTEDFPLRLLSA | NO: 170 |
| (C2072) | APSQTSVYFCASSYVGNTGELFFGEGSRLTVLEDLKNVFPPEVAVFEPSE | |
| | AEISHTQKATLVCLATGFYPDHVELSWWVNGKEVHSGVCTDPQPLKEQPA | |
| | LNDSRYCLSSRLRVSATFWQNPRNHFRCQVQFYGLSENDEWTQDRAKPVT | |
| | QIVSAEAWGRADCGFTSESYQQGVLSATILYEILLGKATLYAVLVSALVL | |
| | MAMVKRKDSRGGGGGSGGGGSGGGGSRHRRQGKHWTSTQRKADFQHPAGA | |
| | VGPEPTDRGLQWRSSPAADAQEENLYAAVKHTQPEDGVEMDTRSPHDEDP | |
| | QAVTYAEVKHSRPRREMASPPSPLSGEFLDTKDRQAEEDRQMDTEAAASE | |
| | APQDVTYAQLHSLTLRREATEPPPSQEGPSPAVPSIYATLAIH (SEQ | |
| | ID NO: 163) | |
| Synthetic | MSIGLLCCAALSLLWAGPVNAGVTQTPKFQVLKTGQSMTLQCAQDMNHEY | ND |
| invCD3z | MSWYRQDPGMGLRLIHYSVGAGITDQGEVPNGYNVSRSTTEDFPLRLLSA | |
| (C2073) | APSQTSVYFCASSYVGNTGELFFGEGSRLTVLEDLKNVFPPEVAVFEPSE | |
| | AEISHTQKATLVCLATGFYPDHVELSWWVNGKEVHSGVCTDPQPLKEQPA | |
| | LNDSRYCLSSRLRVSATFWQNPRNHFRCQVQFYGLSENDEWTQDRAKPVT | |
| | QIVSAEAWGRADCGFTSESYQQGVLSATILYEILLGKATLYAVLVSALVL | |
| | MAMVKRKDSRGGGGGSGGGGSGGGGSRVKFSRSADAPAYQQPDPQEVTYA | |
| | QLDKRRGRDPEMGGKPRRKPDPQEVTYAQLGMKGERRRGKGHPDPQEVTY | |
| | AQLHMQALPPR (SEQ ID NO: 164) | |

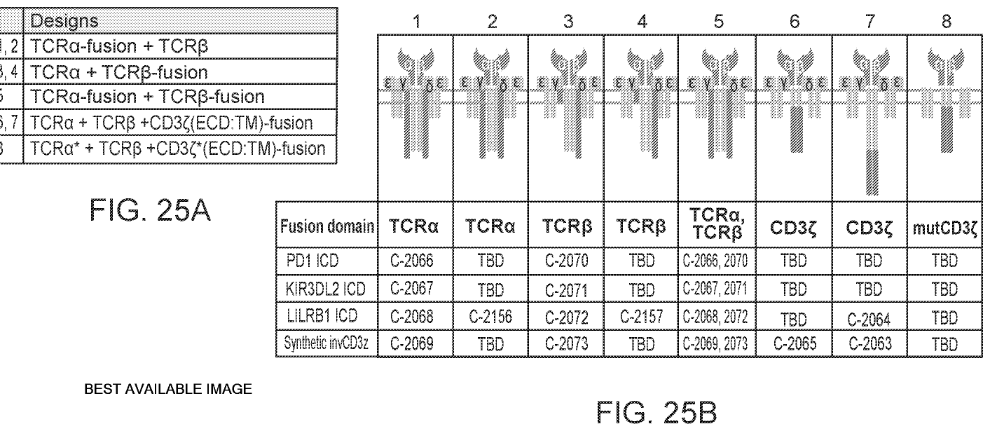

Constructs in Table 4 are also summarized in FIGS. 25A and 25B. Underlining indicates intracellular domains.

### Example 16: Inhibitor TCR Constructs in Jurkat Cells with No Endogenous TCR Activity

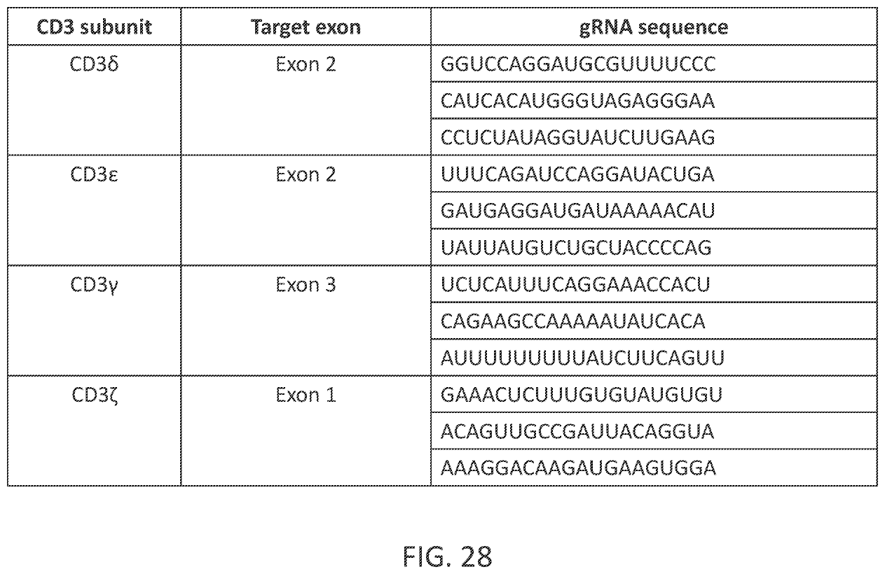

Cas9-mediated gene editing was used to knockout CD3D, CD3E, CD3G, CD3Z in a Jurkat-NFAT-Luciferase reporter line. gRNAs were designed and generated by Synthego (using the Synthego Gene Knockout Kit V2), and the sequences of gRNAs used to induce double strand breaks and deletions in CD3D, CD3E, CD3G or CD3Z via non-homologous end joining are shown in FIG. 28. RNP complex was generated by mixing gRNA (3.6 uM) with spCas9 (2.6 uM) in Neon Resuspension buffer (total volume 14 uL). The RNP complex was incubated at room temperature for 15 min. The RNP complex was mixed with 14 uL of Jurkat NFAT luciferase cells at 40 E6/mL. Jurkat cells were transfected with gRNA-spCas9 complex using the Neon electroporator (1400 V, 10 mx, 3 pulses). CD3D/E RNPs were combined. After enriching for CD3E negative cells, CD3G/Z gRNA-spCas9 RNPs were combined and transfected as described above.

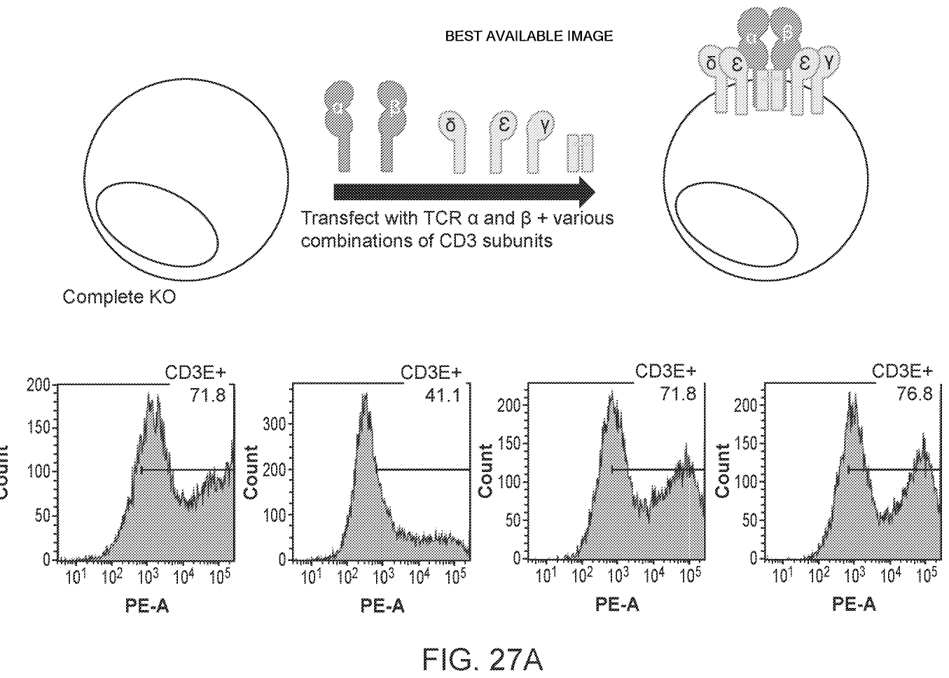

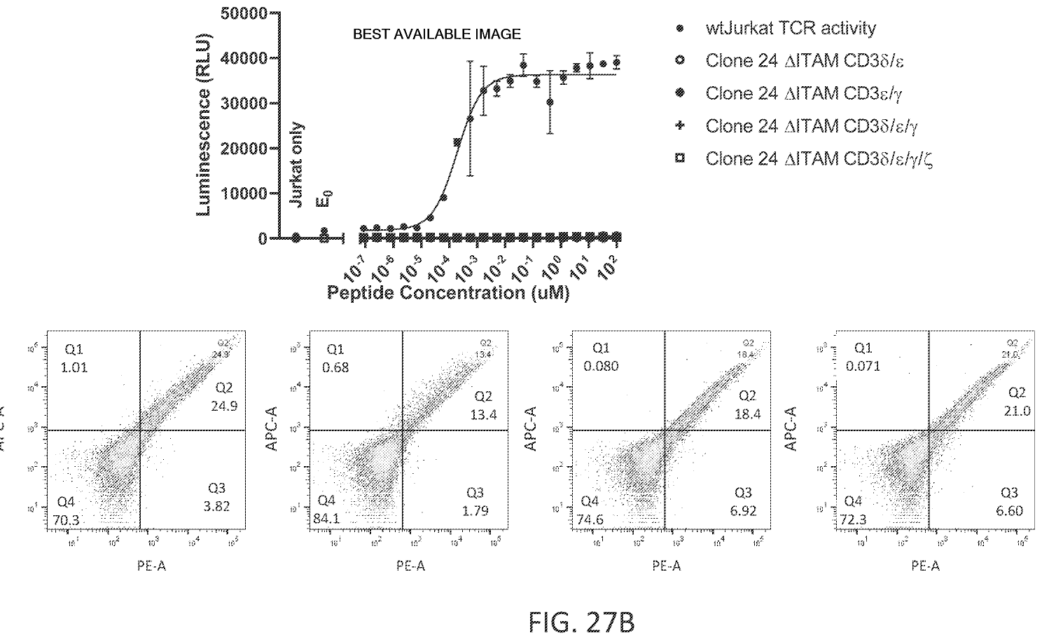

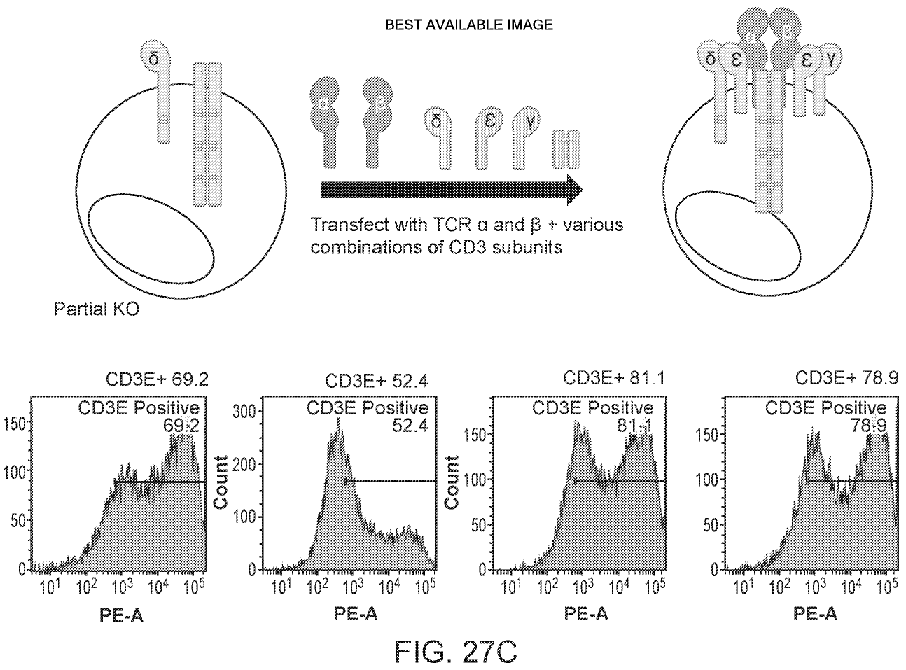

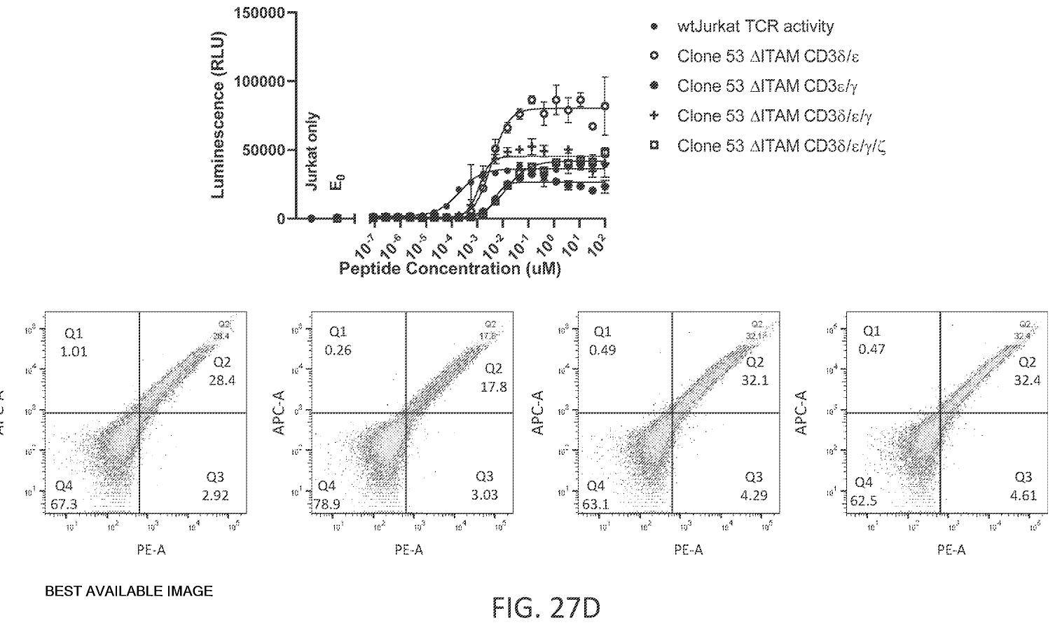

A Jurkat-NFAT-Luciferase reporter line with a complete knockout of endogenous TCR subunits was generated using Cas9-mediated gene editing. This line was then transfected with TCR alpha and TCR beta, and various combinations of CD3 subunits as shown in FIGS. 27A-27B. The activator receptor recognized an NY-ESO-1 1G4 95aLY ligand, and all inhibitory TCR ligand binding domains were the same. HLA-A*0201-NYESO was stained using APC, and TCR Vbeta 13.1 (TCRb) was stained with PE and the expression was determined using fluorescence activated cell sorting. CD3 subunits that lack an activating intracellular ITAM domain (CD3ΔITAM) were expressed in complete functional knockout clone (FIGS. 27A-27B) or in a partial knockout clone (FIGS. 27C-27D), and the resulting NFAT-luciferase activity was assayed. As seen in FIG. 27B, clone 24 showed no TCR mediated activity when transfected with various combinations of CD3ΔITAM TCR subunits, while clone 53, a partial TCR knockout, showed partial activity when transfected with combinations of CD3ΔITAM TCR subunits (FIG. 27D). Constructs used in these experiments are shown in Table 5 below.

TABLE 5
CD3 subunit constructs with ITAM deletions

| Gene/ | | Nucleotide |
|---|---|---|
| Name | Amino Acid Sequence | Sequence |
| AITAM | MEHSTFLSGLVLATLLSQVS | SEQ ID |
| CD3D | PFKIPIEELEDRVFVNCNTS | NO: 175 |
| | ITWVEGTVGTLLSDITRLDL | |
| | GKRILDPRGIYRCNGTDIYK | |
| | DKESTVQVHYRMCQSCVELD | |
| | PATVAGIIVTDVIATLLLAL | |
| | GVFCFAGHETGR | |
| | (SEQ ID NO: 171) | |
| AITAM | MQSGTHWRVLGLCLLSVGVW | SEQ ID |
| CD3E | GQDGNEEMGGITQTPYKVSI | NO: 176 |
| | SGTTVILTCPQYPGSEILWQ | |
| | HNDKNIGGDEDDKNIGSDED | |
| | HLSLKEFSELEQSGYYVCYP | |
| | RGSKPEDANFYLYLRARVCE | |
| | NCMEMDVMSVATIVIVDICI | |
| | TGGLLLLVYYWSKNRKAKAK | |
| | (SEQ ID NO: 172) | |
| AITAM | MEQGKGLAVLILAIILLQGT | SEQ ID |
| CD3G | LAQSIKGNHLVKVYDYQEDG | NO: 177 |
| | SVLLTCDAEAKNITWFKDGK | |
| | MIGFLTEDKKKWNLGSNAKD | |
| | PRGMYQCKGSQNKSKPLQVY | |
| | YRMCQNCIELNAATIS GFL | |
| | FAEIVSIFVLAVGVYFIAG | |
| | QDGVR | |
| | (SEQ ID NO: 173) | |
| AITAM | MKWKALFTAAILQAQLPITE | SEQ ID |
| CD3Z | AQSFGLLDPKLCYLLDGILF | NO: 178 |
| | IYGVILTALFLRVKFSRSA | |
| | (SEQ ID NO: 174) | |

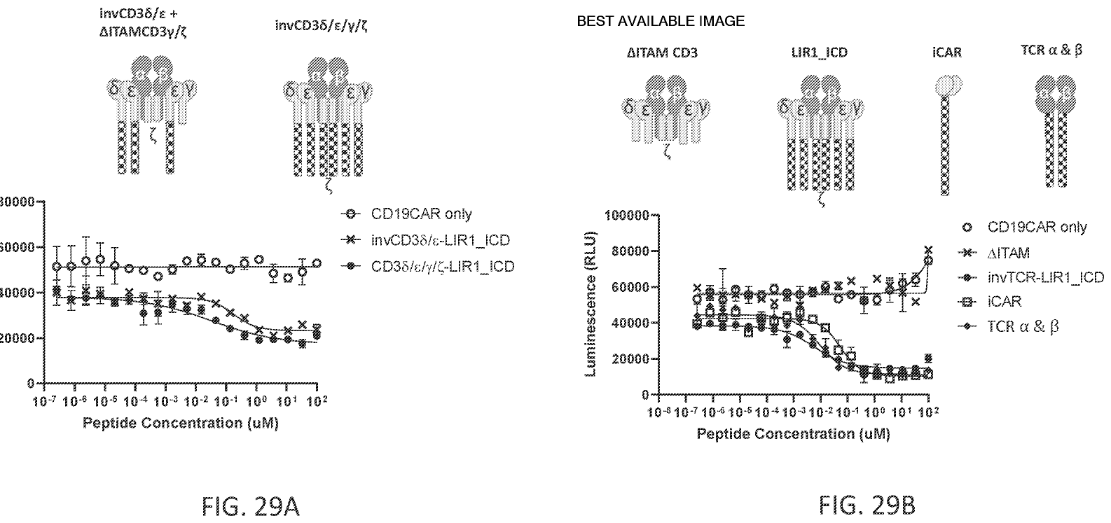

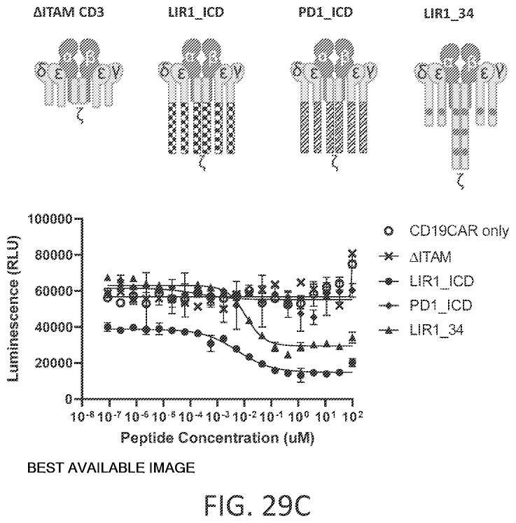

FIGS. 29A-29C show the activity of inhibitory TCRs reconstituted in the functionally CD3-null Jurkat NFAT luciferase cell line. In FIG. 29A, the ICD of CD3 delta and CD3 epsilon, and in one case, also CD3 gamma and CD3 zeta, was replaced with the ICD of LIR1. These LIR1 CD3 subunits were expressed in the functionally CD3 null clone 24 of the Jurkat NFAT-luciferase line along with TCRa and TCRb specific to A*02:01-NY-ESO-1. To determine the IC50 of the inhibitory TCR, Jurkat reporter cells co-expressed the inhibitory TCR along with an activating CD19 CAR, which reacts to CD19 expressed on the surface of T2 target cells. In the absence of wild type CD3 subunits, reconstituted inhibitory TCR was able to block CD19 CAR in a ligand dependent manner (FIG. 29A).

FIG. 29B compares the IC50 of the NY-ESO-1 inhibitory TCR with LIR1 inhibitory domains on CD3 delta, epsilon, gamma and zeta subunits to an NY-EOS-1 inhibitory TCR with a LIR1 ICD fused to the TCR alpha and beta subunits, and inhibitory CAR against A*02:01-NY-ESO-1 pMHC. Inhibitory activity against the CD19 CAR was very similar between both inhibitory TCR architectures and the inhibitory CAR.

FIG. 29C compares the activity of three types of intracellular domains (ICD) that were appended to CD3 delta, epsilon, gamma and zeta subunits of the TCR. A LIR1 CD, or a PD1 ICD, were fused to the CD3 subunits, or the region of LIR1 that contains ITIM 3 and 4 was swapped for the ITAM containing region in the native CD3 backbone, and the ability of these inhibitory receptors to inhibit Jurkat receptor cell activation by the CD19 CAR was compared. When LIR1 ITIMs were used, blocking was observed regardless of what ICD backbone was used (FIG. 29C, compare LIR1_ICD and LIR1_34). LIR1 domains were more effective at blocking activity than the PD1 ICD in this context.

TABLE 6
Sequences of CD3D, CD3E, CD3G and CD3Z fusion Constructs

| | | Nucleotide |
|---|---|---|
| Name | Amino Acid Sequence | Sequence |
| CD3D- | MEHSTFLSGLVLATLLSQVSPFKIPIEELEDRVFVNCNTSITWVEGTVG | SEQ ID |
| PD1 ICD | TLLSDITRLDLGKRILDPRGIYRCNGTDIYKDKESTVQVHYRMCQSCVE | NO: 191 |
| | LDPATVAGIIVTDVIATLLLALGVFCFAGHETGRTERRAEVPTAHPSPS | |
| | PRPAGQFQTLVVGVVGGLLGSLVLLVWVLAVICSRAARGTIGARRTGQP | |
| | LKEDPSAVPVFSVDYGELDFQWREKTPEPPVPCVPEQTEYATIVFPSGM | |
| | GTSSPARRGSADGPRSAQPLRPEDGHCSWPL (SEQ ID NO: 179) | |
| CD3E- | MQSGTHWRVLGLCLLSVGVWGQDGNEEMGGITQTPYKVSISGTTVILTC | SEQ ID |
| PD1 ICD | PQYPGSEILWQHNDKNIGGDEDDKNIGSDEDHLSLKEFSELEQSGYYVC | NO: 193 |
| | YPRGSKPEDANFYLYLRARVCENCMEMDVMSVATIVIVDICITGGLLLL | |
| | VYYWSKNRKAKAKTERRAEVPTAHPSPSPRPAGQFQTLVVGVVGGLLGS | |
| | LVLLVWVLAVICSRAARGTIGARRTGQPLKEDPSAVPVFSVDYGELDFQ | |
| | WREKTPEPPVPCVPEQTEYATIVFPSGMGTSSPARRGSADGPRSAQPLR | |
| | PEDGHCSWPL (SEQ ID NO: 180) | |
| CD3G- | MEQGKGLAVLILAIILLQGTLAQSIKGNHLVKVYDYQEDGSVLLTCDAE | SEQ ID |
| PD1 ICD | AKNITWFKDGKMIGFLTEDKKKWNLGSNAKDPRGMYQCKGSQNKSKPLQ | NO: 194 |
| | VYYRMCQNCIELNAATISGFLFAEIVSIFVLAVGVYFIAGQDGVRTERR | |
| | AEVPTAHPSPSPRPAGQFQTLVVGVVGGLLGSLVLLVWVLAVICSRAAR | |
| | GTIGARRTGQPLKEDPSAVPVFSVDYGELDFQWREKTPEPPVPCVPEQT | |
| | EYATIVFPSGMGTSSPARRGSADGPRSAQPLRPEDGHCSWPL (SEQ | |
| | ID NO: 181) | |
| CD3Z- | MKWKALFTAAILQAQLPITEAQSFGLLDPKLCYLLDGILFIYGVILTAL | SEQ ID |
| PD1 ICD | FLRVKFSRSATERRAEVPTAHPSPSPRPAGQFQTLVVGVVGGLLGSLVL | NO: 195 |
| | LVWVLAVICSRAARGTIGARRTGQPLKEDPSAVPVFSVDYGELDFQWRE | |
| | KTPEPPVPCVPEQTEYATIVFPSGMGTSSPARRGSADGPRSAQPLRPED | |
| | GHCSWPL (SEQ ID NO: 182) | |
| CD3D- | MEHSTFLSGLVLATLLSQVSPFKIPIEELEDRVFVNCNTSITWVEGTVG | SEQ ID |
| LIRI | TLLSDITRLDLGKRILDPRGIYRCNGTDIYKDKESTVQVHYRMCQSCVE | NO: 196 |
| ICD | LDPATVAGIIVTDVIATLLLALGVFCFAGHERHRRQGKHWTSTQRKADF | |
| | QHPAGAVGPEPTDRGLQWRSSPAADAQEENLYAAVKHTQPEDGVEMDTR | |
| | SPHDEDPQAVTYAEVKHSRPRREMASPPSPLSGEFLDTKDRQAEEDRQM | |
| | DTEAAASEAPQDVTYAQLHSLTLRREATEPPPSQEGPSPAVPSIYATLA | |
| | IH (SEQ ID NO: 183) | |
| CD3E- | MQSGTHWRVLGLCLLSVGVWGQDGNEEMGGITQTPYKVSISGTTVILTC | SEQ ID |
| LIR1 | PQYPGSEILWQHNDKNIGGDEDDKNIGSDEDHLSLKEFSELEQSGYYVC | NO: 197 |
| ICD | YPRGSKPEDANFYLYLRARVCENCMEMDVMSVATIVIVDICITGGLLLL | |
| | VYYWSKNRRHRRQGKHWTSTQRKADFQHPAGAVGPEPTDRGLQWRSSPA | |
| | ADAQEENLYAAVKHTQPEDGVEMDTRSPHDEDPQAVTYAEVKHSRPRRE | |
| | MASPPSPLSGEFLDTKDRQAEEDRQMDTEAAASEAPQDVTYAQLHSLTL | |
| | RREATEPPPSQEGPSPAVPSIYATLAIH (SEQ ID NO: 184) | |
| CD3G- | MEQGKGLAVLILAIILLQGTLAQSIKGNHLVKVYDYQEDGSVLLTCDAE | SEQ ID |
| LIR1 | AKNITWFKDGKMIGFLTEDKKKWNLGSNAKDPRGMYQCKGSQNKSKPLQ | NO: 198 |
| ICD | VYYRMCQNCIELNAATISGFLFAEIVSIFVLAVGVYFIAGRHRRQGKHW | |
| | TSTQRKADFQHPAGAVGPEPTDRGLQWRSSPAADAQEENLYAAVKHTQP | |
| | EDGVEMDTRSPHDEDPQAVTYAEVKHSRPRREMASPPSPLSGEFLDTKD | |
| | RQAEEDRQMDTEAAASEAPQDVTYAQLHSLTLRREATEPPPSQEGPSPA | |
| | VPSIYATLAIH (SEQ ID NO: 185) | |
| CD3Z- | MKWKALFTAAILQAQLPITEAQSFGLLDPKLCYLLDGILFIYGVILTAL | SEQ ID |
| LIR1 | FLRVKFRHRRQGKHWTSTQRKADFQHPAGAVGPEPTDRGLQWRSSPAAD | NO: 199 |
| ICD | AQEENLYAAVKHTQPEDGVEMDTRSPHDEDPQAVTYAEVKHSRPRREMA | |
| | SPPSPLSGEFLDTKDRQAEEDRQMDTEAAASEAPQDVTYAQLHSLTLRR | |
| | EATEPPPSQEGPSPAVPSIYATLAIH (SEQ ID NO: 186) | |
| CD3D- | MEHSTFLSGLVLATLLSQVSPFKIPIEELEDRVFVNCNTSITWVEGTVG | SEQ ID |
| LIR1_IT | TLLSDITRLDLGKRILDPRGIYRCNGTDIYKDKESTVQVHYRMCQSCVE | NO: 200 |
| IM3 4 | LDPATVAG11VTDVIATLLLALGVFCFAGHETGRLSGAADTQALLRNDV | |
| | TYAQLHSLTLRREATEPPPSQEGPSPAVPSIYATLGGNWARNK(SEQ | |
| | ID NO: 187) | |
| CD3E- | MQSGTHWRVLGLCLLSVGVWGQDGNEEMGGITQTPYKVSISGTTVILTC | SEQ ID |
| LIR1_IT | PQYPGSEILWQHNDKNIGGDEDDKNIGSDEDHLSLKEFSELEQSGYYVC | NO: 201 |
| IM3 4 | YPRGSKPEDANFYLYLRARVCENCMEMDVMSVATIVIVDICITGGLLLL | |
| | VYYWSKNRKAKAKPVTRGAGAGGRQRGQNKERPPPVPNVTYAQLHSLTL | |
| | RREATEPPPSQEGPSPAVPSIYATLNQRRI (SEQ ID NO: 188) | |
| CD3G- | MEQGKGLAVLILAIILLQGTLAQSIKGNHLVKVYDYQEDGSVLLTCDAE | SEQ ID |
| LIR1_IT | AKNITWFKDGKMIGFLTEDKKKWNLGSNAKDPRGMYQCKGSQNKSKPLQ | NO:202 |
| IM3 4 | VYYRMCQNCIELNAATISGFLFAEIVSIFVLAVGVYFIAGQDGVRQSRA | |
| (C2700) | SDKQTLLPNDVTYAQLHSLTLRREATEPPPSQEGPSPAVPSIYATLQGN | |
| | QLRRN (SEQ ID NO: 189) | |
| CD3Z- | MKWKALFTAAILQAQLPITEAQSFGLLDPKLCYLLDGILFIYGVILTAL | SEQ ID |
| LIR1_IT | FLRVKFSRSADAPAYQQGQNVTYAQLHSLTLRREATEPPPSQEGPSPAV | NO: 203 |
| IM3 4 | PSIYATLDKRRGRDPEMGGKPRRKNPQEVTYAQLHSLTLRREATEPPPS | |
| (C2701) | QEGPSPAVPSIYATLGMKGERRRGKGHDVTYAQLHSLTLRREATEPPPS | |
| | QEGPSPAVPSIYATLHMQALPPR (SEQ ID NO: 190) | |

Inhibitory intracellular domains are underlined in Table 6.

### Example 17: Transmembrane Domain Interactions to Generate Distinct Inhibitor and Activator TCR Complexes

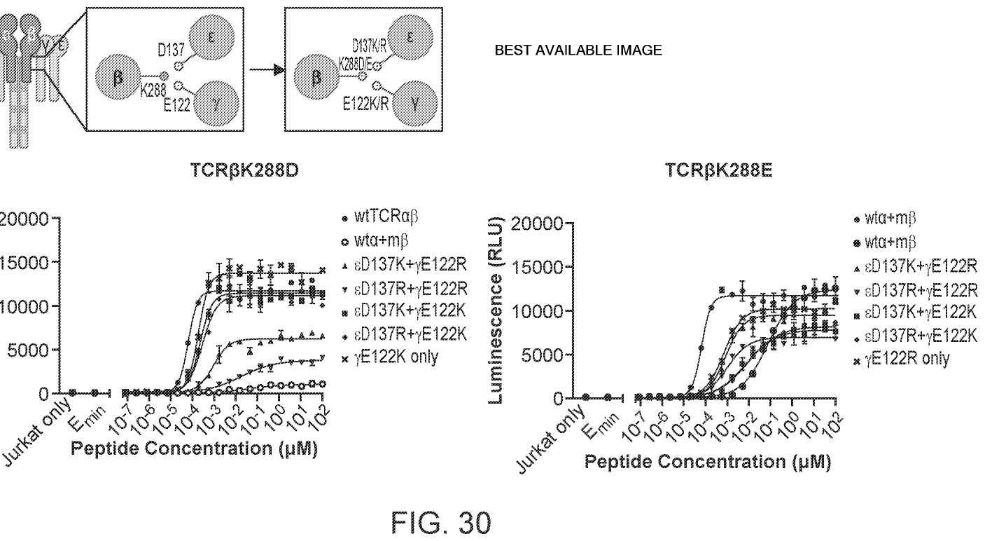

The transmembrane domains of TCRs can be engineered to generate distinct activator and inhibitor complexes in a cell with multiple variants of TCR subunits through transmembrane domain interactions. For all experiments, HLA-A*0201-NY-ESO-1 TCR 1G4 95LY was used as the activator. Transfected Jurkat cells were co-cultured with NY-ESO-1 peptide loaded T2 cells, as described above for Example 16. In FIG. 30, TCRb that contained either K288D or K288E was paired with corresponding charge-swapped CD3e and CD3g mutants.

In FIG. 30, the TCR beta transmembrane domain contained either a K288D or K288E mutant, and was paired with corresponding charge-swapped CD3 epsilon and CD3 gamma mutants (D137K or D137 for CD3 epsilon, E122K or E122R for CD3 gamma). The positively charged K288 residue of the TCR beta subunit in the transmembrane domain interacts with corresponding negatively charged residues of the CD3 epsilon (D137) and CD3 gamma (E122) transmembrane domains. These charge pairs were charge swapped to generate distinct TCRb-CD3e/g complexes. Jurkat NFAT-luciferase activity of a charge swapped TCRb-CD3δ/γ complex is shown in the lower panels of FIG. 30. In wild type Jurkat NFAT-luciferase reporter cells, either wild type TCRa/b or wild type TCRa and K288D TCRb were co-expressed with the indicated charge swapped CD3 epsilon and gamma subunits, and activity was assayed using the NFAT-luciferase reporter system.

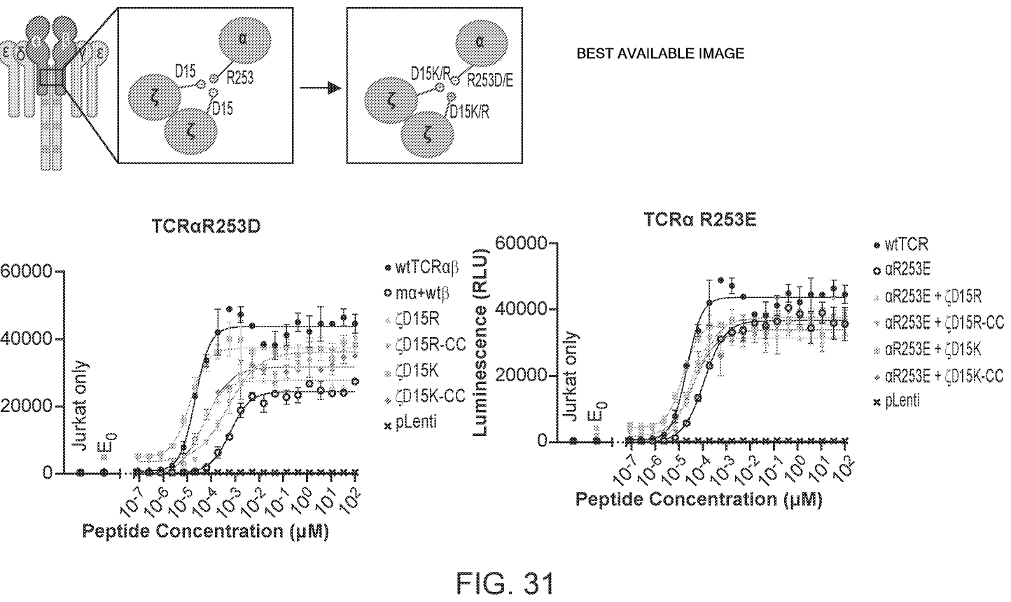

FIG. 31 shows that the positively charged R253 residue of TCRa interacts with corresponding negatively charged residue of CD3ζ (D15). In FIG. 31, TCR alpha that contained either R253D or R253E was paired with corresponding charge-swapped CD3 zeta. The charge pair was charge swapped to generate distinct TCRa-CD3ζ complexes. The lower panels of FIG. 31 show Jurkat NFAT-luciferase activity of the charge swapped TCRa-CD3ζ complexes. In wild type Jurkat cells, either wild type TCRa/b or R253D TCRa and wild type TCRb were co-expressed with or without the corresponding CD3ζ charge-swapped mutant, and the activity was assayed using the NFAT-Luciferase reporter system.

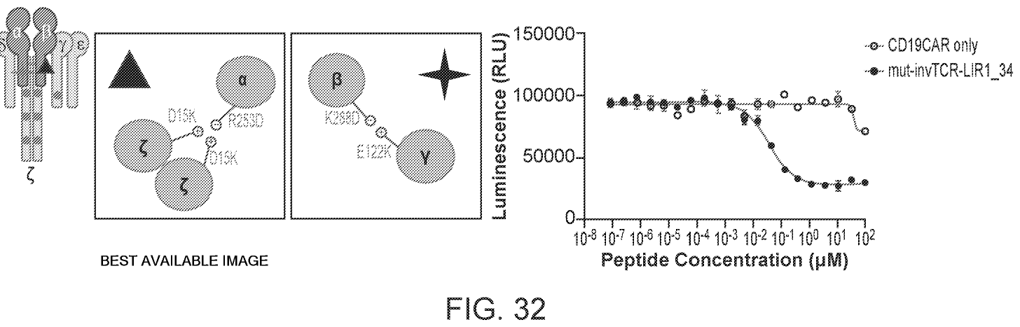

FIG. 32 shows the activity of a TCR complex including the charge-swapped TM-containing TCRa and TCRb pair, as well as the corresponding charge swapped mutant CD3z and CD3g subunits, can inhibit Jurkat cell activation by CD19 CAR. CD3z TM mutation (triangle) and CD3g TM mutation (black star) are described in the boxes at left. In FIG. 32, the well-performing mutants from FIGS. 30 and 31 were combined (TCRaR253D-CD3ZD15K and TCRbK288D-CD3GE122K) in the context of an inhibitory TCR and tested for their ability to inhibit Jurkat cell activation by a CD19 CAR using the assays described above.

TABLE 7
Constructs with transmembrane domain mutations

| | | Nucleotide |
|---|---|---|
| Name | Amino Acid Sequence | Sequence |
| CD3Z(D | MKWKALFTAAILQAQLPITEAQSFGLLDPKLCYLL [symbol] GILFIYGVILTALF | SEQ ID |
| 15K)- | LRVKFSRSADAPAYQQGQNVTYAQLHSLTLRREATEPPPSQEGPSPAVPS | NO: 218 |
| LIR_IT | IYATLDKRRGRDPEMGGKPRRKNPQEVTYAQLHSLTLRREATEPPPSQEG | |
| IM34 | PSPAVPSIYATLGMKGERRRGKGHDVTYAQLHSLTLRREATEPPPSQEGP | |
| | SPAVPSIYATLHMQALPPR (SEQ ID NO: 204) | |
| CD3G(E | MEQGKGLAVLILAIILLQGTLAQSIKGNHLVKVYDYQEDGSVLLTCDAEA | SEQ ID |
| 122K)- | KNITWFKDGKMIGFLTEDKKKWNLGSNAKDPRGMYQCKGSQNKSKPLQVY | NO: 219 |
| LIR_IT | YRMCQNCIELNAATISGFLFA [symbol] IVSIFVLAVGVYFIAGQDGVRQSRASDK | |
| IM34 | QTLLPNDVTYAQLHSLTLRREATEPPPSQEGPSPAVPSIYATLQGNQLRR | |
| | N (SEQ ID NO: 205) | |
| TCRa | METLLGLLILWLQLQWVSSKQEVTQIPAALSVPEGENLVLNCSFTDSAIY | SEQ ID |
| (R253D) | NLQWFRQDPGKGLTSLLLIQSSQREQTSGRLNASLDKSSGRSTLYIAASQ | NO: 220 |
| | PGDSATYLCAVRPLYGGSYIPTFGRGTSLIVHPYIQNPDPAVYQLRDSKS | |
| | SDKSVCLFTDFDSQTNVSQSKDSDVYITDKCVLDMRSMDFKSNSAVAWSN | |
| | KSDFACANAFNNSIIPEDTFFPSPESSCDVKLVEKSFETDTNLNFQNLSV | |
| | IGF [symbol] ILLLKVAGFNLLMTLRLWSS (SEQ ID NO: 206) | |
| TCRb | MSIGLLCCAALSLLWAGPVNAGVTQTPKFQVLKTGQSMTLQCAQDMNHEY | SEQ ID |
| (K288D) | MSWYRQDPGMGLRLIHYSVGAGITDQGEVPNGYNVSRSTTEDFPLRLLSA | NO: 221 |
| | APSQTSVYFCASSYVGNTGELFFGEGSRLTVLEDLKNVFPPEVAVFEPSE | |
| | AEISHTQKATLVCLATGFYPDHVELSWWVNGKEVHSGVCTDPQPLKEQPA | |
| | LNDSRYCLSSRLRVSATFWQNPRNHFRCQVQFYGLSENDEWTQDRAKPVT | |
| | QIVSAEAWGRADCGFTSESYQQGVLSATILYEILLG [symbol] ATLYAVLVSALVL | |
| | MAMVKRKDSRG (SEQ ID NO: 207) | |
| CD3Z | MKWKALFTAAILQAQLPITEAQSFGLLDPKLCYLL [symbol] GILFIYGVILTALF | SEQ ID |
| (D15R) | LRVKFSRSADAPAYQQGQNQLYNELNLGRREEYDVLDKRRGRDPEMGGKP | NO: 222 |
| | RRKNPQEGLYNELQKDKMAEAYSEIGMKGERRRGKGHDGLYQGLSTATKD | |
| | TYDALHMQALPPR (SEQ ID NO: 208) | |
| CD3Z | MKWKALFTAAILQAQLPITEAQSFGLLDPKLCYLL [symbol] GILFIYGVILTALF | SEQ ID |
| (D15K) | LRVKFSRSADAPAYQQGQNQLYNELNLGRREEYDVLDKRRGRDPEMGGKP | NO: 223 |
| | RRKNPQEGLYNELQKDKMAEAYSEIGMKGERRRGKGHDGLYQGLSTATKD | |
| | TYDALHMQALPPR (SEQ ID NO: 209) | |
| CD3Z(D | MKWKALFTAAILQAQLPITECQSFGLLDPKLGYLL [symbol] GILFIYGVILTALF | SEQ ID |
| 15R)- | LRVKFSRSADAPAYQQGQNQLYNELNLGRREEYDVLDKRRGRDPEMGGKP | NO: 224 |
| disulfide | RRKNPQEGLYNELQKDKMAEAYSEIGMKGERRRGKGHDGLYQGLSTATKD | |
| mutant | TYDALHMQALPPR (SEQ ID NO: 210) | |
| CD3Z(D | MKWKALFTAAILQAQLPITECQSFGLLDPKLGYLL [symbol] GILFIYGVILTALF | SEQ ID |
| 15K)- | LRVKFSRSADAPAYQQGQNQLYNELNLGRREEYDVLDKRRGRDPEMGGKP | NO: 225 |
| disulfide | RRKNPQEGLYNELQKDKMAEAYSEIGMKGERRRGKGHDGLYQGLSTATKD | |
| mutant | TYDALHMQALPPR (SEQ ID NO: 211) | |
| CD3D | MEHSTFLSGLVLATLLSQVSPFKIPIEELEDRVFVNCNTSITWVEGTVGT | SEQ ID |
| D137K | LLSDITRLDLGKRILDPRGIYRCNGTDIYKDKESTVQVHYRMCQSCVELD | NO: 226 |
| | PATVAGIIVT [symbol] VIATLLLALGVFCFAGHETGRLSGAADTQALLRNDQVYQ | |
| | PLRDRDDAQYSHLGGNWARNK (SEQ ID NO: 212) | |
| CD3D | MEHSTFLSGLVLATLLSQVSPFKIPIEELEDRVFVNCNTSITWVEGTVGT | SEQ ID |
| D137R | LLSDITRLDLGKRILDPRGIYRCNGTDIYKDKESTVQVHYRMCQSCVELD | NO: 227 |
| | PATVAGIIVT [symbol] VIATLLLALGVFCFAGHETGRLSGAADTQALLRNDQVYQ | |
| | PLRDRDDAQYSHLGGNWARNK (SEQ ID NO: 213) | |
| CD3E | MQSGTHWRVLGLCLLSVGVWGQDGNEEMGGITQTPYKVSISGTTVILTCP | SEQ ID |
| D137R | QYPGSEILWQHNDKNIGGDEDDKNIGSDEDHLSLKEFSELEQSGYYVCYP | NO: 228 |
| | RGSKPEDANFYLYLRARVCENCMEMDVMSVATIVIV [symbol] ICITGGLLLLVYY | |
| | WSKNRKAKAKPVTRGAGAGGRQRGQNKERPPPVPNPDYEPIRKGQRDLYS | |
| | GLNQRRI (SEQ ID NO: 214) | |
| CD3E | MQSGTHWRVLGLCLLSVGVWGQDGNEEMGGITQTPYKVSISGTTVILTCP | SEQ ID |
| D137K | QYPGSEILWQHNDKNIGGDEDDKNIGSDEDHLSLKEFSELEQSGYYVCYP | NO: 229 |
| | RGSKPEDANFYLYLRARVCENCMEMDVMSVATIVIV [symbol] ICITGGLLLLVYY | |
| | WSKNRKAKAKPVTRGAGAGGRQRGQNKERPPPVPNPDYEPIRKGQRDLYS | |
| | GLNQRRI (SEQ ID NO: 215) | |
| CD3G | MEQGKGLAVLILAIILLQGTLAQSIKGNHLVKVYDYQEDGSVLLTCDAEA | SEQ ID |
| E122R | KNITWFKDGKMIGFLTEDKKKWNLGSNAKDPRGMYQCKGSQNKSKPLQVY | NO: 230 |
| | YRMCQNCIELNAATISGFLFA [symbol] IVSIFVLAVGVYFIAGQDGVRQSRASDK | |
| | QTLLPNDQLYQPLKDREDDQYSHLQGNQLRRN (SEQ ID NO: 216) | |
| CD3G | MEQGKGLAVLILAIILLQGTLAQSIKGNHLVKVYDYQEDGSVLLTCDAEA | SEQ ID |
| E122K | KNITWFKDGKMIGFLTEDKKKWNLGSNAKDPRGMYQCKGSQNKSKPLQVY | NO: 231 |
| | YRMCQNCIELNAATISGFLFA [symbol] IVSIFVLAVGVYFIAGQDGVRQSRASDK | |
| | QTLLPNDQLYQPLKDREDDQYSHLQGNQLRRN | |
| | (SEQ ID NO: 217) | |

Select inhibitory domains are underlined in Table 7, and mutant amino acid residues in the transmembrane domain are underlined and in bold.

What is claimed is:

1. An engineered T-cell receptor, comprising a fusion protein, the fusion protein comprising an extracellular domain, a transmembrane domain and a first intracellular domain capable of providing an inhibitory signal.

2. The engineered T-cell receptor of claim 1, wherein the fusion protein comprises at least one TCR subunit in which an intracellular portion of the TCR subunit is fused to the first intracellular domain.

3. The engineered T-cell receptor of claim 1, wherein, when the engineered T-cell receptor is present in a T-cell, the intracellular domain inhibits endogenous TCR signaling of the T-cell.

4. The engineered T-cell receptor of any one of claims 1-3, comprising a second intracellular domain.

5. The engineered T-cell receptor of any one of claims 1-4, wherein the first intracellular domain comprises an immunoreceptor tyrosine-based inhibitory motif (ITIM).

6. The engineered T-cell receptor of claim 5, wherein the ITIM is synthetic or non-naturally occurring.

7. The engineered T-cell receptor of claim 5 or 6, wherein the first intracellular domain comprises a programmed cell death 1 (PD-1) intracellular domain, a cytotoxic T-lymphocyte associated protein 4 (CTLA-4) intracellular domain, a killer cell immunoglobulin like receptor, three Ig domains and long cytoplasmic tail 2 (KIR3DL2) intracellular domain, a killer cell immunoglobulin like receptor, three Ig domains and long cytoplasmic tail 3 (KIR3DL3) intracellular domain, a zeta chain of T cell receptor associated protein kinase 70 (ZAP70) domain comprising a Src Homology 2 (SH2) domain, a ZAP70 inactivated kinase domain, a leukocyte immunoglobulin like receptor B1 (LIR1) intracellular domain, an Fc gamma receptor IIB (FcgRIIB) intracellular domain, or a killer cell lectin like receptor K1 (NKG2D) intracellular domain.

8. The engineered T-cell receptor of claim 7, wherein the first intracellular domain comprises a LIR1 intracellular domain, a PD-1 intracellular domain or a KIR3DL2 intracellular domain.

9. The engineered T-cell receptor of claim 7 or 8, wherein the LIR intracellular domain comprises a sequence of SEQ ID NOs: 61 or 67-69.

10. The engineered T-cell receptor of claim 7 or 8, wherein the PD-1 intracellular domain comprises a sequence of SEQ ID NOs: 57, 66 or 74.

11. The engineered T-cell receptor of claim 7 or 8, wherein the KIR3DL2 intracellular domain comprises a sequence of SEQ ID NO: 52.

12. The engineered T-cell receptor of claim 7, wherein the ZAP70 domain comprising an SH2 domain comprises the N terminal ZAP70 SH2 domain, the C terminal ZAP70 SH2, or both the N and C terminal ZAP70 SH2 domains.

13. The engineered T-cell receptor of claim 7, wherein the ZAP70 inactivated kinase comprises a substitution of alanine for lysine at position 369 of SEQ ID NO: 17.

14. The engineered T-cell receptor of any one of claims 4-13, wherein the second intracellular domain is selected from a CD3D molecule (CD3 delta) intracellular domain (ICD), a CD3E molecule (CD3 epsilon) ICD, a CD3G molecule (CD3 gamma), and a CD247 molecule (CD3Z or CD3 zeta) intracellular domain, or fragments or functional derivatives thereof.

15. The engineered T-cell receptor of claim 14, wherein the second intracellular domain comprises a deletion of at least one ITAM.

16. The engineered T-cell receptor of claim 15, wherein the fusion protein comprises a CD3 delta extracellular domain, transmembrane domain and second intracellular domain.

17. The engineered T-cell receptor of claim 16, comprising a sequence of SEQ ID NO: 171.

18. The engineered T-cell receptor of claim 15, wherein the fusion protein comprises a CD3 epsilon extracellular domain, transmembrane domain and second intracellular domain.

19. The engineered T-cell receptor of claim 18, comprising a sequence of SEQ ID NO: 172.

20. The engineered T-cell receptor of claim 15, wherein the fusion protein comprises a CD3 gamma extracellular domain, transmembrane domain and second intracellular domain.

21. The engineered T-cell receptor of claim 20, comprising a sequence of SEQ ID NO: 173.

22. The engineered T-cell receptor of claim 15, wherein the fusion protein comprises a CD3 zeta extracellular domain, transmembrane domain and second intracellular domain.

23. The engineered T-cell receptor of claim 22, comprising a sequence of SEQ ID NO: 174.

24. The engineered T-cell receptor of any one of claims 4-14, wherein the second intracellular domain is selected from a T-cell receptor (TCR) alpha intracellular domain (ICD), TCR beta ICD, or functional derivatives thereof.

25. The engineered T-cell receptor of any one of claims 1-24, wherein the transmembrane domain is selected from a TCR alpha transmembrane domain (TM), a TCR beta TM, a CD3 delta TM, a CD3 epsilon TM, a CD3 gamma TM, a CD3 zeta TM or functional derivatives thereof.

26. The engineered T-cell receptor of claim 25, wherein the transmembrane domain is a TCR alpha transmembrane domain comprising a sequence of SEQ ID NO: 9.

27. The engineered T-cell receptor of claim 25, wherein the transmembrane domain is a TCR beta transmembrane domain comprising a sequence of SEQ ID NO: 10.

28. The engineered T-cell receptor of claim 25, wherein the transmembrane domain is a CD3 delta transmembrane domain comprising a sequence of SEQ ID NO: 106.

29. The engineered T-cell receptor of claim 25, wherein the transmembrane domain is a CD3 epsilon transmembrane domain comprising a sequence of SEQ ID NO: 7.

30. The engineered T-cell receptor of claim 25, wherein the transmembrane domain comprises a CD3 gamma transmembrane domain comprising a sequence of SEQ ID NO:
8.

31. The engineered T-cell receptor of claim 25, wherein the transmembrane domain is a CD3 zeta transmembrane comprising a sequence of SEQ ID NO: 76.

32. The engineered T-cell receptor of any one of claims 4-14, wherein the first intracellular domain, optionally the second intracellular domain, and the transmembrane domain, comprise domains that are isolated or derived from PD-1, CTLA-4, KIR3DL2, KIR3DL3, ZAP70, LIR1, FcgRIIB or NKG2D.

33. The engineered T-cell receptor of any one of claims 1-32, wherein the transmembrane domain and the first intracellular domain are connected by a polypeptide linker.

34. The engineered T-cell receptor of any one of claims 4-33, wherein the first intracellular domain and the second intracellular domain are connected by polypeptide linker.

35. The engineered T-cell receptor of claim 33 or 34, wherein the polypeptide linker comprises at most 3, 4, 5, 6, 8, 9, 10, 12, 15, 16, 18 or 20 amino acids.

36. The engineered T-cell receptor of claim 33, wherein the polypeptide linker comprises a sequence of GS, GGS, SEQ ID NO: 240, SEQ ID NO: 18, SEQ ID NO: 19, SEQ ID NO:
20, or SEQ ID NO: 21.

37. The engineered T-cell receptor of any one of claims 1-36, wherein the domains are arranged in an order of: extracellular domain, transmembrane domain, first intracellular domain, from N to C terminus.

38. The engineered T-cell receptor of any one of claims 4-36, wherein the domains are arranged in an order from N to C terminus of: extracellular domain, transmembrane domain, first intracellular domain.

39. The engineered T-cell receptor of any one of claims 4-36, wherein the domains, are arranged in an order from N to C terminus of: extracellular domain, transmembrane domain, second intracellular domain, first intracellular domain.

40. The engineered T-cell receptor of any one of claims 4-36, wherein the domains, are arranged in an order from N to C terminus of: extracellular domain, transmembrane domain, first intracellular domain, second intracellular domain.

41. The engineered T-cell receptor any one of claims 1-40, wherein the engineered T-cell receptor comprises TCR alpha, TCR beta, CD3 delta, CD3 epsilon, CD3 gamma and CD3 zeta subunits, wherein one of the subunits is the fusion protein.

42. The engineered T-cell receptor of any one of claims 1-41, comprising an extracellular antigen binding domain fused to an extracellular domain of a subunit of the TCR.

43. The engineered T-cell receptor of claim 42, wherein the extracellular antigen binding domain comprises an antibody fragment, a single chain variable fragment (ScFv), or a single domain antibody (sdAb).

44. The engineered T-cell receptor of claim 42, wherein the extracellular antigen binding domain comprises an extracellular antigen binding domain isolated or derived from a T cell receptor.

45. The engineered T-cell receptor of claim 44, wherein the extracellular antigen binding domain comprises a TCR alpha extracellular domain and a TCR beta extracellular domain.

46. The engineered T-cell receptor of claim 41-45, wherein the subunit of the TCR comprises TCR alpha, TCR beta, CD3 delta, CD3 epsilon, CD3 gamma or CD3 zeta.

47. The engineered T-cell receptor of any one of claims 41-46, wherein the engineered T-cell receptor comprises a first fusion protein comprising a TCR alpha transmembrane domain and a LIR1 intracellular domain, and a second fusion protein comprising a TCR beta transmembrane domain and a LIR1 intracellular domain.

48. The engineered T-cell receptor of any one of claims 41-46, wherein the engineered T-cell receptor comprises a first fusion protein comprising a CD3 delta transmembrane domain and a LIR1 intracellular domain, a second fusion protein comprising a CD3 epsilon transmembrane domain and a LIR1 inhibitory domain, and a third fusion protein comprising a CD3 gamma transmembrane domain and a LIR1 inhibitory domain.

49. The engineered T-cell receptor of claim 48, wherein the first fusion protein comprises SEQ ID NO: 183 or 187, the second fusion protein comprises SEQ ID NO: 184 or 188, and the third fusion protein comprises 185 or 189.

50. The engineered T-cell receptor of claim 48 or 49, further comprising a fourth fusion protein comprising a CD3 zeta transmembrane domain and a LIR1 inhibitory domain.

51. The engineered T-cell receptor of claim 50, wherein the fourth fusion protein comprises a sequence of 186 or 190.

52. A polynucleotide comprising a coding sequence encoding the engineered T-cell receptor and/or the fusion protein of the engineered T-cell receptor of any one of claims 1-51.

53. A vector comprising a coding sequence encoding the fusion protein of the engineered T-cell receptor of any one of claims 1-40, wherein the coding sequence is operatively linked to a promoter.

54. A pharmaceutical composition comprising the polynucleotide of claim 52 or the vector of claim 53.

55. An immune cell, comprising the polynucleotide sequence of claim 52, wherein the coding sequence operatively linked to a promoter.

56. An immune cell, comprising the engineered T-cell receptor of any one of claims 1-51.

57. The immune cell of claim 56, wherein the immune cell is a T cell.

58. The immune cell of any one of claims 55-57, wherein an activity of the immune cell is inhibited when the immune cell is contacted with a target cell expressing an antigen recognized by the engineered T-cell receptor.

59. The immune cell of claim 58, wherein the activity that is inhibited comprises proliferation, cytokine production, cytotoxicity or a combination thereof.

60. The immune cell of any one of claims 55-59, wherein the immune cell comprises a deletion in one or more subunits of an endogenous T cell receptor.

61. The immune cell of claim 60, wherein the immune cell comprises deletions in CD3 delta, CD3 epsilon and CD3 gamma.

62. The immune cell of claim 60, wherein the immune cell comprises deletions in CD3 delta, CD3 epsilon, CD3 gamma and CD3 zeta.

63. The immune cell of any one of claims 60-62, wherein the immune cell does not express a functional endogenous TCR.

64. The immune cell of any one of claims 56-63, further comprising an activator receptor.

65. The immune cell of claim 64, wherein the activator receptor is a TCR or a chimeric antigen receptor (CAR).

66. A pharmaceutical composition comprising the immune cell of any one of claims 55-65.

67. A kit comprising the polynucleotide of claim 52 or the vector of claim 53.

68. A kit comprising the immune cell of any one of claims 55-65.

69. A method of generating immune cells comprising an engineered T-cell receptor, comprising:
a. providing a plurality of immune cells;
b. transforming the plurality of immune cells with the vector of claim 51 to generate a plurality of transformed immune cells; and
c. culturing the plurality of transformed immune cells under conditions sufficient to express the engineered TCR from vector;
thereby generating immune cells comprising an engineered TCR.

70. The method of claim 69, wherein the immune cells are T cells.

71. The method of claim 69 or 70, wherein the T cell are autologous or allogeneic.

72. The method of any one of claims 69-71, comprising (d) activating the plurality of immune cells.

73. A method of inhibiting the activity of a T cell by expressing the fusion protein of any one of claims 1-40 in the T cell.

74. A method of inhibiting the activity of a T cell by expressing the engineered TCR of any one of claims 1-51 in the T cell.

75. The method of claim 73 or 74, wherein the activity of the T cell comprises TCR-mediated signaling in response to a cognate antigen.

76. The method of claim 75, wherein the TCR-mediated signaling comprises activation of one or more genes operatively linked to an NFAT promoter.

77. The engineered T-cell receptor of any one of claims 1-51, the polynucleotide of claim 51, the vector of claim 52, the pharmaceutical composition of claim 54 or claim 66, the immune cell of any one of claims 55-65, or the kit of claim 67 or claim 68 for use in a method according any one of claims 69-76.
